(19) United States
(12) Patent Application Publication
(10) Pub. No.: US 2024/0317711 A1
(43) Pub. Date: Sep. 26, 2024

(54) AURKA SELECTIVE DEGRADATION INDUCING COMPOUND

(71) Applicant: UPPTHERA INC., Incheon (KR)

(72) Inventors: Soo Hee RYU, Incheon (KR); Im Suk MIN, Gyeonggi-do (KR); Chan Ho KIM, Incheon (KR); Jung Chul PARK, Seoul (KR); Seong Hoon KIM, Incheon (KR); Jun Kyu LEE, Gyeonggi-do (KR); Ha Na JEONG, Gyeonggi-do (KR); Seung Hyun JO, Incheon (KR)

(21) Appl. No.: 18/569,677

(22) PCT Filed: Jun. 24, 2022
(86) PCT No.: PCT/KR2022/009074

§ 371 Date: Dec. 13, 2023

Related U.S. Application Data
(60) Provisional application No. 63/214,600, filed on Jun. 24, 2021.

(51) Int. Cl. C07D 401/14; A61K 31/506; A61K 47/55; C07D 417/14

(52) U.S. Cl. CPC C07D 401/14; A61K 31/506; A61K 47/55; C07D 417/14

(57) ABSTRACT

The present invention relates to a novel compound that induces selective degradation of AURKA, and specifically provides a bifunctional compound in which an AURKA binding moiety and an E3 ubiquitin ligase binding moiety are linked by a chemical linker, a method for preparing same, and a use thereof. In addition, the compound according to the present invention can be effectively used for the prevention or treatment of AURKA-related diseases.

### TECHNICAL FIELD

The present invention relates to an AURKA selective degradation inducing compound and a pharmaceutical composition for preventing or treating cancer including the same as an active ingredient.

### BACKGROUND ART

Aurora kinase is a kinase belonging to Serine/Threonine Kinase, which regulates cell division. Three human structural isoforms Aurora A, Aurora B, and Aurora C of Aurora kinase have functions distinguished in mitosis of cells and different intracellular distributions and show different functions. Among them, Aurora kinase A (AURKA) is located in the centrosome of interphase cells, involved in the centrosome maturity and the formation of bipolar spindles, and its activity is known to be the highest in the G2/M phase. In addition, AURKA is a chromosomal passenger protein kinase and controls the phosphorylation of histone H3 in the tenth serine.

The overexpression of AURKA causes hyperphosphorylation of normal cell division cycle targets and mutant phosphorylation of cytoplasmic targets to cause chromosomal instability, oncogenic transformation, tumor progression, and the development of anticancer agent resistance. Accordingly, the overexpression of AURKA is often observed in various cancer cells, such as colon, pancreas, breast, lung, thyroid cancer and leukemia. Recently, in many notable reports, it have been demonstrated that Aurora kinase was an attractive anticancer agent target. The inhibition of AURKA stops mitosis by forming unipolar spindles during mitosis through abnormal formation of the spindles and formation of immature centrosomes.

Based on this evidence, various compounds are being tested in clinical trials as AURKA inhibitors targeting cancer treatment, and known AURKA inhibitors include VE465, Tozasertib (VX-680), MK-0457, MK-5108, Alisertib (MLN-8237), LY3295668, etc. However, despite these recent studies, there are no AURKA-inhibiting drugs yet approved by the FDA.

Meanwhile, recently, proteolysis targeting chimera (PROTAC) has been proposed as small molecule compound-based platform technology capable of inducing in vivo proteolysis of target proteins. The PROTAC is a bifunctional compound in which a ligand molecule binding to a disease-related target protein and an E3 ubiquitin ligase binding moiety are linked by a chemical linker. In theory, a PROTAC compound may induce degradation of target proteins by locating disease-related target proteins near E3 ubiquitin ligase. Specifically, referring to FIG. 4, the PROTAC may selectively bind to a target protein by a ligand molecule that binds to the target protein (e.g., AURKA), and induce ubiquitination and subsequent degradation of the target protein through the proteasome by collecting the E3 ubiquitin ligase as the target protein by the E3 ubiquitin ligase binding moiety.

In the case of PROTAC compounds with AURKA as the target protein, in Non-patent Document 1 [Adhikari, Bikash, et al. Nature chemical biology 16.11 (2020): 1179-1188] and Non-patent Document 2 [Wang, Richard, et al. Communications biology 4.1 (2021): 1-15.], there a disclosed a bifunctional compound for linking Alisertib (MLN-8237) known as an AURKA inhibitor and a binding moiety for E3 ubiquitin ligase by a chemical linker. However, the PROTAC compound disclosed in Non-Patent Document 1 was confirmed to have AURKA degradation of 60% or less, and the PROTAC compound disclosed in Non-Patent Document 2 was confirmed to have AURKA degradation of 50% or less, so that PROTAC compounds with improved AURKA degradation and selectivity are still required.

Therefore, the present inventors completed the present invention by focusing on the idea that when a compound capable of effectively inducing the selective degradation of AURKA could be developed, the compound could be applied to the use for the treatment of cancer targeting AURKA.

### DISCLOSURE

### Technical Problem

The present invention is directed in consideration of the above problems, and an object of the present invention is to provide a compound for inducing selective degradation of AURKA.

Another object of the present invention is to provide a method for preparing a compound for inducing selective degradation of AURKA.

Yet another object of the present invention is to provide a use of a compound for inducing selective degradation of AURKA.

The objects to be solved by the present invention are not limited to the aforementioned object(s), and other object(s), which are not mentioned above, will be apparent to those skilled in the art from the following description.

### Technical Solution

### Aurka Selective Degradation Inducing Compound

The present invention provides a novel compound that induces degradation or selective degradation of Aurora kinase A (AURKA). Specifically, the present invention provides a bifunctional compound in which an AURKA binding moiety and an E3 ubiquitin ligase binding moiety are linked by a chemical linker.

According to one embodiment of the present invention, there is provided a compound represented by Chemical Formula I below, a stereoisomer thereof, or a pharmaceutically acceptable salt thereof:

ULM-Linker-PTM [Chemical Formula I]

In Chemical Formula I,

ULM is an E3 ubiquitin ligase binding moiety represented by Chemical Formula A or B below,

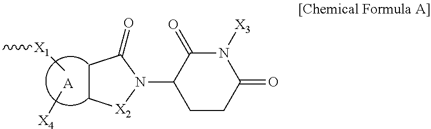

{in Chemical Formula A,

[symbol] is a ring selected from the group consisting of

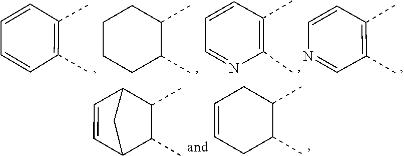

X₁ is a single bond, —CH₂—, —NH—, —O—, —CH₂CH₂—, —CC—, —CO—, —COO—, —NHCO— or —CONH—;

X₂ is —CH₂—, —CH(C₁₋₄alkyl)-, —NH—, —N(C₁₋₄alkyl)-, —O—, —CO—, —CH₂—CH₂—, —NH—CH₂—, —NH—CH(C₁₋₄alkyl)-, —N═CH—, —N═C(C₁₋₄ alkyl)- or —N═N—,

X₃ is hydrogen; and

X₄ is hydrogen, halogen, C₁₋₆alkyl, CN, NH₂, NO₂, OH, COH, COOH or CF₃.}

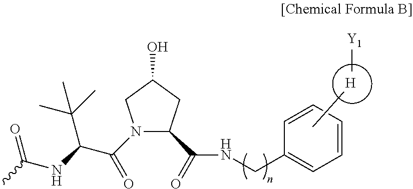

{in Chemical Formula B,

n is an integer of 1 to 3,

[symbol] is a 5- to 6-membered cycloalkyl, phenyl, 5- to 6-membered heterocycloalkyl, or 5- to 6-membered heteroaryl ring, and

Y₁ is hydrogen or C₁₋₃alkyl

PTM is an AURKA binding moiety represented by Chemical Formula II below,

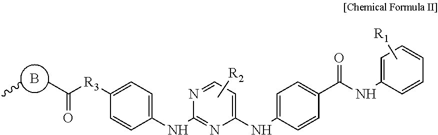

{in Chemical Formula II,

R₁ and R₂ are each independently hydrogen, —NO₂, —CN, —OH, C₁₋₆alkyl, C₂₋₆alkenyl, C₂₋₆alkynyl, C₁₋₆alkoxy, C₁₋₆thioalkoxy, C₃₋₆cycloalkyl, C₃₋₆heterocycloalkyl, C₃₋₆heteroaryl, phenyl or halogen,

R₃ is C₁₋₆alkylene, —O—, —S—, —NH—, or a direct bond, and

[symbol] is a 4- to 10-membered cycloalkyl, phenyl, 4- to 10-membered heterocycloalkyl, 5- to 6-membered heteroaryl ring or direct bond.

Linker is a group for chemically linking ULM and PTM.

In Chemical Formulas A and B, [symbol] represents a covalent bond for linking the ULM to the linker.

In Chemical Formula II, [symbol] represents a covalent bond for linking the PTM to the linker.

### (1) E3 Ubiquitin Ligase Binding Moiety

According to one embodiment of the present invention, the ULM is a CRBN E3 ubiquitin ligase binding moiety represented by Chemical Formula A. In the present invention, CRBN refers to Cereblon E3 ubiquitin ligase. The CRBN constitute an E3 ubiquitin ligase complex with DDB 1, Cul4A and ROC 1, in which the CRBN is a substrate recognition subunit of the complex. Some compounds capable of binding to the CRBN E3 ubiquitin ligase are known in the art. For example, since it was known that thalidomide binds to the CRBN E3 ubiquitin ligase, it has been reported that a plurality of immunomodulatory imide drugs (IMiDs), including lenalidomide and pomalidomide, have CRBN binding ability.

According to one embodiment of the present invention, the ULM may be an E3 ubiquitin ligase ligand represented by Chemical Formula A-1 below.

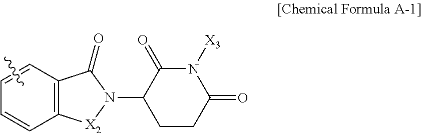

In Chemical Formula A-1,

X₂ is —CH₂—, —CH(C₁₋₄alkyl)-, —CO—, or —N═N—, and

X₃ is hydrogen.

According to one embodiment of the present invention, Chemical Formula A-1 may be selected from the group consisting of the following moieties:

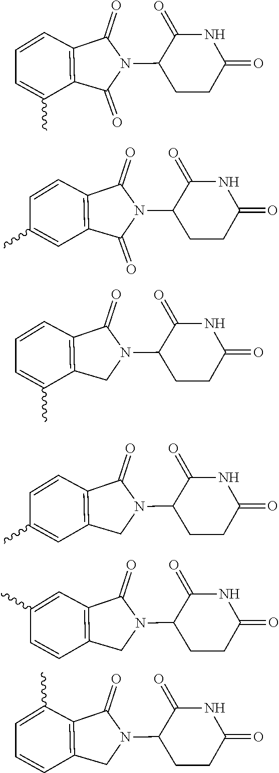

Examples of the CRBN E3 ubiquitin ligase ligand according to the present invention are as follows (literatures [Chamberlain and Brian. 2019] and [Akuffo et al. 2018]):

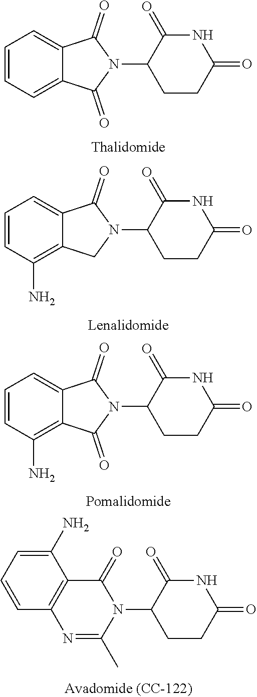 [SMILES: O=C1CCC(N2C(=O)c3ccccc3C2=O)C(=O)N1 ; Nc1cccc2c1CN(C1CCC(=O)NC1=O)C2=O ; Nc1cccc2c1C(=O)N(C1CCC(=O)NC1=O)C2=O ; Cc1nc2cccc(N)c2c(=O)n1C1CCC(=O)NC1=O]

Another example of the CRBN E3 ubiquitin ligase ligand according to the present invention is as follows (literature [Burslem et al. 2018]):

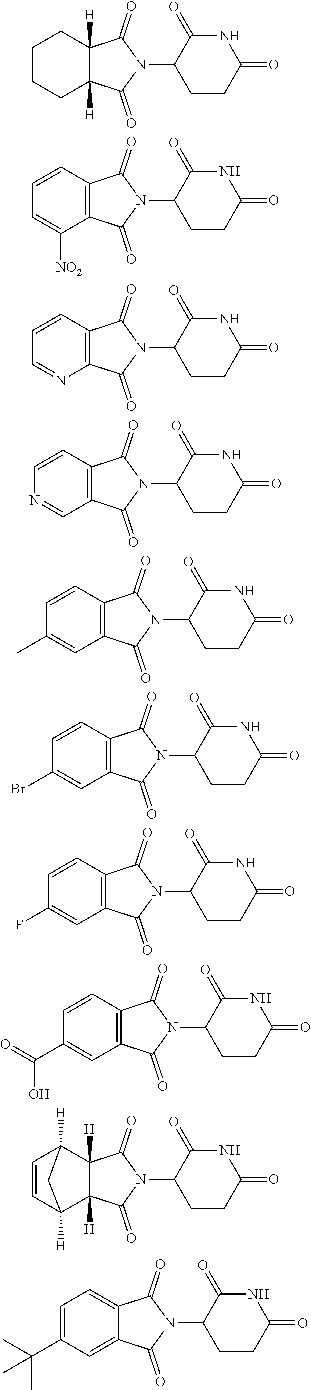

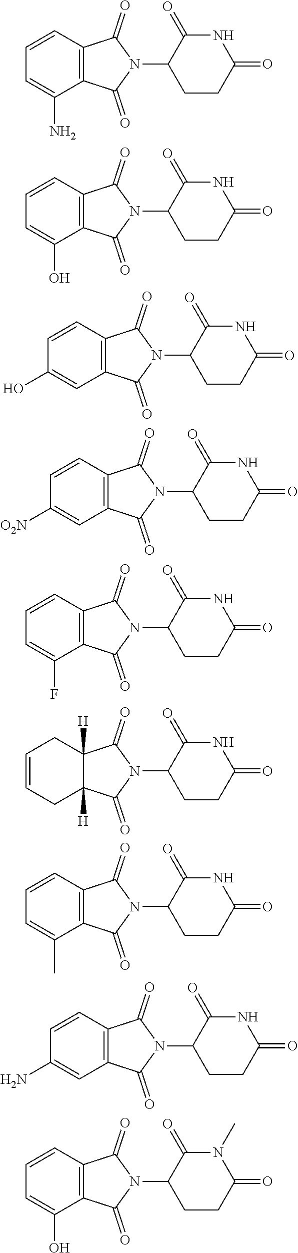

In a specific embodiment, the CRBN E3 ubiquitin ligase binding moiety of the present invention is as follows:

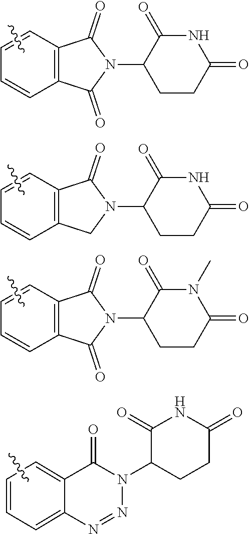

According to one embodiment of the present invention, in Chemical Formula A-1, X₂ may be —CO— and X₃ may be H.

According to one embodiment of the present invention, the ULM is a VHL E3 ubiquitin ligase binding moiety represented by Chemical Formula B. In the present invention, VHL refers to a von Hippel-Lindau tumor suppressor. The VHL constitute a VCB E3 ubiquitin ligase complex with Elongin B, Elongin C, CUL2 and Rbx1, in which the VHL is a substrate recognition subunit of the complex. Some compounds capable of binding to the VHL E3 ubiquitin ligase are known in the art. For example, after peptides such as Ala-Leu-Ala-(Hy)Pro-Tyr-Ile-Pro heptapeptide and Leu-Ala-(Hy)Pro-Tyr-Ile pentapeptide were known, improved small-molecular VHL E3 Ubiquitin ligase binding compounds have been reported.

According to one embodiment of the present invention, in Chemical Formula B, n may be an integer of 1 to 3, an integer of 1 to 2, or 1.

According to one embodiment of the present invention, in Chemical Formula B, [symbol] may be a 5- to 6-membered heterocycloalkyl or 5- to 6-membered heteroaryl ring containing at least one heteroatom selected from the group consisting of N, O and S. Specifically, the [symbol] may be a 5-membered heteroaryl ring selected from the group consisting of oxazole, isoxazole, thiazole, isothiazole, imidazole, pyrazole, triazole, oxadiazole, pyrrole, pyrrolidine, furan, dihydrofuran and tetrahydrofuran, and more specifically, the [symbol] may be a 5-membered heteroaryl ring containing N and S.

According to one embodiment of the present invention, in Chemical Formula B, Y₁ may be C₁₋₃ alkyl. Specifically, Y₁ may be C₁ alkyl.

According to an embodiment of the present invention, the Chemical Formula B may be selected from the group consisting of the following moieties:

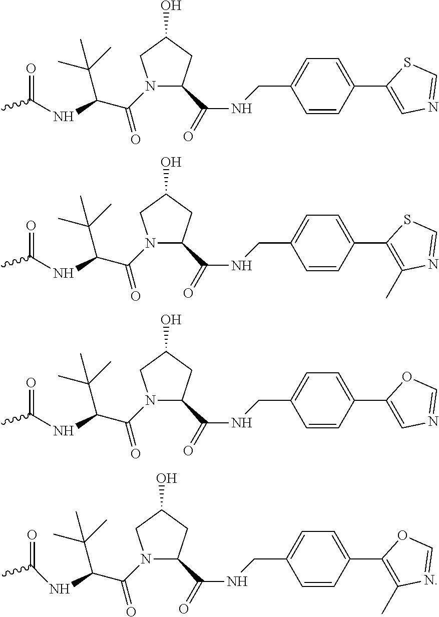

### (2) Target Protein Ligand (PTM)

In the compound represented by Chemical Formula I according to the present invention, PTM, a moiety that performs a target protein ligand function, is an AURKA binding moiety represented by Chemical Formula II described above.

Chemical Formula II, which constitutes some moieties of the compounds of Chemical Formula I according to the present invention, may bind alone to an active site of AURKA.

According to one embodiment of the present invention, in Chemical Formula II, R₁ and R₂ may each independently be a halogen selected from the group consisting of F, Cl, Br, and I. Specifically, R₁ may be Cl, and R₂ may be F.

According to one embodiment of the present invention, in Chemical Formula II, R₃ may be C₁ alkylene or direct bond. The direct bond may mean a case where R₃ is null.

According to one embodiment of the present invention, in Chemical Formula II, [symbol] may be 4- to 10-membered heterocycloalkyl containing one or more heteroatoms selected from the group consisting of N, O, and S. Specifically, [symbol] may be a 4-membered to 9-membered, 4-membered, 5-membered, 6-membered or 9-membered heterocycloalkyl containing at least one N.

The cycloalkyl or heterocycloalkyl refers to a non-aromatic monocyclic or multicyclic (ring system) hydrocarbon ring, and as a multicyclic ring, may include a double ring group such as a bridgehead, a fused ring, and a spiro ring.

According to one embodiment of the present invention, in Chemical Formula II, [symbol] may be a direct bond. The direct bond may mean a case where [symbol] is null.

According to an embodiment of the present invention, Chemical Formula II may be selected from the group consisting of the following moieties:

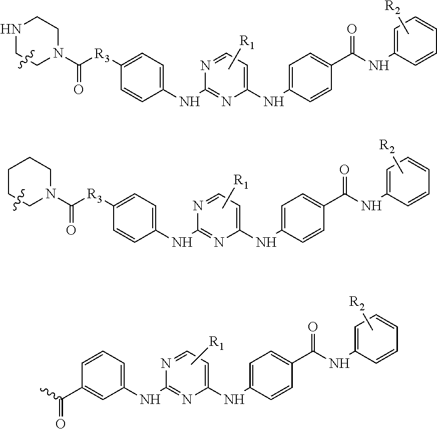

In the group consisting of the moieties, R₁, R₂ and R₃ are the same as defined in Chemical Formula II.

Specifically, Chemical Formula II may be selected from the group consisting of the following moieties:

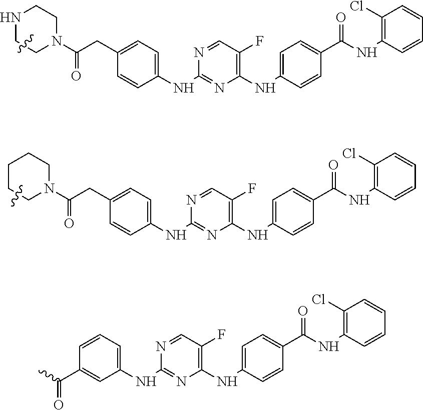

### (3) Linker

Linker is a group for chemically linking ULM and PTM, and is represented by Chemical Formula L below:

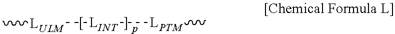

in Chemical Formula L, [symbol] and [symbol] are bonds,

L_{ULM} binds to a ULM moiety through [symbol] linked thereto,

L_{PTM} binds to a PTM moiety through [symbol] linked thereto,

L_{ULM} and L_{PTM} are each independently a single bond, —CH₂—, —NH—, —O—, —CO—, —OCO—, —CONH—, —NHCO—, —O(CH)ₙCONH— {wherein, n is an integer of 1 to 5}, or a direct bond,

L_{INT} is selected from the group consisting of C₁₋₁₀ alkylene, —CH₂—, —NH—, —O(CH)ₙCONH— {where n is an integer of 1 to 5}, —(CH₂)ₗO(CH₂)ₘO(CH₂)_{q}— {wherein, l, m and q are independently integers of 1 to 5}, —CHCH—, —CC—, —CH₂CH₂O—, —OCH₂CH₂—, —CH₂CH₂S—, —SCH₂CH₂—, —COO—, —CONH—, —NHCO—, [symbol] and direct bond {wherein [symbol] is a ring selected from the group consisting of aryl, heteroaryl, 3 to 10 membered cycloalkyl, 4 to 10 membered heterocycloalkyl, 4 to 10 membered cycloalkenyl, and 4 to 10 membered heterocycloalkenyl},

L_{ULM}, L_{PTM} and L_{INT} may each independently be substituted with one or more of C₁₋₆alkyl, C₃₋₈cycloalkyl, C₃₋₈heterocycloalkyl, halogen, hydroxy, amine, nitro, cyano or C₁₋₈haloalkyl, and

p is an integer of 1 to 20, an integer of 1 to 10, an integer of 1 to 6, or an integer of 1 to 3.

The cycloalkyl, heterocycloalkyl or heterocycloalkenyl refers to a non-aromatic monocyclic or multicyclic (ring system) hydrocarbon ring, and as a multicyclic ring, may include a double ring group such as a bridgehead, a fused ring, and a spiro ring.

In the L_{ULM}, L_{PTM}, or L_{INT}, the direct bond may mean the case where L_{ULM}, L_{PTM}, or L_{INT} is null.

According to one embodiment of the present invention, the [symbol] is

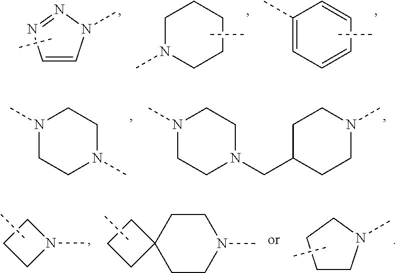

According to one embodiment of the present invention, the Linker is a Linker included in compounds 1 to 38 shown in Table 1 below.

According to one embodiment of the present invention, the compound represented by Chemical Formula I is one or more compounds selected from the group consisting of compounds 1 to 38 shown in Table 1 below, stereoisomers thereof, or pharmaceutically acceptable salts thereof.

In the present invention, the pharmaceutically acceptable salt is a concentration that is a concentration having relatively non-toxic and harmless effective effects to patients, and is any organic acid or inorganic acid addition salt in which side effects caused by the salt do not reduce the beneficial efficacy of the compound represented by Chemical Formula I. For example, in the pharmaceutically acceptable salt, the inorganic acid may be hydrochloric acid, phosphoric acid, sulfuric acid, nitric acid, etc., and the organic acid may be methanesulfonic acid, p-toluenesulfonic acid, formic acid, acetic acid, trifluoroacetic acid, maleic acid, succinic acid, oxalic acid, benzoic acid, tartaric acid, fumaric acid, manderic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid, hydroiodic acid, etc., but are not limited thereto.

### Method for Preparing AURKA Selective Degradation Inducing Compound

In the present invention, the compound represented by Chemical Formula I described above, the stereoisomer thereof, or the pharmaceutically acceptable salt thereof may be prepared by reactions such as the following Reaction Schemes 1 to 3 by synthetic methods known in the technical field of organic chemistry or modification and derivatization methods obvious to those skilled in the art.

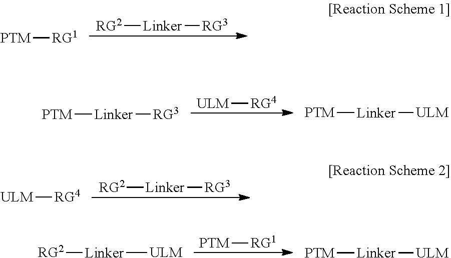

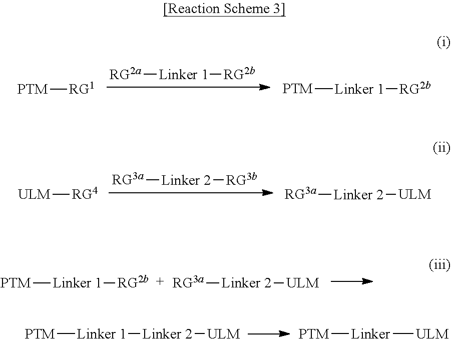

In Reaction Schemes 1 to 3, the PTM, Linker and ULM are groups defined above or suitable derivatives thereof, and RG¹, RG², RG^{2a}, RG^{2b}, RG³, RG^{3a}, RG^{3b} and RG⁴ are moieties containing suitable reactive groups that may link PROTAC compound intermediates represented by Chemical Formula I together through covalent bond formation in the field of organic synthesis. The covalent bonds may be formed through synthetic reactions such as amide formation, ester formation, carbamate formation, urea formation, ether formation, amine formation, various carbon-to-carbon single bond formation, double bond formation, and click chemistry, depending on a specific reaction group, but are not limited thereto.

In Reaction Schemes, modification of each step may include one or multiple synthesis steps. The isolation and purification of the product may be achieved by standard procedures known to those skilled in the art of organic chemistry.

In the present invention, the preparation methods of Examples 1, 2, 4 to 8, and 10 to 38 are presented as examples of Reaction Scheme 2, and Examples 3 and 9 are presented as examples of Reaction Scheme 3.

In Reaction Schemes, the reactant presented as PTM and the reactant presented as ULM may be easily synthesized by those skilled in the art by referring to literatures known in the field of organic chemistry and descriptions of Examples of the present invention.

The present invention includes compounds in the form of PTM-Linker-RG³ or PTM-Linker 1-RG^{2b}, which correspond to the reaction intermediates of Chemical Formula I.

### Use of AURKA Selective Degradation Inducing Compound

According to one embodiment of the present invention, there is provided a composition for inducing degradation of AURKA including the compound represented by Chemical Formula I, a stereoisomer thereof, or a pharmaceutically acceptable salt thereof. Chemical Formula I is as defined above.

In the terms of a mechanism of action, since a PROTAC compound for inducing degradation of AURKA according to the present invention may fundamentally degrade a target protein, AURKA, the PROTAC compound achieves an excellent AURKA inhibition effect compared to conventional AURKA small molecular inhibitors that simply inhibit the activity of AURKA.

Therefore, a composition containing the compound represented by Chemical Formula I according to the present invention, the stereoisomer thereof, or the pharmaceutically acceptable salt thereof may be usefully used for selective degradation of AURKA.

According to one embodiment of the present invention, there is provided a pharmaceutical composition for preventing or treating AURKA-related diseases including the compound represented by Chemical Formula I, a stereoisomer thereof, or a pharmaceutically acceptable salt thereof as an active ingredient. Chemical Formula I is as defined above.

In the present invention, the AURKA-related disease refers to any disease or condition that may be treated, alleviated, delayed, inhibited, or prevented by inducing the degradation or inhibiting the activity of AURKA. Specifically, the AURKA-related disease may be cancer (malignant tumor) or benign tumor.

The cancer includes all cancers that may exhibit preventive or therapeutic efficacy due to inhibition of the activity of AURKA, and may be solid cancer or blood cancer. For example, the cancer may be at least one selected from the group consisting of squamous cell carcinoma, small cell lung cancer, non-small cell lung cancer, adenocarcinoma of lung, squamous cell carcinoma of lung, peritoneal cancer, skin cancer, skin or intraocular melanoma, rectal cancer, anal cancer, esophageal cancer, small intestine cancer, endocrine cancer, parathyroid cancer, adrenal cancer, soft tissue sarcoma, urethral cancer, chronic or acute leukemia, lymphocytic lymphoma, myelofibrosis, hepatocellular cancer, stomach cancer, gastric cancer, pancreatic cancer, glioblastoma, cervical cancer, ovarian cancer, liver cancer, bladder cancer, liver tumor, breast cancer, colon cancer, colorectal cancer, endometrial or uterine cancer, salivary gland cancer, kidney cancer, prostate cancer, vulvar cancer, thyroid cancer, head and neck cancer, brain cancer, osteosarcoma, etc., but is not limited thereto. The cancer includes not only primary cancer but also metastatic cancer.

The benign tumor includes all benign tumors capable of exhibiting preventive or therapeutic efficacy due to inhibition of the activity of AURKA, such as benign tumors in the pre-cancerous stage, and may be solid tumors or blood tumors. For example, the tumor may be at least one selected from the group consisting of Barrett's esophagus, colonic adenoma and polyp, breast fibroadenoma and cyst, monoclonal gammopathy (MGUS), monoclonal lymphocytosis, etc., but is not limited thereto.

According to one embodiment of the present invention, there is provided a method for degrading a AURKA protein by administering the compound represented by Chemical Formula I, a stereoisomer thereof, or a pharmaceutically acceptable salt thereof to a subject.

According to one embodiment of the present invention, there is provided a method for degrading a AURKA protein by administering the compound represented by Chemical Formula I, or a pharmaceutically acceptable salt thereof to a sample in vitro. The sample may be cells, cell cultures, body fluids or tissues of mammals including humans, but is not limited thereto.

According to one embodiment of the present invention, there is provided a method for preventing or treating cancer including administering the compound represented by Chemical Formula I, a stereoisomer thereof, or a pharmaceutically acceptable salt thereof to a subject in need thereof.

As used in the present invention, the “subject” refers to a subject in need of treatment for diseases, and more particularly, refers to mammals such as humans or non-human primates, mice, dogs, cats, horses and cattle.

As used in the present invention, the term “prevention” refers to all actions that delay the progression of cancer by inhibiting the growth of tumor or cancer cells or inducing apoptosis of tumor or cancer cells by administering the pharmaceutical composition of the present invention.

As used in the present invention, the term “treatment” refers to all actions that improve or beneficially change cancer by inhibiting the growth of tumor or cancer cells or inducing apoptosis of tumor or cancer cells by administering the pharmaceutical composition of the present invention, and means attempts for obtaining useful or desirable results, including clinical results. Although detectable or not, the useful or desirable clinical results may include alleviation or improvement of one or more symptoms or conditions, reduction of the range of diseases, stabilization of the disease condition, inhibition of the occurrence of the disease, inhibition of the spread of the disease, delay or slowing of the progression of the disease, delay or slowing of the onset of the disease, improvement or alleviation of the disease condition, and decay (partial or total), and is not necessarily limited thereto. In addition, the “treatment” may mean prolonging the patient's survival beyond what is expected in the absence of treatment. In addition, the “treatment” may refer to inhibiting the progression of the disease, and temporarily slowing the progression of the disease, and more preferably, permanently stopping the progression of the disease. In the present invention, the “treatment” may mean improving patient survival by enhancing an anticancer effect.

The pharmaceutical composition of the present invention may be formulated and used in the form of oral formulations such as powders, granules, tablets, capsules, suspensions, emulsions, syrups, aerosols, etc., external preparations, suppositories, and sterile injectable solutions according to conventional methods, respectively. The carrier, the excipient, and the diluent that may be included in the pharmaceutical composition may include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil. When the pharmaceutical composition is formulated, the formulation may be prepared by using diluents or excipients, such as a filler, an extender, a binder, a wetting agent, a disintegrating agent, and a surfactant, which are generally used. Solid formulations for oral administration include tablets, pills, powders, granules, capsules, and the like, and these solid formulations may be prepared by mixing at least one excipient, for example, starch, calcium carbonate, sucrose or lactose, gelatin, and the like with the compound of the present invention. Further, lubricants such as magnesium stearate and talc are used in addition to simple excipients. Liquid formulations for oral administration may correspond to suspensions, oral liquids, emulsions, syrups, and the like, and may include various excipients, for example, a wetting agent, a sweetener, an aromatic agent, a preservative, and the like, in addition to water and liquid paraffin which are commonly used as simple diluents. Formulations for parenteral administration include sterilized aqueous solutions, non-aqueous solvents, suspensions, emulsions, lyophilized agents, and suppositories. As the non-aqueous solution and the suspension, propylene glycol, polyethylene glycol, vegetable oil such as olive oil, injectable ester such as ethyl oleate, and the like may be used. As a base of the suppository, witepsol, macrogol, tween 61, cacao butter, laurinum, glycerogelatin, and the like may be used.

The dose of the pharmaceutical composition of the present invention will vary depending on the age, sex, and weight of a subject to be treated, a specific disease or pathological condition to be treated, the severity of the disease or pathological condition, a route of administration, and the judgment of a prescriber. The dose based on these factors is determined within a level of those skilled in the art, and in general, the dose is in the range of 0.0001 mg/kg/day to about 3000 mg/kg/day. A more preferable dose is 0.1 mg/kg/day to 1000 mg/kg/day. The administration may be performed once a day or several times a day. The dose does not limit the scope of the present invention in any aspect.

The pharmaceutical composition of the present invention may be administered to mammals such as mice, rats, livestock, and humans through various routes. All methods of administration may be expected and for example, the pharmaceutical composition may be administered by oral, rectal or intravenous, intramuscular, subcutaneous, intrauterine dural or cerebrovascular injection.

In the present invention, in addition to the active ingredients, the pharmaceutical composition for preventing or treating cancer may further include any compound or natural extract that has already been proven in safety and known to have anticancer activity in order to increase and reinforce the anticancer effect.

According to one embodiment of the present invention, there is provided an anticancer adjuvant for treating cancer including the compound represented by Chemical Formula I, a stereoisomer thereof, or a pharmaceutically acceptable salt thereof as an active ingredient.

As used herein, the term “anticancer adjuvant” refers to a composition that synergistically increases the effect of anticancer treatment by preventing side effects caused by an anticancer agent when applied in parallel with treatment with the anticancer agent. Therefore, the therapeutic adjuvant is an anticancer adjuvant and is able to be administered together with the anticancer agent, simultaneously or sequentially.

The type of anticancer agent that may be used with the anticancer adjuvant of the present invention is not particularly limited. The anticancer agent may be selected based on general principles considered when selecting anticancer agents, such as a type of cancer cell, an absorption rate of the anticancer agent (treatment period and route of anticancer agent administration), a location of the tumor, and a size of the tumor.

The above description just illustrates the technical spirit of the present invention and various changes and modifications may be made by those skilled in the art to which the present invention pertains without departing from an essential characteristic of the present invention. Accordingly, the various exemplary examples disclosed in the present invention are not intended to limit the technical spirit but describe the present invention and the technical spirit of the present invention is not limited by the following exemplary examples. The protective scope of the present invention should be construed based on the following claims, and all the techniques in the equivalent scope thereof should be construed as falling within the scope of the present invention.

### Advantageous Effects

According to the present invention, the compound may have an effect of inducing selective degradation of AURKA. Therefore, the compound according to the present invention can be effectively used for the prevention or treatment of AURKA-related diseases.

It should be understood that the effects of the present invention are not limited to the effects described above, but include all effects that can be deduced from the detailed description of the present invention or configurations of the disclosure described in claims.

### DESCRIPTION OF DRAWINGS

FIG. 1 illustrates Western blot images showing AURKA degradation and AURKA selectivity when the concentrations of Example Compounds are 0.1 μM and 1 μM[FIG. 1A: Intermediate 1 (PTM only (=inhibitor)), Compounds 1, 2, 3, 11 and 13/FIG. 1B: Compounds 4, 5, 12, 13, 14 and 30/FIG. 1C: Compounds 6, 7, 8, 9, and 10/FIG. 1D: Compounds 15, 18, 19, 20 and 31/FIG. 1E: Compounds 13, 16, 17 and 21/FIG. 1F: Compounds 15, 22, 24, 32 and 33/FIG. 1G: Intermediate 1, Compound 13, 25, 26, 27 and 28/FIG. 1H: Intermediate 1, Compounds 13, 35, 36, 37 and 38/N. Control: Negative Control (untreated with compound)].

FIG. 2 illustrates Western blot images showing AURKA degradation when the concentrations of Example Compounds are low of 6 nM and 30 nM [FIG. 2A: Intermediate 1 (PTM only (=inhibitor)), Compounds 13, 14, 15, 26 and 29/FIG. 2B: Intermediate 1, Compounds 13, 35, 36 and 38/FIG. 2C: Compounds 2, 11, 14, 15 and 17/FIG. 2D: Compounds 15, 16, 18, 20 and 21/FIG. 2E: Compounds 15, 26, 36 and 37/N. Control: Negative Control (untreated with compound)].

FIG. 3 illustrates Western blot images showing AURKA degradation when the concentrations of Alisertib-based PROTAC of Comparative Examples 1 to 3 are 0.1 μM and 1 μM [FIG. 3A: Comparative Example 1/FIG. 3B: Comparative Example 2/FIG. 3C: Comparative Example 3].

FIG. 4 is a schematic diagram showing an operating principle of PROTAC.

### MODES OF THE INVENTION

Hereinafter, exemplary examples of the present invention will be described in detail with reference to the accompanying drawings. The examples of the present invention may be modified in various forms, and it should not be construed that the scope of the present invention is limited to examples to be described below. The exemplary examples will be provided for more completely explaining the present invention to those skilled in the art. Therefore, shapes of components in the drawings will be exaggerated to emphasize a clearer description.

The present invention provides synthetic methods for Compounds 1 to 38 shown in Table 1 below.

TABLE 1

| Comp. | Structure |
|---|---|
| 1 | 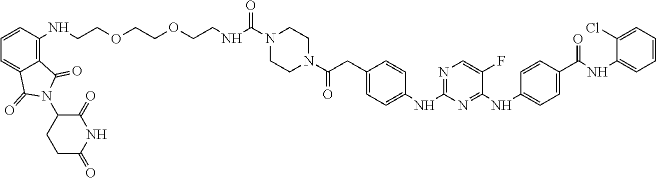 [SMILES: O=C1CCC(N2C(=O)c3cccc(NCCOCCOCCNC(=O)N4CCN(C(=O)Cc5ccc(Nc6ncc(F)c(Nc7ccc(C(=O)Nc8ccccc8Cl)cc7)n6)cc5)CC4)c3C2=O)C(=O)N1] |
| 2 | 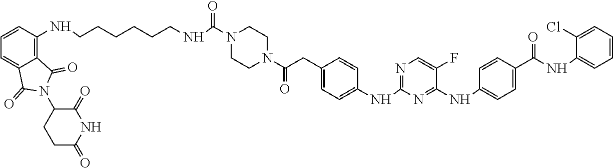 [SMILES: O=C1CCC(N2C(=O)c3cccc(NCCCCCCNC(=O)N4CCN(C(=O)Cc5ccc(Nc6ncc(F)c(Nc7ccc(C(=O)Nc8ccccc8Cl)cc7)n6)cc5)CC4)c3C2=O)C(=O)N1] |
| 3 | 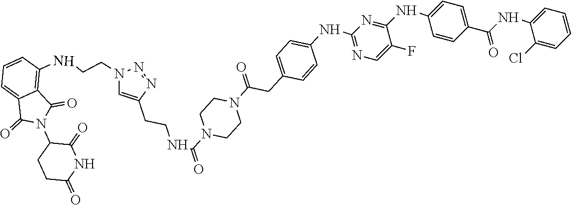 [SMILES: O=C1CCC(N2C(=O)c3cccc(NCCn4cc(CCNC(=O)N5CCN(C(=O)Cc6ccc(Nc7ncc(F)c(Nc8ccc(C(=O)Nc9ccccc9Cl)cc8)n7)cc6)CC5)nn4)c3C2=O)C(=O)N1] |
| 4 | 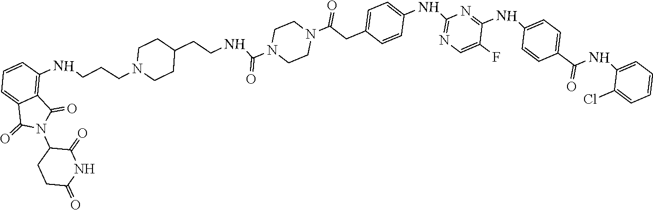 [SMILES: O=C1CCC(N2C(=O)c3cccc(NCCCN4CCC(CCNC(=O)N5CCN(C(=O)Cc6ccc(Nc7ncc(F)c(Nc8ccc(C(=O)Nc9ccccc9Cl)cc8)n7)cc6)CC5)CC4)c3C2=O)C(=O)N1] |
| 5 | 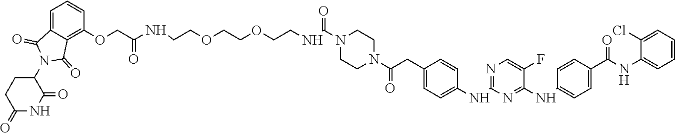 [SMILES: O=C(COc1cccc2c1C(=O)N(C1CCC(=O)NC1=O)C2=O)NCCOCCOCCNC(=O)N1CCN(C(=O)Cc2ccc(Nc3ncc(F)c(Nc4ccc(C(=O)Nc5ccccc5Cl)cc4)n3)cc2)CC1] |
| 6 | 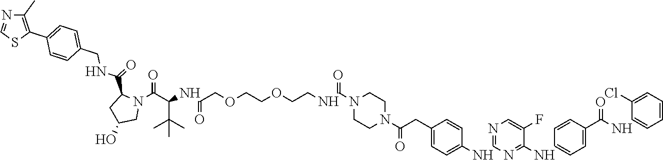 [SMILES: Cc1ncsc1-c1ccc(CNC(=O)[C@@H]2C[C@@H](O)CN2C(=O)[C@@H](NC(=O)COCCOCCNC(=O)N2CCN(C(=O)Cc3ccc(Nc4ncc(F)c(Nc5ccc(C(=O)Nc6ccccc6Cl)cc5)n4)cc3)CC2)C(C)(C)C)cc1] |
| 7 | 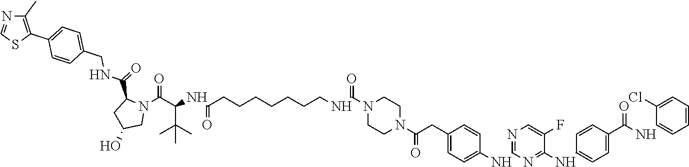 [SMILES: Cc1ncsc1-c1ccc(CNC(=O)[C@@H]2C[C@@H](O)CN2C(=O)[C@@H](NC(=O)CCCCCCCNC(=O)N2CCN(C(=O)Cc3ccc(Nc4ncc(F)c(Nc5ccc(C(=O)Nc6ccccc6Cl)cc5)n4)cc3)CC2)C(C)(C)C)cc1] |
| 8 | 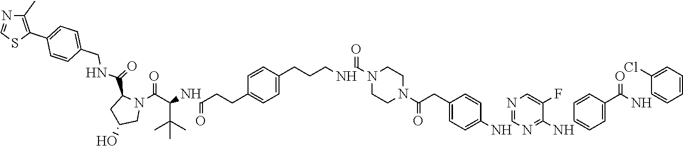 [SMILES: Cc1ncsc1-c1ccc(CNC(=O)[C@@H]2C[C@@H](O)CN2C(=O)[C@@H](NC(=O)CCc2ccc(CCCNC(=O)N3CCN(C(=O)Cc4ccc(Nc5ncc(F)c(Nc6ccc(C(=O)Nc7ccccc7Cl)cc6)n5)cc4)CC3)cc2)C(C)(C)C)cc1] |
| 9 | 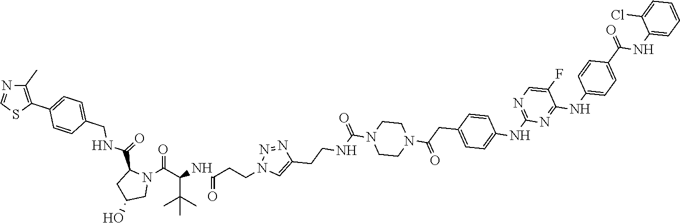 [SMILES: Cc1ncsc1-c1ccc(CNC(=O)[C@@H]2C[C@@H](O)CN2C(=O)[C@@H](NC(=O)CCn2cc(CCNC(=O)N3CCN(C(=O)Cc4ccc(Nc5ncc(F)c(Nc6ccc(C(=O)Nc7ccccc7Cl)cc6)n5)cc4)CC3)nn2)C(C)(C)C)cc1] |
| 10 | 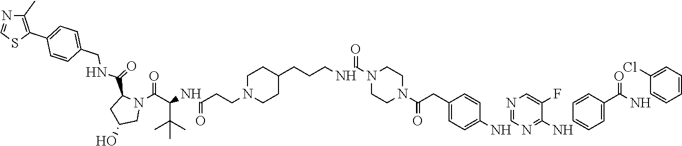 [SMILES: Cc1ncsc1-c1ccc(CNC(=O)[C@@H]2C[C@@H](O)CN2C(=O)[C@@H](NC(=O)CCN2CCC(CCCNC(=O)N3CCN(C(=O)Cc4ccc(Nc5ncc(F)c(Nc6ccc(C(=O)Nc7ccccc7Cl)cc6)n5)cc4)CC3)CC2)C(C)(C)C)cc1] |
| 11 | 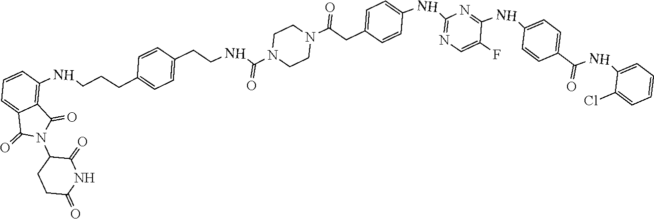 [SMILES: O=C1CCC(N2C(=O)c3cccc(NCCCc4ccc(CCNC(=O)N5CCN(C(=O)Cc6ccc(Nc7ncc(F)c(Nc8ccc(C(=O)Nc9ccccc9Cl)cc8)n7)cc6)CC5)cc4)c3C2=O)C(=O)N1] |
| 12 | 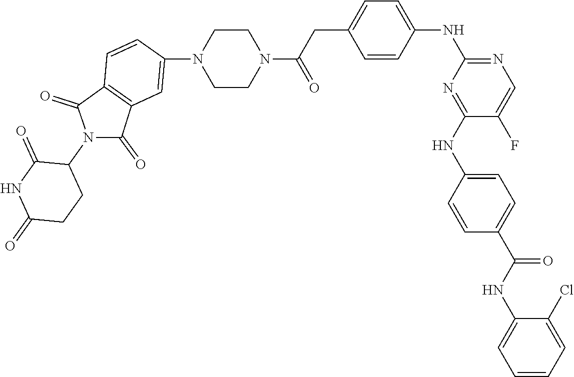 [SMILES: O=C1CCC(N2C(=O)c3ccc(N4CCN(C(=O)Cc5ccc(Nc6ncc(F)c(Nc7ccc(C(=O)Nc8ccccc8Cl)cc7)n6)cc5)CC4)cc3C2=O)C(=O)N1] |
| 13 | 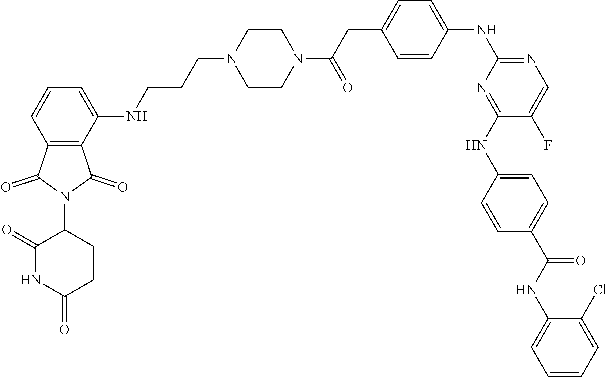 [SMILES: O=C1CCC(N2C(=O)c3cccc(NCCCN4CCN(C(=O)Cc5ccc(Nc6ncc(F)c(Nc7ccc(C(=O)Nc8ccccc8Cl)cc7)n6)cc5)CC4)c3C2=O)C(=O)N1] |
| 14 | 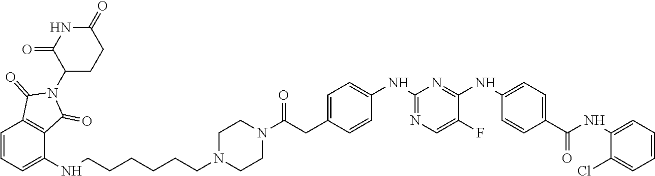 [SMILES: O=C1CCC(N2C(=O)c3cccc(NCCCCCCN4CCN(C(=O)Cc5ccc(Nc6ncc(F)c(Nc7ccc(C(=O)Nc8ccccc8Cl)cc7)n6)cc5)CC4)c3C2=O)C(=O)N1] |
| 15 | 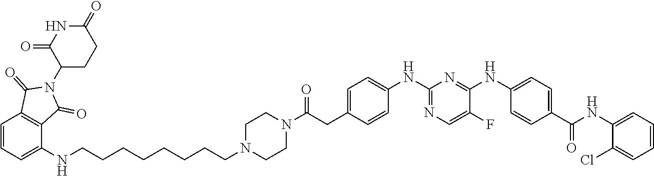 [SMILES: O=C1CCC(N2C(=O)c3cccc(NCCCCCCCCN4CCN(C(=O)Cc5ccc(Nc6ncc(F)c(Nc7ccc(C(=O)Nc8ccccc8Cl)cc7)n6)cc5)CC4)c3C2=O)C(=O)N1] |
| 16 | 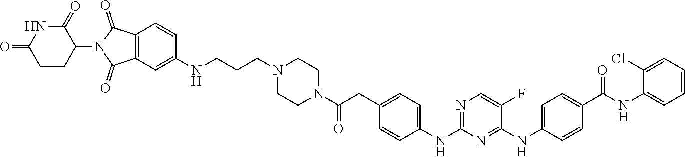 [SMILES: O=C1CCC(N2C(=O)c3ccc(NCCCN4CCN(C(=O)Cc5ccc(Nc6ncc(F)c(Nc7ccc(C(=O)Nc8ccccc8Cl)cc7)n6)cc5)CC4)cc3C2=O)C(=O)N1] |
| 17 | 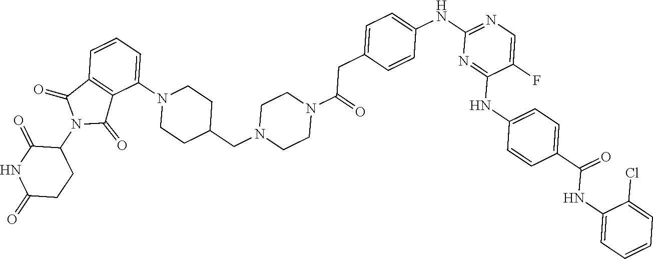 [SMILES: O=C1CCC(N2C(=O)c3cccc(N4CCC(CN5CCN(C(=O)Cc6ccc(Nc7ncc(F)c(Nc8ccc(C(=O)Nc9ccccc9Cl)cc8)n7)cc6)CC5)CC4)c3C2=O)C(=O)N1] |
| 18 | 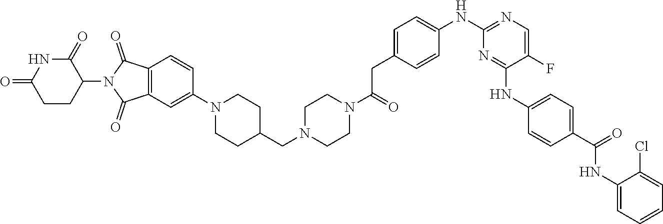 [SMILES: O=C1CCC(N2C(=O)c3ccc(N4CCC(CN5CCN(C(=O)Cc6ccc(Nc7ncc(F)c(Nc8ccc(C(=O)Nc9ccccc9Cl)cc8)n7)cc6)CC5)CC4)cc3C2=O)C(=O)N1] |
| 19 | 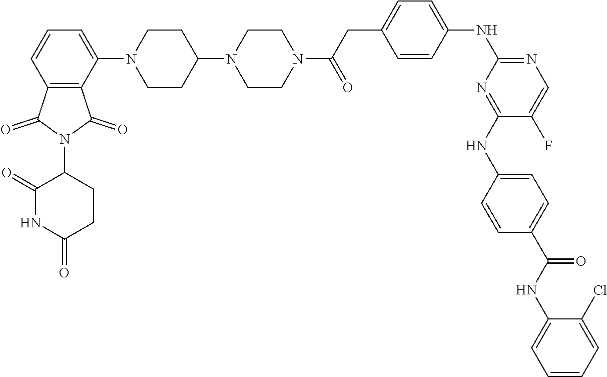 [SMILES: O=C1CCC(N2C(=O)c3cccc(N4CCC(N5CCN(C(=O)Cc6ccc(Nc7ncc(F)c(Nc8ccc(C(=O)Nc9ccccc9Cl)cc8)n7)cc6)CC5)CC4)c3C2=O)C(=O)N1] |
| 20 | 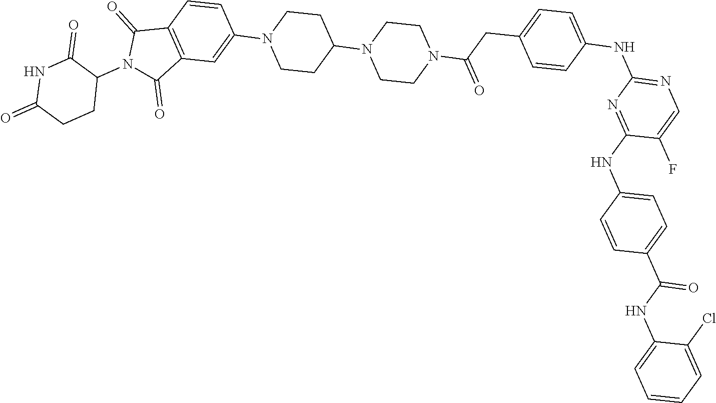 [SMILES: O=C1CCC(N2C(=O)c3ccc(N4CCC(N5CCN(C(=O)Cc6ccc(Nc7ncc(F)c(Nc8ccc(C(=O)Nc9ccccc9Cl)cc8)n7)cc6)CC5)CC4)cc3C2=O)C(=O)N1] |
| 21 | 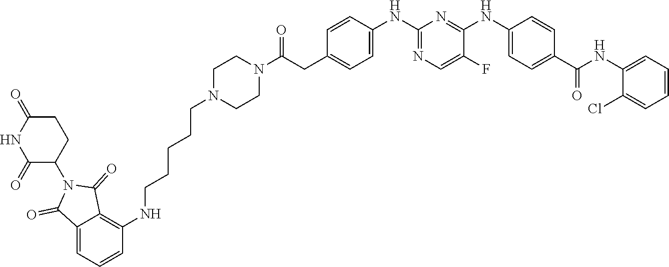 [SMILES: O=C1CCC(N2C(=O)c3cccc(NCCCCCN4CCN(C(=O)Cc5ccc(Nc6ncc(F)c(Nc7ccc(C(=O)Nc8ccccc8Cl)cc7)n6)cc5)CC4)c3C2=O)C(=O)N1] |
| 22 | 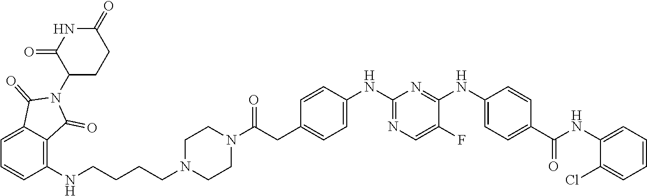 [SMILES: O=C1CCC(N2C(=O)c3cccc(NCCCCN4CCN(C(=O)Cc5ccc(Nc6ncc(F)c(Nc7ccc(C(=O)Nc8ccccc8Cl)cc7)n6)cc5)CC4)c3C2=O)C(=O)N1] |
| 23 | 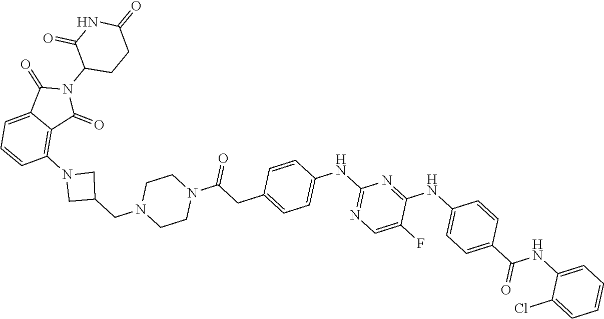 [SMILES: O=C1CCC(N2C(=O)c3cccc(N4CC(CN5CCN(C(=O)Cc6ccc(Nc7ncc(F)c(Nc8ccc(C(=O)Nc9ccccc9Cl)cc8)n7)cc6)CC5)C4)c3C2=O)C(=O)N1] |
| 24 | 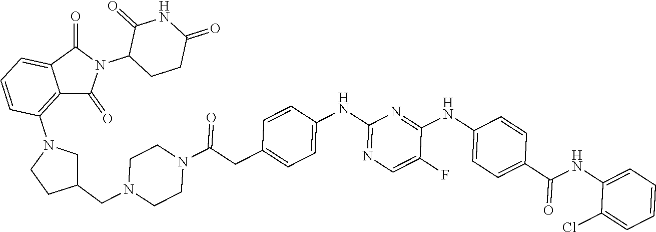 [SMILES: O=C1CCC(N2C(=O)c3cccc(N4CCC(CN5CCN(C(=O)Cc6ccc(Nc7ncc(F)c(Nc8ccc(C(=O)Nc9ccccc9Cl)cc8)n7)cc6)CC5)C4)c3C2=O)C(=O)N1] |
| 25 | 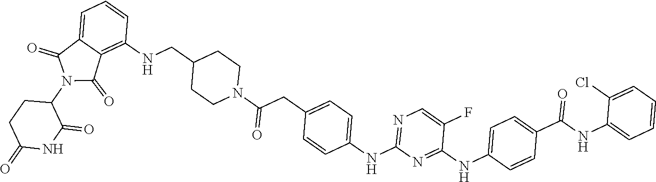 [SMILES: O=C1CCC(N2C(=O)c3cccc(NCC4CCN(C(=O)Cc5ccc(Nc6ncc(F)c(Nc7ccc(C(=O)Nc8ccccc8Cl)cc7)n6)cc5)CC4)c3C2=O)C(=O)N1] |
| 26 | 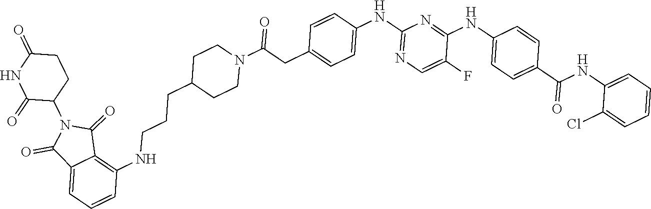 [SMILES: O=C1CCC(N2C(=O)c3cccc(NCCCC4CCN(C(=O)Cc5ccc(Nc6ncc(F)c(Nc7ccc(C(=O)Nc8ccccc8Cl)cc7)n6)cc5)CC4)c3C2=O)C(=O)N1] |
| 27 | 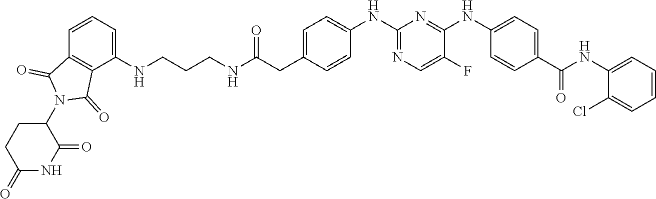 [SMILES: O=C(Cc1ccc(Nc2ncc(F)c(Nc3ccc(C(=O)Nc4ccccc4Cl)cc3)n2)cc1)NCCCNc1cccc2c1C(=O)N(C1CCC(=O)NC1=O)C2=O] |
| 28 | 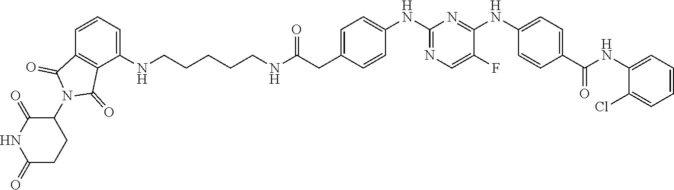 [SMILES: O=C(Cc1ccc(Nc2ncc(F)c(Nc3ccc(C(=O)Nc4ccccc4Cl)cc3)n2)cc1)NCCCCCNc1cccc2c1C(=O)N(C1CCC(=O)NC1=O)C2=O] |
| 29 | 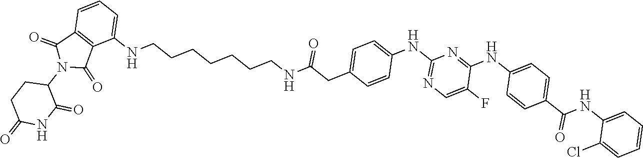 [SMILES: O=C(Cc1ccc(Nc2ncc(F)c(Nc3ccc(C(=O)Nc4ccccc4Cl)cc3)n2)cc1)NCCCCCCCNc1cccc2c1C(=O)N(C1CCC(=O)NC1=O)C2=O] |
| 30 | 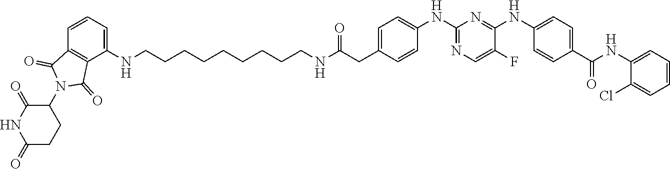 [SMILES: O=C(Cc1ccc(Nc2ncc(F)c(Nc3ccc(C(=O)Nc4ccccc4Cl)cc3)n2)cc1)NCCCCCCCCCNc1cccc2c1C(=O)N(C1CCC(=O)NC1=O)C2=O] |
| 31 | 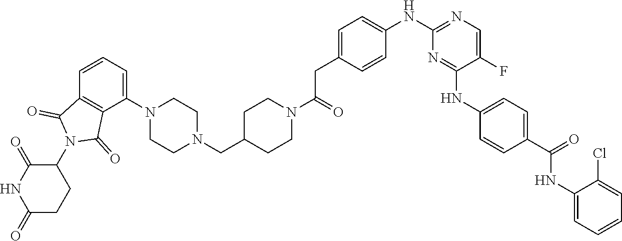 [SMILES: O=C1CCC(N2C(=O)c3cccc(N4CCN(CC5CCN(C(=O)Cc6ccc(Nc7ncc(F)c(Nc8ccc(C(=O)Nc9ccccc9Cl)cc8)n7)cc6)CC5)CC4)c3C2=O)C(=O)N1] |
| 32 | 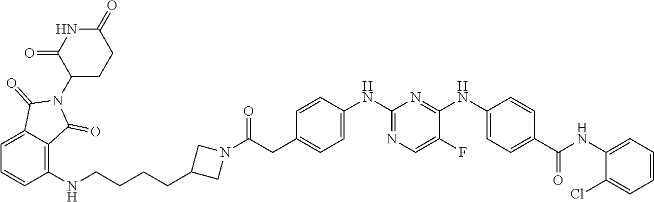 [SMILES: O=C1CCC(N2C(=O)c3cccc(NCCCCC4CN(C(=O)Cc5ccc(Nc6ncc(F)c(Nc7ccc(C(=O)Nc8ccccc8Cl)cc7)n6)cc5)C4)c3C2=O)C(=O)N1] |
| 33 | 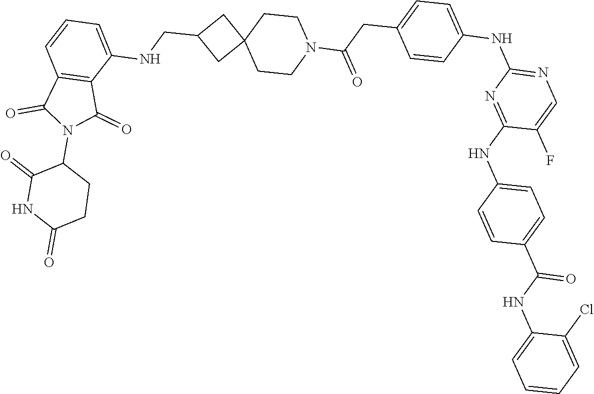 [SMILES: O=C1CCC(N2C(=O)c3cccc(NCC4CC5(CCN(C(=O)Cc6ccc(Nc7ncc(F)c(Nc8ccc(C(=O)Nc9ccccc9Cl)cc8)n7)cc6)CC5)C4)c3C2=O)C(=O)N1] |
| 34 | 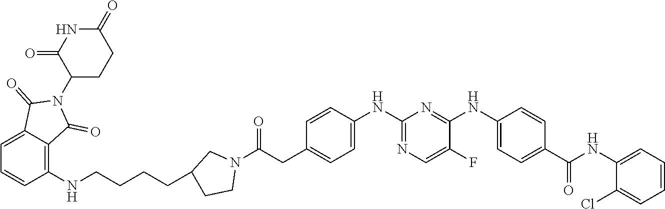 [SMILES: O=C1CCC(N2C(=O)c3cccc(NCCCCC4CCN(C(=O)Cc5ccc(Nc6ncc(F)c(Nc7ccc(C(=O)Nc8ccccc8Cl)cc7)n6)cc5)C4)c3C2=O)C(=O)N1] |
| 35 | 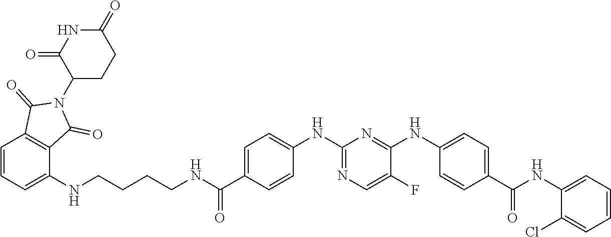 [SMILES: O=C1CCC(N2C(=O)c3cccc(NCCCCNC(=O)c4ccc(Nc5ncc(F)c(Nc6ccc(C(=O)Nc7ccccc7Cl)cc6)n5)cc4)c3C2=O)C(=O)N1] |
| 36 | 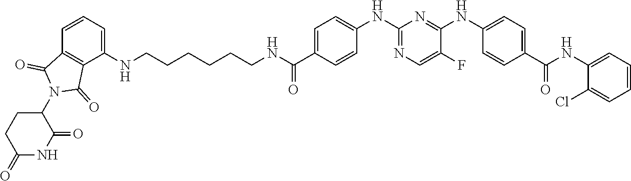 [SMILES: O=C1CCC(N2C(=O)c3cccc(NCCCCCCNC(=O)c4ccc(Nc5ncc(F)c(Nc6ccc(C(=O)Nc7ccccc7Cl)cc6)n5)cc4)c3C2=O)C(=O)N1] |
| 37 | 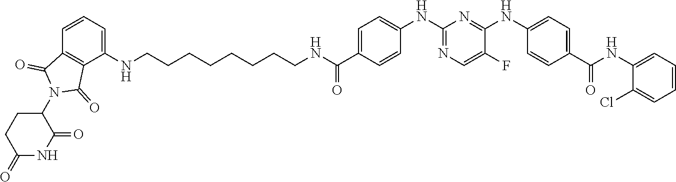 [SMILES: O=C1CCC(N2C(=O)c3cccc(NCCCCCCCCNC(=O)c4ccc(Nc5ncc(F)c(Nc6ccc(C(=O)Nc7ccccc7Cl)cc6)n5)cc4)c3C2=O)C(=O)N1] |
| 38 | 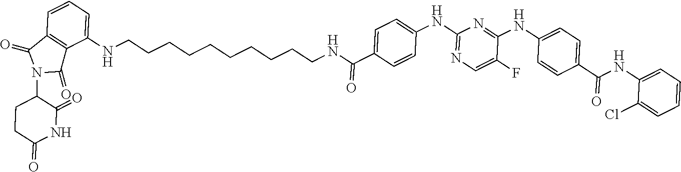 [SMILES: O=C1CCC(N2C(=O)c3cccc(NCCCCCCCCCCNC(=O)c4ccc(Nc5ncc(F)c(Nc6ccc(C(=O)Nc7ccccc7Cl)cc6)n5)cc4)c3C2=O)C(=O)N1] |

The compound of the present invention were purified according to the following methods and the structures thereof were analyzed.

### Analysis Devices

LCMS: Agilent 1290 Infinity II, Agilent 1200 series, SHIMADZU LCMS-2020

HPLC: Agilent 1260 Infinity II

NMR: BRUKER AVANCE Neo or III/400 MHz

### LCMS Analysis Method

For LCMS data, Agilent 1200 series, Agilent 1290 Infinity II, or SHIMADZU LCMS-2020 equipped with a multimode source (simultaneously with ESI and APCI) were used. 0.1% FA in water (Solvent A) and acetonitrile (Solvent B), 0.1% TFA in water (Solvent A) and 0.1% TFA acetonitrile in water (Solvent B), 10 mM NH₄HCO₃ in water (Solvent A) and acetonitrile (Solvent B), or 0.0375% TFA in water (Solvent A) and 0.01875% TFA in acetonitrile (Solvent B) were used as mobile phases. A column was used with Atlantis dC18 (50×4.6 mm) 5 μm, X-Bridge C₈ (50×4.6 mm) 3.5 μm, or Kinetex® EVO C18 2.1×30 mm 5 μm.

### HPLC Analysis Method

For HPLC data, 1260 Infinity II G1311B, G7111B or G6410B was used. 0.1% TFA in water (Solvent A) and acetonitrile (Solvent B), 10 mM NH₄HCO₃ in water (Solvent A) and acetonitrile (Solvent B), or 0.0375% TFA in water (Solvent A) and 0.01875% TFA in acetonitrile (Solvent B) were used as mobile phases. A column was used with X-Bridge C8 (150×4.6) mm, 3.5 μm, Atlantis dC18 (250×4.6) mm, 5 μm or Zobrax Eclipse Plus C18 (4.6×150) mm, 3.5 μm.

### NMR Analysis Method

¹H NMR spectra were recorded with a Bruker AVANCE Neo or III 400 MHz/5 mm Probe (BBO).

### PREPARATION EXAMPLES

### Preparation Example 1: Synthesis of Intermediate 1 (UPP-L1)

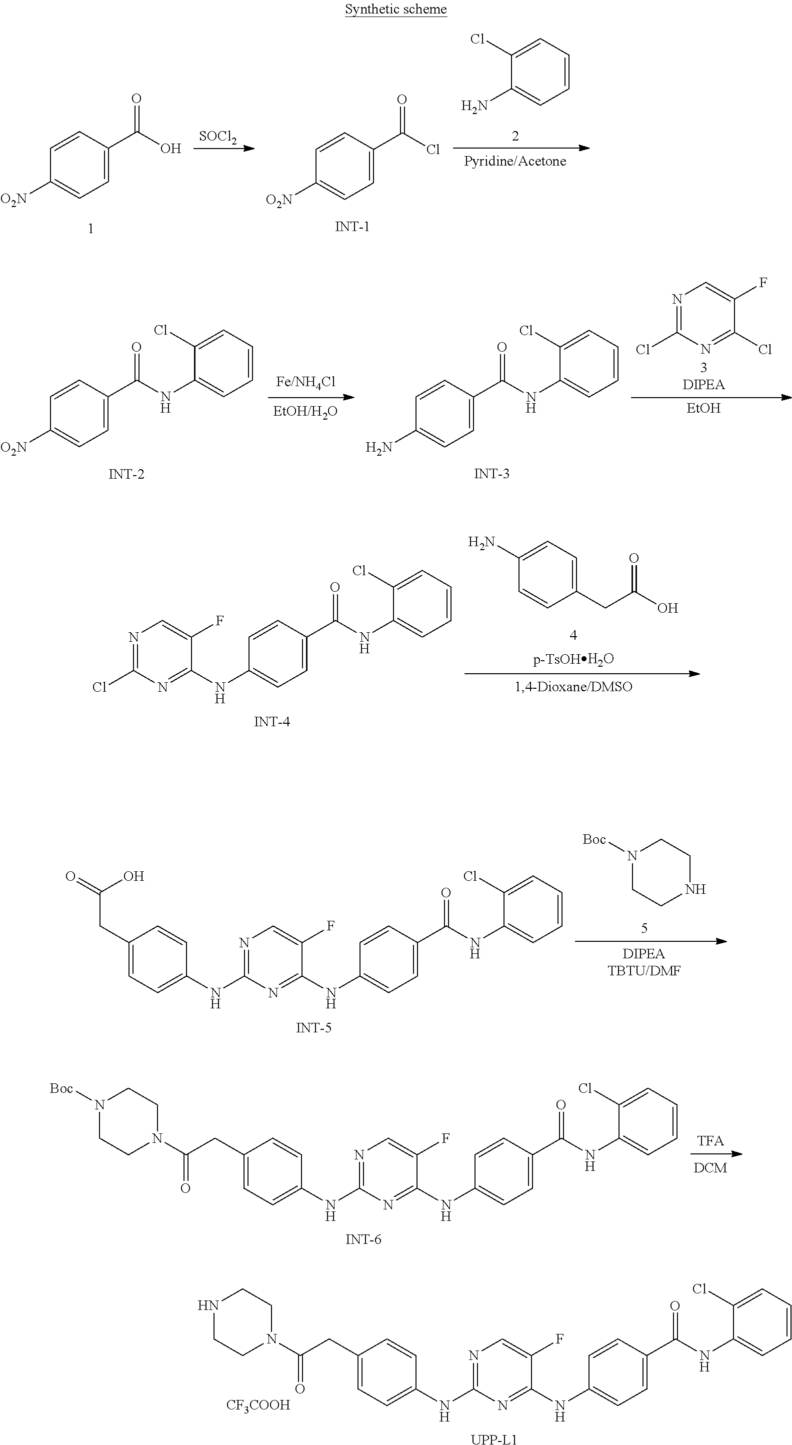

### Experimental Process

### Step 1: Synthesis of 4-nitrobenzoyl chloride (INT-1)

In a 250 mL round bottom flask, charged 4-nitrobenzoic acid (10 g, 59.8 mmol) and SOCl₂ (50 mL) at 25° C. under N₂ atmosphere. The reaction mixture was refluxed at 110° C. for 12 hours and the reaction was monitored by GCMS. After the reaction completion, the reactant was concentrated under reduced pressure and co-evaporated with n-hexane. The obtained compound was stirred with n-hexane (50 mL) for 30 minutes. The obtained solid was filtered and washed with n-hexane (20 mL) to obtain 4-nitrobenzoyl chloride (10.2 g, 54.1 mmol, 90% yield) as yellow solid. GCMS: [m/z]=185.

### Step 2: Synthesis of N-(2-chlorophenyl)-4-nitrobenzamide (INT-2)

4-nitrobenzoyl chloride (10 g, 53.9 mmol) in acetone (100 ml) was added dropwise at 0 to 5° C. under N₂ in a stirred solution of 2-chloroaniline (8.25 g, 64.7 mmol) in pyridine (50 mL). The reaction mixture was stirred at 25° C. for 2 hours. The reaction process was observed by LCMS, and after the reaction completion, the reactant was added in ice water (200 mL). The precipitated solid was obtained by filtration, washed with water (50 mL) and dried under vacuum to obtain N-(2-chlorophenyl)-4-nitrobenzamide (13.01 g, 46.1 mmol, 86% yield) as off-white solid. LCMS: (MM-ES+APCI)(M+H)+=277.1

### Step 3: Synthesis of 4-amino-N-(2-chlorophenyl)benzamide (INT-3)

Fe (13.12 g, 235 mmol) and NH₄Cl (5.03 g, 94 mmol) were added to a suspension of N-(2-chlorophenyl)-4-nitrobenzamide (13 g, 47.0 mmol) in a mixture of EtOH (200 mL) and water (50.0 mL) at 25° C. The reaction mixture was heated at 80° C. for 3 hours. The reaction process was observed by LCMS. After the reaction completion, the reactant was filtered through a celite pad and washed sequentially with water (100 mL) and MeOH (200 mL). A low boiling solvent was evaporated from the filtrate and filtered to obtain 4-amino-N-(2-chlorophenyl)benzamide (11.3 g, 45.6 mmol, 97% yield) as off-white solid. LCMS: (MM-ES+APCI)(M+H)⁺246.9; Purity: 99.63% by LCMS.

### Step 4: Synthesis of 4-((2-chloro-5 fluoropyrimidin-4-yl)amino)-N-(2-chlorophenyl)benzamide (INT-4)

DIPEA (13.49 g, 104 mmol) was added to a suspension of 4-amino-N-(2-chlorophenyl)benzamide (10.3 g, 41.8 mmol) and 2,4-dichloro-5-fluoropyrimidine (10.46 g, 62.6 mmol) in EtOH (100 ml) at 25° C. under N₂ atmosphere. The reaction mixture was refluxed at 95° C. for 16 hours. The reaction process was observed by LCMS. After completion of the reaction, the precipitated solid was obtained by filtration and washed with EtOH (30 mL) to obtain 4-((2-chloro-5-fluoropyrimidin-4-yl)amino)-N-(2-chlorophenyl)benzamide (11.8 g, 31.2 mmol, 74.6% yield) as off-white solid. LCMS: (MM-ES+APCI)(M+H)⁺=377.0

### Step 5: Synthesis of 2-(4-((4-((4-((2-chlorophenyl)carbamoyl)phenyl)amino)-5 fluoropyrimidin-2-yl)amino)phenyl)acetic acid (INT-5)

p-TsOH·H₂O (5.29 g, 27.8 mmol) was added to a suspension of 4-((2-chloro-5-fluoropyrimidin-4-yl)amino)-N-(2-chlorophenyl)benzamide (10.5 g, 27.8 mmol) and 2-(4-aminophenyl)acetic acid (4.21 g, 27.8 mmol) in dioxane (100 ml) and DMSO (20 ml) at 25° C. under N₂ atmosphere. The reaction mixture was refluxed at 110° C. for 24 hours. The reaction process was observed by LCMS. After completion of the reaction, the reaction mixture was added into ice water (150 mL) and the precipitated solid was filtered to obtain 2-(4-((4-((4-((2-chlorophenyl)carbamoyl)phenyl)amino)-5-fluoropyrimidin-2-yl)amino)phenyl)acetic acid (12.62 g, 22.32 mmol, 80% yield) as off-white solid. LCMS: (MM-ES+APCI)(M+H)⁺=492.0

### Step 6: Synthesis of tert-butyl 4-(2-(4-((4-((4-((2-chlorophenyl)carbamoyl)phenyl)amino)-5-fluoropyrimidin-2-yl)amino)phenyl)acetyl)piperazine-1-carboxylate (INT-6)

DIPEA (12.75 ml, 73.2 mmol) and TBTU (9.40 g, 29.3 mmol) were added to a suspension of 2-(4-((4-((4-((2-chlorophenyl)carbamoyl)phenyl)amino)-5-fluoropyrimidin-2-yl)amino)phenyl)acetic acid (12 g, 24.39 mmol) and tert-butyl piperazine-1-carboxylate (5.00 g, 26.8 mmol) in DMF (120 ml) at 10 to 15° C. under N₂ atmosphere. The reaction mixture was stirred at 25° C. for 16 hours. The reaction process was observed by LCMS. After completion of the reaction, the reaction mixture was poured into ice water (300 mL) and filtered and the precipitated solid was obtained and washed with water (100 mL). The obtained solid was washed with Pet ether and dried under vacuum for 12 hours at 25° C. to obtain tert-butyl 4-(2-(4-((4-((4-((2-chlorophenyl)carbamoyl)phenyl)amino)-5-fluoropyrimidin-2-yl)amino)phenyl)acetyl)piperazine-1-carboxylate (16.68 g, 23.69 mmol, 97% yield) as off-white solid. LCMS: (MM-ES+APCI)(M+H)⁺=660.3; HPLC Purity: 93.76%

### Step 7: Synthesis of N-(2-chlorophenyl)-4-((5-fluoro-2-((4-(2-oxo-2-(piperazin-1-yl)ethyl) phenyl) amino) pyrimidin-4-yl) amino) benzamide 2,2,2-trifluoroacetate (UPP-L1)

TFA (4.49 g, 39.4 mmol) was added dropwise to a suspension of tert-butyl 4-(2-(4-((4-((4-((2-chlorophenyl)carbamoyl)phenyl)amino)-5-fluoropyrimidin-2-yl)amino)phenyl)acetyl)piperazine-1-carboxylate (1.3 g, 1.969 mmol) in DCM (25 ml) at 0 to 5° C. under N₂. The reaction mixture was stirred at 25° C. for 12 hours. The reaction process was observed by LCMS. After completion of the reaction, the reaction mixture was evaporated and dried. The obtained compound was dissolved in THE (25 mL) and MeOH (25 mL) and added with Si-carbonate (3.0 g). The suspension was stirred for 2 hours, filtered with a celite pad and washed with MeOH (25 mL). The filtrate was concentrated under reduced pressure to obtain N-(2-chlorophenyl)-4-((5-fluoro-2-((4-(2-oxo-2-(piperazin-1-yl)ethyl) phenyl) amino) pyrimidin-4-yl) amino) benzamide 2,2,2-trifluoroacetate (1.0 g, 96.9% yield) as brown solid. LCMS (MM-ES+APCI)(M+H)⁺=560.2; HPLC: Rt-3.39; Purity: 98.85%.

### Preparation Example 2: Synthesis of Intermediate 2 (UPP-L2)

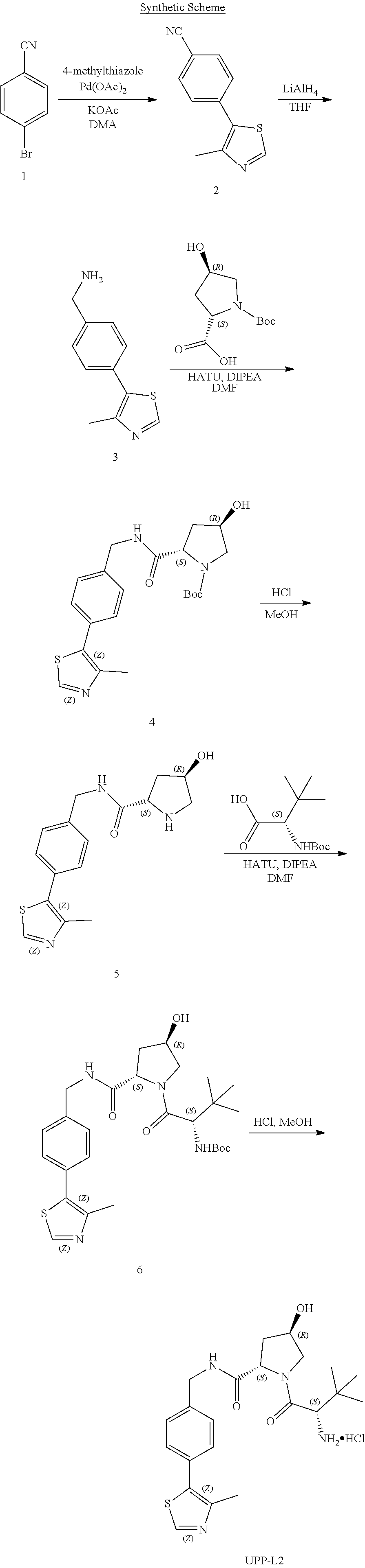

### Experimental Process

### Step 1: Synthesis of 4-(4-methylthiazol-5-yl) benzonitrile (2)

KOAc (5.39 g, 54.9 mmol) and Pd(OAc)₂ (0.062 g, 0.275 mmol) were added to a degassed solution of 4-bromobenzonitrile (5 g, 27.5 mmol) and 4-methylthiazole (5.45 g, 54.9 mmol) in DMA (50 ml) at 25° C. The reaction mixture was stirred at 145° C. for 15 hours under N₂ atmosphere, and the reaction progress was observed by TLC and eluted with 20% Pet ether/EtOAc. After completion of the reaction, the reaction mixture was cooled to 25° C., diluted with water and then extracted with EtOAc (3×100 mL). The organic phase was washed with water (3×100 mL) and brine (1×150 mL). The obtained organic phase was dried over Na₂SO₄, and filtered and concentrated under reduced pressure. The residue was purified by flash column chromatography, and eluted with 25-30% Pet ether/EtOAc. Desired fractions were concentrated under reduced pressure to obtain 4-(4-methylthiazol-5-yl) benzonitrile (4.52 g, 78% yield) as yellow solid. LCMS: (MM-ES+APCI)(M+H)⁺=201.1; Purity: 95.2% by LCMS.

### Step 2: Synthesis of (4-(4-methylthiazol-5-yl)phenyl)methanamine (3)

LiAlH₄ (33.7 mL, 67.4 mmol) was added dropwise to a solution of 4-(4-methylthiazol-5-yl)benzonitrile (4.5 g, 22.47 mmol) in THF (90 mL) at 0 to 5° C. under N₂ atmosphere. The reaction mixture was refluxed at 70° C. for 5 hours. The reaction process was observed by TLC. After completion of the reaction, the reaction mixture was cooled to 0 to 5° C., and quenched by a 10% NaOH solution (15 mL) under N₂ atmosphere. The inorganic salt was collected by filtration and washed with THF (2×100 mL). The filtrate was dried over Na₂SO₄, and filtered and concentrated under reduced pressure. The residue was co-evaporated with toluene (2×50 mL) to obtain (4-(4-methylthiazol-5-yl)phenyl)methanamine as yellow orange liquid. LCMS: (MM-ES+APCI)(M+H)⁺=205.1; Purity: 80.5% by LCMS.

### Step 3: Synthesis of tert-butyl (2S,4R)-4-hydroxy-2-((4-(4-methylthiazol-5 yl) benzyl) carbamoyl) pyrrolidine-1-carboxylate (4)

(4-(4-methylthiazol-5-yl) phenyl)methanamine (1.4 g, 6.85 mmol), DIPEA (1.771 g, 13.71 mmol) and HATU (3.91 g, 10.28 mmol) were added to a solution of (2S,4R)-1-(tert-butoxycarbonyl)-4-hydroxypyrrolidine-2-carboxylic acid (1.585 g, 6.85 mmol) in DMF (15 mL) at 25° C. under N₂ atmosphere. The reaction mixture was stirred at 25° C. for 16 hours and the reaction process was observed by LCMS. After completion of the reaction, the reaction mixture was diluted with water (50 mL) and extracted with EtOAc (3×30 mL). The combined organic phase was washed with brine (3×30 mL), dried over Na₂SO₄, and filtered and concentrated under reduced pressure. The residue was purified by flash column chromatography, eluted at 3-5% of MeOH/CH₂Cl₂ to obtain tert-butyl (2S,4R)-4-hydroxy-2-((4-(4-methylthiazol-5-yl)benzyl)carbamoyl)pyrrolidine-1-carboxylate (1.4 g, 48.9% yield) as yellow solid. LCMS: (MM-ES+APCI)(M+H)⁺=418.2; Purity: 87.21% by LCMS.

### Step 4: Synthesis of (2S,4R)-4-hydroxy-N-(4-(4-methylthiazol-5-yl) benzyl) pyrrolidine-2-carboxamide hydrochloride (5)

4 M HCl in dioxane (4.19 ml, 16.77 mmol) was added dropwise to a stirred solution of tert-butyl (2S,4R)-4-hydroxy-2-((4-(4-methylthiazol-5-yl) benzyl) carbamoyl) pyrrolidine-1-carboxylate (1.4 g, 3.35 mmol) in MeOH (10 mL) at 0 to 10° C. under N₂ atmosphere. The reaction mixture was stirred at 25° C. for 3 hours. The reaction process was observed by LCMS. After completion of the reaction, the reaction mixture was concentrated under reduced pressure to obtain (2S,4R)-4-hydroxy-N-(4-(4-methylthiazol-5-yl)benzyl)pyrrolidine-2-carboxamide hydrochloride (1.01 g, 77% yield) as off-white solid. LCMS: (MM-ES+APCI)(M+H)⁺=318.2; Purity: 90.68% by LCMS.

### Step 5: Synthesis of tert-butyl ((S)-1-((2S,4R)-4-hydroxy-2-((4-(4-methylthiazol-5-yl) benzyl) carbamoyl) pyrrolidin-1 yl)-3,3-dimethyl-1-oxobutan-2-yl) carbamate (6)

DIPEA (1.481 ml, 8.48 mmol), (S)-2-((tert-butoxycarbonyl)amino)-3,3-dimethylbutanoic acid (0.719 g, 3.11 mmol) and HATU (1.612 g, 4.24 mmol) were added to a suspension of (2S,4R)-4-hydroxy-N-(4-(4-methylthiazol-5-yl) benzyl) pyrrolidine-2-carboxamide hydrochloride (1.0 g, 2.83 mmol) in DMF (10 ml) at 25° C. under N₂ atmosphere. The reaction mixture was stirred at 25° C. for 12 hours. The reaction process was observed by LCMS. After completion of the reaction, the reactant was diluted with water and extracted with EtOAc (2×30 mL). The organic phase was washed with a 10% NaHCO₃ solution (30 mL), brine (30 mL) and dried over Na₂SO₄. The obtained organic phase was concentrated under reduced pressure, purified by flash column chromatography, and eluted with 70-80% Pet ether/EtOAc to obtain tert-butyl ((S)-1-((2S,4R)-4-hydroxy-2-((4-(4-methylthiazol-5-yl)benzyl) carbamoyl)pyrrolidin-1-yl)-3,3-dimethyl-1-oxobutan-2-yl)carbamate (1.03 g, 65.1% yield) as beige solid. LCMS: (MM-ES+APCI)(M+H)⁺=531.2; Purity: 94.8% by LCMS.

### Step 6: Synthesis of (2S,4R)-1-((S)-2-amino-3,3-dimethylbutanoyl)-4-hydroxy-N-(4-(4-methylthiazol-5 yl)benzyl)pyrrolidine-2-carboxamide hydrochloride (UPP-L2)

4 M HCl/dioxane (4.71 mL, 18.84 mmol) was added dropwise to a stirred solution of tert-butyl ((S)-1-((2S,4R)-4-hydroxy-2-((4-(4-methylthiazol-5-yl)benzyl) carbamoyl)pyrrolidin-1-yl)-3,3-dimethyl-1-oxobutan-2-yl)carbamate (1.0 g, 1.884 mmol) in MeOH (10 mL) at 0 to 5° C. under N₂ atmosphere. The reaction mixture was stirred at 25° C. for 3 hours. The reaction process was observed by TLC. After completion of the reaction, the residue solvent was removed by rotary evaporation to obtain (2S,4R)-1-((S)-2-amino-3,3-dimethylbutanoyl)-4-hydroxy-N-(4-(4-methylthiazol-5-yl)benzyl)pyrrolidine-2-carboxamide hydrochloride (930 mg, 100% yield) as off-white solid. LCMS (MM-ES+APCI)(M+H)⁺=431.2, Purity by HPLC: 94.83%.

### EXAMPLES

### Example 1: Synthesis of Compound 1

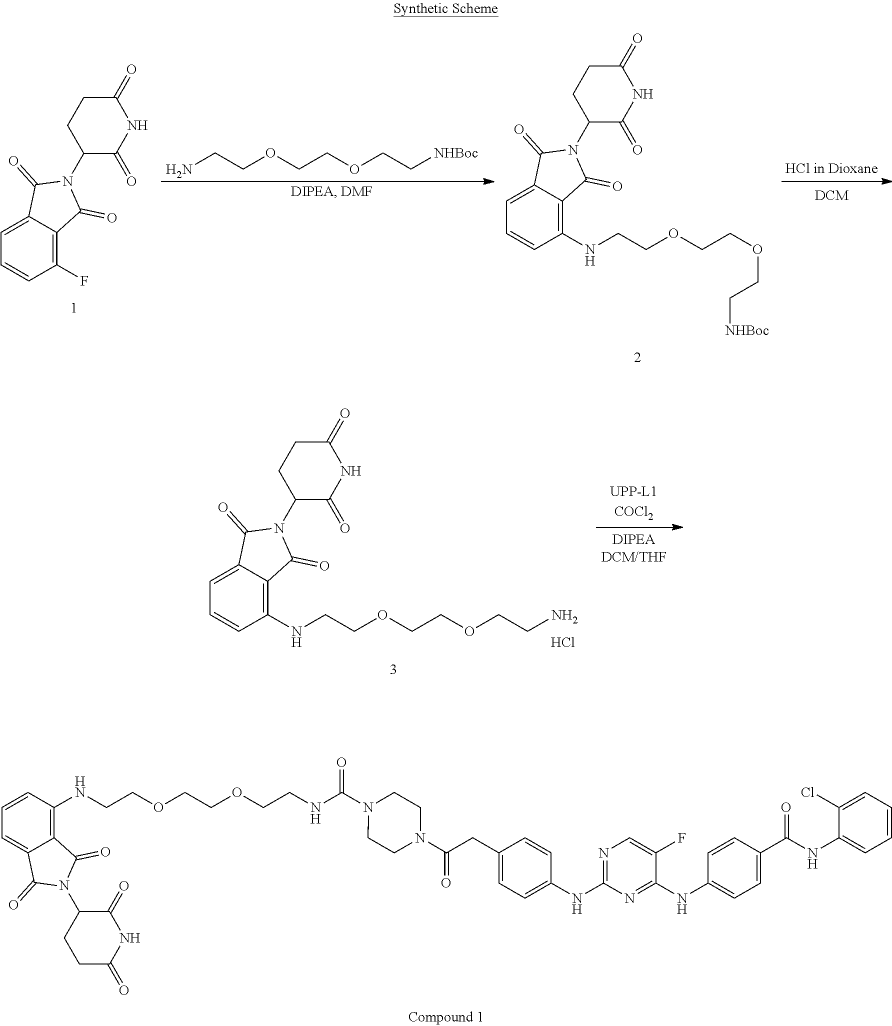

### Experimental Process

### Step 1: Synthesis of tert-butyl (2-(2-(2-((2-(2,6-dioxopiperidin-3 yl)-1,3-dioxoisoindolin-4-yl)amino)ethoxy)ethoxy)ethyl)carbamate (2)

Tert-butyl (2-(2-(2-aminoethoxy)ethoxy)ethyl)carbamate (0.989 g, 3.98 mmol) and DIPEA (1.404 g, 10.86 mmol) were added to a stirred solution of 2-(2,6-dioxopiperidin-3-yl)-4-fluoroisoindoline-1,3-dione (1 g, 3.62 mmol) in DMF (10 mL) at 25° C. The reaction mixture was heated at 90° C. for 16 hours. The reaction process was observed by LCMS, and after completion of the reaction, the reactant was concentrated under reduced pressure. The residue was diluted with water and extracted with EtOAc (2×30 mL). The combined organic layer was washed with a 10% NaHCO₃ solution (30 mL) and then washed with brine (30 mL). Thereafter, the organic phase was dried over Na₂SO₄, and filtered and concentrated under reduced pressure. The mixture was purified by flash column chromatography (80-100% EtOAc/Petroleum ether) and then concentrated under reduced pressure to obtain tert-butyl (2-(2-(2-((2-(2,6-dioxopiperidin-3-yl)-1,3-dioxoisoindolin-4-yl)amino)ethoxy)ethoxy)ethyl)carbamate (658 mg, 31.0% yield) as yellow gum.

LCMS (MM-ES+APCI) (M+H-100)⁺=405.2; Purity: 86% by LCMS.

### Step 2: Synthesis of 4-((2-(2-(2-aminoethoxy)ethoxy)ethyl)amino)-2-(2,6-dioxopiperidin-3 yl)isoindoline-1,3-dione hydrochloride (3)

HCl/1,4-dioxane (2.344 ml, 9.37 mmol) was added dropwise to a solution of tert-butyl (2-(2-(2-((2-(2,6-dioxopiperidin-3-yl)-1,3-dioxoisoindolin-4-yl)amino)ethoxy)ethoxy)ethyl)carbamate (275 mg, 0.469 mmol) in DCM (5 mL) at 0 to 5° C. under N₂ atmosphere, and then the reaction mixture was stirred at 25° C. for 12 hours. The reaction process was observed by LCMS. After completion of the reaction, the reactant was concentrated under reduced pressure and washed with diethyl ether (5 mL) to obtain 4-((2-(2-(2-aminoethoxy)ethoxy)ethyl)amino)-2-(2,6-dioxopiperidin-3-yl)isoindoline-1,3-dione hydrochloride (250 mg) as yellow solid and the compound was directly used in the next step without further purification.

LCMS (MM-ES+APCI) (M+H)⁺=405.2; Purity: 87.4% by LCMS.

### Step 3: Synthesis of 4-(2-(4-((4-((4-((2-chlorophenyl)carbamoyl)phenyl)amino)-5-fluoropyrimidin-2-yl)amino)phenyl)acetyl)-N-(2-(2-(2-((2-(2,6-dioxopiperidin-3-yl)-1,3-dioxoisoindolin-4-yl)amino)ethoxy)ethoxy)ethyl)piperazine-1-carboxamide (Compound 1)

DIPEA (147 mg, 1.134 mmol) and Phosgene 20%/toluene (421 mg, 0.851 mmol) were added to a suspension of 4-((2-(2-(2-aminoethoxy)ethoxy)ethyl)amino)-2-(2,6-dioxopiperidin-3-yl)isoindoline-1,3-dione hydrochloride (250 mg, 0.567 mmol) in DCM (5 mL) at 0 to 5° C. under N₂ atmosphere. The reaction mixture was stirred at 0 to 5° C. for 40 minutes. The reaction mixture was added dropwise with a solution of N-(2-chlorophenyl)-4-((5-fluoro-2-((4-(2-oxo-2-(piperazin-1-yl)ethyl)phenyl)amino)pyrimidin-4-yl)amino)benzamide 2,2,2-trifluoroacetate (UPP-L1) (229 mg, 0.340 mmol) and TEA (0.158 ml, 1.134 mmol) in THE (3 mL) at 0 to 5° C. The reactant was slowly warmed to 25° C. and stirred for 1 hour. The reaction process was observed by LCMS, and after completion of the reaction, the reactant was quenched with a 10% NaHCO₃ solution (10 mL) and extracted with DCM (2×20 mL). The combined organic phase was dried over Na₂SO₄, and filtered and concentrated under reduced pressure. The obtained reactant was purified by prep-HPLC and then freeze-dried for 48 hours to obtain Compound 1 (56 mg, 9.68% yield) as yellow solid. LCMS (MM-ES+APCI) (M+H)⁺=990.3, HPLC: Rt-11.52; Purity: 97.07%

¹H NMR (400 MHz, DMSO-d₆) δ=11.10 (br s, 1H), 9.91 (br s, 1H), 9.67 (br s, 1H), 9.30 (br s, 1H), 8.19 (d, J=3.6 Hz, 1H), 8.04 (d, J=8.8 Hz, 2H), 7.97 (d, J=8.8 Hz, 2H), 7.66 (dd, J1=7.6, J2=1.2 Hz 1H), 7.63-7.56 (m, 4H), 7.40 (td, J1=8.8, J2=1.60 Hz, 1H), 7.30 (td, J1=8.0, J2=1.60, Hz, 1H), 7.13 (m, 3H), 7.04 (d, J=7.2 Hz, 1H), 6.61 (t, J=5.6 Hz, 1H), 6.65 (t, J=5.2 Hz, 1H), 5.06 (dd, J1=12.8, J2=5.2 Hz, 1H), 3.66 (s, 2H), 3.61 (t, J=5.6 Hz, 2H), 3.56-3.54 (m, 2H), 3.51-3.50 (m, 2H), 3.46-3.38 (m, 8H), 3.23-3.14 (m, 6H), 2.93-2.83 (m, 1H), 2.64-2.58 (m, 2H),), 2.04-2.02 (m, 1H)

### Example 2: Synthesis of Compound 2

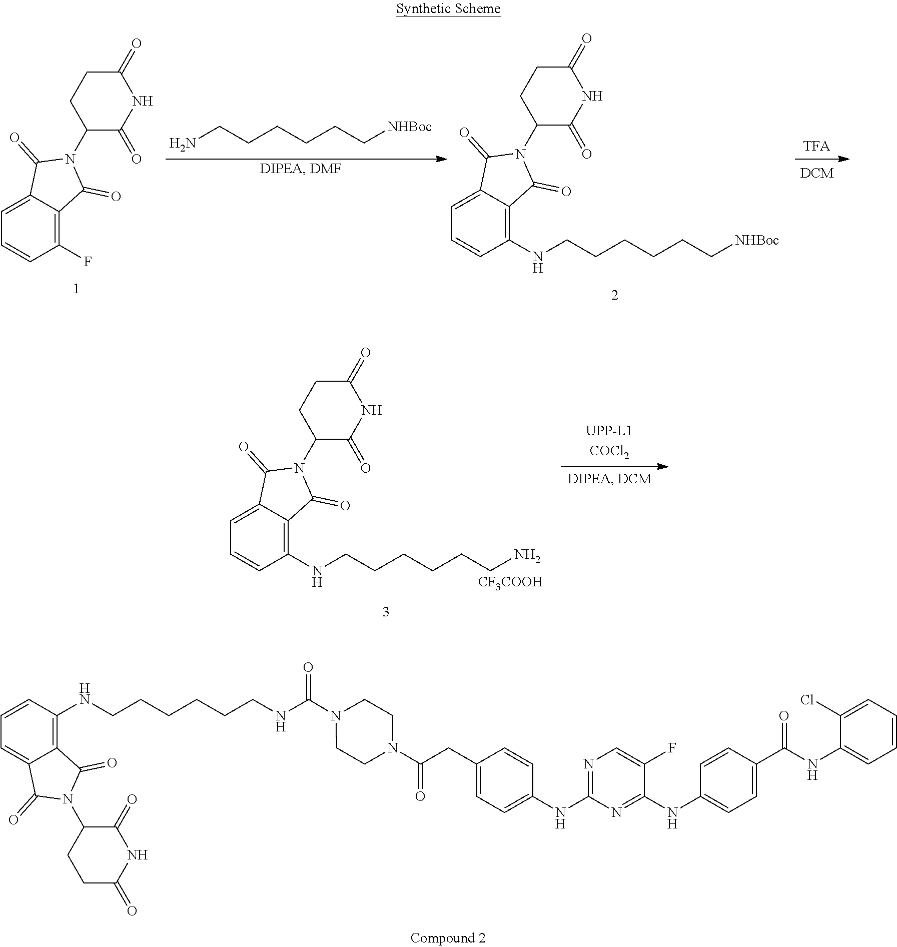

### Experimental Process

### Step 1: Synthesis of tert-butyl (6-((2-(2,6-dioxopiperidin-3 yl)-1,3-dioxoisoindolin-4-yl)amino)hexyl)carbamate (2)

Tert-butyl (6-aminohexyl)carbamate (0.783 g, 3.62 mmol) and DIPEA (0.946 m, 5.43 mmol) were added to a solution of 2-(2,6-dioxopiperidin-3-yl)-4-fluoroisoindoline-1,3-dione (1.0 g, 3.62 mmol) in DMF (10 ml) at 25° C. The reaction mixture was heated at 90° C. for 16 hours. The reaction process was observed by LCMS, and after completion of the reaction, the reactant was concentrated under reduced pressure. The residue was diluted with water and extracted with EtOAc (2×30 mL). The combined organic phase was washed with brine (30 mL), dried, and filtered and concentrated under reduced pressure. The residue was purified by reverse phase column chromatography, concentrated under reduced pressure, neutralized with a 10% NaHCO₃ solution, and then extracted with DCM (2×30 mL). The combined organic phase was dried over Na₂SO₄, and filtered and concentrated under reduced pressure to obtain tert-butyl (6-((2-(2,6-dioxopiperidin-3-yl)-1,3-dioxoisoindolin-4-yl)amino)hexyl)carbamate (0.502 g, 29.3% yield) as yellow foam. LCMS (MM-ES+APCI)(M+H-100)⁺=373.8; Purity: 99.26% by LCMS.

### Step 2: Synthesis of 4-((6-aminohexyl)amino)-2-(2,6-dioxopiperidin-3-yl)isoindoline-1,3-dione 2,2,2-trifluoroacetate (3)

TFA (1.630 ml, 21.16 mmol) was added dropwise to a solution of tert-butyl (6-((2 (2,6-dioxopiperidin-3-yl)-1,3-dioxoisoindolin-4-yl)amino)hexyl)carbamate (0.5 g, 1.058 mmol) in DCM (10 ml) at 0 to 5° C. The reaction mixture was stirred at 25° C. for hours. The reaction process was observed by TLC, and after completion of the reaction, the reaction mixture was concentrated under reduced pressure. The obtained compound was dissolved in DCM (30 mL) and added with Si-carbonate (2 g). The suspension was filtered through celite, washed with DCM (50 mL), and then concentrated under reduced pressure to obtain 4-((6-aminohexyl)amino)-2-(2,6-dioxopiperidin-3-yl)isoindoline-1,3-dione 2,2,2-trifluoroacetate (508 mg, 98% yield) as yellow gum. LCMS (MM-ES+APCI)(M+H)⁺=373.8; Purity: 99.87% by LCMS.

### Step 3: Synthesis of 4-(2-(4-((4-((4-((2-chlorophenyl)carbamoyl)phenyl)amino)-5-fluoropyrimidin-2-yl)amino)phenyl)acetyl)-N-(6-((2-(2,6-dioxopiperidin-3 yl)-1,3-dioxoisoindolin-4-yl) amino) hexyl) piperazine-1-carboxamide (Compound 2)

DIPEA (0.133 g, 1.028 mmol) was added and Phosgene 20%/toluene (0.517 m, 1.028 mmol) were added dropwise to a solution of 4-((6-aminohexyl)amino)-2-(2,6-dioxopiperidin-3-yl)isoindoline-1,3-dione 2,2,2-trifluoroacetate (0.5 g, 1.028 mmol) in DCM (5 mL) at 0 to 5° C. under N₂ atmosphere. The reaction mixture was stirred at 0 to 5° C. for 45 minutes, and then added with a solution of N-(2-chlorophenyl)-4-((5-fluoro-2-((4-(2-oxo-2-(piperazin-1-yl)ethyl)phenyl)amino)pyrimidin-4-yl)amino)benzamide 2,2,2-trifluoroacetate (0.485 g, 0.720 mmol) and DIPEA (0.133 g, 1.028 mmol) in DCM (5 ml) at 0 to 5° C. Thereafter, the reaction mixture was stirred at 25° C. for 1 hour. The reaction process was observed by LCMS, and after completion of the reaction, the reaction mixture was quenched with a 10% NaHCO₃ solution (25 mL) and extracted with DCM (2×30 mL). The organic material was dried over Na₂SO₄, filtered and then concentrated under reduced pressure. The reactant was purified by prep-HPLC and then the obtained compound was freeze-dried to obtain Compound 2 (43 mg, 4.36% yield) as yellow solid. LCMS (MM-ES+APCI)(M+H)⁺=958.3, HPLC: Rt-14.67; Purity: 88.75%

¹H NMR (400 MHz, DMSO-d₆): δ=11.11 (br s, 1H), 9.92 (br s, 1H), 9.67 (br s, 1H), 9.30 (br s, 1H), 8.19 (d, J=3.6 Hz, 1H), 8.04 (d, J=9.2 Hz, 2H), 7.98 (d, J=8.8 Hz, 2H), 7.66 (dd, J1=8.0, J2=1.6 Hz 1H), 7.63-7.55 (m, 4H), 7.40 (td, J1=9.2, J2=1.60 Hz, 1H), 7.30 (td, J1=8.8, J2=1.20, Hz, 1H), 7.12 (d, J=8.8 Hz, 2H), 7.08 (d, J=8.8 Hz, 1H), 7.02 (d, J=6.8 Hz, 1H) 6.54-6.47 (m, 2H), 5.05 (dd, J1=12.8, J2=5.2 Hz, 1H), 3.67 (s, 2H), 3.47-3.42 (m, 4H), 3.30-3.25 (m, 2H), 3.22-3.18 (m, 4H), 3.01-2.96 (m, 2H), 2.93-2.84 (m, 1H), 2.61-2.58 (m, 2H), 2.05-2.00 (m, 1H), 1.59-1.58 (m, 2H), 1.40-1.26 (m, 6H)

### Example 3: Synthesis of Compound 3

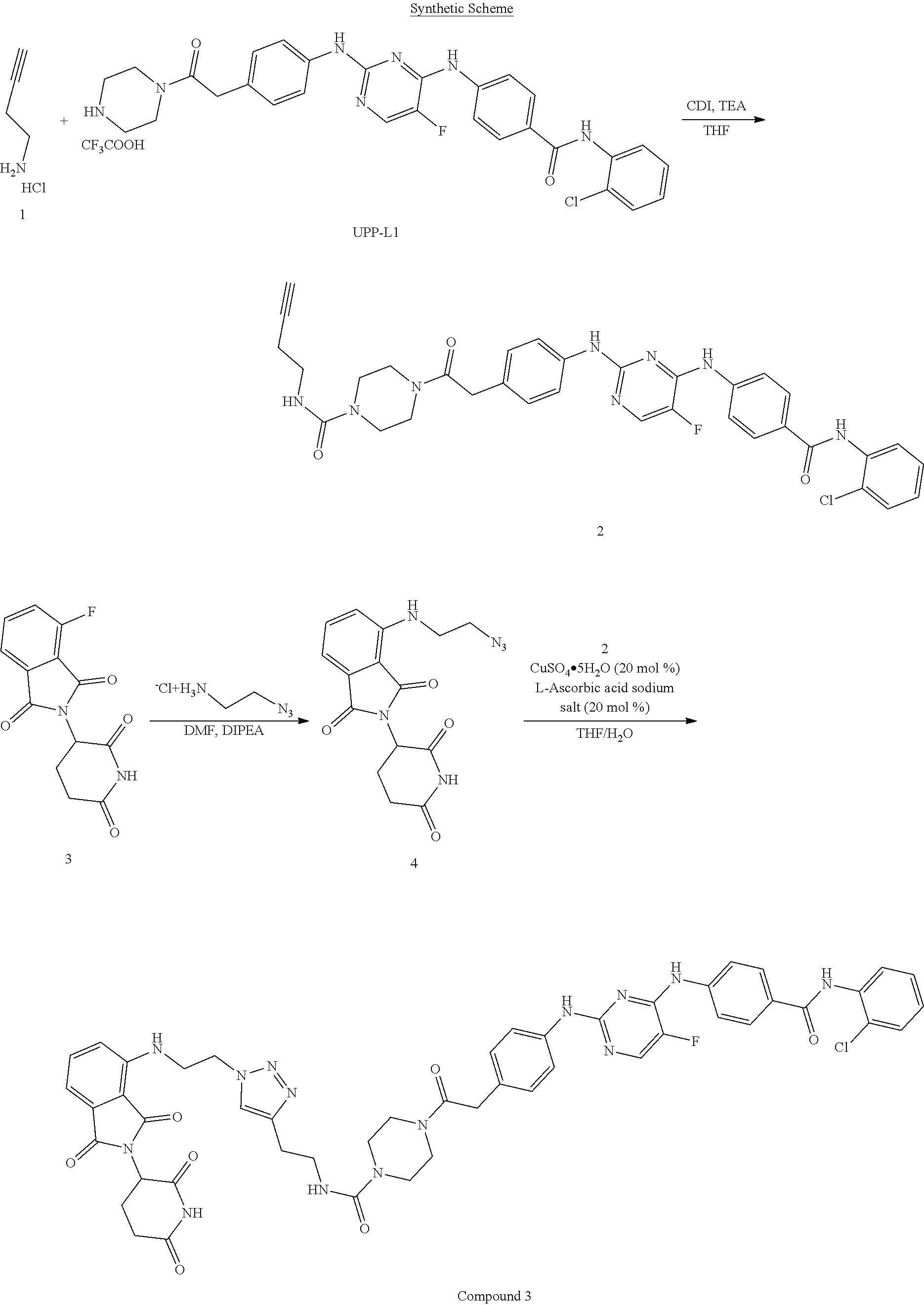

### Experimental Process

### Step 1: Synthesis of N-(but-3 yn-1-yl)-4-(2-(4-((4-((4-((2-chlorophenyl)carbamoyl)phenyl)amino)-5-fluoro pyrimidin-2-yl)amino)phenyl)acetyl)piperazine-1-carboxamide (2)

TEA (0.279 mL, 1.989 mmol) and CDI (161 mg, 0.995 mmol) were added to a solution of but-3-yn-1-amine HCl (70 mg, 0.663 mmol) in THF (6 mL) at 25° C. under N₂ atmosphere. The reaction mixture was stirred for 30 minutes. The reaction mixture was added with N-(2-chlorophenyl)-4-((5-fluoro-2-((4-(2-oxo-2-(piperazin-1-yl)ethyl)phenyl)amino)pyrimidin-4-yl)amino)benzamide 2,2,2-trifluoroacetate (313 mg, 0.464 mmol) and TEA (0.279 ml, 1.989 mmol) in THF (6 ml), and then stirred at 25° C. for 16 hours. The reactant was checked by TLC, and after completion of the reaction, the reactant was washed with water (15 mL) and concentrated at 25° C. to remove THF. The compound was extracted from an aqueous phase with a mixture of 20% MeOH in DCM (2×20 mL). The obtained organic layer was dried with Na₂SO₄ and concentrated to obtain N-(but-3-yn-1-yl)-4-(2-(4-((4-((4-((2-chlorophenyl)carbamoyl)phenyl)amino)-5-fluoro pyrimidin-2-yl)amino)phenyl)acetyl)piperazine-1-carboxamide (335 mg, 0.283 mmol, 42.7% yield) as yellow solid. LCMS: (MM-ES+APCI) (M+H)⁺=655.3, Purity: 55.38% by LCMS

### Step 2: Synthesis of 4-((2-azidoethyl)amino)-2-(2,6-dioxopiperidin-3-yl)isoindoline-1,3-dione (4)

DIPEA (0.948 ml, 5.43 mmol) and 2-(2,6-dioxopiperidin-3-yl)-4-fluoroisoindoline-1,3-dione (500 mg, 1.810 mmol) were added to a stirred solution of 2-azidoethan-1-aminium chloride (222 mg, 1.810 mmol) in DMF (10 ml) at 10 to 15° C. under N₂ atmosphere. The reaction solution was heated to 50° C. and maintained for 48 hours. The reactant was monitored by UPLC-MS. After completion of the reaction, the reaction mixture was diluted with water (50 mL) and extracted with EtOAc (3×20 mL). The combined organic phase was washed with brine (2×20 mL), dried with Na₂SO₄ and concentrated under reduced pressure at 25° C. to obtain 4-((2-azidoethyl)amino)-2-(2,6-dioxopiperidin-3-yl)isoindoline-1,3-dione (285 mg, 0.349 mmol, 19.30% yield).

LCMS: (MM-ES+APCI) (M+H)⁺=343.0; Purity: 41.96% by LCMS

### Step 3: Synthesis of 4-(2-(4-((4-((4-((2-chlorophenyl)carbamoyl)phenyl)amino)-5-fluoropyrimidin-2-yl)amino)phenyl)acetyl)-N-(2-(1-(2-((2-(2,6-dioxopiperidin-3-yl)-1,3-dioxoisoindolin-4-yl)amino)ethyl)-1H-1,2,3-triazol-4-yl)ethyl)piperazine-1-carboxamide (Compound 3)

L-ascorbic acid sodium (32.4 mg, 0.164 mmol), N-(but-3-yn-1-yl)-4-(2-(4-((4-((4-((2-chlorophenyl)carbamoyl)phenyl)amino)-5-fluoropyrimidin-2-yl)amino)phenyl)acetyl)piperazine-1-carboxamide (322 mg, 0.491 mmol) and CuSO₄·5H₂O (40.8 mg, 0.164 mmol) were sequentially added to a solution of 4-((2-azidoethyl)amino)-2-(2,6-dioxopiperidin-3-yl)isoindoline-1,3-dione (280 mg, 0.818 mmol) in a mixture of THF (10 mL) and water (5 mL). The reaction suspension was stirred at 25° C. for 12 hours. The reactant was monitored by UPLC-MS. After completion of the reaction, the reactant was filtered with celite and washed with THF (20 mL). The obtained filtrate was concentrated under reduced pressure at 25° C. to obtain Compound 3 (624 mg) as yellow solid. LCMS: (MM-ES+APCI)(M+H)⁺=997.3, Purity: 13.98% by LCMS

¹H NMR (400 MHz, DMSO-d₆): δ=11.09 (br s, 1H), 9.90 (br s, 1H), 9.67 (br s, 1H), 9.28 (s, 1H), 8.17 (d, J=3.6 Hz, 1H), 8.03 (d, J=8.8 Hz, 2H), 7.98 (d, J=8.8 Hz, 2H), 7.90 (s, 1H), 7.67 (dd, J1=8.0, J2=1.6 Hz 1H), 7.62 (d, J=8.4 Hz, 2H), 7.58-7.52 (m, 2H), 7.40 (td, J1=7.6, J2=1.2 Hz, 1H), 7.29 (td, J1=7.6, J2=1.6, Hz, 1H), 7.13 (d, J=8.4 Hz, 2H), 7.05-7.00 (m, 2H), 6.74 (t, J=6.0 Hz, 1H), 6.69 (t, J=4.8 Hz, 1H), 5.06-4.97 (m, 1H), 4.53 (t, J=5.2 Hz, 2H), 3.80-3.73 (m, 2H), 3.67 (s, 2H), 3.46-3.41 (m, 4H), 3.23-3.20 (m, 6H), 2.89-2.81 (m, 1H), 2.73-2.69 (m, 2H), 2.62-2.56 (m, 2H), 2.05-1.96 (m, 1H)

### Example 4: Synthesis of Compound 4

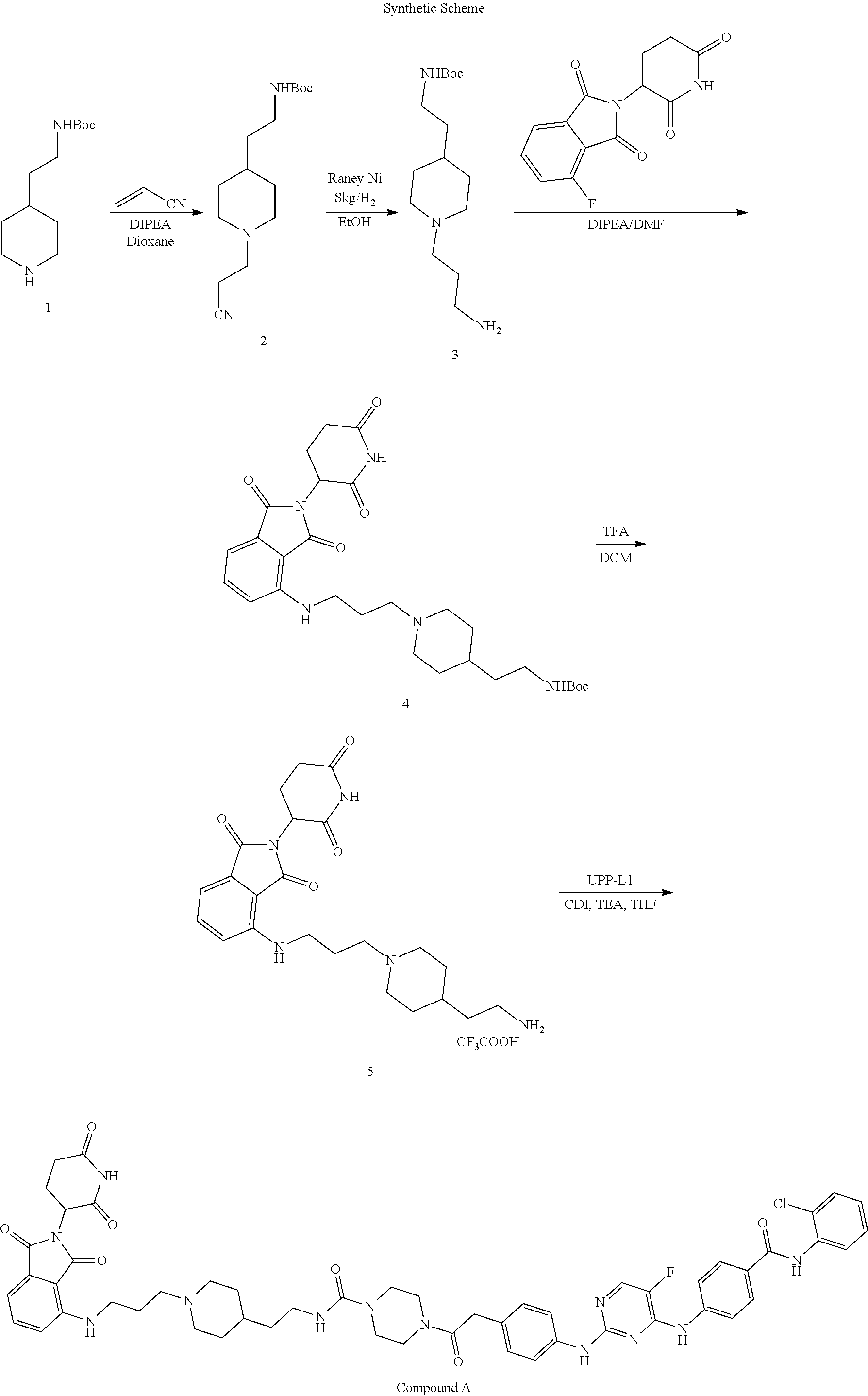

### Experimental Process

### Step 1: Synthesis of tert-butyl (2-(1-(2-cyanoethyl)piperidin-4-yl)ethyl)carbamate (2)

In a 20 ml vial, acrylonitrile (0.465 g, 8.76 mmol) was added to a solution of tert-butyl (2-(piperidin-4-yl)ethyl)carbamate (1 g, 4.38 mmol) and DIPEA (2.295 mL, 13.14 mmol) in dioxane (10 mL) at 25° C. The closed vial was heated at 100° C. for 12 hours. The reaction process was observed by LCMS, and after completion of the reaction, the reactant was diluted with water and extracted with EtOAc (2×20 mL). The combined organic phase was dried over Na₂SO₄, and filtered and concentrated under reduced pressure to obtain tert-butyl (2-(1-(2-cyanoethyl)piperidin-4-yl)ethyl)carbamate (1.2 g, 98% yield) as brown oil.

LCMS (MM-ES+APCI) (M+H)⁺=282.3; Purity: 98.58% by ELSD-LCMS.

### Step 2: Synthesis of tert-butyl (2-(1-(3-aminopropyl)piperidin-4-yl)ethyl)carbamate (3)

In a tiny clave, Raney/Ni (0.209 g, 3.55 mmol) was added to a degassed solution of tert-butyl (2-(1-(2-cyanoethyl)piperidin-4-yl)ethyl)carbamate (1.0 g, 3.55 mmol) in EtOH (15 mL) at 25° C. under N₂ atmosphere. The reaction mixture was heated with 5 kg/H₂ at 60° C. for 16 hours. The reaction process was observed by LCMS. After completion of the reaction, the reaction mixture was filtered with celite, washed with EtOH (30 mL), and concentrated under reduced pressure to obtain tert-butyl (2-(1-(3-aminopropyl)piperidin-4-yl)ethyl)carbamate (1.02 g, 68.5% yield) as pale brown gum.

LCMS: (MM-ES+APCI) (M+H)⁺=286.3; Purity: 66.2% by ELSD-LCMS.

### Step 3: Synthesis of tert-butyl (2-(1-(3-((2-(2,6-dioxopiperidin-3 yl)-1,3-dioxoisoindolin-4-yl)amino)propyl)piperidin-4-yl)ethyl)carbamate (4)

DIPEA (0.612 ml, 3.50 mmol) and 2-(2,6-dioxopiperidin-3-yl)-4-fluoroisoindoline-1,3-dione (0.968 g, 3.50 mmol) were added to a solution of tert-butyl (2-(1-(3-aminopropyl)piperidin-4-yl)ethyl)carbamate (1.0 g, 3.50 mmol) in DMF (10 mL) at 25° C. under N₂ atmosphere. The reaction mixture was heated at 100° C. for 16 hours. The reaction process was observed by LCMS. After completion of the reaction, the reactant was diluted with cold water (30 mL) and extracted with EtOAc (2×30 mL). The combined organic phase was washed with brine (2×30 mL), dried over Na₂SO₄, and filtered and concentrated under reduced pressure. The residue was purified by flash column chromatography, eluted at 3-5% of MeOH/CH₃C₁₂ to obtain tert-butyl (2-(1-(3-((2-(2,6-dioxopiperidin-3-yl)-1,3-dioxoisoindolin-4-yl)amino)propyl)piperidin-4-yl)ethyl)carbamate (620 mg, 25.3% yield) as yellow gum.

LCMS (MM-ES+APCI) (M+H)⁺=542.3; Purity: 77.4% by LCMS.

### Step 4: Synthesis of 4-((3-(4-(2-aminoethyl)piperidin-1-yl)propyl)amino)-2-(2,6-dioxopiperidin-3 yl)isoindoline-1,3-dione 2,2,2-trifluoroacetate (5)

TFA (1.707 ml, 22.15 mmol) was added dropwise to a solution of tert-butyl (2-(1-(3-((2-(2,6-dioxopiperidin-3-yl)-1,3-dioxoisoindolin-4-yl)amino)propyl)piperidin-4-yl)ethyl)carbamate (0.6 g, 1.108 mmol) in DCM (10 mL) at 0° C. under N₂ atmosphere. The reaction mixture was stirred at 25° C. for 6 hours. The reaction process was observed by LCMS. After completion of the reaction, the reaction mixture was concentrated under reduced pressure, and the residue was dissolved in a mixture of DCM/MeOH (1/1, 30 mL). Si-carbonate (˜1 g) was added to the solution and stirred at 25° C. for 2 hours. The resin was filtered through celite and washed with MeOH (25 mL). The filtrate was concentrated under reduced pressure to obtain 4-((3-(4-(2-aminoethyl)piperidin-1-yl)propyl)amino)-2-(2,6-dioxopiperidin-3-yl)isoindoline-1,3-dione 2,2,2-trifluoroacetate (0.72 g, 100% yield) as greenish yellow gum.

LCMS (MM-ES+APCI) (M+H)⁺=442.3; Purity: 85.7% by LCMS.

### Step 5: Synthesis of 4-(2-(4-((4-((4-((2-chlorophenyl)carbamoyl)phenyl)amino)-5-fluoropyrimidin-2-yl) amino)phenyl)acetyl)-N-(2-(1-(3-((2-(2,6-dioxopiperidin-3-yl)-1,3-dioxoisoindolin-4-yl)amino)propyl)piperidin-4-yl)ethyl)piperazine-1-carboxamide (Compound 4)

TEA (153 mg, 1.510 mmol) and CDI (184 mg, 1.132 mmol) were added to a solution of 4-((3-(4-(2-aminoethyl)piperidin-1-yl)propyl)amino)-2-(2,6-dioxopiperidin-3-yl)isoindoline-1,3-dione 2,2,2-trifluoroacetate (490 mg, 0.755 mmol) in THE (5 mL) at 20 to 25° C. under N₂ atmosphere. The reaction mixture was stirred at 20 to 25° C. for 30 minutes. The reaction mixture was added with a solution of N-(2-chlorophenyl)-4-((5-fluoro-2-((4-(2-oxo-2-(piperazin-1-yl)ethyl)phenyl)amino)pyrimidin-4-yl)amino)benzamide 2,2,2-trifluoroacetate (305 mg, 0.453 mmol) and TEA (153 mg, 1.510 mmol) in THE (5 mL) at 20 to 25° C. The reaction mixture was stirred at 25° C. for 3 hours. The reaction process was observed by LCMS. After completion of the reaction, the reactant was diluted with water and extracted with EtOAc (2×30 mL). The combined organic material was dried over Na₂SO₄, and filtered and concentrated under reduced pressure. The residue was purified by wash column and eluted with 12-15% MeOH/DCM. The obtained purified material was purified again by prep-HPLC and freeze-dried to obtain Compound 4 (142 mg, 16.75% yield) as yellow solid. LCMS (MM-ES+APCI)(M+H)⁺=1027.3, HPLC: Rt-8.34; Purity=91.53%

¹H NMR (400 MHz, DMSO-d₆): δ=11.09 (br s, 1H), 9.92 (br s, 1H), 9.67 (br s, 1H), 9.30 (br s, 1H), 8.19 (d, J=3.6 Hz, 1H), 8.04 (d, J=8.8 Hz, 2H), 7.98 (d, J=8.8 Hz, 2H), 7.66 (dd, J1=8.0, J2=1.2 Hz 1H), 7.62 (d, J=8.4 Hz, 2H), 7.59-7.55 (m, 2H), 7.40 (td, J1=8.8, J2=1.60 Hz, 1H), 7.30 (td, J1=8.0, J2=1.60, Hz, 1H), 7.12 (m, 3H), 7.02 (d, J=7.2 Hz, 1H), 6.88 (t, J=6.0 Hz, 1H), 6.44 (t, J=5.6 Hz, 1H), 5.05 (dd, J1=12.4, J2=5.2 Hz, 1H), 3.67 (s, 2H), 3.44-3.40 (m, 5H), 3.22-3.18 (m, 5H), 3.05-3.00 (m, 3H), 2.91-2.83 (m, 4H), 2.61-2.56 (m, 2H) 2.04-2.00 (m, 1H), 1.83-1.79 (m, 2H), 1.73-1.70 (m, 2H), 1.59-1.57 (m, 2H), 1.31 (s, 2H), 1.21 (s, 3H)

### Example 5: Synthesis of Compound 5

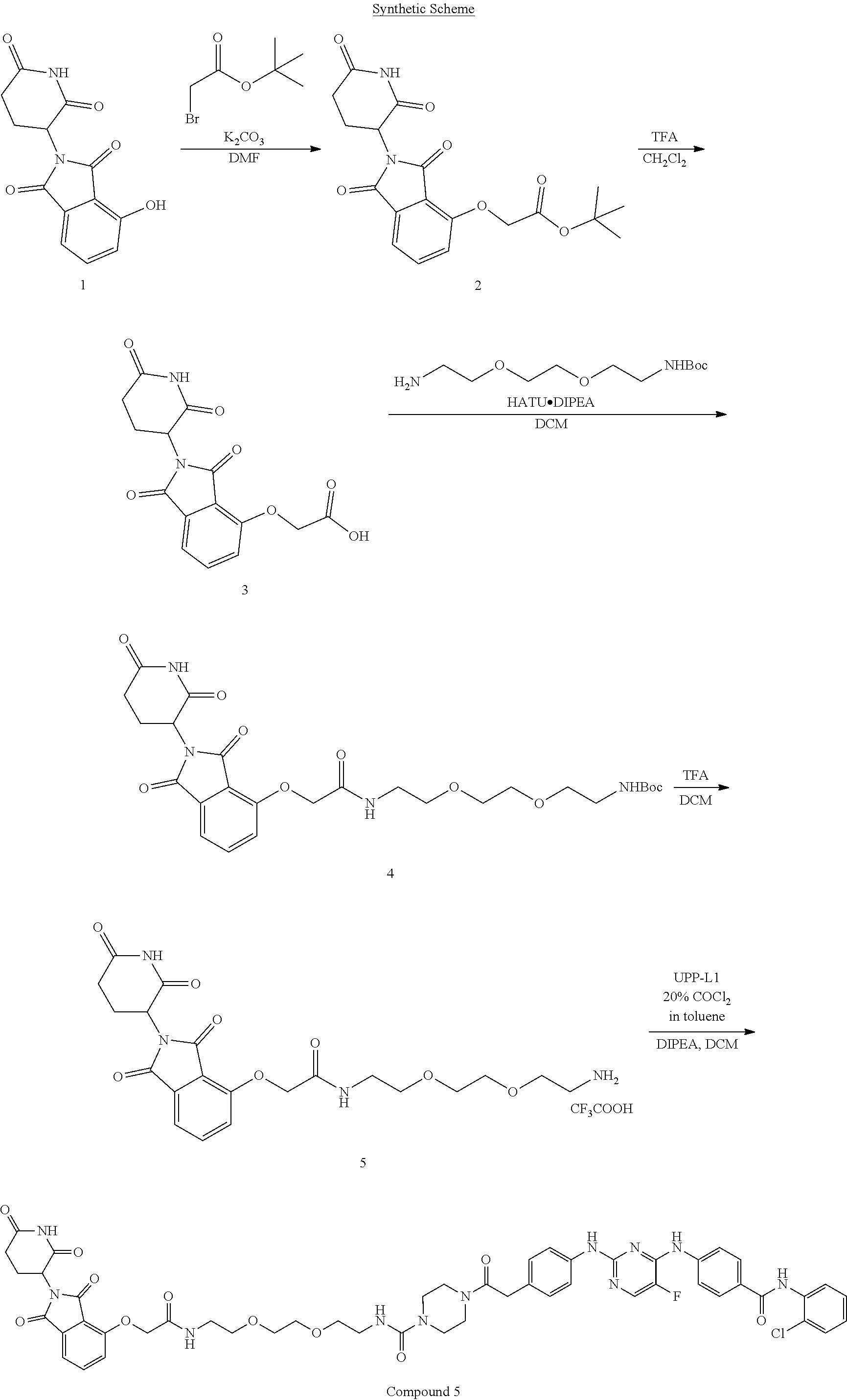

### Experimental Process

### Step 1: Synthesis of tert-butyl 2-((2-(2,6-dioxopiperidin-3 yl)-1,3-dioxoisoindolin-4-yl)oxy)acetate (2)

K₂CO₃ (1.512 g, 10.94 mmol) and tert-butyl 2-bromoacetate (1.063 ml, 7.29 mmol) were added to a stirred solution of 2-(2,6-dioxopiperidin-3-yl)-4-hydroxyisoindoline-1,3-dione (2.0 g, 7.29 mmol) in DMF (50 mL) at 25° C. The reaction mixture was stirred at 25° C. for 19 hours and the reaction was monitored by UPLC-MS. After completion of the reaction, the reactant was filtered and washed with EtOAc (10 mL). The filtrate was diluted with water (100 mL) and extracted with EtOAc (2×30 mL). The combined organic phase was concentrated under reduced pressure and the residue was purified by flash column and eluted with 30-40% EtOAc/hexane to obtain tert-butyl 2-((2-(2,6-dioxopiperidin-3-yl)-1,3-dioxoisoindolin-4-yl)oxy)acetate (2.7 g, 90% yield) as colorless solid.

UPLC-MS (MS ES−)(M−H)=387.2; Purity: 93.96% by UPLC-MS.

### Step 2: Synthesis of 2-((2-(2,6-dioxopiperidin-3-yl)-1,3-dioxoisoindolin-4-yl)oxy)acetic acid (3)

TFA (5.0 mL, 2.57 mmol) was added dropwise to a stirred solution of tert-butyl 2-((2-(2,6-dioxopiperidin-3-yl)-1,3-dioxoisoindolin-4-yl)oxy)acetate (1.0 g, 2.57 mmol) in DCM (20 mL) at 25° C. The reactant was maintained at 25° C. for 5 hours. The reaction process was observed by UPLC-MS, and after completion of the reaction, the reactant was concentrated under reduced pressure to obtain 2-((2-(2,6-dioxopiperidin-3-yl)-1,3-dioxoisoindolin-4-yl)oxy)acetic acid (830 mg, 76.45% yield) as brown solid.

UPLC-MS (MS ES+) (M+H)⁺=333.9; Purity: 78.85% by UPLC-MS.

### Step 3: Synthesis of tert-butyl (2-(2-(2-(2-((2-(2,6-dioxopiperidin-3 yl)-1,3-dioxoisoindolin-4-yl)oxy)acetamido)ethoxy)ethoxy)ethyl)carbamate (4)

DIPEA (1.314 ml, 7.52 mmol), tert-butyl (2-(2-(2-aminoethoxy)ethoxy)ethyl)carbamate (374 mg, 1.505 mmol) and HATU (858 mg, 2.257 mmol) were added to 2-((2-(2,6-dioxopiperidin-3-yl)-1,3-dioxoisoindolin-4-yl)oxy)acetic acid (500 mg, 1.505 mmol) in DCM (10 mL) at 0 to 10° C. under N₂ atmosphere. The reaction mixture was stirred at 25° C. for 16 hours. The reactant was monitored by UPLC-MS, and after completion of the reaction, the reactant was added with 20 mL of water. The organic phase was separated and the aqueous phase was extracted with EtOAc (2×10 mL). The combined organic phase was concentrated under reduced pressure, purified by flash column, and eluted with 15% EtOAc/pet ether to obtain tert-butyl (2-(2-(2-(2-((2-(2,6-dioxopiperidin-3-yl)-1,3-dioxoisoindolin-4-yl)oxy)acetamido)ethoxy)ethoxy)ethyl)carbamate (410 mg, 36.94% yield) as brown gum.

LCMS (MM-ES+APCI) (M+H)⁺=563.2; Purity: 76.31% by LCMS.

### Step 4: Synthesis of N-(2-(2-(2-aminoethoxy)ethoxy)ethyl)-2-((2-(2,6-dioxopiperidin-3 yl)-1,3-dioxoisoindolin-4-yl)oxy)acetamide 2,2,2-trifluoroacetate (5)

TFA (0.822 mL, 10.67 mmol) was added dropwise to a stirred solution of tert-butyl (2-(2-(2-(2-((2-(2,6-dioxopiperidin-3-yl)-1,3-dioxoisoindolin-4-yl)oxy)acetamido)ethoxy)ethoxy)ethyl)carbamate (400 mg, 0.711 mmol) in DCM (10 mL) at 0 to 10° C. under N₂ atmosphere. The reaction mixture was stirred at 25° C. for 3 hours. The reactant was observed by UPLC-MS. After completion of the reaction, the reactant was concentrated under reduced pressure to obtain N-(2-(2-(2-aminoethoxy)ethoxy)ethyl)-2-((2-(2,6-dioxopiperidin-3-yl)-1,3-dioxoisoindolin-4-yl)oxy)acetamide 2,2,2-trifluoroacetate (0.66 g) as brown gum.

LCMS (MM-ES+APCI) (M+H)⁺=463.1; Purity: 96.05% by LCMS.

### Step 5: Synthesis of 4-(2-(4-((4-((4-((2-chlorophenyl)carbamoyl)phenyl)amino)-5-fluoropyrimidin-2-yl)amino) phenyl)acetyl)-N-(2-(2-(2-(2-((2-(2,6-dioxopiperidin-3-yl)-1,3-dioxoisoindolin-4-yl)oxy)acetamido)ethoxy)ethoxy)ethyl)piperazine-1-carboxamide (Compound 5)

DIPEA (0.182 mL, 1.041 mmol) and Phosgene 20%/toluene (0.263 mL, 0.520 mmol) were added to a stirred solution of N-(2-(2-(2-aminoethoxy)ethoxy)ethyl)-2-((2-(2,6-dioxopiperidin-3-yl)-1,3-dioxoisoindolin-4-yl)oxy)acetamide 2,2,2-trifluoroacetate (300 mg, 0.520 mmol) in DCM (9 mL) at 0 to 10° C. under N₂ atmosphere. The reaction mixture was stirred at 0 to 10° C. for 30 minutes. The reaction mixture was added with a solution of N-(2-chlorophenyl)-4-((5-fluoro-2-((4-(2-oxo-2-(piperazin-1-yl)ethyl)phenyl)amino)pyrimidin-4-yl)amino)benzamide 2,2,2-trifluoroacetate (351 mg, 0.520 mmol) and DIPEA (0.182 mL, 1.041 mmol) in DCM (6 mL) at 0 to 10° C. The reaction mixture was stirred at 25° C. and observed by TLC. After completion of the reaction, the reactant was quenched with 15 mL of a NaHCO₃ solution. The organic phase was separated, and the aqueous phase was extracted with DCM (10 mL). The combined organic phase was concentrated under reduced pressure to obtain 320 mg of a compound. The compound was purified by Prep-HPLC and freeze-dried for 40 hours to obtain Compound 5 (108.0 mg, 18.98% yield) as pale yellow solid. LCMS (MM-ES+APCI)(M+H)⁺=1048, HPLC: Rt-7.39; Purity: 95.91%

¹H NMR (400 MHz, DMSO-d₆): δ=11.12 (br s, 1H), 9.89 (br s, 1H), 9.65 (br s, 1H), 9.28 (br s, 1H), 8.19 (d, J=3.6 Hz, 1H), 8.05-7.97 (m, 5H), 7.81 (dd, J1=8.4, J2=7.2 Hz, 1H), 7.66 (dd, J1=8.0, J2=1.6 Hz, 1H), 7.62 (d, J=8.8 Hz, 2H), 7.57 (td, J1=8.0, J2=1.2 Hz, 1H), 7.50 (d, J=6.8 Hz, 1H), 7.42-7.39 (m, 2H), 7.30 (td, J1=8.0, J2=1.6 Hz, 1H), 7.12 (d, J=8.4 Hz, 2H), 6.55 (t, J=4.8 Hz, 1H), 5.12 (dd, J1=12.8, J2=5.2 Hz, 1H), 4.79 (s, 2H), 3.67 (s, 2H), 3.50-3.35 (m, 14H), 3.40-3.12 (m, 6H), 2.91-2.85 (m, 1H), 2.62-2.55 (m, 2H), 2.05-2.04 (m, 1H)

### Example 6: Synthesis of Compound 6

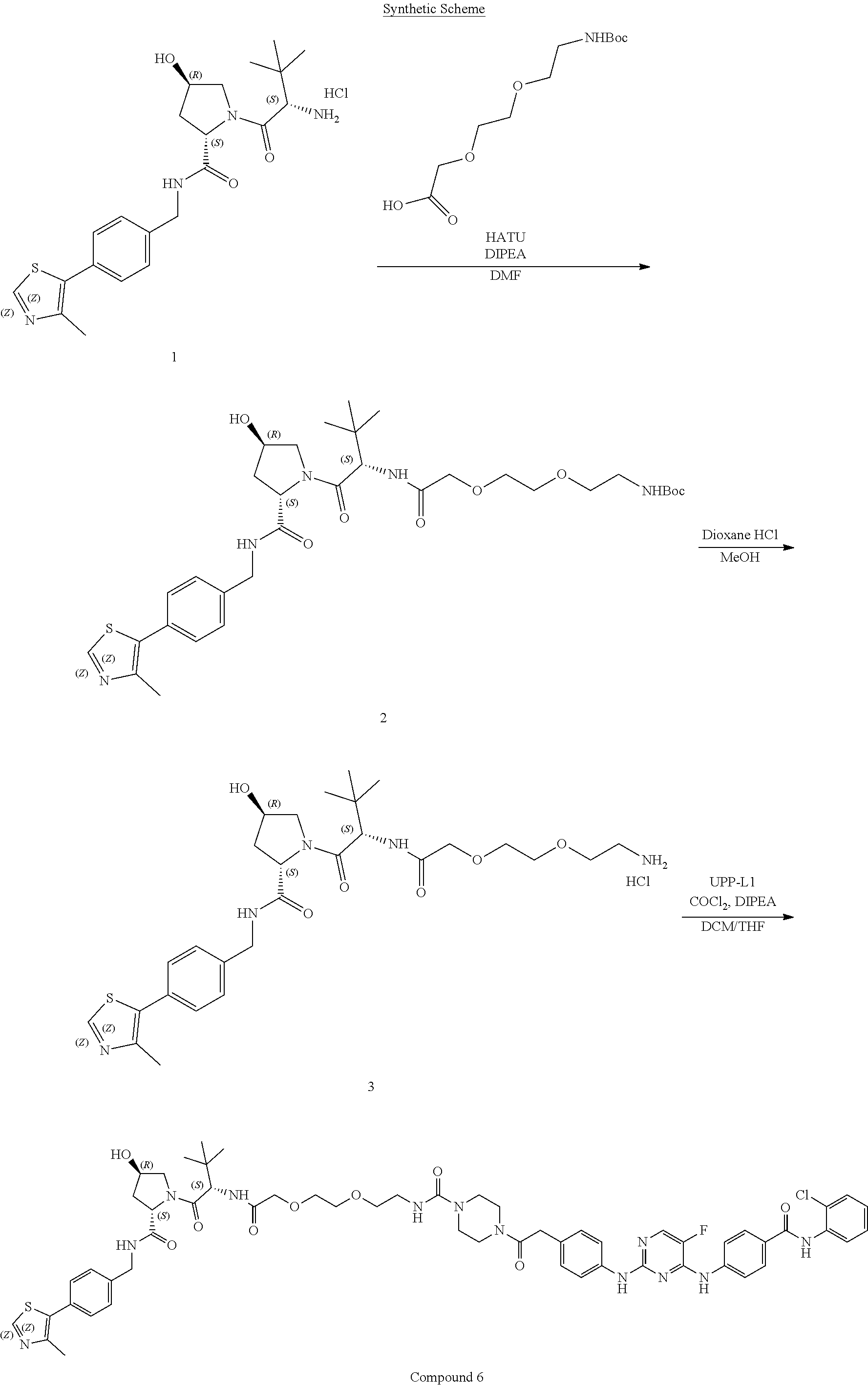

### Experimental Process

### Step 1: Synthesis of tert-butyl (2-(2-(2-(((S)-1-((2S,4R)-4-hydroxy-2-((4-(4-methylthiazol-5-yl)benzyl)carbamoyl)pyrrolidin-1 yl)-3,3-dimethyl-1-oxobutan-2-yl)amino)-2-oxoethoxy)ethoxy) ethyl) carbamate (2)

DIPEA (0.981 mL, 5.62 mmol), 2,2-dimethyl-4-oxo-3,8,11-trioxa-5-azatridecan-13-oic acid (542 mg, 2.059 mmol) and HATU (1068 mg, 2.81 mmol) were added to (2S,4R)-1-((S)-2-amino-3,3-dimethylbutanoyl)-4-hydroxy-N-(4-(4-methylthiazol-5-yl)benzyl) pyrrolidine-2-carboxamide hydrochloride (930 mg, 1.872 mmol) in DMF (10 mL) at 0 to 10° C. under N₂ atmosphere. The reaction mixture was stirred at 25° C. for 16 hours. The reaction was observed by LCMS, and after completion of the reaction, the reaction mixture was diluted with water (20 mL) and extracted with EtOAc (25 mL×2). The combined organic phase was washed with 10% Na₂SO₄ (20 mL) and concentrated under reduced pressure, and the obtained residue was purified by flash column chromatography and eluted with 5-7% MeOH/DCM to obtain tert-butyl(2-(2-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethoxy)ethyl)carbamate (650 mg, 49.8% yield) as off-white solid.

LCMS: (MM-ES+APCI) (M+H)⁺=676.3; Purity: 96.97% by LCMS.

### Step 2: Synthesis of (2S,4R)-1-((S)-2-(2-(2-(2-aminoethoxy)ethoxy)acetamido)-3,3-dimethylbutanoyl)-4-hydroxy-N-(4-(4-methylthiazol-5-yl)benzyl)pyrrolidine-2-carboxamide hydrochloride (3)

4 M HCl/dioxane (0.925 mL, 3.70 mmol) was added dropwise to tert-butyl (2-(2-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethoxy)ethyl)carbamate (250 mg, 0.370 mmol) in MeOH (5 mL) at 0 to 5° C. The reaction mixture was stirred at 25 to 30° C. for 3 hours. The reaction process was observed by TLC. After completion of the reaction, the reactant was concentrated under reduced pressure to obtain (2S,4R)-1-((S)-2-(2-(2-(2-aminoethoxy)ethoxy) acetamido)-3,3-dimethylbutanoyl)-4-hydroxy-N-(4-(4-methylthiazol-5-yl)benzyl)pyrrolidine-2-carboxamide hydrochloride (200 mg, 77.4% yield) as off-white solid.

LCMS: (MM-ES+APCI) (M+H)⁺=576.5; Purity: 84.21% by LCMS.

### Step 3: Synthesis of 4-(2-(4-((4-((4-((2-chlorophenyl) carbamoyl) phenyl) amino)-5 fluoropyrimidin-2 yl)amino)phenyl)acetyl)-N-(2-(2-(2-(((S)-1-((2S,4R)-4-hydroxy-2-((4-(4-methylthiazol-5 yl)benzyl)carbamoyl)pyrrolidin-1 yl)-3,3-dimethyl-1-oxobutan-2 yl)amino)-2-oxoethoxy)ethoxy)ethyl)piperazine-1-carboxamide (Compound 6)

DIPEA (84 mg, 0.653 mmol) and Phosgene 20%/toluene (242 mg, 0.490 mmol) were added to (2S,4R)-1-((S)-2-(2-(2-(2-aminoethoxy)ethoxy)acetamido)-3,3-dimethylbutanoyl)-4-hydroxy-N-(4-(4-methylthiazol-5-yl)benzyl)pyrrolidine-2-carboxamide hydrochloride (200 mg, 0.327 mmol) in DCM (5 mL) at 0 to 5° C. under N₂ atmosphere. The reaction mixture was stirred at 0 to 5° C. for 35 minutes. The reactant was added dropwise with N-(2-chlorophenyl)-4-((5-fluoro-2-((4-(2-oxo-2-(piperazin-1-yl)ethyl)phenyl)amino)pyrimidin-4-yl)amino)benzamide 2,2,2-trifluoroacetate (UPP-L1) (200 mg, 0.297 mmol) and DIPEA (84 mg, 0.653 mmol) in THE (5 mL) at 0 to 5° C. The reactant was slowly warmed to 25° C. and stirred for 1 hour. The reaction process was observed by UPLC-MS. After completion of the reaction, the reactant was quenched with a 10% NaHCO₃ (10 mL) solution and extracted with DCM (2×20 mL). The combined organic phase was dried over Na₂SO₄, and filtered and concentrated under reduced pressure. The residue was purified by prep-HPLC to obtain Compound 6 (123 mg, 30.8% yield) as off-white solid. LCMS (MM-ES+APCI)(M+H)⁺=1161.4, HPLC: Rt-7.74; Purity: 95.02%

¹H NMR (400 MHz, DMSO-d₆): δ=9.90 (br s, 1H), 9.65 (br s, 1H), 9.28 (br s, 1H), 8.97 (s, 1H), 8.58 (t, J=4 Hz, 1H), 8.18 (d, J=3.6 Hz, 1H), 8.04 (d, J=8.8 Hz, 2H), 7.98 (d, J=9.2 Hz, 2H), 7.66 (dd, J1=8.0, J2=1.6 Hz 1H), 7.62 (d, J=8.4 Hz, 2H), 7.56 (dd, J1=8.0, J2=1.2 Hz, 1H), 7.44-7.38 (m, 6H), 7.31 (td, J1=8.0, J2=1.60 Hz, 1H), 7.12 (d, J=8.8 Hz, 2H), 6.56 (t, J=5.6 Hz, 1H), 5.15 (d, J=3.2 Hz, 1H), 4.58 (d, J=9.6 Hz, 1H), 4.47-4.36 (m, 3H), 4.26 (dd, J1=16.0, J2=6 Hz 1H), 3.97 (s, 2H), 3.69-3.66 (m, 3H), 3.62-3.58 (m, 3H), 3.55-3.53 (m, 2H), 3.44-3.40 (m, 6H), 3.21-3.17 (m, 6H), 2.44 (s, 3H), 2.09-2.04 (m, 1H), 1.94-1.87 (m, 1H), 0.94 (s, 9H)

### Example 7: Synthesis of Compound 7

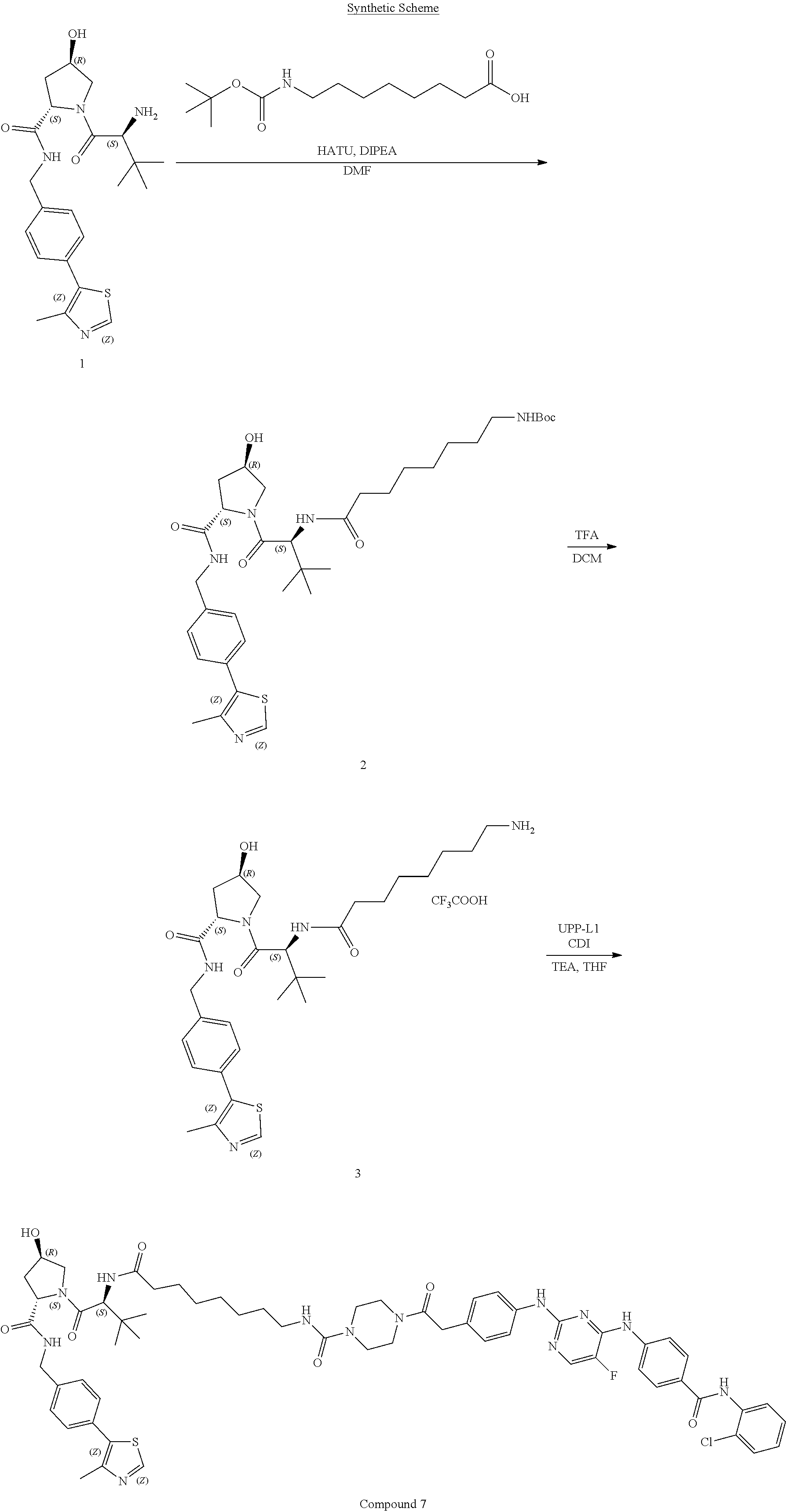

### Experimental Process

### Step 1: Synthesis of tert-butyl (8-(((S)-1-((2S,4R)-4-hydroxy-2-((4-(4-methylthiazol-5-yl)benzyl)carbamoyl)pyrrolidin-1 yl)-3,3-dimethyl-1-oxobutan-2-yl)amino)-8-oxooctyl) carbamate (2)

DIPEA (1.2 mL, 6.42 mmol) and HATU (1.2 g, 3.2 mmol) were added to a stirred solution of (2S,4R)-1-((S)-2-amino-3,3-dimethylbutanoyl)-4-hydroxy-N-(4-(4-methylthiazol-5-yl)benzyl)pyrrolidine-2-carboxamide hydrochloride (1.0 g, 2.14 mmol) and 8-((tert-butoxy carbonyl)amino) octanoic acid (0.554 g, 2.14 mmol) in DMF (10 mL) at 0 to 10° C. The reactant was stirred at 25° C. for 16 hours and the reaction process was observed by LCMS. After completion of the reaction, excess DMF was removed, diluted with water (30 mL) and extracted with EtOAc (2×30 mL). The organic phase was washed with brine (30 mL), dried over Na₂SO₄, and filtered and concentrated under reduced pressure. The obtained residue was purified by flash column chromatography and eluted with 2-5% MeOH/DCM. Desired fractions were concentrated under reduced pressure to obtain tert-butyl (8-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-8-oxooctyl)carbamate (0.9 g, 58.01% yield) as brown gum.

LCMS: (MM-ES+APCI) (M+H)⁺=672¬0.2; Purity: 93.39% by LCMS

### Step 2: Synthesis of (2S,4R)-1-((S)-2-(8-aminooctanamido)-3,3-dimethylbutanoyl)-4-hydroxy-N-(4-(4-methylthiazol-5 yl)benzyl)pyrrolidine-2-carboxamide 2,2,2-trifluoroacetate (3)

TFA (2.064 mL, 26.8 mmol) was added dropwise to a solution of tert-butyl (8 (((S)-1-((2S,4R)-4-hydroxy-2-((4-(4-methylthiazol-5-yl)benzyl)carbamoyl) pyrrolidin-1-yl)-3,3-dimethyl-1-oxobutan-2-yl)amino)-8-oxooctyl)carbamate (0.9 g, 1.339 mmol) in DCM (10 mL) at 0 to 5° C. under N₂ atmosphere. The reaction mixture was stirred at 25° C. for 6 hours. The reaction process was observed by TLC. After completion of the reaction, the reaction mixture was concentrated under reduced pressure to obtain (2S,4R)-1-((S)-2-(8-aminooctanamido)-3,3-dimethylbutanoyl)-4-hydroxy-N-(4-(4-methylthiazol-5-yl)benzyl)pyrrolidine-2-carboxamide 2,2,2-trifluoroacetate (1.0 g, 68.1% yield) as brown gum.

LCMS: (MM-ES+APCI) (M+H)⁺=572.0, Purity: 62.68% by LCMS

### Step 3: Synthesis of 4-(2-(4-((4-((4-((2-chlorophenyl) carbamoyl) phenyl) amino)-5-fluoropyrimidin-2 yl)amino)phenyl)acetyl)-N-(8-(((S)-1-((2S,4R)-4-hydroxy-2-((4-(4-methylthiazol-5 yl)benzyl)carbamoyl)pyrrolidin-1 yl)-3,3-dimethyl-1-oxobutan-2 yl)amino)-8-oxooctyl)piperazine-1-carboxamide (Compound 7)

TEA (139 mg, 1.371 mmol) and CDI (167 mg, 1.028 mmol) were added to a stirred solution of (2S,4R)-1-((S)-2-(8-aminooctanamido)-3,3-dimethylbutanoyl)-4-hydroxy-N-(4-(4-methylthiazol-5-yl)benzyl)pyrrolidine-2-carboxamide 2,2,2-trifluoroacetate (470 mg, 0.685 mmol) in THF (5 mL) at 20 to 25° C. under N₂ atmosphere. The reaction mixture was stirred at 20 to 25° C. for 30 minutes. After 30 minutes, the mixture was added with a solution of N-(2-chlorophenyl)-4-((5-fluoro-2-((4-(2-oxo-2-(piperazin-1-yl)ethyl)phenyl)amino)pyrimidin-4-yl)amino)benzamide 2,2,2-trifluoroacetate (370 mg, 0.548 mmol) (UPP-L1) and TEA (139 mg, 1.371 mmol) in THF (5 mL) at 20 to 25° C. The reaction mixture was stirred at 25° C. for 3 hours and the reaction process was observed by LCMS. After completion of the reaction, the reactant was diluted with water and extracted with EtOAc (2×30 mL). The combined organic phase was dried over Na₂SO₄, and filtered and concentrated under reduced pressure. The residue was purified by prep-HPLC to obtain Compound 7 (92 mg, 11.07% yield) as off-white solid. LCMS (MM-ES+APCI)(M+H)⁺=1157.4, HPLC: Rt-8.26; Purity: 95.48%

¹H NMR (400 MHz, DMSO-d₆): δ=9.92 (br s, 1H), 9.67 (br s, 1H), 9.30 (br s, 1H), 8.99 (s, 1H), 8.58 (t, J=5.6 Hz, 1H), 8.18 (d, J=3.6 Hz, 1H), 8.04 (d, J=8.8 Hz, 2H), 7.98 (d, J=8.8 Hz, 2H), 7.85 (d, J=9.2 Hz, 1H), 7.67 (dd, J1=8.0, J2=1.2 Hz 1H), 7.62 (d, J=8.4 Hz, 2H), 7.56 (dd, J1=6.8, J2=1.2 Hz, 1H), 7.44-7.37 (m, 5H), 7.31 (td, J1=8.8, J2=1.2 Hz, 1H), 7.13 (d, J=8.4 Hz, 2H), 6.48 (t, J=4.8 Hz, 1H), 5.13 (d, J=3.2 Hz, 1H), 4.54 (d, J=9.6 Hz, 1H), 4.47-4.41 (m, 2H), 4.35 (s, 1H), 4.22 (dd, J1=15.6, J2=5.2 Hz 1H), 3.67 (s, 3H), 3.44-3.38 (m, 5H), 3.21-3.18 (m, 4H), 2.99-2.94 (m, 2H), 2.45 (s, 3H), 2.29-2.21 (m, 1H), 1.14-2.10 (m, 1H), 2.06-2.01 (m, 1H), 1.93-1.88 (m, 1H), 1.51-1.44 (m, 2H), 1.37-1.34 (m, 2H), 1.22 (s, 6H), 0.93 (s, 9H)

### Example 8: Synthesis of Compound 8

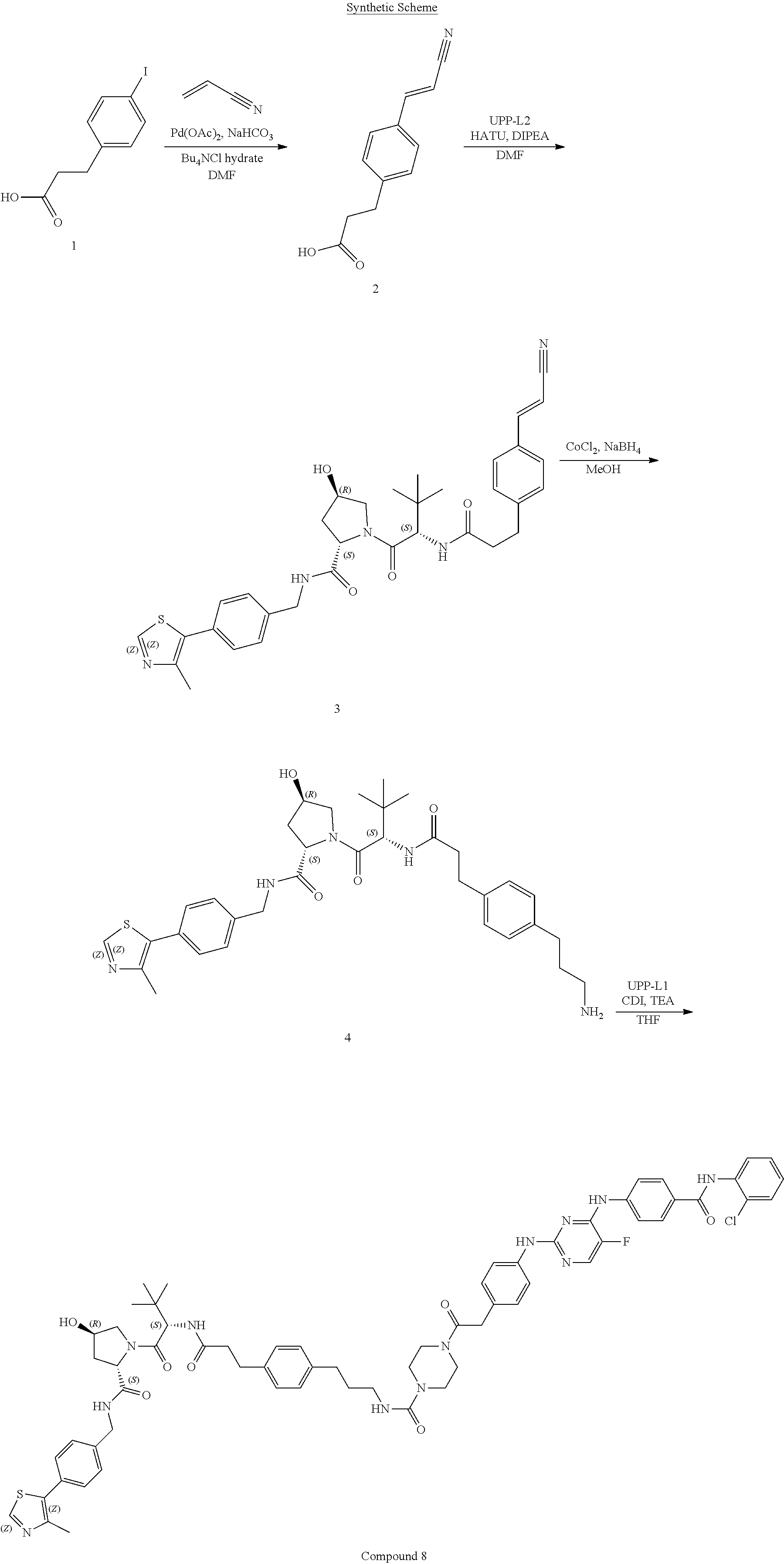

### Experimental Process

### Step 1: Synthesis of 3-(4-(2-cyanovinyl)phenyl)propanoic acid (2)

In a 50 mL sealed tube, NaHCO₃ (1.826 g, 21.73 mmol), Bu₄NCl hydrate (1.072 g, 3.62 mmol), ACN (1.153 g, 21.73 mmol) and Pd(OAc)₂ (0.163 g, 0.724 mmol) were added to a vacuum solution of 3-(4-iodophenyl)propanoic acid (2 g, 7.24 mmol) in DMF (25 mL) at 25° C. The reaction mixture was heated at 50° C. for 16 hours. The reaction process was observed by LCMS. After completion of the reaction, the reactant was filtered with celite and extracted with EtOAc (2×20 mL). The organic phase was washed with water (2×20 mL) and then washed with brine (20 mL). The obtained organic phase was concentrated under reduced pressure and the obtained residue was purified by flash column chromatography and eluted with 25-30% EtOH/pet ether. Desired fractions were concentrated under reduced pressure to obtain 3-(4-(2-cyanovinyl)phenyl)propanoic acid (251 mg, 17.15% yield) as off-white solid.

LCMS: (MM-ES+APCI) (M−H)⁻=200.4; Purity: 99.62% by LCMS

### Step 2: Synthesis of (2S,4R)-1-((S)-2-(3-(4-(2-cyanovinyl)phenyl)propanamido)-3,3-dimethylbutanoyl)-4-hydroxy-N-(4-(4-methylthiazol-5-yl)benzyl)pyrrolidine-2-carboxamide (3)

DIPEA (674 mg, 5.22 mmol), 3-(4-(2-cyanovinyl)phenyl)propanoic acid (350 mg, 1.739 mmol) and HATU (992 mg, 2.61 mmol) were added to a solution of (2S,4R)-1-((S)-2-amino-3,3-dimethylbutanoyl)-4-hydroxy-N-(4-(4-methylthiazol-5-yl)benzyl)pyrrolidine-2-carboxamide hydrochloride (812 mg, 1.739 mmol) (UPP-L2) in DMF (6 mL) at 0 to 5° C. under N₂ atmosphere. The reactant was stirred at 25° C. for 3 hours. The reaction process was observed by LCMS. After completion of the reaction, the reaction mixture was diluted with cold water (20 mL) and extracted with EtOAc (2×20 mL). The combined organic phase was washed with a 10% NaHCO₃ (30 mL) solution and brine (2×20 mL). The obtained organic phase was dried over Na₂SO₄, and filtered and concentrated under reduced pressure. The obtained residue was purified by flash column chromatography (100-200 mesh) and eluted with 3-5% MeOH/DCM to obtain (2S,4R)-1-((S)-2-(3-(4-(2-cyanovinyl)phenyl)propanamido)-3,3-dimethylbutanoyl)-4-hydroxy-N-(4-(4-methylthiazol-5-yl)benzyl)pyrrolidine-2-carboxamide (650 mg, 56.8% yield) as off-white solid.

LCMS: (MM-ES+APCI)(M+H)⁺=614.2; Purity: 93.29% by LCMS

### Step 3: Synthesis of (2S,4R)-1-((S)-2-(3-(4-(3-aminopropyl)phenyl)propanamido)-3,3-dimethylbutanoyl)-4-hydroxy-N-(4-(4-methylthiazol-5-yl)benzyl)pyrrolidine-2-carboxamide (4)

CoCl₂ (190 mg, 1.466 mmol) and NaBH₄ (92 mg, 2.444 mmol) were added to a solution of (2S,4R)-1-((S)-2-(3-(4-(2-cyanovinyl)phenyl)propanamido)-3,3-dimethylbutanoyl)-4-hydroxy-N-(4-(4-methylthiazol-5-yl)benzyl)pyrrolidine-2-carboxamide (300 mg, 0.489 mmol) in MeOH (10 mL) at 0 to 5° C. under N₂ atmosphere. The reaction mixture was stirred at 25° C. for 16 hours. The reaction process was observed by LCMS. After completion of the reaction, the reactant was filtered through celite and concentrated under reduced pressure. The obtained residue was diluted with an ammonium hydroxide solution (20 mL) and extracted with DCM (2×20 mL). The organic phase was dried over Na₂SO₄, filtered, and concentrated under reduced pressure to obtain (2S,4R)-1-((S)-2-(3-(4-(3-aminopropyl)phenyl)propanamido)-3,3-dimethylbutanoyl)-4-hydroxy-N-(4-(4-methylthiazol-5-yl)benzyl)pyrrolidine-2-carboxamide (202 mg, 40.0% yield) as pale brown gum.

LCMS: (MM-ES+APCI) (M+H)⁺=620.3; Purity: 60.05% by LCMS

### Step 4: Synthesis of 4-(2-(4-((4-((4-((2-chlorophenyl)carbamoyl)phenyl)amino)-5 fluoropyrimidin-2-yl)amino)phenyl)acetyl)-N-(3-(4-(3-(((S)-1-((2S,4R)-4-hydroxy-2-((4-(4-methylthiazol-5-yl)benzyl)carbamoyl)pyrrolidin-1 yl)-3,3-dimethyl-1-oxobutan-2-yl)amino)-3-oxopropyl)phenyl)propyl)piperazine-1-carboxamide (Compound 8)

TEA (0.067 mL, 0.484 mmol) and CDI (78 mg, 0.484 mmol) were added to a solution of (2S,4R)-1-((S)-2-(3-(4-(3-aminopropyl)phenyl)propanamido)-3,3-dimethylbutanoyl)-4-hydroxy-N-(4-(4-methylthiazol-5-yl)benzyl)pyrrolidine-2-carboxamide (200 mg, 0.323 mmol) in THF (5 mL) at 0 to 5° C. under N₂ atmosphere. The reaction solution was stirred at 25° C. for 30 minutes. The reaction mixture was added dropwise with a solution of N-(2-chlorophenyl)-4-((5-fluoro-2-((4-(2-oxo-2-(piperazin-1-yl)ethyl)phenyl)amino)pyrimidin-4-yl)amino)benzamide 2,2,2-trifluoroacetate (UPP-L1) (130 mg, 0.194 mmol) and TEA (0.067 mL, 0.484 mmol) in THF (5 mL) at 0 to 5° C. The reaction solution was stirred at 25° C. for 12 hours. The reaction process was observed by LCMS. After completion of the reaction, the reactant was diluted with water (20 mL) and extracted with EtOAc:THF (1:1) (2×20 mL). The combined organic phase was dried over Na₂SO₄, and filtered and concentrated under reduced pressure to obtain Compound 8 (340 mg, 16.6% by LCMS) as yellow solid. LCMS: (MM-ES+APCI)(M+H)⁺=1205.4; Purity: 16.40% by LCMS

¹H NMR (400 MHz, DMSO-d₆): δ=9.91 (br s, 1H), 9.30 (br s, 1H), 8.99 (s, 1H), 8.58 (t, 1H), 8.19 (d, J=3.6 Hz, 1H), 8.04 (d, J=8.8 Hz, 2H), 7.98 (d, J=8.8 Hz, 2H), 7.93 (d, J=9.6 Hz, 1H), 7.66 (dd, J1=8.0, J2=1.6 Hz, 1H), 7.62 (d, J=8.8 Hz, 2H), 7.56 (dd, J1=8.0 Hz, J2=1.6 Hz, 1H), 7.43-7.37 (m, 5H), 7.30 (td, J1=8.0, J2=1.60 Hz, 1H), 7.13-7.06 (m, 6H), 6.52 (t, J=5.2 Hz, 1H), 6.09 (s, 1H), 5.15 (d, J=9.6 Hz, 1H), 4.55 (m, 1H), 4.47-4.41 (m, 2H), 4.36 (s, 1H), 4.22 (dd, J1=16.0, J2=5.6 Hz, 1H), 3.67 (s, 3H), 3.45-3.40 (m, 5H), 3.23-3.19 (m, 4H), 3.03-2.98 (m, 2H), 2.82-2.72 (m, 4H), 2.61-2.58 (m, 2H), 2.44 (s, 3H), 2.07-2.01 (m, 1H), 1.94-1.88 (m, 1H), 1.69-1.61 (m, 2H), 0.89 (s, 9H)

### Example 9: Synthesis of Compound 9

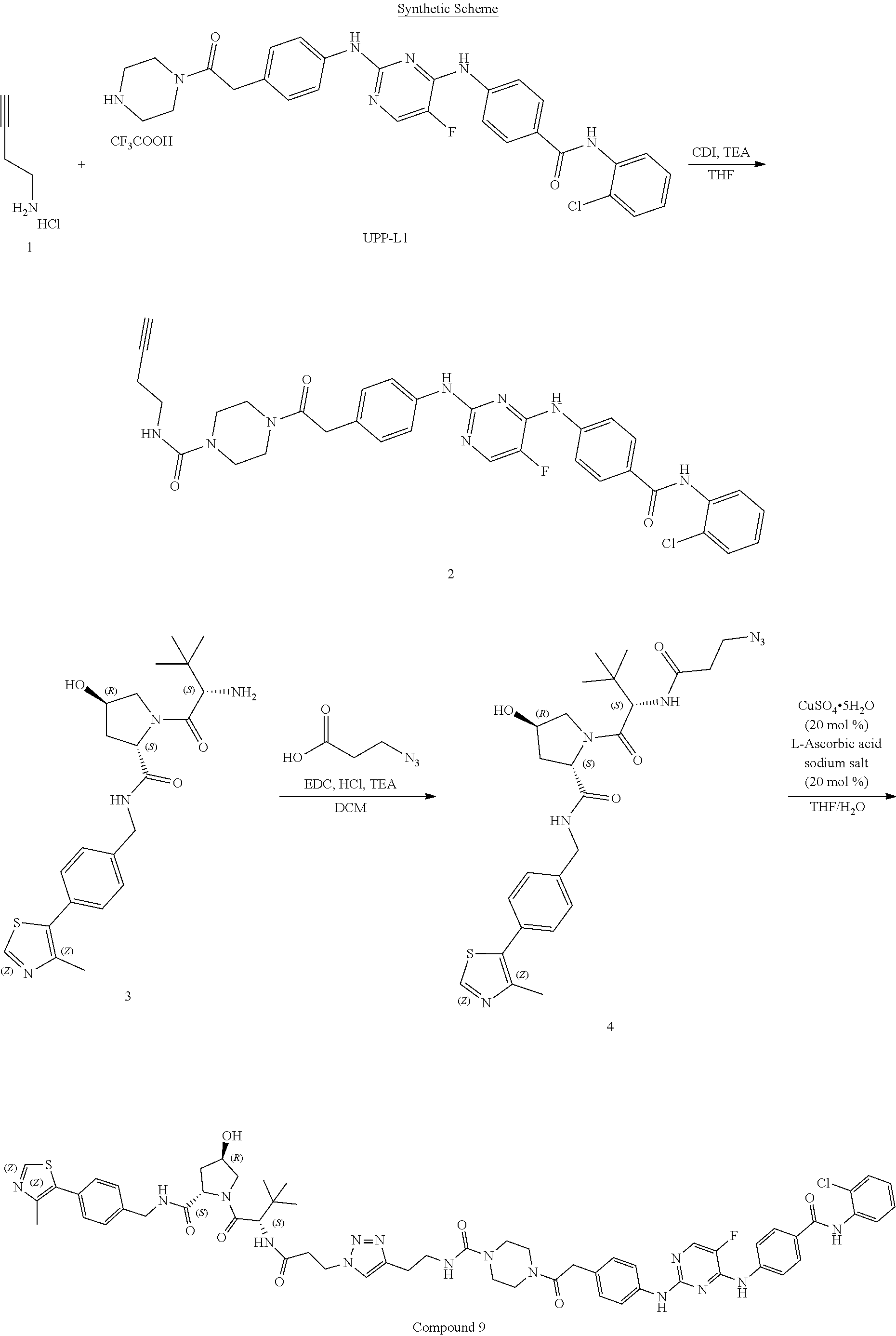

### Experimental Process

### Step 1: Synthesis of N-(but-3 yn-1-yl)-4-(2-(4-((4-((4-((2-chlorophenyl)carbamoyl)phenyl)amino)-5-fluoro pyrimidin-2-yl)amino)phenyl)acetyl)piperazine-1-carboxamide (2)

TEA (0.139 mL, 0.995 mmol) and CDI (81 mg, 0.497 mmol) were added to a stirred solution of but-3-yn-1-amine hydrochloride (35 mg, 0.332 mmol) in THE (3 mL) at 25° C. under N₂ atmosphere. The reaction mixture was stirred for 30 minutes. The reaction mixture was added with a solution of N-(2-chlorophenyl)-4-((5-fluoro-2-((4-(2-oxo-2-(piperazin-1-yl)ethyl)phenyl)amino)pyrimidin-4-yl)amino)benzamide 2,2,2-trifluoroacetate (156 mg, 0.232 mmol) (UPP-L1) in THE (3 mL) and TEA (0.139 mL, 0.995 mmol), and then stirred at 25° C. for 16 hours. The reaction process was observed by TLC. After completion of the reaction, the reaction mixture was quenched with water (10 mL). The aqueous phase and the organic phase were separated. The aqueous phase was extracted with 20% MeOH/DCM (3×10 mL). The combined organic phase was dried over Na₂SO₄, and filtered and concentrated under reduced pressure to obtain N-(but-3-yn-1-yl)-4-(2-(4-((4-((4-((2-chlorophenyl)carbamoyl)phenyl)amino)-5-fluoro pyrimidin-2-yl)amino)phenyl)acetyl)piperazine-1-carboxamide (125 mg, 0.191 mmol, 57.5% yield) as yellow solid.

LCMS: (MM-ES+APCI) (M+H)⁺=655.2; Purity: 94.44% by LCMS

### Step 2: Synthesis of (2S,4R)-1-((S)-2-(3-azidopropanamido)-3,3-dimethylbutanoyl)-4-hydroxy-N-(4-(4-methylthiazol-5 yl)benzyl)pyrrolidine-2-carboxamide (4)

TEA (0.090 mL, 0.642 mmol), 3-azidopropanoic acid (37.0 mg, 0.321 mmol), and EDC·HCl (61.6 mg, 0.321 mmol) were added to a solution of (2S,4R)-1-((S)-2-amino-3,3-dimethylbutanoyl)-4-hydroxy-N-(4-(4-methylthiazol-5-yl)benzyl)pyrrolidine-2-carboxamide hydrochloride (100 mg, 0.214 mmol) in DCM (3 mL) at 0 to 5° C. under N₂ atmosphere. The reaction solution was stirred at 25 to 30° C. for 3 hours. The reaction process was observed by UPLC-MS. After completion of the reaction, the reactant was diluted with water (10 mL) and extracted with DCM (2×10 mL). The combined organic phase was washed with 10% NaHCO₃ (20 mL), dried over Na₂SO₄ and concentrated under reduced pressure to obtain (2S,4R)-1-((S)-2-(3-azidopropanamido)-3,3-dimethylbutanoyl)-4-hydroxy-N-(4-(4-methylthiazol-5-yl)benzyl)pyrrolidine-2-carboxamide (108 mg, 79% yield) as colorless gum.

LCMS: (MM-ES+APCI) (M+H)⁺=528.2; Purity: 82.41% by LCMS

### Step 3: Synthesis of 4-(2-(4-((4-((4-((2-chlorophenyl)carbamoyl)phenyl)amino)-5-fluoropyrimidin-2-yl)amino)phenyl)acetyl)-N-(2-(1-(3-(((S)-1-((2S,4R)-4-hydroxy-2-((4-(4-methylthiazol-5-yl)benzyl)carbamoyl)pyrrolidin-1 yl)-3,3-dimethyl-1-oxobutan-2-yl)amino)-3-oxopropyl)-1H-1,2,3-triazol-4-yl)ethyl)piperazine-1-carboxamide (Compound 9)

L-ascorbic acid sodium salt (7.88 mg, 0.040 mmol), N-(but-3-yn-1-yl)-4-(2-(4-((4-((4-((2-chlorophenyl)carbamoyl)phenyl)amino)-5-fluoropyrimidin-2-yl)amino) phenyl)acetyl) piperazine-1-carboxamide (78 mg, 0.119 mmol), and CuSO₄·5H₂O (9.94 mg, 0.040 mmol) were sequentially added to a stirred solution of (2S,4R)-1-((S)-2-(3-azidopropanamido)-3,3-dimethylbutanoyl)-4-hydroxy-N-(4-(4-methylthiazol-5-yl)benzyl)pyrrolidine-2-carboxamide (105 mg, 0.199 mmol) in THF (3 mL) and water (1.5 mL) at 25° C. The reaction suspension was stirred at 25° C. for 16 hours. The reaction process was observed by LCMS. After completion of the reaction, the reaction mixture was filtered and washed with THF (10 mL). The filtrate was concentrated under reduced pressure to obtain Compound 9 (150 mg) as yellow solid. LCMS: (MM-ES+APCI)(M+H)⁺=1182.1; Purity: 25.24% by LCMS

¹H NMR (400 MHz, DMSO-d₆): δ=9.91 (br s, 1H), 9.66 (br s, 1H), 9.29 (br s, 1H), 8.98 (s, 1H), 8.60-8.56 (m, 1H), 8.18 (d, J=3.6 Hz, 1H), 8.14 (d, J=9.2 Hz, 1H), 8.04 (d, J=9.3 Hz, 2H), 7.98 (d, J=8.8 Hz, 2H), 7.71 (s, 1H), 7.67-7.61 (m, 3H), 7.56 (dd, J1=8.0, J2=1.6 Hz, 1H), 7.42-7.36 (m, 5H), 7.31 (td, J1=8.0, J2=1.60 Hz, 1H), 7.13 (d, J=8.4 Hz, 2H), 6.71 (t, J=5.2 Hz, 1H), 5.15 (d, J=3.6 Hz, 1H), 4.58-4.42 (m, 5H), 4.36 (s, 1H), 4.22 (dd, J1=16.0, J2=5.6 Hz, 1H), 3.67 (s, 3H), 3.51-3.42 (m, 5H), 3.23-3.17 (m, 6H), 2.91-2.79 (m, 1H), 2.77-2.71 (m, 3H), 2.44 (s, 3H), 2.08-2.03 (m, 1H), 1.94-1.87 (m, 1H), 0.88 (s, 9H)

### Example 10: Synthesis of Compound 10

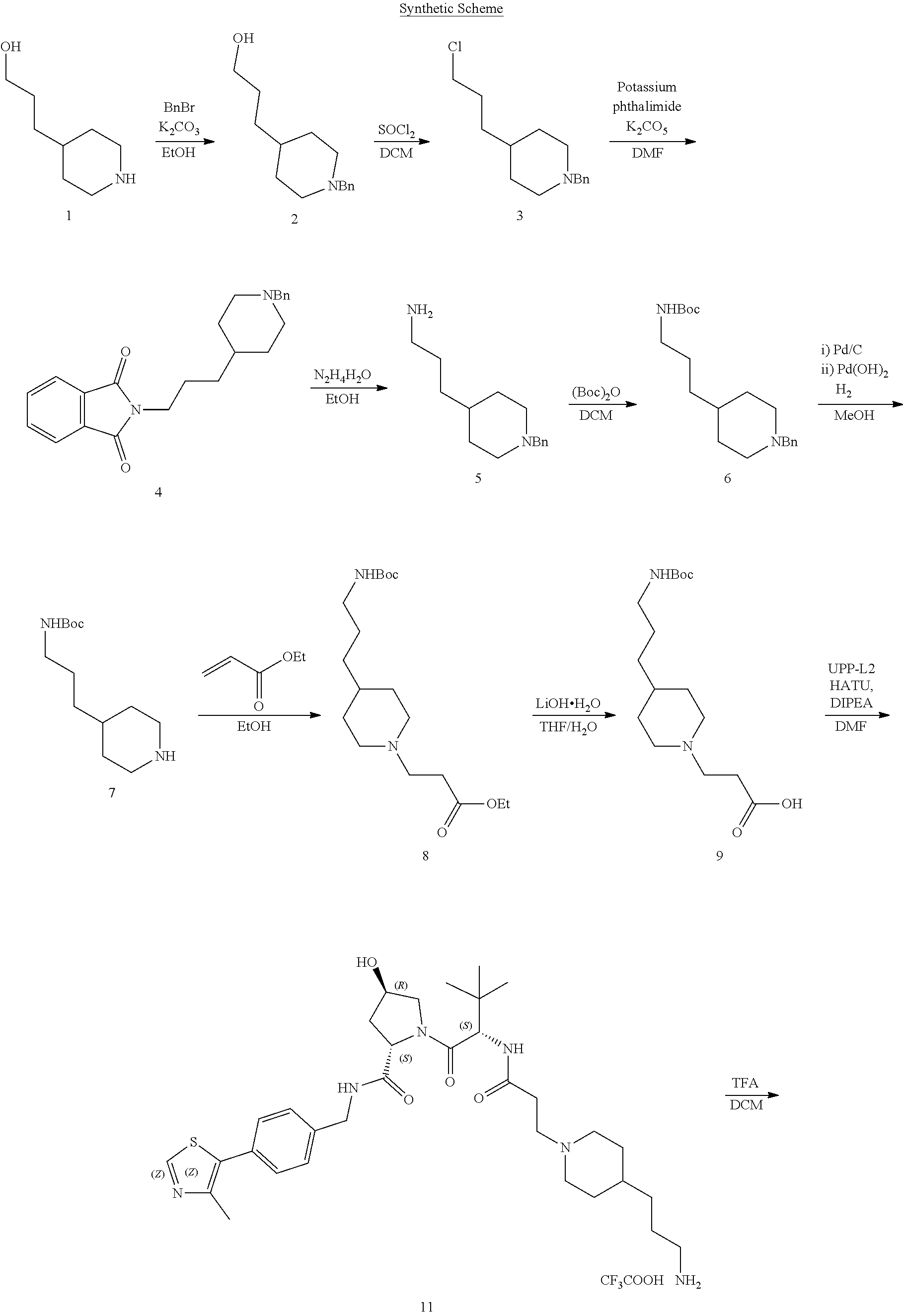

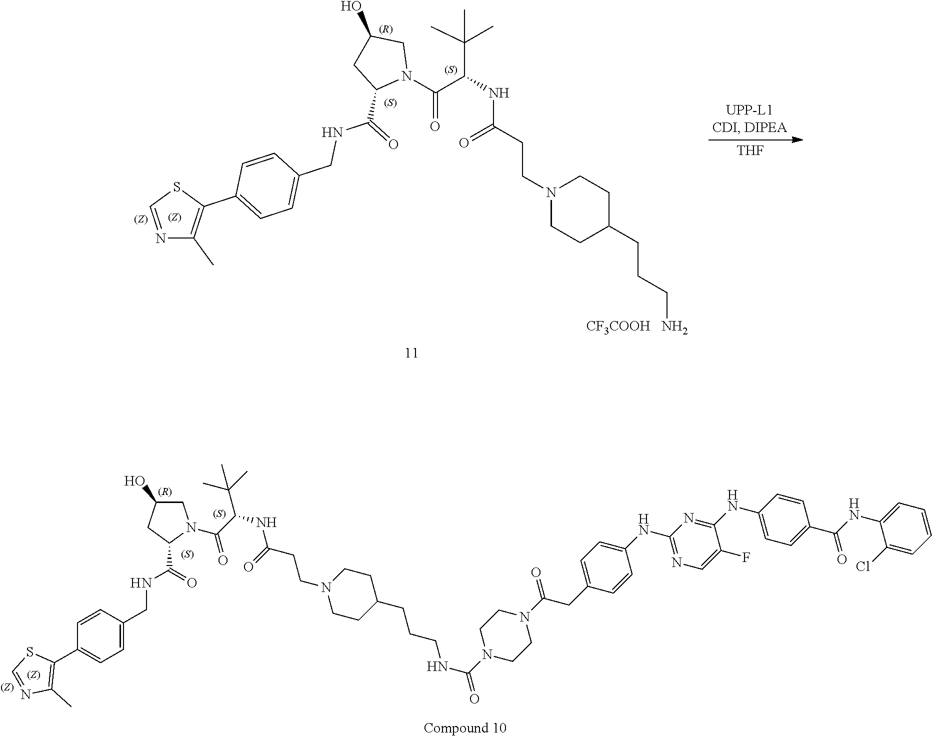

### Experimental Process

### Step 1: Synthesis of 3-(1-benzylpiperidin-4-yl)propan-1-ol (2)

K₂CO₃ (8.68 g, 62.8 mmol) and (bromomethyl)benzene (5.91 g, 34.6 mmol) were added to a suspension of 3-(piperidin-4-yl)propan-1-ol (4.5 g, 31.4 mmol) in EtOH (45 mL) at 25° C. under N₂ atmosphere. The reaction mixture was refluxed at 90° C. for 3 hours. The reaction process was observed by TLC. After completion of the reaction, the excess solvent was removed in a vacuum state at 45° C. The obtained residue was diluted with water (50 mL) and extracted with DCM (2×50 mL). The combined organic phase was washed with brine (50 mL), dried over Na₂SO₄, and filtered and concentrated under reduced pressure to obtain 3-(1-benzylpiperidin-4-yl)propan-1-ol (7.3 g, 59.5% yield) as brown gum. LCMS: (MM-ES+APCI) (M+H)⁺=234.2; Purity: 59.8% by LCMS

### Step 2: Synthesis of 1-benzyl-4-(3-chloropropyl)piperidine (3)

SOCl₂ (6.59 g, 55.4 mmol) was added dropwise to a solution of 3-(1-benzylpiperidin-4-yl)propan-1-ol (7.3 g, 18.46 mmol) in DCM (75 mL) at 20 to 25° C. under N₂ atmosphere. The reactant was heated at 45° C. for 3 hours and the reaction process was observed by TLC. After completion of the reaction, the reaction mixture was concentrated under reduced pressure. The residue was diluted with water and extracted with DCM (2×50 mL). The combined organic phase was washed with a bicarbonate solution (2×50 mL). The obtained organic phase was dried over Na₂SO₄, and filtered and concentrated under reduced pressure. The residue was purified by flash column chromatography, and eluted with 17-20% EtOAc/PE. Desired fractions were concentrated under reduced pressure to obtain 1-benzyl-4-(3-chloropropyl)piperidine (3.23 g, 62.3% yield). LCMS: (MM-ES+APCI) (M+H)⁺=252.2; Purity: 89.7% by LCMS

### Step 3: Synthesis of 2-(3-(1-benzylpiperidin-4-yl)propyl)isoindoline-1,3-dione (4)

K₂CO₃ (4.39 g, 31.8 mmol) and potassium phthalimide (1.765 g, 9.53 mmol) were added to a suspension of 1-benzyl-4-(3-chloropropyl)piperidine (1.6 g, 6.35 mmol) in DMF (25 mL) at 25° C. under N₂ atmosphere. The reaction mixture was stirred at 100° C. for 16 hours and the reaction process was observed by LCMS. To 81.8% of a desired compound was confirmed in LCMS. The excess solvent was removed and concentrated. The obtained residue was diluted with water (30 mL) and extracted with EtOAc (2×30 mL). The combined organic phase was washed with water (30 mL) and brine (30 mL). The organic phase was dried over Na₂SO₄, and filtered and concentrated under reduced pressure to obtain 2-(3-(1-benzylpiperidin-4-yl)propyl)isoindoline-1,3-dione (2.05 g, 53.2% yield) as off-white solid. LCMS: (MM-ES+APCI) (M+H)⁺=363.2; Purity: 59.7% by LCMS

### Step 4: Synthesis of 3-(1-benzylpiperidin-4-yl)propan-1-amine (5)

N₂H₄·H₂O was added to a solution of 2-(3-(1-benzylpiperidin-4-yl)propyl)isoindoline-1,3-dione (2.0 g, 3.26 mmol) in EtOH (30 mL) at 25° C. The reaction mixture was heated at 80° C. for 3 hours. The reaction process was monitored by LCMS. After completion of the reaction, DCM (30 mL) was added to the mixture at 25° C. The residue was filtered through celite and washed with DCM (30 mL), and the filtrate was concentrated under reduced pressure. The residue was added in DCM (30 mL) and filtered through cotton, and then the filtrate was concentrated under reduced pressure. The obtained compound was purified by neutral alumina column chromatography and eluted with 10-12% MeOH/DCM to obtain 3-(1-benzylpiperidin-4-yl)propan-1-amine (280 mg, 27.4% yield) as brown gum. LCMS: (MM-ES+APCI) (M+H)⁺=233.2; Purity: 73.9% by LCMS.

### Step 5: Synthesis of tert-butyl (3-(1-benzylpiperidin-4-yl)propyl)carbamate (6)

Boc-anhydride (0.275 mL, 1.183 mmol) was added dropwise to a solution of 3-(1-benzylpiperidin-4-yl)propan-1-amine (275 mg, 1.183 mmol) in DCM (5 mL) at 25° C. under N₂ atmosphere. The reaction solution was stirred at 25° C. for 16 hours and the reaction process was observed by LCMS. After completion of the reaction, the reaction mixture was diluted with water (20 mL) and extracted with DCM (2×20 mL). The combined organic phase was dried over Na₂SO₄ and concentrated under reduced pressure to obtain tert-butyl (3-(1-benzylpiperidin-4-yl)propyl)carbamate (370 mg, 90% yield) as brown gum. LCMS: (MM-ES+APCI) (M+H)⁺=333.2; Purity: 95.3% by LCMS

### Step 6: Synthesis of tert-butyl (3-(piperidin-4-yl)propyl)carbamate (7)

In 50 mL RBF, 10% Pd(OH)₂ (78 mg, 0.556 mmol) and Pd/C (59.2 mg, 0.556 mmol) were added to a degassed solution of tert-butyl (3-(1-benzylpiperidin-4-yl)propyl)carbamate (370 mg, 1.113 mmol) in MeOH (25 mL) at 25° C. under N₂ atmosphere. The reaction mixture was stirred with a H₂ bladder at 25° C. for 16 hours. The reaction process was observed by LCMS. After completion of the reaction, the reactant was filtered with celite and washed with MeOH (20 mL). The filtrate was concentrated under reduced pressure to obtain tert-butyl (3-(piperidin-4-yl)propyl)carbamate (260 mg, 79% yield) as colorless gum. LCMS: (MM-ES+APCI) (M+H)⁺=243.2; Purity: 81.5% by LCMS

### Step 7: Synthesis of ethyl 3-(4-(3-((tert-butoxycarbonyl)amino)propyl)piperidin-1-yl)propanoate (8)

Ethyl acrylate (215 mg, 2.146 mmol) was added to a suspension of tert-butyl (3-(piperidin-4-yl)propyl)carbamate (260 mg, 1.073 mmol) in EtOH (10 mL) at 25° C. under N₂ atmosphere. The reaction mixture was refluxed at 90° C. for 16 hours and the reaction process was monitored by LCMS. After completion of the reaction, the reactant was concentrated under reduced pressure and the obtained residue was diluted with water (10 mL) and extracted with DCM (2×10 mL). The combined organic phase was dried over Na₂SO₄ and concentrated under reduced pressure to obtain ethyl 3-(4-(3-((tert-butoxycarbonyl)amino)propyl)piperidin-1-yl)propanoate (362 mg, 87% yield) as brown gum. LCMS: (MM-ES+APCI) (M+H)⁺=343.3; Purity: 88.5% by LCMS

### Step 8: Synthesis of 3-(4-(3-((tert-butoxycarbonyl)amino)propyl)piperidin-1-yl)propanoic acid (9)

LiOH·H₂O was added to a solution of ethyl 3-(4-(3-((tert-butoxycarbonyl)amino)propyl)piperidin-1-yl)propanoate (360 mg, 1.051 mmol) in THE (3 mL) and water (1.5 mL) at 25° C. The reaction mixture was stirred at 25° C. for 2 hours and the reaction process was observed by LCMS. After completion of the reaction, the reaction mixture was acidified with a 1.5 N HCl solution (1 mL) and concentrated under reduced pressure. The residue was concentrated under reduced pressure with toluene (3×10 mL) to obtain 3-(4-(3-((tert-butoxycarbonyl)amino)propyl)piperidin-1-yl)propanoic acid (302 mg, 91% yield) as off-white solid. LCMS: (MM-ES+APCI) (M+H)⁺=315.3

### Step 9: Synthesis of tert-butyl (3-(1-(3-(((S)-1-((2S,4R)-4-hydroxy-2-((4-(4-methylthiazol-5-yl)benzyl)carbamoyl)pyrrolidin-1 yl)-3,3-dimethyl-1-oxobutan-2-yl)amino)-3-oxopropyl)piperidin-4-yl)propyl)carbamate (10)

DIPEA (0.370 g, 2.86 mmol) and HATU (0.472 g, 1.240 mmol) were added to a stirred solution of (2S,4R)-1-((S)-2-amino-3,3-dimethylbutanoyl)-4-hydroxy-N-(4-(4-methylthiazol-5-yl)benzyl)pyrrolidine-2-carboxamide hydrochloride (0.446 g, 0.954 mmol) (UPP-L2) and 3-(4-(3-((tert-butoxycarbonyl)amino)propyl)piperidin-1-yl)propanoic acid (0.3 g, 0.954 mmol) in DMF (5 mL) at 25° C. under N₂ atmosphere. The reaction mixture was stirred at 25° C. for 3 hours and the reaction was observed by UPLC-MS. After completion of the reaction, the reaction mixture was diluted with water (20 mL) and extracted with EtOAc (2×20 mL). The combined organic phase was washed with a 10% NaHCO₃ solution (20 mL), dried over Na₂SO₄, and concentrated under reduced pressure. The obtained compound was purified by flash column chromatography and eluted with 3-5% MeOH/CH₂C₁₂. Desired fractions were concentrated under reduced pressure to obtain tert-butyl (3-(1-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropyl)piperidin-4-yl)propyl)carbamate (326 mg, 43.9% yield) as brown gum. LCMS: (MM-ES+APCI) (M+H)⁺=727.5; Purity: 93.5% by LCMS

### Step 10: Synthesis of (2S,4R)-1-((S)-2-(3-(4-(3-aminopropyl)piperidin-1-yl)propanamido)-3,3-dimethylbutanoyl)-4-hydroxy-N-(4-(4-methylthiazol-5-yl)benzyl)pyrrolidine-2-carboxamide 2,2,2-trifluoroacetate (11)

TFA (0.678 mL, 8.80 mmol) was added dropwise to a stirred solution of tert-butyl (3-(1-(3-(((S)-1-((2S,4R)-4-hydroxy-2-((4-(4-methylthiazol-5-yl)benzyl) carbamoyl)pyrrolidin-1-yl)-3,3-dimethyl-1-oxobutan-2-yl)amino)-3-oxopropyl)piperidin-4-yl)propyl)carbamate (0.32 g, 0.440 mmol) in DCM (5 mL) at 0 to 5° C. under N₂ atmosphere. The reaction solution was stirred at 25° C. for 2 hours and the reaction process was observed by TLC. After completion of the reaction, the reactant was concentrated under reduced pressure to obtain (2S,4R)-1-((S)-2-(3-(4-(3-aminopropyl)piperidin-1-yl)propanamido)-3,3-dimethylbutanoyl)-4-hydroxy-N-(4-(4-methylthiazol-5-yl)benzyl)pyrrolidine-2-carboxamide 2,2,2-trifluoroacetate as brown gum. LCMS: (MM-ES+APCI) (M+H)⁺=627.4; Purity: 92.62% by LCMS.

### Step 11: Synthesis of 4-(2-(4-((4-((4-((2-chlorophenyl)carbamoyl)phenyl)amino)-5-fluoropyrimidin-2-yl)amino)phenyl)acetyl)-N-(3-(1-(3-(((S)-1-((2S,4R)-4-hydroxy-2-((4-(4-methylthiazol-5-yl)benzyl)carbamoyl)pyrrolidin-1 yl)-3,3-dimethyl-1-oxobutan-2-yl)amino)-3-oxopropyl)piperidin-4-yl)propyl)piperazine-1-carboxamide (Compound 10)

TEA (0.066 mL, 0.472 mmol) and CDI (0.077 g, 0.472 mmol) were added to a stirred solution of (2S,4R)-1-((S)-2-(3-(4-(3-aminopropyl)piperidin-1-yl)propanamido)-3,3-dimethylbutanoyl)-4-hydroxy-N-(4-(4-methylthiazol-5-yl)benzyl)pyrrolidine-2-carboxamide 2,2,2-trifluoroacetate (0.35 g, 0.472 mmol) in DCM (3 mL) at 0 to 5° C. under N₂. The reaction solution was stirred at 25° C. for 1 hour. In addition, the reactant was added dropwise with a solution of N-(2-chlorophenyl)-4-((5-fluoro-2-((4-(2-oxo-2-(piperazin-1-yl)ethyl)phenyl)amino)pyrimidin-4-yl)amino)benzamide 2,2,2-trifluoroacetate (0.159 g, 0.236 mmol) (UPP-L1) and TEA (0.066 mL, 0.472 mmol) in DCM (3 mL) at 0 to 5° C. Thereafter, the reactant was stirred at 25° C. for 3 hours. The reaction process was observed by UPLC-MS. After completion of the reaction, the reactant was diluted with water (10 mL) and extracted with DCM (2×20 mL). The combined organic phase was dried over Na₂SO₄, and filtered and concentrated under reduced pressure to obtain Compound 10 (0.38 g) as yellow solid. LCMS: (MM-ES+APCI)(M−H)⁻=1210.8; Purity: ˜10% by UPLC-MS

¹H NMR (400 MHz, DMSO-d₆): δ=9.90 (br s, 1H), 9.66 (br s, 1H), 9.29 (br s, 1H), 8.97 (s, 1H), 8.67 (d, J=9.6 Hz, 1H), 8.60 (t, J=6.4 Hz, 1H), 8.18 (d, J=3.6 Hz, 1H), 8.04 (d, J=8.8 Hz, 2H), 7.97 (d, J=8.8 Hz, 2H), 7.67 (dd, J1=8.0, J2=1.6 Hz, 1H), 7.62 (d, J=8.4 Hz, 2H), 7.56 (dd, J1=8.0, J2=1.2 Hz, 1H), 7.45-7.36 (m, 5H), 7.29 (td, J1=7.6, J2=1.6 Hz, 1H), 7.13 (d, J=8.4 Hz, 2H), 6.46 (t, J=4.8 Hz, 1H), 5.13 (d, J=3.6 Hz, 1H), 4.54 (d, J=9.6 Hz, 1H), 4.47-4.42 (m, 2H), 4.36 (s, 1H), 4.21 (dd, J1=16.0, J2=5.6 Hz, 1H), 3.69-3.63 (m, 3H), 3.44-3.41 (m, 5H), 3.21-3.17 (m, 4H), 2.92-2.85 (m, 4H), 2.52-2.50 (m, 4H), 2.44 (s, 3H), 2.33-2.24 (m, 1H), 2.06-2.10 (m, 1H), 1.94-1.87 (m, 2H), 1.64-1.59 (m, 2H), 1.39-1.30 (m, 2H), 1.19-1.16 (m, 2H), 1.12-1.10 (m, 3H), 0.95 (s, 9H)

### Example 11: Synthesis of Compound 11

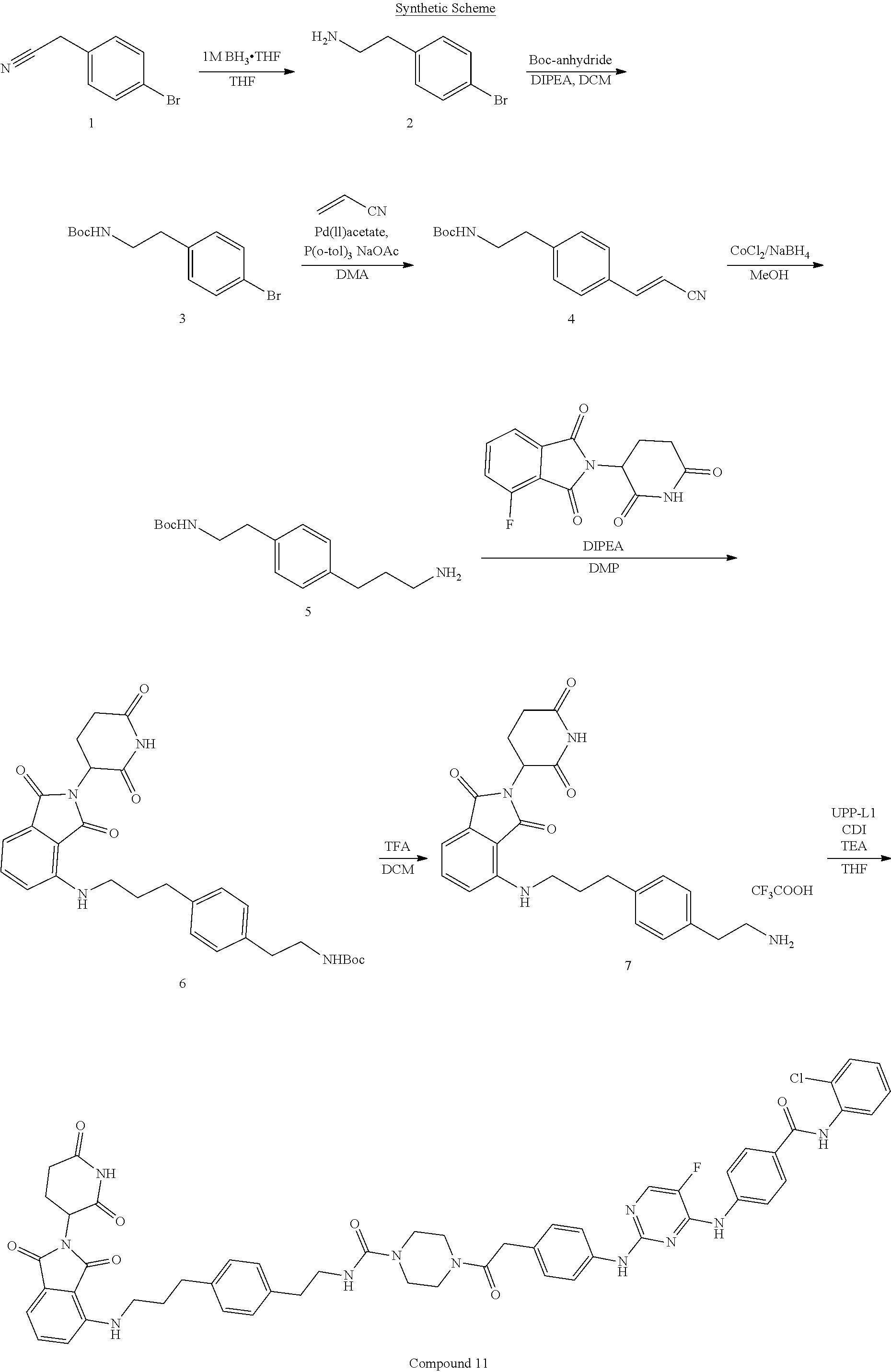

### Experimental Process

### Step 1: Synthesis of 2-(4-bromophenyl)ethane-1-amine (2)

BH₃·THF (30.6 mL, 30.6 mmol) was added dropwise to a stirred solution of 2-(4-bromophenyl)acetonitrile (2.0 g, 10.20 mmol) in THE (30 mL) under vacuum at 0° C. under N₂ atmosphere. Thereafter, the reactant was maintained at 25° C. and heated to 66° C. for 20 hours. The reactant was observed by TLC. After completion of the reaction, the reactant was quenched with MeOH (30 mL) and concentrated under reduced pressure to obtain 2-(4-bromophenyl)ethane-1-amine (2.38 g). LCMS: (MM-ES+APCI) (M+H)⁺=200.0; Purity: 97.18% by LCMS

### Step 2: Synthesis of tert-butyl (4-bromophenethyl)carbamate (3)

DIPEA (4.02 mL, 22.99 mmol) and Boc-anhydride (2.94 mL, 12.65 mmol) were added to a stirred solution of 2-(4-bromophenyl)ethane-1-amine (2.3 g, 11.50 mmol) in DCM (25 mL) under vacuum at 25° C. under N₂ atmosphere. The reaction mixture was maintained at 25° C. for 2 hours and observed by TLC. After completion of the reaction, the reactant was diluted with water (20 mL) and the aqueous phase and the organic phase were separated. The aqueous phase was extracted with DCM (3×15 mL). The combined organic phase was dried over Na₂SO₄, and concentrated under reduced pressure. The obtained compound was purified by flash column chromatography, and eluted with 1-20% Pet ether/EtOAc. Desired fractions were concentrated under reduced pressure to obtain tert-butyl (4-bromophenethyl)carbamate (1.800 g, 6.00 mmol, 52.2% yield). LCMS: (MM-ES+APCI) (M+H−100)⁺=200.0; Purity: 99.72% by LCMS

### Step 3: Synthesis of tert-butyl (EIZ)-(4-(2-cyanovinyl)phenethyl)carbamate (4)

A 10 mL vacuum-sealed tube was filled with tert-butyl (4-bromophenethyl) carbamate (90 mg, 0.300 mmol), DMA (3.0 mL), and NaOAc (29.5 mg, 0.360 mmol). The suspension was purged with N₂, and added with acrylonitrile (0.059 mL, 0.899 mmol), P(o-tol)₃ (9.13 mg, 0.030 mmol), and Pd(II) acetate (3.37 mg, 0.015 mmol). The suspension was stirred at 140° C. for 20 hours. The reaction was observed by TLC. After completion of the reaction, the reactant was filtered with Celite and washed with EtOH (20 mL). The filtrate was washed with brine (2×20 mL). The organic phase was dried over Na₂SO₄, and filtered and concentrated under reduced pressure to obtain tert-butyl (E/Z)-(4-(2-cyanovinyl)phenethyl)carbamate (107 mg) as brown gum liquid.

### Step 4: Synthesis of tert-butyl (4-(3-aminopropyl)phenethyl)carbamate (5)

CoCl₂ (47.7 mg, 0.367 mmol) and NaBH₄ (13.89 mg, 0.367 mmol) were added to a solution of tert-butyl (E/Z)-(4-(2-cyanovinyl)phenethyl)carbamate (100 mg, 0.367 mmol) in MeOH (5 mL) at 0 to 5° C. under N₂ atmosphere. The reaction mixture was stirred at 25° C. for 24 hours. After completion of the reaction, the reactant was filtered through celite and washed with MeOH (10 mL), and the obtained filtrate was concentrated under reduced pressure. The obtained concentrate was filled with NH₃·H₂O (10 mL) and the compound was extracted with DCM (2×10 mL). The combined organic phase was dried over Na₂SO₄, and filtered and concentrated under reduced pressure to obtain tert-butyl (4-(3-aminopropyl)phenethyl)carbamate (55 mg, 0.052 mmol, 14.05% yield). LCMS: (MM-ES+APCI) (M+H)⁺=279.2; Purity: 26.11% by LCMS

### Step 5: Synthesis of tert-butyl (4-(3-((2-(2,6-dioxopiperidin-3 yl)-1,3-dioxoisoindolin-4-yl)amino)propyl)phenethyl)carbamate (6)

2-(2,6-dioxopiperidin-3-yl)-4-fluoroisoindoline-1,3-dione (49.6 mg, 0.180 mmol) and DIPEA (0.094 mL, 0.539 mmol) were added to a solution of tert-butyl (4-(3-aminopropyl)phenethyl)carbamate (50 mg, 0.180 mmol) in DMF (1 mL) at 25° C. The reactant was heated to 90° C. and maintained for 16 hours. The reactant was observed by UPLC-MS. After completion of the reaction, the reactant was diluted with EtOAc at 25° C. and washed with brine (3×10 mL). The obtained organic phase was dried over Na₂SO₄, filtered, and concentrated under reduced pressure to obtain tert-butyl (4-(3-((2-(2,6-dioxopiperidin-3-yl)-1,3-dioxoisoindolin-4-yl)amino)propyl)phenethyl)carbamate (68 mg, 0.019 mmol, 10.70% yield) as yellow gum. LCMS: (MM-ES+APCI) (M−H)⁺=533.3; Purity: 15.11% by UPLC-MS

### Step 6: Synthesis of 4-((3-(4-(2-aminoethyl)phenyl)propyl)amino)-2-(2,6-dioxopiperidin-3-yl)isoindoline-1,3-dione 2,2,2-trifluoroacetate (7)

TFA (0.173 mL, 2.245 mmol) was added dropwise to a stirred solution of tert-butyl (4-(3-((2-(2,6-dioxopiperidin-3-yl)-1,3-dioxoisoindolin-4-yl)amino)propyl)phenethyl)carbamate (60 mg, 0.112 mmol) in DCM (5 mL) at 0 to 5° C. The reaction solution was stirred and warmed to 25° C. The reactant was maintained for 2 hours and then observed by UPLC-MS. After completion of the reaction, the reactant was concentrated under reduced pressure to obtain 4-((3-(4-(2-aminoethyl)phenyl)propyl)amino)-2-(2,6-dioxopiperidin-3-yl)isoindoline-1,3-dione 2,2,2-trifluoroacetate (55 mg, 0.013 mmol, 11.84% yield) as yellow gum. LCMS: (MM-ES+APCI) (M+H)⁺=435.3; Purity: 13.25% by UPLC-MS

### Step 7: Synthesis of 4-(2-(4-((4-((4-((2-chlorophenyl)carbamoyl)phenyl)amino)-5-fluoropyrimidin-2-yl)amino)phenyl)acetyl)-N-(4-(3-((2-(2,6-dioxopiperidin-3-yl)-1,3-dioxoisoindolin-4-yl)amino)propyl)phenethyl)piperazine-1-carboxamide (Compound 11)

TEA (0.499 mL, 3.65 mmol) and CDI (177 mg, 1.094 mmol) were added to a stirred solution of 4-((3-(4-(2-aminoethyl)phenyl)propyl)amino)-2-(2,6-dioxopiperidin-3-yl)isoindoline-1,3-dione 2,2,2-trifluoroacetate (400 mg, 0.729 mmol) in THF (10 mL) at 0 to 10° C. The reaction solution was stirred for 1 hour and warmed to 25° C. The reactant was added with a solution of N-(2-chlorophenyl)-4-((5-fluoro-2-((4-(2-oxo-2-(piperazin-1-yl)ethyl)phenyl)amino)pyrimidine-4-yl)amino)benzamide 2,2,2-trifluoroacetate (246 mg, 0.365 mmol) in THF (10 mL) and TEA (0.499 mL, 3.65 mmol) at 25° C. The reactant was maintained for 4 hours and then observed by UPLC-MS. After completion of the reaction, the reactant was diluted with water (30 mL) and the compounds were sequentially extracted with DCM (10 mL) and a mixture of THF:DCM (2×20 mL) (2:8). The combined organic phase was dried over Na₂SO₄, and filtered and concentrated under reduced pressure to obtain brown gum. The obtained compound was purified by flash column chromatography and eluted with 1-15% MeOH in DCM. Desired fractions were concentrated under reduced pressure, purified by prep-HPLC, and freeze-dried for 30 hours to obtain Compound 11 (40 mg, 0.039 mmol, 5.37% yield) as yellow solid.

¹H NMR (400 MHz, DMSO-d₆): δ=11.10 (br s, 1H), 9.91 (br s, 1H), 9.66 (br s, 1H), 9.30 (br s, 1H), 8.19 (d, J=3.6 Hz, 1H), 8.04 (d, J=8.8 Hz, 2H), 7.98 (d, J=8.8 Hz, 2H), 7.66 (dd, J1=7.6, J2=1.2 Hz, 1H), 7.63 (d, 2H), 7.58-7.54 (m, 2H), 7.40 (td, J1=7.6, J2=1.2 Hz, 1H), 7.30 (td, J1=8.0, J2=1.6, Hz, 1H), 7.13 (d, J=8.4 Hz, 4H), 7.09-7.01 (m, 4H), 6.63 (t, J=5.2 Hz, 1H), 6.58 (t, J=5.6 Hz, 1H), 5.06 (dd, J1=12.8, J2=5.6 Hz, 1H), 3.67 (s, 2H), 3.43-3.39 (m, 4H), 3.31-3.27 (m, 2H), 3.22-3.18 (m, 6H), 2.93-2.84 (m, 1H), 2.68-2.57 (m, 6H), 2.06-2.01 (m, 1H), 1.89-1.82 (m, 2H), LCMS (MM-ES+APCI)(M+H)⁺=1020.3, HPLC: Rt-15.298; Purity: 97.38%

### Example 12: Synthesis of Compound 12

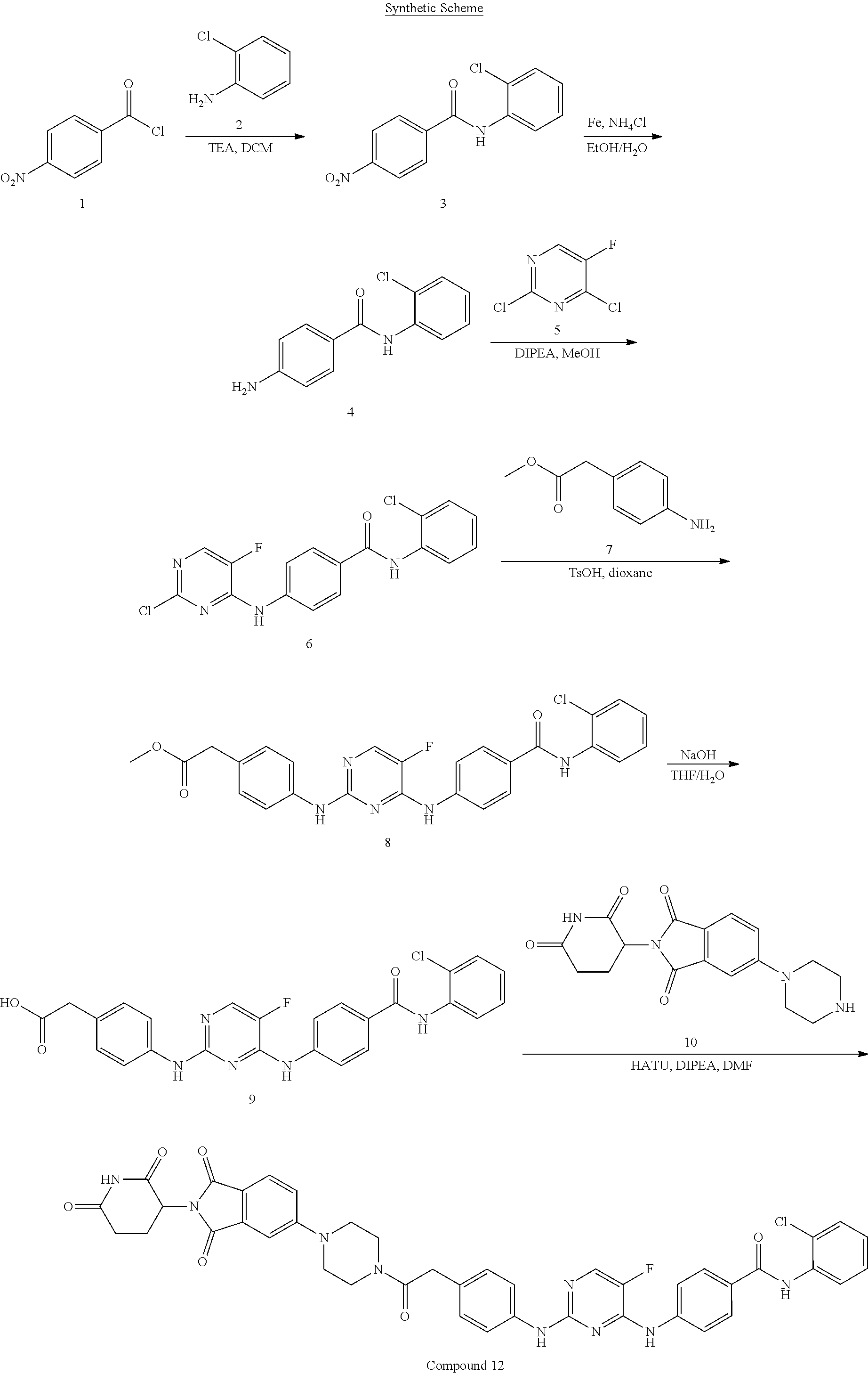

### Experimental Process

### Step 1: Synthesis of N-(2-chlorophenyl)-4-nitrobenzamide (3)

A solution of 2-chloroaniline (7.22 g, 56.58 mmol) in DCM (50 mL) was added dropwise to a solution of 4-nitrobenzoyl chloride (10 g, 53.89 mmol) and TEA (8.18 g, 80.83 mmol) in DCM (50 mL) at 0° C. The mixture was stirred at 20° C. for 14 hours. The desired mass was detected by LCMS. The mixture layers were separated by adding water (100 mL). The organic phase was dried over Na₂SO₄ and concentrated under reduced pressure. The product was pulverized with petroleum ether/EtOAc (100 mL, 10:1) to obtain N-(2-chlorophenyl)-4-nitrobenzamide (8 g, 28.91 mmol, 53.66% yield) as yellow solid. MS(M+H)⁺=277.7.

### Step 2: Synthesis of 4-amino-N-(2-chlorophenyl)benzamide (4)

Fe (3.23 g, 57.83 mmol) was added to a solution of N-(2-chlorophenyl)-4-nitrobenzamide (4 g, 14.46 mmol) and NH₄Cl (3.09 g, 57.83 mmol) in EtOH (30 mL) and H₂O (30 mL) at 20° C. The mixture was stirred at 80° C. for 1 hour. It was confirmed through LCMS that the starting material was completely consumed, and a main peak with the desired mass was detected. The mixture was added with a NaHCO₃ solution (200 mL) and then extracted with EtOAc (50 mL×4). The combined organic layer was dried with Na₂SO₄ to obtain 4-amino-N-(2-chlorophenyl)benzamide (2.3 g, 9.32 mmol, 64.49% yield) as yellow solid. MS(M+H)⁺=247.1.

### Step 3: Synthesis of 4-((2-chloro-5 fluoropyrimidin-4-yl)amino)-N-(2-chlorophenyl)benzamide (6)

A solution of 4-Amino-N-(2-chlorophenyl)benzamide (2.3 g, 9.32 mmol), 2,4-dichloro-5-fluoro-pyrimidine (4.67 g, 27.97 mmol) and DIPEA (1.20 g, 9.32 mmol) in MeOH (50 mL) was stirred at 70° C. for 14 hours. It was confirmed through LCMS that the starting material was completely consumed, and the desired mass was detected. (Reference: a large amount of white solid was formed). After the temperature was lowered to room temperature, the mixture was filtered. The solid portion was collected and dried to obtain 4-((2-chloro-5-fluoropyrimidin-4-yl)amino)-N-(2-chlorophenyl)benzamide (2.4 g, 6.36 mmol, 68.24% yield) as white solid. MS(M+H)⁺=377.1.

¹H NMR (400 MHz, DMSO-d₆) δ=10.26 (s, 1H), 9.95 (s, 1H), 8.40 (d, J=3.4 Hz, 1H), 8.01 (d, J=8.7 Hz, 2H), 7.88 (d, J=8.8 Hz, 2H), 7.62 (dd, J=1.5, 7.9 Hz, 1H), 7.56 (dd, J=1.3, 7.9 Hz, 1H), 7.39 (dt, J=1.4, 7.7 Hz, 1H), 7.32-7.27 (m, 1H).

### Step 4: Synthesis of methyl 2-(4-((4-((4-((2-chlorophenyl)carbamoyl)phenyl)amino)-5 fluoropyrimidin-2-yl)amino)phenyl)acetate (8)

TsOH·H₂O (1.66 g, 8.75 mmol) was added to a solution of 4-((2-chloro-5-fluoropyrimidin-4-yl)amino)-N-(2-chlorophenyl)benzamide (1.1 g, 2.92 mmol) and methyl 2-(4-aminophenyl)acetate (578.07 mg, 3.50 mmol) in dioxane (12 mL) at 20° C. The mixture was stirred at 100° C. for 12 hours. It was confirmed through LCMS that the starting material was completely consumed, and the desired mass was detected. The mixture was concentrated to obtain methyl 2-(4-((4-((4-((2-chlorophenyl)carbamoyl)phenyl)amino)-5-fluoropyrimidin-2-yl)amino)phenyl)acetate (2 g, crude) as dark brown oil, which was immediately used in the next reaction. MS(M+H)⁺=506.1.

### Step 5: Synthesis of 2-(4-((4-((4-((2-chlorophenyl)carbamoyl)phenyl)amino)-5-fluoropyrimidin-2-yl)amino)phenyl)acetate (9)

A solution of NaOH (790.57 mg, 19.77 mmol) in water (10 mL) was added to a solution of methyl 2-(4-((4-((4-((2-chlorophenyl)carbamoyl)phenyl)amino)-5-fluoropyrimidin-2-yl)amino)phenyl)acetate (2 g, 3.95 mmol) in THE (20 mL) at 20° C. The mixture was stirred at 20° C. for 1 hour. It was confirmed through LCMS that the starting material was completely consumed, and a main peak with the desired mass was detected. The mixture was concentrated to remove the organic solvent. The remaining aqueous layer was acidified to pH=2 using a 1 N HCl solution. The mixture was filtered and then washed with EtOAc (10 mL). The solid portion was collected and dried to obtain 2-(4-((4-((4-((2-chlorophenyl)carbamoyl)phenyl)amino)-5-fluoropyrimidin-2-yl)amino)phenyl)acetate (1.5 g, crude) as gray solid. MS(M+H)⁺=492.2.

### Step 6: Synthesis of N-(2-chlorophenyl)-4-((2-((4-(2-(4-(2-(2,6-dioxopiperidin-3 yl)-1,3-dioxoisoindoline-5 yl)piperazin-1 yl)-2-oxoethyl)phenyl)amino)-5-fluoropyrimidin-4-yl)amino)benzamide (Compound 12)

HATU (85.03 mg, 223.62 μmol) was added to a solution of 2-(4-((4-((4-((2-chlorophenyl)carbamoyl)phenyl)amino)-5-fluoropyrimidin-2-yl)amino)phenyl)acetate (100 mg, 203.29 μmol), 2-(2,6-dioxopiperidin-3-yl)-5-(piperazin-1-yl)isoindoline-1,3-dione (77.01 mg, 203.29 μmol, HCl) and DIPEA (131.37 mg, 1.02 mmol) in DMF (2 mL) at 20° C., and then the mixture was stirred for 14 hours. It was confirmed through LCMS that the starting material was completely consumed, and the desired mass was detected. The mixture was neutralized with AcOH, and then purified by prep-HPLC (column: Phenomenex luna C18 150*25 mm*10 um; mobile phase: [water (FA)-ACN]; B %: 32%-62%, 10 min) and freeze-dried. The product was repurified by prep-HPLC (column: Waters Xbridge 150*25 mm*Sum; mobile phase: [water (NH₄HCO₃)-ACN]; B %: 34%-64%, 9 min) and then freeze-dried to obtain Compound 12 (18.9 mg, 21.07 μmol, 10.36% yield, 91% purity) as yellow solid. MS(M+H)⁺=816.4.

¹H NMR (400 MHz, DMSO-d₆) δ=11.08 (br s, 1H), 9.89 (s, 1H), 9.65 (br s, 1H), 9.28 (s, 1H), 8.18 (d, J=3.2 Hz, 1H), 8.06-7.93 (m, 4H), 7.63 (br dd, J=8.5, 17.2 Hz, 4H), 7.55 (br d, J=8.1 Hz, 1H), 7.38 (br t, J=7.6 Hz, 1H), 7.32-7.26 (m, 2H), 7.22-7.11 (m, 3H), 5.07 (br dd, J=5.4, 12.8 Hz, 1H), 3.71 (br s, 2H), 3.67-3.64 (m, 2H), 3.64-3.58 (m, 2H), 3.46-3.38 (m, 4H), 2.93-2.82 (m, 1H), 2.63-2.55 (m, 2H), 2.05-1.98 (m, 1H).

### Example 13: Synthesis of Compound 13

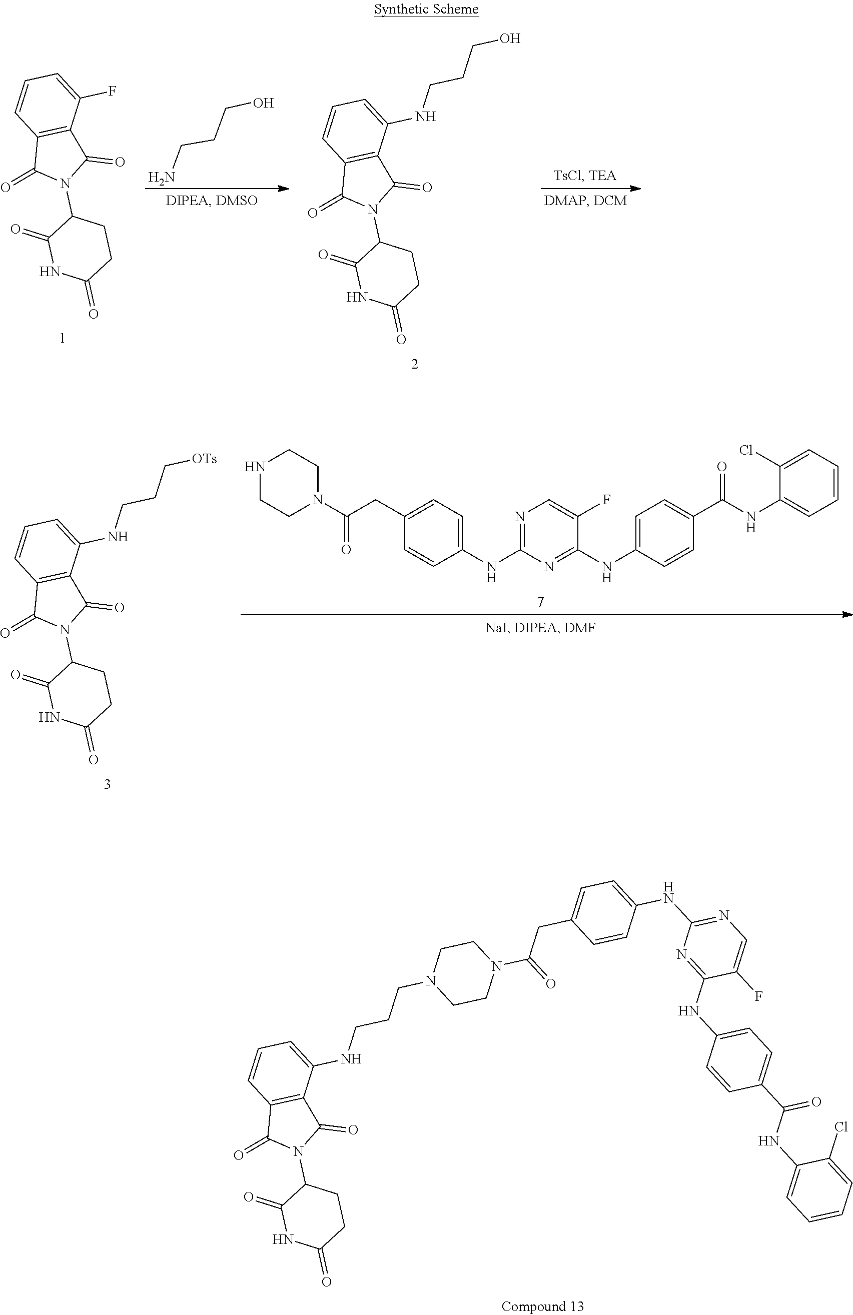 [SMILES: O=C1CCC(N2C(=O)c3cccc(F)c3C2=O)C(=O)N1 ; NCCCO ; O=C1CCC(N2C(=O)c3cccc(NCCCO)c3C2=O)C(=O)N1 ; Cc1ccc(S(=O)(=O)OCCCNc2cccc3c2C(=O)N(C2CCC(=O)NC2=O)C3=O)cc1 ; O=C(Nc1ccccc1Cl)c1ccc(Nc2nc(Nc3ccc(CC(=O)N4CCNCC4)cc3)ncc2F)cc1 ; O=C1CCC(N2C(=O)c3cccc(NCCCN4CCN(C(=O)Cc5ccc(Nc6ncc(F)c(Nc7ccc(C(=O)Nc8ccccc8Cl)cc7)n6)cc5)CC4)c3C2=O)C(=O)N1]

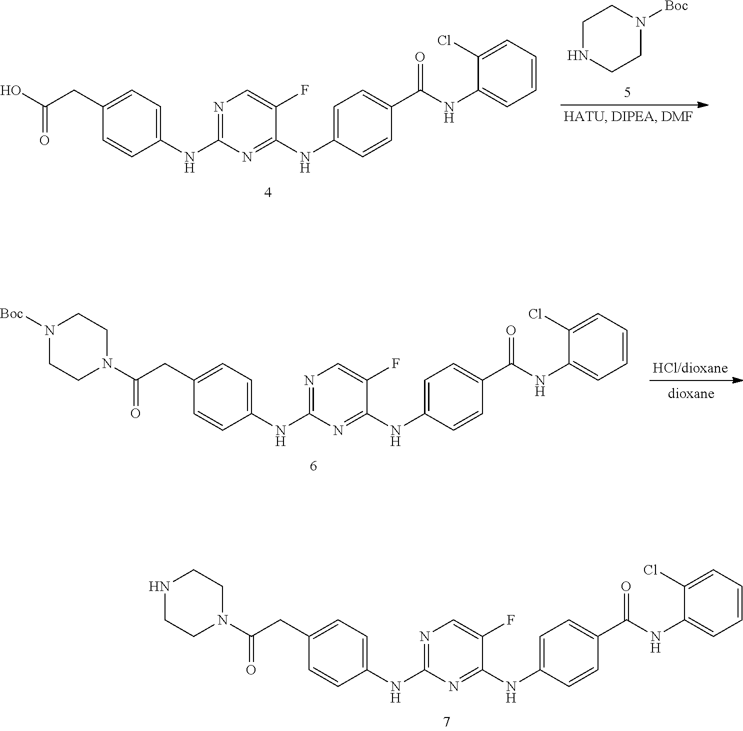 [SMILES: O=C(O)Cc1ccc(Nc2ncc(F)c(Nc3ccc(C(=O)Nc4ccccc4Cl)cc3)n2)cc1 ; CC(C)(C)OC(=O)N1CCNCC1 ; CC(C)(C)OC(=O)N1CCN(C(=O)Cc2ccc(Nc3ncc(F)c(Nc4ccc(C(=O)Nc5ccccc5Cl)cc4)n3)cc2)CC1 ; O=C(Nc1ccccc1Cl)c1ccc(Nc2nc(Nc3ccc(CC(=O)N4CCNCC4)cc3)ncc2F)cc1]

### Experimental Process

### Step 1: Synthesis of 2-(2,6-dioxopiperidin-3-yl)-4-((3-hydroxypropyl)amino)isoindoline-1,3-dione (2)

DIPEA (935.80 mg, 7.24 mmol, 1.26 mL) and 3-aminopropan-1-ol (299.11 mg, 3.98 mmol, 307.10 μL) were added to a solution of 2-(2,6-dioxopiperidin-3-yl)-4-fluoroisoindoline-1,3-dione (1 g, 3.62 mmol) in DMSO (10 mL) and then stirred at 80° C. for 12 hours. The main peak of desired mass was detected by LCMS. The mixture was diluted with water (30 mL) and extracted with EtOAc (20 mL×3). The combined organic phase was washed with saturated brine (20 mL) and dried over Na₂SO₄. The organic phase was filtered and concentrated under reduced pressure to obtain 2-(2,6-dioxopiperidin-3-yl)-4-((3-hydroxypropyl)amino)isoindoline-1,3-dione (1.3 g, 2.98 mmol, 82.37% yield, 76% purity) as yellow oil. MS(M+H)⁺=332.1.

### Step 2: Synthesis of 3-((2-(2,6-dioxopiperidin-3-yl)-1,3-dioxoisoindolin-4-yl)amino)propyl 4-methylbenzenesulfonate (3)

TsCl (1.12 g, 5.89 mmol), DMAP (95.87 mg, 784.73 μmol) and TEA (794.06 mg, 7.85 mmol, 1.09 mL) were added to a solution of 2-(2,6-dioxopiperidin-3-yl)-4-((3-hydroxypropyl)amino)isoindoline-1,3-dione (1.3 g, 3.92 mmol) in DCM (10 mL). The mixture was stirred at 25° C. for 2 hours. 58% of the desired mass was detected by LCMS. The mixture was diluted with water (50 mL) and then extracted with DCM (50 mL×3). The combined organic phase was washed with saturated brine (50 mL) and then dried over Na₂SO₄. After filtration and concentration under reduced pressure, the residue was purified by flash silica gel chromatography (ISCO®; 20 g SepaFlash® Silica Flash Column, Eluent of 0˜50% petroleum ether:EtOAc gradient @ 60 mL/min) to obtain 3-((2-(2,6-dioxopiperidin-3-yl)-1,3-dioxoisoindolin-4-yl)amino)propyl 4-methylbenzenesulfonate (460 mg, 820.50 μmol, 20.91% yield, 86.6% purity) as yellow solid. MS(M+H)⁺=486.1.

### Step 3: Synthesis of tert-butyl 4-(2-(4-((4-((4-((2-chlorophenyl)carbamoyl)phenyl)amino)-5-fluoropyrimidin-2-yl)amino)phenyl)acetyl)piperazine-1-carboxylate (6)

HATU (425.14 mg, 1.12 mmol) was added to a solution of 2-(4-((4-((4-((2-chlorophenyl)carbamoyl)phenyl)amino)-5-fluoropyrimidin-2-yl) amino)phenyl) acetic acid (500 mg, 1.02 mmol), tert-butyl piperazine-1-carboxylate (189.32 mg, 1.02 mmol) and DIPEA (656.86 mg, 5.08 mmol) in DMF (5 ml) at 20° C. The mixture was stirred at 20° C. for 14 hours. It was confirmed through LCMS that the starting material was completely consumed, and a peak with the desired mass was detected. The mixture was poured with water (15 mL) and extracted with EtOAc (10 mL×3). The combined organic phase was washed with brine (10 mL), dried over Na₂SO₄, and concentrated under reduced pressure. The product was pulverized with petroleum ether/EtOAc (10:1, 30 mL) to obtain tert-butyl 4-(2-(4-((4-((4-((2-chlorophenyl)carbamoyl)phenyl)amino)-5-fluoropyrimidin-2-yl)amino)phenyl)acetyl)piperazine-1-carboxylate (450 mg, 681.68 μmol, 67.06% yield) as gray solid. MS(M+H)⁺=660.3.

### Step 4: Synthesis of N-(2-chlorophenyl)-4-((5-fluoro-2-((4-(2-oxo-2-(piperazin-1-yl)ethyl)phenyl)amino)pyrimidine-4-yl)amino)benzamide (7)

HCl/dioxane (4 M, 5 mL) was added to a solution of tert-butyl 4-(2-(4-((4-((4-((2-chlorophenyl)carbamoyl)phenyl) amino)-5-fluoropyrimidin-2-yl)amino)phenyl)acetyl)piperazine-1-carboxylate (450 mg, 681.68 μmol) in dioxane (2 mL). The mixture was stirred at 20° C. for 1 hour. The 73% peak of desired mass was detected by LCMS. The mixture was concentrated under reduced pressure to obtain N-(2-chlorophenyl)-4-((5-fluoro-2-((4-(2-oxo-2-(piperazin-1-yl)ethyl)phenyl)amino)pyrimidine-4-yl)amino)benzamide (430 mg, crude, HCl) as yellow solid. MS(M+H)⁺=560.3.

### Step 5: Synthesis of N-(2-chlorophenyl)-4-((2-((4-(2-(4-(3-((2-(2,6-dioxopiperidin-3 yl))-1,3-dioxoisoindolin-4-yl)amino)propyl)piperazin-1 yl)-2-oxoethyl)phenyl)amino)-5-fluoropyrimidin-4-yl)amino)benzamide (Compound 13)

N-(2-chlorophenyl)-4-((5-fluoro-2-((4-(2-oxo-2-(piperazin-1-yl)ethyl)phenyl)amino)pyrimidine-4-yl)amino)benzamide (135.14 mg, 226.57 μmol, HCl), NaI (6.17 mg, 41.19 μmol) and DIPEA (133.10 mg, 1.03 mmol, 179.38 μL) were added to a solution of 3-((2-(2,6-dioxopiperidin-3-yl)-1,3-dioxoisoindolin-4-yl)amino)propyl 4-methylbenzenesulfonate (100 mg, 205.97 μmol) in DMF (2 mL). The mixture was stirred at 80° C. for 12 hours. 50% of the desired mass was detected by LCMS. The mixture was diluted with water (20 mL) and then extracted with EtOAc (10 mL×3). The combined organic phase was washed with saturated brine (10 mL), dried over Na₂SO₄, and filtered and concentrated under reduced pressure. The residue was purified by prep-HPLC (column: Phenomenex luna C18 150×25 mm×10 μm; mobile phase: [water (FA)-ACN]; B %: 8%-38%, 2 min) and prep-HPLC (column: Waters Xbridge 150×25 mm×5 μm; mobile phase: [water (NH₄HCO₃)-ACN]; B %: 45%-75%, 8 min) and then freeze-dried to obtain Compound 13 (20.2 mg, 21.72 μmol, 10.54% yield, 93.9% purity) as yellow solid. MS(M+H)⁺=873.5.

¹H NMR (400 MHz, DMSO-d₆) δ=11.08 (s, 1H), 9.87 (s, 1H), 9.65 (s, 1H), 9.27 (s, 1H), 8.18 (d, J=3.7 Hz, 1H), 8.05-8.01 (m, 2H), 7.99-7.95 (m, 2H), 7.68-7.65 (m, 1H), 7.60 (d, J=8.6 Hz, 2H), 7.55 (d, J=7.6 Hz, 2H), 7.42-7.36 (m, 1H), 7.31-7.25 (m, 1H), 7.14-7.08 (m, 3H), 7.00 (d, J=7.0 Hz, 1H), 6.80-6.74 (m, 1H), 5.04 (dd, J=5.3, 12.7 Hz, 1H), 3.64 (s, 2H), 3.50-3.49 (m, 4H), 3.30 (s, 2H), 2.94-2.82 (m, 1H), 2.60-2.58 (m, 2H), 2.33-2.31 (m, 2H), 2.27-2.25 (m, 4H), 2.05-1.97 (m, 1H), 1.71-1.70 (m, 2H).

### Example 14: Synthesis of Compound 14

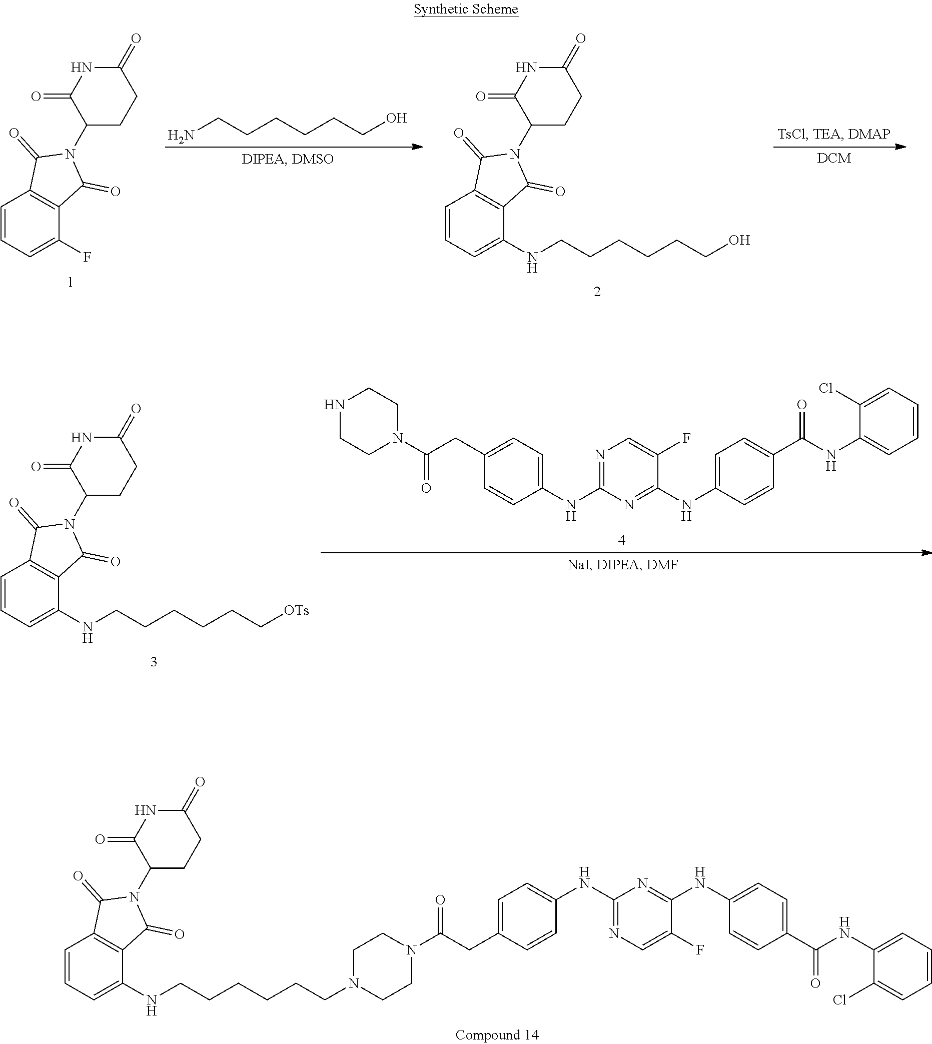 [SMILES: O=C1CCC(N2C(=O)c3cccc(F)c3C2=O)C(=O)N1 ; NCCCCCCO ; O=C1CCC(N2C(=O)c3cccc(NCCCCCCO)c3C2=O)C(=O)N1 ; Cc1ccc(S(=O)(=O)OCCCCCCNc2cccc3c2C(=O)N(C2CCC(=O)NC2=O)C3=O)cc1 ; O=C(Nc1ccccc1Cl)c1ccc(Nc2nc(Nc3ccc(CC(=O)N4CCNCC4)cc3)ncc2F)cc1 ; O=C1CCC(N2C(=O)c3cccc(NCCCCCCN4CCN(C(=O)Cc5ccc(Nc6ncc(F)c(Nc7ccc(C(=O)Nc8ccccc8Cl)cc7)n6)cc5)CC4)c3C2=O)C(=O)N1]

### Experimental Process

### Step 1: Synthesis of 2-(2,6-dioxopiperidin-3-yl)-4-((6-hydroxyhexyl)amino)isoindoline-1,3-dione (2)

DIPEA (1.40 g, 10.86 mmol, 1.89 mL) and 6-aminohexan-1-ol (509.11 mg, 4.34 mmol) were added to a solution of 2-(2,6-dioxopiperidin-3-yl)-4-fluoroisoindoline-1,3-dione (1 g, 3.62 mmol) in DMSO (10 mL) and then stirred at 80° C. for 12 hours. 50% of the desired mass was detected by LCMS. The mixture was diluted with water (50 mL) and then extracted with EtOAc (30 mL×3). The combined organic phase was washed with saturated brine (20 mL), dried over Na₂SO₄, and filtered and concentrated under reduced pressure. The residue was purified by flash silica gel chromatography (ISCO®; 20 g SepaFlash® Silica Flash Column, Eluent of 0˜60% petroleum ether:EtOAc gradient @ 60 mL/min) to obtain 2-(2,6-dioxopiperidin-3-yl)-4-((6-hydroxyhexyl)amino)isoindoline-1,3-dione (0.63 g, 1.37 mmol, 37.75% yield, 81% purity) as yellow oil. MS(M+H)⁺=374.2.

### Step 2: Synthesis of 6-((2-(2,6-dioxopiperidin-3-yl)-1,3-dioxoisoindolin-4-yl)amino)hexyl 4-methylbenzenesulfonate (3)

TsCl (482.49 mg, 2.53 mmol), TEA (512.17 mg, 5.06 mmol, 704.50 μL) and DMAP (20.61 mg, 168.72 μmol) were added to a solution of 2-(2,6-dioxopiperidin-3-yl)-4-((6-hydroxyhexyl)amino)isoindoline-1,3-dione (0.63 g, 1.69 mmol) in DCM (6 mL) and then stirred at 20° C. for 2 hours. 44% of the desired mass was detected by LCMS. The mixture was diluted with water (30 mL) and then extracted with EtOAc (30 mL×3). The combined organic phase was washed with saturated brine (50 mL), dried over Na₂SO₄, and filtered and concentrated under reduced pressure. The residue was purified by flash silica gel chromatography (ISCO®; 20 g SepaFlash® Silica Flash Column, Eluent of 0˜40% petroleum ether:EtOAc gradient @ 80 mL/min) to obtain 6-((2-(2,6-dioxopiperidin-3-yl)-1,3-dioxoisoindolin-4-yl)amino)hexyl 4-methylbenzenesulfonate (0.7 g, 987.13 μmol, 58.51% yield, 74.4% purity) as yellow oil. MS(M+H)⁺=528.3.

### Step 3: Synthesis of N-(2-chlorophenyl)-4-((2-((4-(2-(4-(6-((2-(2,6-dioxopiperidin-3 yl))-1,3-dioxoisoindolin-4-yl)amino)hexyl)piperazin-1-yl)-2-oxoethyl)phenyl)amino)-5-fluoropyrimidin-4-yl)amino)benzamide (Compound 14)

N-(2-chlorophenyl)-4-((5-fluoro-2-((4-(2-oxo-2-(piperazin-1-yl)ethyl)phenyl)amino)pyrimidine-4-yl)amino)benzamide (124.36 mg, 208.50 μmol, HCl), DIPEA (134.73 mg, 1.04 mmol, 181.58 μL) and NaI (6.25 mg, 41.70 μmol) were added to a solution of 6-((2-(2,6-dioxopiperidin-3-yl)-1,3-dioxoisoindolin-4-yl)amino)hexyl 4-methylbenzenesulfonate (110 mg, 208.50 μmol) in DMF (3 mL) and then stirred at 80° C. for 12 hours. It was confirmed through LCMS that the starting material was completely consumed, and the desired mass was detected. The mixture was diluted with water (20 mL) and then extracted with EtOAc (10 mL×3). The combined organic phase was washed with saturated brine (10 mL), dried over Na₂SO₄, and filtered and concentrated under reduced pressure. The residue was purified by prep-HPLC (column: Phenomenex C18 75×30 mm×3 μm; mobile phase: [water (FA)-ACN]; B %: 18%-48%, 7 min) and prep-HPLC (column: Waters Xbridge 150×25 mm×5 μm; mobile phase: [water (NH₄HCO₃)-ACN]; B %: 45%-75%, 8 min) and freeze-dried to obtain Compound 14 (14.5 mg, 14.30 μmol, 6.86% yield, 90.3% purity) as yellow solid. MS(M+H)⁺=915.5.

¹H NMR (400 MHz, DMSO-d₆) δ=11.09 (s, 1H), 9.90-9.86 (m, 1H), 9.64 (br s, 1H), 9.27 (s, 1H), 8.17 (d, J=3.5 Hz, 1H), 8.06-8.00 (m, 2H), 8.00-7.93 (m, 2H), 7.68-7.63 (m, 1H), 7.62-7.51 (m, 4H), 7.43-7.36 (m, 1H), 7.32-7.27 (m, 1H), 7.15-7.05 (m, 3H), 7.01 (d, J=7.0 Hz, 1H), 6.55-6.48 (m, 1H), 5.08-5.01 (m, 1H), 3.64-3.61 (m, 2H), 3.46-3.42 (m, 4H), 3.34-3.33 (m, 2H), 3.29 (s, 2H), 2.87-2.86 (m, 1H), 2.62-2.58 (m, 2H), 2.24-2.22 (m, 4H), 2.05-2.00 (m, 1H), 1.58-1.51 (m, 2H), 1.41-1.27 (m, 6H).

### Example 15: Synthesis of Compound 15

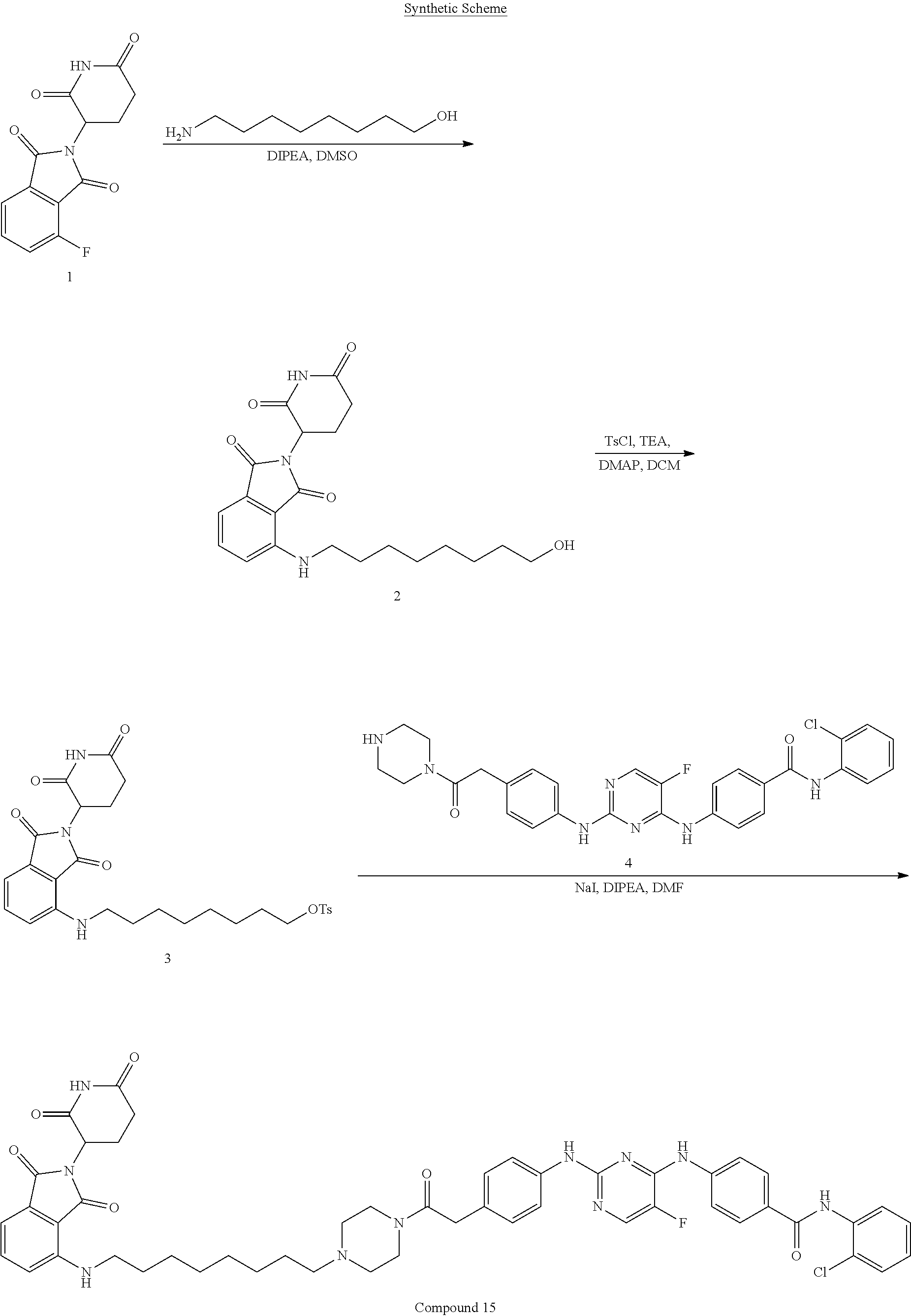 [SMILES: O=C1CCC(N2C(=O)c3cccc(F)c3C2=O)C(=O)N1 ; NCCCCCCCCO ; O=C1CCC(N2C(=O)c3cccc(NCCCCCCCCO)c3C2=O)C(=O)N1 ; Cc1ccc(S(=O)(=O)OCCCCCCCCNc2cccc3c2C(=O)N(C2CCC(=O)NC2=O)C3=O)cc1 ; O=C(Nc1ccccc1Cl)c1ccc(Nc2nc(Nc3ccc(CC(=O)N4CCNCC4)cc3)ncc2F)cc1 ; O=C1CCC(N2C(=O)c3cccc(NCCCCCCCCN4CCN(C(=O)Cc5ccc(Nc6ncc(F)c(Nc7ccc(C(=O)Nc8ccccc8Cl)cc7)n6)cc5)CC4)c3C2=O)C(=O)N1]

### Experimental Process

### Step 1: Synthesis of 2-(2,6-dioxopiperidin-3-yl)-4-((8-hydroxyoctyl)amino)isoindoline-1,3-dione (2)

A solution of 2-(2,6-dioxo-3-piperidyl)-4-fluoro-isoindoline-1,3-dione (500 mg, 1.81 mmol), 8-aminooctane-1-ol (262.91 mg, 1.81 mmol) and DIPEA (467.90 mg, 3.62 mmol) in DMSO (5 mL) was stirred at 100° C. for 14 hours. The main peak of desired mass was detected by LCMS. The mixture was poured with water (15 mL) and extracted with EtOAc (8 mL×3). The combined organic phase was washed with brine (5 mL×3), dried over Na₂SO₄, and concentrated under reduced pressure to obtain 2-(2,6-dioxopiperidin-3-yl)-4-((8-hydroxyoctyl)amino)isoindoline-1,3-dione (620 mg, crude) as dark green solid. MS(M+H)⁺=402.3.

### Step 2: Synthesis of 8-((2-(2,6-dioxopiperidin-3-yl)-1,3-dioxoisoindolin-4-yl)amino)octyl 4-methylbenzenesulfonate (3)

TEA (504.05 mg, 4.98 mmol) and DMAP (20.28 mg, 166.00 μmol) were added to a solution of 2-(2,6-dioxopiperidin-3-yl)-4-((8-hydroxyoctyl)amino)isoindoline-1,3-dione (620 mg, 1.54 mmol) and TsCl (474.83 mg, 2.49 mmol) in DCM (15 mL) at 20° C. and then stirred for 14 hours. It was confirmed through LCMS that the starting material was completely consumed, and a main peak with the desired mass was detected. The mixture was concentrated under reduced pressure and then purified by flash silica gel chromatography (10 g silica gel column, EtOAc/petroleum ether=20-80%, 60 mL/min) to obtain 8-((2-(2,6-dioxopiperidin-3-yl)-1,3-dioxoisoindolin-4-yl)amino)octyl 4-methylbenzenesulfonate (450 mg, 809.88 μmol, 52.44% yield) as yellow solid. MS(M+H)⁺=556.2.

### Step 3: Synthesis of N-(2-chlorophenyl)-4-((2-((4-(2-(4-(8-((2-(2,6-dioxopiperidin-3 yl))-1,3-dioxoisoindolin-4-yl)amino)octyl)piperazin-1-yl)-2-oxoethyl)phenyl)amino)-5-fluoropyrimidin-4-yl)amino)benzamide (Compound 15)

DIPEA (104.67 mg, 809.88 μmol) and NaI (8.09 mg, 53.99 μmol) were added to a solution of 8-((2-(2,6-dioxopiperidin-3-yl)-1,3-dioxoisoindolin-4-yl)amino)octyl 4-methylbenzenesulfonate (150 mg, 269.96 μmol) and N-(2-chlorophenyl)-4-((5-fluoro-2-((4-(2-oxo-2-(piperazin-1-yl)ethyl)phenyl)amino)pyrimidin-4-yl)amino)benzamide (130 mg, 217.94 μmol, HCl salt) in DMF (3 mL) at 20° C. and then stirred at 80° C. for 14 hours. The main peak was detected by LCMS. The mixture was poured with water (10 mL) and extracted with EtOAc (8 mL×3). The combined organic phase was washed with brine (5 mL), dried over Na₂SO₄, and concentrated under reduced pressure. The product was purified by prep-HPLC (column: Waters Xbridge 150*25 mm*5 um; mobile phase: [water (FA)-ACN]; B %: 6%-36%, 10 min) and freeze-dried, and then further purified by prep-HPLC (column: Waters Xbridge C18 150*50 mm*10 um; mobile phase: [water (NH₄HCO₃)-ACN]; B %: 52%-82%, 10 min) and freeze-dried to obtain Compound 15 (14.8 mg, 14.43 μmol, 5.35% yield, 92% purity) as yellow solid. MS(M+H)⁺=943.2.

¹H NMR (400 MHz, DMSO-d₆) δ=11.09 (s, 1H), 9.88 (s, 1H), 9.65 (s, 1H), 9.28 (s, 1H), 8.18 (d, J=3.5 Hz, 1H), 8.06-8.01 (m, 2H), 7.99-7.95 (m, 2H), 7.65 k (dd, J=1.5, 7.9 Hz, 1H), 7.62-7.54 (m, 4H), 7.39 (dt, J=1.3, 7.7 Hz, 1H), 7.32-7.26 (m, 1H), 7.14-7.06 (m, 3H), 7.01 (d, J=7.0 Hz, 1H), 6.51 (br t, J=5.4 Hz, 1H), 5.04 (dd, J=5.3, 12.8 Hz, 1H), 3.62 (s, 2H), 3.46-3.38 (m, 4H), 3.30-3.24 (m, 2H), 2.93-2.82 (m, 1H), 2.62-2.53 (m, 2H), 2.25-2.16 (m, 6H), 2.06-1.99 (m, 1H), 1.59-1.51 (m, 2H), 1.37-1.21 (m, 10H).

### Example 16: Synthesis of Compound 16

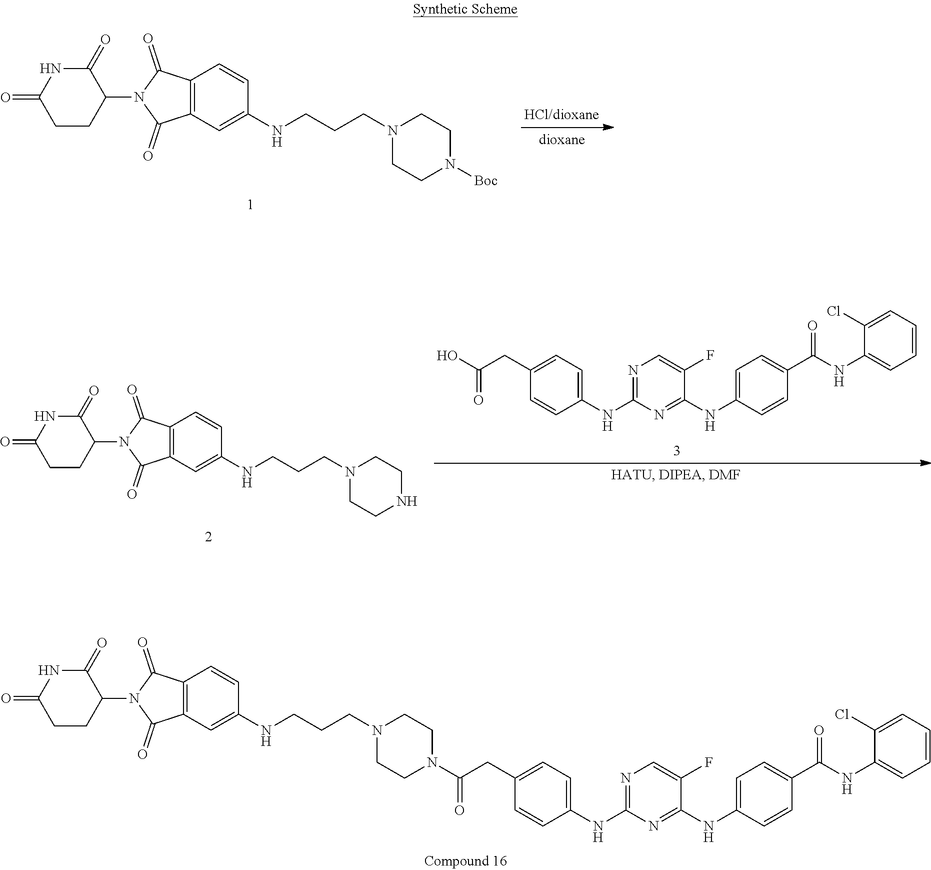 [SMILES: CC(C)(C)OC(=O)N1CCN(CCCNc2ccc3c(c2)C(=O)N(C2CCC(=O)NC2=O)C3=O)CC1 ; O=C1CCC(N2C(=O)c3ccc(NCCCN4CCNCC4)cc3C2=O)C(=O)N1 ; O=C(O)Cc1ccc(Nc2ncc(F)c(Nc3ccc(C(=O)Nc4ccccc4Cl)cc3)n2)cc1 ; O=C1CCC(N2C(=O)c3ccc(NCCCN4CCN(C(=O)Cc5ccc(Nc6ncc(F)c(Nc7ccc(C(=O)Nc8ccccc8Cl)cc7)n6)cc5)CC4)cc3C2=O)C(=O)N1]

### Experimental Process

### Step 1: Synthesis of 2-(2,6-dioxopiperidin-3 yl)-5-((3-(piperazin-1-yl)propyl)amino)isoindoline-1,3-dione (2)

HCl/dioxane (4 M, 3 mL) was added to a solution of tert-butyl 4-(3-((2-(2,6-dioxopiperidin-3-yl)-1,3-dioxoisoindoline-5-yl)amino)propyl)piperazine-1-carboxylate (100 mg, 200.18 μmol) in dioxane (3 mL) and then stirred at 20° C. for 30 minutes. The main peak of desired mass was detected by LCMS. The mixture was concentrated under reduced pressure to obtain 2-(2,6-dioxopiperidin-3-yl)-5-((3-(piperazin-1-yl)propyl)amino)isoindoline-1,3-dione (87 mg, crude, HCl) as yellow solid. MS(M+H)⁺=400.2.

### Step 2: Synthesis of N-(2-chlorophenyl)-4-((2-((4-(2-(4-(3-((2-(2,6-dioxopiperidin-3 yl))-1,3-dioxoisoindoline-5-yl)amino)propyl)piperazin-1 yl)-2-oxoethyl)phenyl)amino)-5-fluoropyrimidin-4-yl)amino)benzamide (Compound 16)

HATU (113.83 mg, 299.38 μmol) and DIPEA (77.39 mg, 598.76 μcool, 104.29 μL) were added to a solution of 2-(4-((4-((4-((2-chlorophenyl)carbamoyl)phenyl)amino)-5-fluoropyrimidin-2-yl)amino)phenyl)acetic acid (98.18 mg, 199.59 μcool) in DMF (1 mL). The mixture was stirred at 20° C. for 30 minutes. 2-(2,6-dioxopiperidin-3-yl)-5-((3-(piperazin-1-yl)propyl)amino)isoindoline-1,3-dione (87 mg, 199.59 μmol, HCl) was added and stirred at 20° C. for 16 hours. The main peak of desired mass was detected by LCMS. The mixture was diluted with water (10 mL) and then extracted with EtOAc (5 mL×3). The combined organic phase was washed with brine (10 mL×3), dried over Na₂SO₄, and filtered. The filtrate was concentrated under reduced pressure and then the residue was purified by prep-TLC (SiO₂, DCM:MeOH=9:1) and then repurified by reverse phase HPLC (column: Phenomenex luna C18 150*25 mm*10 um; mobile phase: [water (FA)-ACN]; B %: 10%-40%, 2 min) and freeze-dried to obtain Compound 16 (22.5 mg, 23.70 μmol, 11.88% yield, 92% purity) as yellow solid. MS(M+H)⁺=873.3.

¹H NMR (400 MHz, DMSO-d₆) δ=11.05 (s, 1H), 9.88 (s, 1H), 9.65 (s, 1H), 9.28 (s, 1H), 8.18 (d, J=3.5 Hz, 1H), 8.07-7.95 (m, 4H), 7.68-7.53 (m, 5H), 7.41-7.35 (m, 1H), 7.31-7.25 (m, 1H), 7.17-7.04 (m, 3H), 6.95 (d, J=1.5 Hz, 1H), 6.86-6.81 (m, 1H), 5.08-4.96 (m, 1H), 3.69-3.59 (m, 2H), 3.53-3.44 (m, 4H), 3.22-3.15 (m, 2H), 2.93-2.81 (m, 1H), 2.69-2.57 (m, 1H), 2.44-2.36 (m, 2H), 2.35-2.30 (m, 2H), 2.29-2.25 (m, 3H), 2.04-1.93 (m, 1H), 1.74-1.63 (m, 2H).

### Example 17: Synthesis of Compound 17

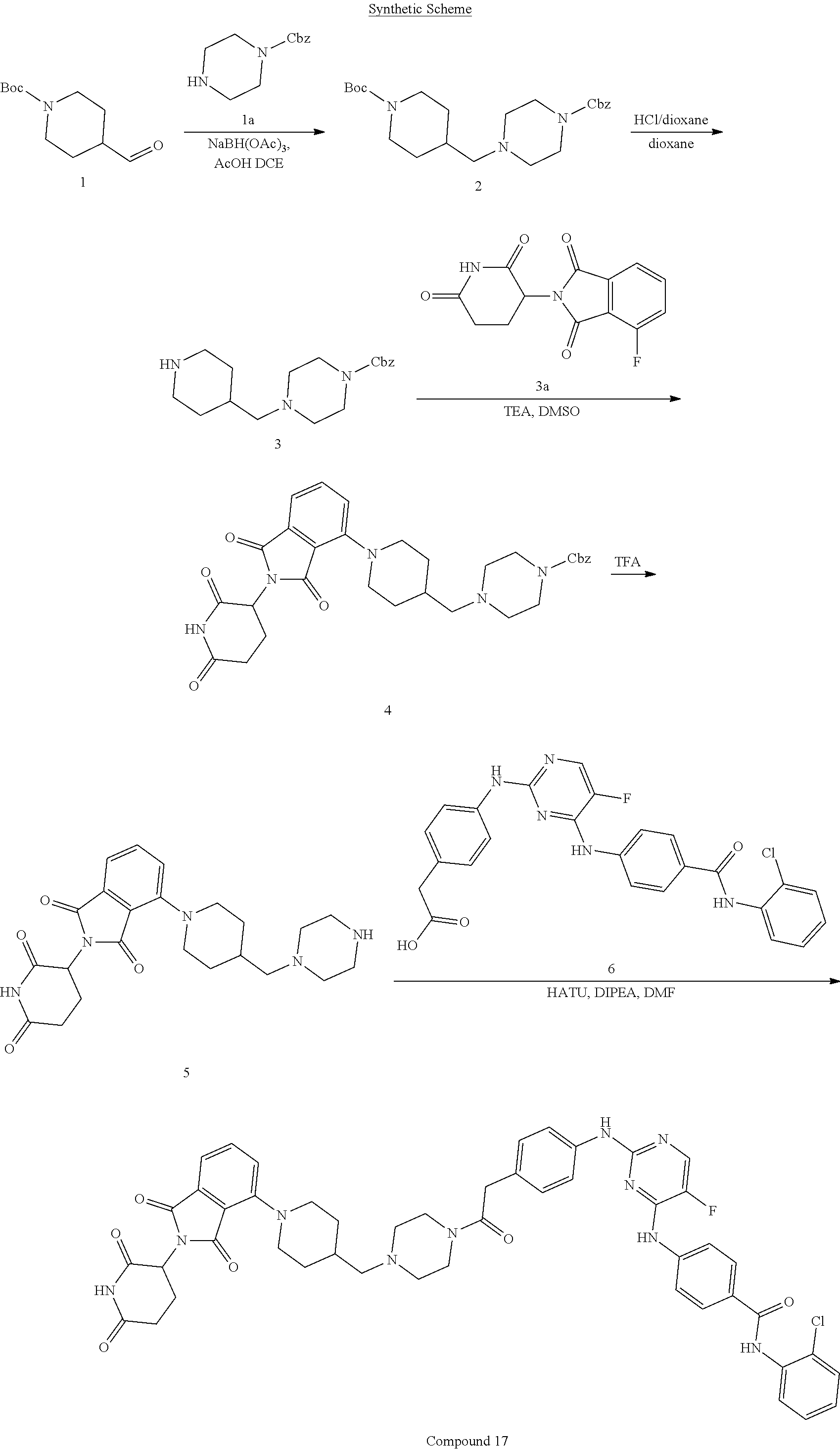

### Experimental Process

### Step 1: Synthesis of 4-((1-(tert-butoxycarbonyl)piperidin-4-yl)methyl)piperazine-1-carboxylate (2)

AcOH (844.69 mg, 14.07 mmol, 804.47 μL) was added to a solution of tert-butyl 4-formylpiperidine-1-carboxylate (3 g, 14.07 mmol) and benzyl piperazine-1-carboxylate (3.10 g, 14.07 mmol, 2.72 mL) in DCE (30 mL) at 25° C. and then stirred for 30 minutes. Thereafter, the mixture was added with NaBH(OAc)₃ (5.96 g, 28.13 mmol) at 25° C. and then stirred for 12 hours. It was confirmed through LCMS that the starting material was completely consumed, and a main peak with the desired mass was detected. The product was diluted with DCM (50 mL), washed with saturated NaHCO₃ (30 mL), dried over Na₂SO₄, filtered, and concentrated under reduced pressure. The residue was purified by flash silica gel chromatography (25 g SepaFlash® Silica Flash Column, Eluent of 0˜25% EtOAc/Petroleum ethergradient @ 120 mL/min) to obtain 4-((1-(tert-butoxycarbonyl)piperidin-4-yl)methyl)piperazine-1-carboxylate (5.3 g, 12.19 mmol, 86.63% yield, 96% purity) as colorless oil, which was immediately used in the next reaction. MS(M+H)⁺=418.2

### Step 2: Synthesis of benzyl 4-(piperidin-4-ylmethyl)piperazine-1-carboxylate (3)

HCl/dioxane (4 M, 41.16 mL) was added to a solution of benzyl 4-((1-(tert-butoxycarbonyl)piperidin-4-yl)methyl)piperazine-1-carboxylate (1.1 g, 2.63 mmol) in dioxane (2 mL) at 25° C. and then stirred for 30 minutes. It was confirmed through LCMS that the starting material was completely consumed. The mixture was concentrated under reduced pressure to obtain benzyl 4-(piperidin-4-ylmethyl)piperazine-1-carboxylate (930 mg, crude, HCl) as brown solid, which was immediately used in the next reaction. MS(M+H)⁺=318.0

### Step 3: Synthesis of benzyl 4-((1-(2-(2,6-dioxopiperidin-3 yl)-1,3-dioxoisoindolin-4-yl)piperidin-4-yl)methyl)piperazine-1-carboxylate (4)

A solution of 2-(2,6-dioxopiperidin-3-yl)-4-fluoroisoindoline-1,3-dione (359.05 mg, 1.30 mmol), benzyl 4-(piperidin-4-ylmethyl)piperazine-1-carboxylate (460 mg, 1.30 mmol, HCl) and TEA (526.13 mg, 5.20 mmol, 723.70 μL) in DMSO (6 mL) was stirred at 100° C. for 12 hours. It was confirmed through LCMS that the starting material was completely consumed, and a peak with the desired mass was detected. The mixture was poured with water (30 mL) and extracted with EtOAc (20 mL×3). The combined organic phase was washed with brine (10 mL×3), dried over Na₂SO₄, filtered, and concentrated under reduced pressure. The residue was purified by flash silica gel chromatography (4 g SepaFlash® Silica Flash Column, Eluent of 0˜70% EtOAc/Petroleum ether gradient @ 80 mL/min) to obtain benzyl 4-((1-(2-(2,6-dioxopiperidin-3-yl)-1,3-dioxoisoindolin-4-yl)piperidin-4-yl)methyl)piperazine-1-carboxylate (0.6 g, crude) as yellow oil, which was immediately used in the next reaction. MS(M+H)⁺=574.3

### Step 4: Synthesis of 2-(2,6-dioxopiperidin-3-yl)-4-(4-(piperazin-1-ylmethyl)piperidin-1 yl)isoindoline-1,3-dione (5)

A solution of benzyl 4-((1-(2-(2,6-dioxopiperidin-3-yl)-1,3-dioxoisoindolin-4-yl)piperidin-4-yl)methyl)piperazine-1-carboxylate (0.6 g, 1.05 mmol) in TFA (5 mL) was stirred at 40° C. for 12 hours. It was confirmed through LCMS that the starting material was completely consumed, and a peak with the desired mass was detected. The mixture was concentrated under reduced pressure to obtain 2-(2,6-dioxopiperidin-3-yl)-4-(4-(piperazin-1-ylmethyl)piperidin-1-yl)isoindoline-1,3-dione (0.5 g, crude, TFA) as brown oil, which was immediately used in the next reaction. MS(M+H)⁺=440.2

### Step 5: Synthesis of N-(2-chlorophenyl)-4-((2-((4-(2-(4-((1-(2-(2,6-dioxopiperidin-3 yl))-1,3-dioxoisoindolin-4-yl)piperidin-4-yl)methyl)piperazin-1-yl)-2-oxoethyl)phenyl)amino)-5-fluoropyrimidin-4-yl)amino)benzamide (Compound 17)

HATU (46.38 mg, 121.98 μmol) and DIPEA (65.68 mg, 508.23 μmol, 88.52 μL) were added to a solution of 2-(4-((4-((4-((2-chlorophenyl)carbamoyl)phenyl)amino)-5-fluoropyrimidin-2-yl)amino)phenyl)acetic acid (50 mg, 101.65 μmol) in DMF (1 mL) and then stirred at 25° C. for 10 minutes. Thereafter, 2-(2,6-dioxopiperidin-3-yl)-4-(4-(piperazin-1-ylmethyl)piperidin-1-yl)isoindoline-1,3-dione (112.53 mg, 203.29 μmol, TFA) was added and stirred at 25° C. for 1 hour. It was confirmed through LCMS that the starting material was completely consumed, and a peak with the desired mass was detected. The mixture was poured with water (30 mL) and extracted with EtOAc (10 mL×3). The combined organic phase was washed with brine (10 mL×3), dried over Na₂SO₄, filtered, and concentrated under reduced pressure. The residue was purified by prep-HPLC (column: Unisil 3-100 C18 Ultra 150*50 mm*3 um; mobile phase: [water(FA)-ACN]; B %: 19%-49%, 7 min) and freeze-dried. The product was repurified by prep-HPLC (SiO₂, DCM:MeOH=10:1) to obtain Compound 17 (14.7 mg, 15.77 umol, 14.41% yield, 98% purity) as yellow solid. MS(M+H)⁺=913.3.

¹H NMR (400 MHz, DMSO-d₆) δ=11.08 (s, 1H), 9.88 (s, 1H), 9.65 (s, 1H), 9.28 (s, 1H), 8.18 (d, J=3.7 Hz, 1H), 8.06-7.95 (m, 4H), 7.70-7.58 (m, 4H), 7.57-7.54 (m, 1H), 7.42-7.37 (m, 1H), 7.34-7.27 (m, 3H), 7.12 (d, J=8.6 Hz, 2H), 5.13-5.05 (m, 1H), 3.71-3.62 (m, 4H), 3.51-3.40 (m, 4H), 3.31-3.29 (m, 2H), 2.90-2.78 (m, 3H), 2.63-2.56 (m, 2H), 2.28-2.21 (m, 3H), 2.16-2.12 (m, 1H), 2.05-1.97 (m, 1H), 1.90-1.60 (m, 3H), 1.34-1.25 (m, 2H).

### Example 18: Synthesis of Compound 18

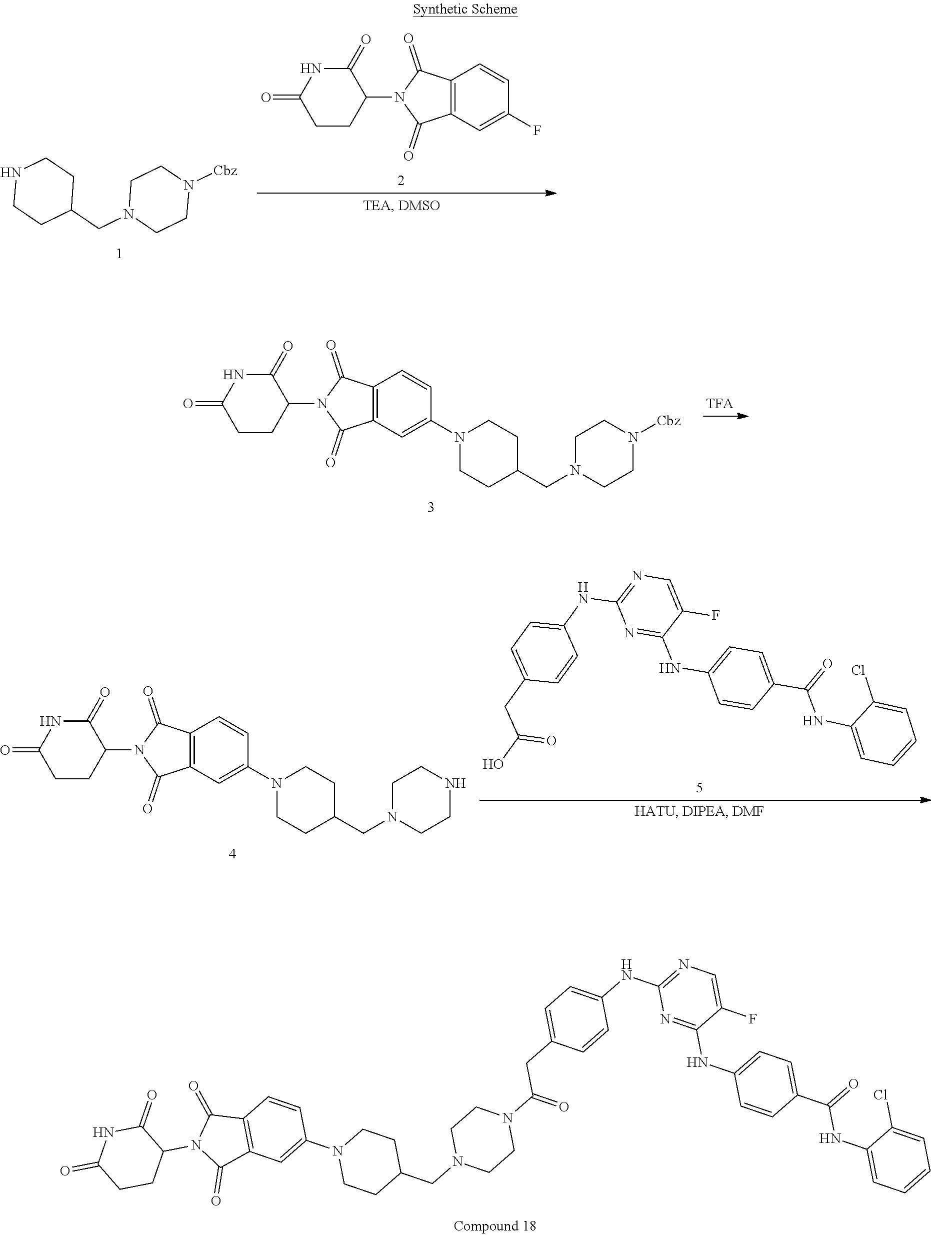 [SMILES: O=C1CCC(N2C(=O)c3ccc(F)cc3C2=O)C(=O)N1 ; O=C(OCc1ccccc1)N1CCN(CC2CCNCC2)CC1 ; O=C1CCC(N2C(=O)c3ccc(N4CCC(CN5CCN(C(=O)OCc6ccccc6)CC5)CC4)cc3C2=O)C(=O)N1 ; O=C1CCC(N2C(=O)c3ccc(N4CCC(CN5CCNCC5)CC4)cc3C2=O)C(=O)N1 ; O=C(O)Cc1ccc(Nc2ncc(F)c(Nc3ccc(C(=O)Nc4ccccc4Cl)cc3)n2)cc1 ; O=C1CCC(N2C(=O)c3ccc(N4CCC(CN5CCN(C(=O)Cc6ccc(Nc7ncc(F)c(Nc8ccc(C(=O)Nc9ccccc9Cl)cc8)n7)cc6)CC5)CC4)cc3C2=O)C(=O)N1]

### Experimental Process

### Step 1: Synthesis of benzyl 4-((1-(2-(2,6-dioxopiperidin-3 yl)-1,3-dioxoisoindoline-5 yl)piperidin-4-yl)methyl)piperazine-1-carboxylate (3)

A solution of benzyl 4-(4-piperidylmethyl)piperazine-1-carboxylate (460 mg, 1.30 mmol, HCl), 2-(2,6-dioxo-3-piperidyl)-5-fluoro-isoindoline-1,3-dione (359.05 mg, 1.30 mmol) and TEA (526.13 mg, 5.20 mmol) in DMSO (6 mL) was stirred at 100° C. for 12 hours. It was confirmed through LCMS that the starting material was completely consumed, and the desired mass was detected. The mixture was poured with water (30 mL) and extracted with EtOAc (10 mL×3). The combined organic phase was washed with brine (10 mL×3), dried over Na₂SO₄, filtered, and concentrated under reduced pressure. The residue was purified by flash silica gel chromatography (4 g Sepa Flash® Silica Flash Column, Eluent of 0˜70% EtOAc/Petroleum ether gradient @ 80 mL/min) to obtain benzyl 4-((1-(2-(2,6-dioxopiperidin-3-yl)-1,3-dioxoisoindoline-5-yl)piperidin-4-yl)methyl)piperazine-1-carboxylate (0.4 g, crude) as yellow solid, which was immediately used in the next reaction. MS(M+H)⁺=574.2

### Step 2: Synthesis of 2-(2,6-dioxopiperidin-3-yl)-5-(4-(piperazin-1-ylmethyl)piperidin-1 yl)isoindoline-1,3-dione (4)

A solution of benzyl 4-((1-(2-(2,6-dioxopiperidin-3-yl)-1,3-dioxoisoindolin-5-yl)piperidin-4-yl)methyl)piperazine-1-carboxylate (0.4 g, 697.30 μmol) in TFA (5 mL) was stirred at 50° C. for 2 hours. It was confirmed through LCMS that the starting material was completely consumed, and a main peak with the desired mass was detected. The mixture was concentrated under reduced pressure to obtain 2-(2,6-dioxopiperidin-3-yl)-5-(4-(piperazin-1-ylmethyl)piperidin-1-yl)isoindoline-1,3-dione (0.4 g, crude, TFA) as yellow oil, which was immediately used in the next reaction. MS(M+H)⁺=440.2

### Step 3: Synthesis of N-(2-chlorophenyl)-4-((2-((4-(2-(4-((1-(2-(2,6-dioxopiperidin-3 yl))-1,3-dioxoisoindoline-5-yl)piperidin-4-yl)methyl)piperazin-1-15 yl)-2-oxoethyl)phenyl)amino)-5-fluoropyrimidin-4-yl)amino)benzamide (Compound 18)

HATU (92.76 mg, 243.95 μmol) and DIPEA (131.37 mg, 1.02 mmol, 177.05 μL) were added to a solution of 2-(4-((4-((4-((2-chlorophenyl)carbamoyl)phenyl)amino)-5-fluoropyrimidin-2-yl) amino)phenyl) acetic acid (100 mg, 203.29 μmol) in DMF (2 mL) and then stirred at 20° C. for 30 minutes. Thereafter, 2-(2,6-dioxopiperidin-3-yl)-5-(4-(piperazin-1-ylmethyl)piperidin-1-yl)isoindoline-1,3-dione (135.71 mg, 203.29 μmol, 2TFA) was added and stirred at 20° C. for 12 hours. The main peak of desired mass was detected by LCMS. The mixture was diluted with water (10 mL) and then extracted with EtOAc (10 mL×3). The combined organic phase was washed with brine (20 mL×2), dried over Na₂SO₄, and filtered. The filtrate was concentrated under reduced pressure and then the residue was purified by prep-TLC (SiO₂, DCM:MeOH=10:1) and then repurified by reverse phase HPLC (column: Waters Xbridge 150*25 mm*5 um; mobile phase: [water (NH₄HCO₃)-ACN]; B %: 40%-70%, 8 min). The eluate was freeze-dried to obtain Compound 18 (30.2 mg, 30.42 μmol, 14.96% yield, 92% purity) as yellow solid. MS(M+H)⁺=913.4.

¹H NMR (400 MHz, DMSO-d₆) δ=11.07 (s, 1H), 9.88 (s, 1H), 9.65 (s, 1H), 9.28 (s, 1H), 8.18 (d, J=3.5 Hz, 1H), 8.06-7.91 (m, 4H), 7.68-7.52 (m, 5H), 7.42-7.36 (m, 1H), 7.31-7.26 (m, 2H), 7.23-7.18 (m, 1H), 7.12 (d, J=8.6 Hz, 2H), 5.09-5.02 (m, 1H), 4.04-3.95 (m, 2H), 3.70-3.58 (m, 2H), 3.50-3.40 (m, 4H), 3.31-3.30 (m, 2H), 2.97-2.83 (m, 3H), 2.62-2.53 (m, 2H), 2.26-2.20 (m, 3H), 2.10-2.07 (m, 1H), 2.04-1.98 (m, 1H), 1.82-1.68 (m, 3H), 1.15-1.04 (m, 2H).

### Example 19: Synthesis of Compound 19

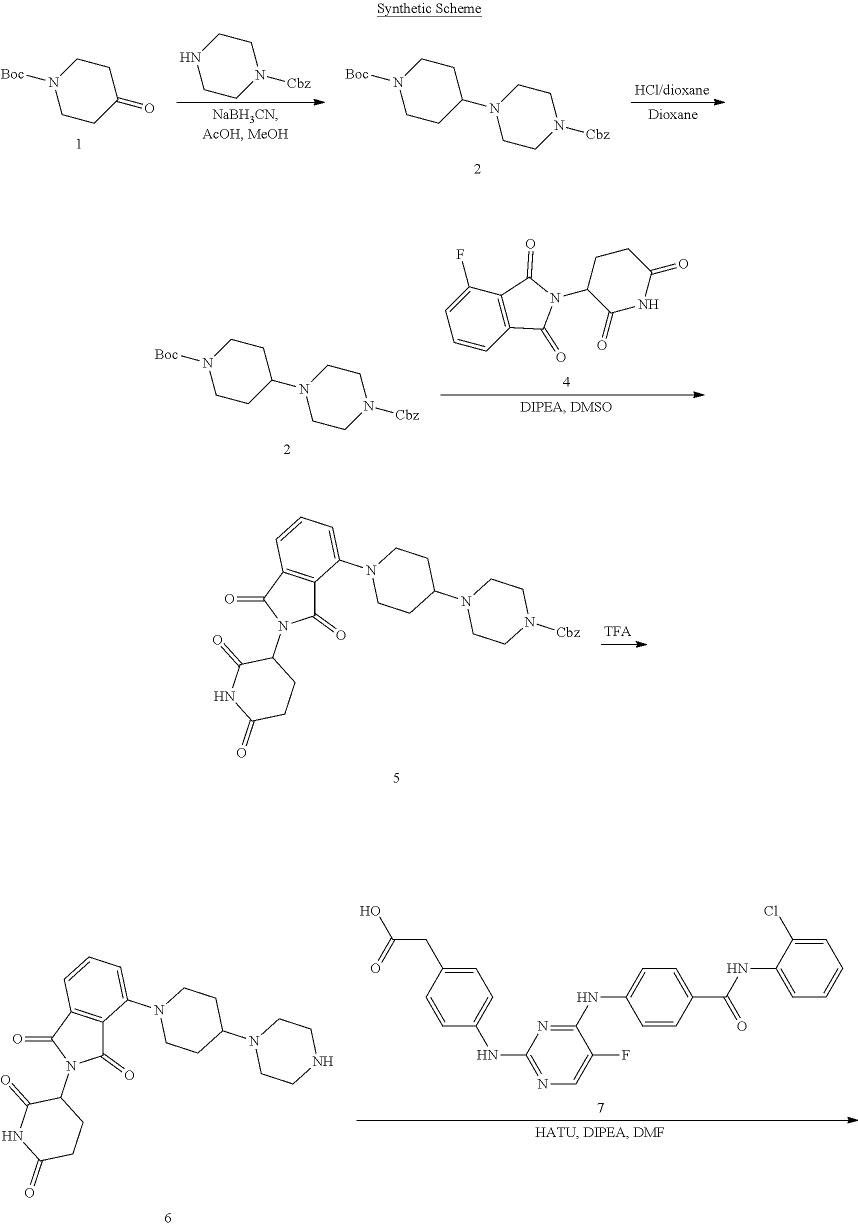

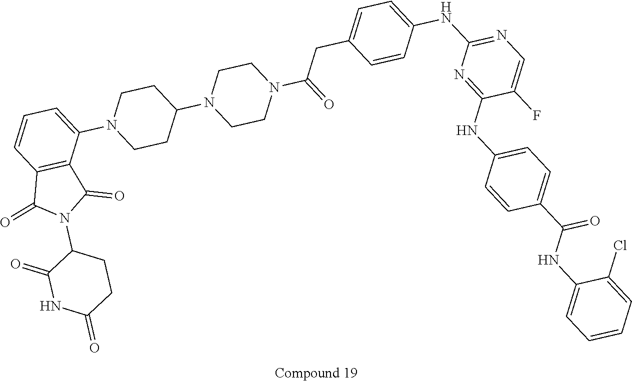 [SMILES: O=C1CCC(N2C(=O)c3cccc(N4CCC(N5CCN(C(=O)Cc6ccc(Nc7ncc(F)c(Nc8ccc(C(=O)Nc9ccccc9Cl)cc8)n7)cc6)CC5)CC4)c3C2=O)C(=O)N1]

### Experimental Process

### Step 1: Synthesis of benzyl 4-(1-(tert-butoxycarbonyl)piperidin-4-yl)piperazine-1-carboxylate (2)

A solution of tert-butyl 4-oxopiperidine-1-carboxylate (2 g, 10.04 mmol), benzyl piperazine-1-carboxylate (2.21 g, 10.04 mmol) and AcOH (301.40 mg, 5.02 mmol) in MeOH (30 mL) was stirred at 20° C. for 1 hour. Thereafter, NaBH₃CN (946.20 mg, 15.06 mmol) was added and then the mixture was stirred at 20° C. for 13 hours. The main peak of desired mass was detected by LCMS. The mixture was quenched with NaHCO₃ (10 mL) and concentrated under reduced pressure to remove MeOH. The residue was dissolved in EtOAc (60 mL), washed with brine (10 mL), dried over Na₂SO₄, and concentrated under reduced pressure. The residue was purified by flash silica gel chromatography (ISCO®; 25 g SepaFlash® Silica Flash Column, Eluent of 5˜50% EtOAc/Petroleum ether gradient @ 100 mL/min) to obtain benzyl 4-(1-(tert-butoxycarbonyl)piperidin-4-yl)piperazine-1-carboxylate (2.4 g, crude) as pale yellow oil. MS(M+H)⁺=404.1

### Step 2: Synthesis of benzyl 4-(piperidin-4-yl)piperazine-1-carboxylate (3)

HCl/dioxane (4 M, 15 mL) was added to a solution of benzyl 4-(1-(tert-butoxycarbonyl)piperidin-4-yl)piperazine-1-carboxylate (2.4 g, 5.95 mmol) in dioxane (15 mL) at 20° C. and then stirred for 1 hour. It was confirmed through LCMS that the starting material was completely consumed, and the desired mass was detected. The mixture was concentrated under reduced pressure to obtain benzyl 4-(piperidin-4-yl)piperazine-1-carboxylate (2.2 g, crude, 2HCl) as white solid, which was immediately used in the next reaction. MS(M+H)⁺=304.3

### Step 3: Synthesis of benzyl 4-(1-(2-(2,6-dioxopiperidin-3 yl)-1,3-dioxoisoindolin-4-yl)piperidin-4-yl)piperazine-1-carboxylate (5)

DIPEA (561.48 mg, 4.34 mmol) was added to a solution of 2-(2,6-dioxo-3-piperidyl)-4-fluoro-isoindoline-1,3-dione (400 mg, 1.45 mmol) and benzyl 4-(piperidin-4-yl)piperazine-1-carboxylate (500 mg, 1.47 mmol, HCl) in DMSO (8 mL) at 20° C. The mixture was stirred at 100° C. for 14 hours. It was confirmed through LCMS that the starting material was completely consumed, and the desired mass was detected. The mixture was poured with water (30 mL) and extracted with EtOAc (10 mL×4). The combined organic phase was washed with brine (10 mL×2), dried over Na₂SO₄, and concentrated under reduced pressure. The residue was purified by flash silica gel chromatography (ISCO®; 4 g SepaFlash® Silica Flash Column, Eluent of 30˜100% EtOAc/Petroleum ether gradient @ 50 mL/min) to obtain benzyl 4-(1-(2-1: (2,6-dioxopiperidin-3-yl)-1,3-dioxoisoindolin-4-yl)piperidin-4-yl)piperazine-1-carboxylate (400 mg, crude) as yellow oil. MS(M+H)⁺=560.3

### Step 4: Synthesis of 2-(2,6-dioxopiperidin-3-yl)-4-(4-(piperazin-1-yl)piperidin-1-yl)isoindoline-1,3-dione (6)

A solution of benzyl 4-(1-(2-(2,6-dioxopiperidin-3-yl)-1,3-dioxoisoindolin-4-yl)piperidin-4-yl) piperazine-1-carboxylate (200 mg, 357.39 μmol) in TFA (4 mL) was stirred at 40° C. for 14 hours. A trace of starting material and the desired mass were detected through LCMS. The mixture was further stirred at 60° C. for 1 hour. The mixture was concentrated under reduced pressure, and the residue was dissolved in water (20 mL) and freeze-dried to obtain 2-(2,6-dioxopiperidin-3-yl)-4-(4-(piperazin-1-yl)piperidin-1-yl)isoindoline-1,3-dione (250 mg, crude, 2TFA) as yellow solid. MS(M+H)⁺=426.3

### Step 5: Synthesis of N-(2-chlorophenyl)-4-((2-((4-(2-(4-(1-(2-(2,6-dioxopiperidin-3 yl))-1,3-dioxoisoindolin-4-yl)piperidin-4-yl)piperazin-1 yl)-2-oxoethyl)phenyl)amino)-5-fluoropyrimidin-4-yl)amino)benzamide (Compound 19)

2-(2,6-dioxopiperidin-3-yl)-4-(4-(piperazin-1-yl)piperidin-1-yl)isoindoline-1,3-dione (132.86 mg, 203.29 μmol, 2TFA) was added to a solution of 2-(4-((4-((4-((2-chlorophenyl)carbamoyl)phenyl)amino)-5-fluoropyrimidin-2-yl)amino)phenyl)acetic acid (100 mg, 203.29 μmol), DIPEA (131.37 mg, 1.02 mmol) and HATU (92.76 mg, 243.95 μmol) in DMF (2 mL) at 20° C. and then the mixture was stirred for 2 hours. The main peak of desired mass was detected by LCMS. The mixture was poured with water (15 mL) and extracted with EtOAc (10 mL×4). The combined organic phase was washed with brine (10 mL) and dried over Na₂SO₄. The product was purified by prep-HPLC (column: Waters Xbridge 150*25 mm*5 um; mobile phase: [water (NH₄HCO₃)-ACN]; B %: 37%-67%, 8 min) and freeze-dried to obtain Compound 19 (44.5 mg, 44.04 μmol, 21.66% yield, 89% purity) as yellow k solid. MS(M+H)⁺=899.2.

¹H NMR (400 MHz, DMSO-d₆) δ=11.08 (s, 1H), 9.88 (s, 1H), 9.65 (s, 1H), 9.28 (s, 1H), 8.18 (d, J=3.5 Hz, 1H), 8.05-8.00 (m, 2H), 7.99-7.95 (m, 2H), 7.70-7.63 (m, 2H), 7.60 (d, J=8.6 Hz, 2H), 7.56 (dd, J=1.3, 8.0 Hz, 1H), 7.39 (dt, J=1.3, 7.7 Hz, 1H), 7.34-7.26 (m, 3H), 7.12 (d, J=8.4 Hz, 2H), 5.08 (dd, J=5.3, 13.0 Hz, 1H), 3.75-3.68 (m, 2H), 3.64 (s, 2H), 3.51-3.41 (m, 4H), 3.31-3.29 (m, 4H), 2.91-2.80 (m, 3H), 2.62-2.57 (m, 1H), 2.44-2.41 (m, 2H), 2.06-1.97 (m, 1H), 1.86-1.75 (m, 2H), 1.63-1.50 (m, 2H).

### Example 20: Synthesis of Compound 20

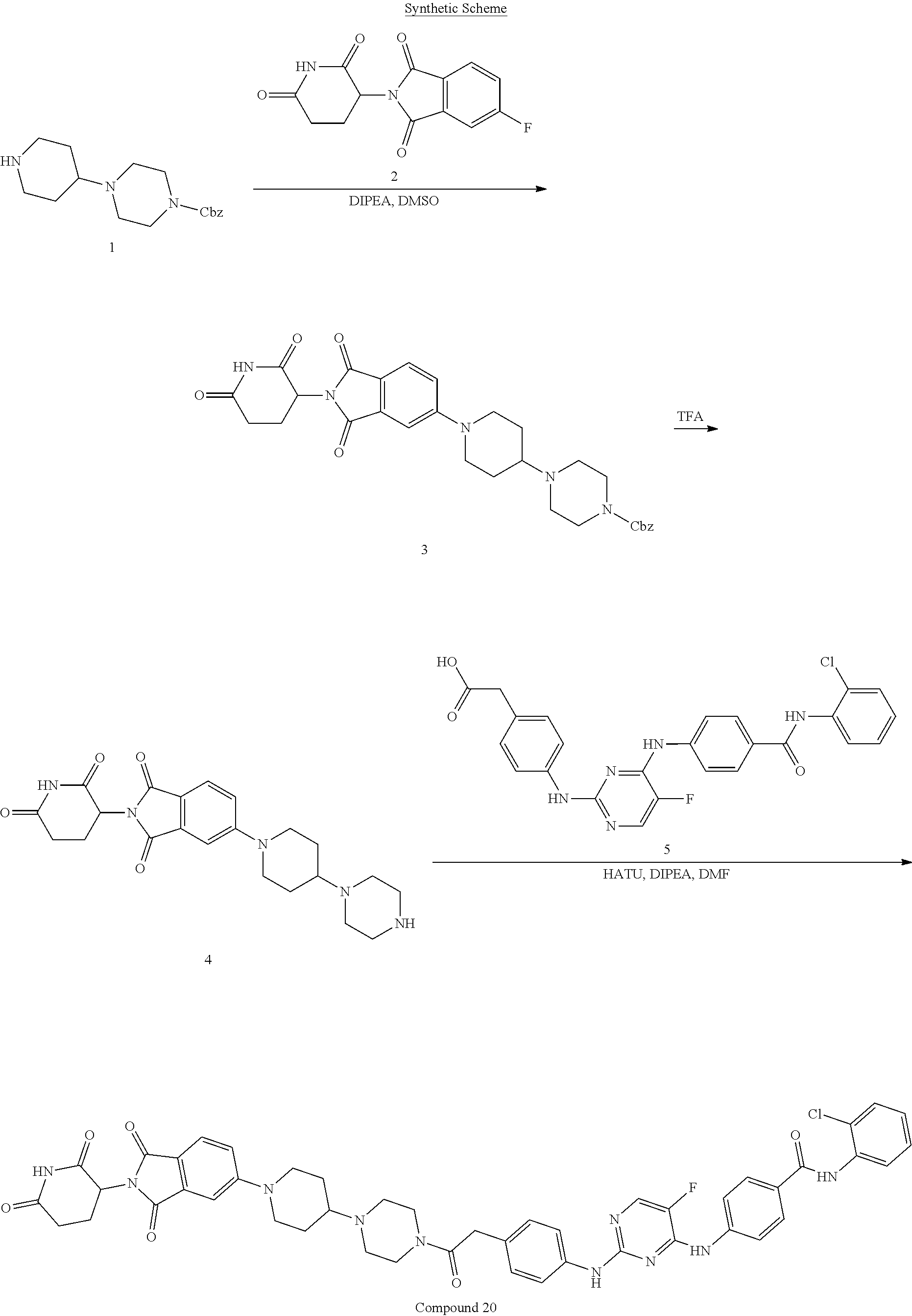 [SMILES: O=C(OCc1ccccc1)N1CCN(C2CCNCC2)CC1 ; O=C1CCC(N2C(=O)c3ccc(F)cc3C2=O)C(=O)N1 ; O=C1CCC(N2C(=O)c3ccc(N4CCC(N5CCN(C(=O)OCc6ccccc6)CC5)CC4)cc3C2=O)C(=O)N1 ; O=C1CCC(N2C(=O)c3ccc(N4CCC(N5CCNCC5)CC4)cc3C2=O)C(=O)N1 ; O=C(O)Cc1ccc(Nc2ncc(F)c(Nc3ccc(C(=O)Nc4ccccc4Cl)cc3)n2)cc1 ; O=C1CCC(N2C(=O)c3ccc(N4CCC(N5CCN(C(=O)Cc6ccc(Nc7ncc(F)c(Nc8ccc(C(=O)Nc9ccccc9Cl)cc8)n7)cc6)CC5)CC4)cc3C2=O)C(=O)N1]

### Experimental Process

### Step 1: Synthesis of benzyl 4-(1-(2-(2,6-dioxopiperidin-3 yl)-1,3-dioxoisoindoline-5 yl)piperidin-4-yl)piperazine-1-carboxylate (3)

DIPEA (561.48 mg, 4.34 mmol) was added to a solution of benzyl 4-(piperidin-4-yl)piperazine-1-carboxylate (400 mg, 1.45 mmol) and benzyl 4-(4-piperidyl)piperazine-1-carboxylate (544.96 mg, 1.45 mmol, 2HCl) in DMSO (8 mL) at 20° C. The mixture was stirred at 100° C. for 14 hours. It was confirmed through LCMS that the starting material was completely consumed, and the desired mass was detected. The mixture was poured with water (30 mL) and extracted with EtOAc (10 mL×4). The combined organic phase was washed with brine (10 mL×2), dried over Na₂SO₄, and concentrated under reduced pressure. The residue was purified by flash silica gel chromatography (ISCO®; 4 g SepaFlash® Silica Flash Column, Eluent of 30-100% EtOAc/Petroleum ether gradient @ 50 mL/min) to obtain benzyl 4-(1-(2-(2,6-dioxopiperidin-3-yl)-1,3-dioxoisoindoline-5-yl)piperidin-4-yl)piperazine-1-carboxylate (400 mg, crude) as yellow oil. MS(M+H)⁺=560.4

### Step 2: Synthesis of 2-(2,6-dioxopiperidin-3-yl)-5-(4-(piperazin-1-yl)piperidin-1-yl)isoindoline-1,3-dione (4)

A solution of benzyl 4-(1-(2-(2,6-dioxopiperidin-3-yl)-1,3-dioxoisoindoline-5-yl)piperidin-4-yl)piperazine-1-carboxylates (200 mg, 357.39 μmol) in TFA (4 mL) was stirred at 40° C. for 14 hours. A trace of starting material and the desired mass were detected by LCMS. The mixture was further stirred at 60° C. for 1 hour. The mixture was concentrated under reduced pressure, and the residue was dissolved in water (20 mL) and freeze-dried to obtain 2-(2,6-dioxopiperidin-3-yl)-5-(4-(piperazin-1-yl)piperidin-1-yl)isoindoline-1,3-dione (250 mg, crude, 2TFA) as yellow solid. MS(M+H)⁺=426.2.

### Step 3: Synthesis of N-(2-chlorophenyl)-4-((2-((4-(2-(4-(1-(2-(2,6-dioxopiperidin-3 yl))-1,3-dioxoisoindoline-5-yl)piperidin-4-yl)piperazin-1-yl)-2-oxoethyl)phenyl)amino)-5-fluoropyrimidin-4-yl)amino)benzamide (Compound 20)

2-(2,6-dioxopiperidin-3-yl)-5-(4-(piperazin-1-yl)piperidin-1-yl)isoindoline-1,3-dione (132.86 mg, 203.29 μmol, 2TFA) was added to a solution of 2-(4-((4-((4-((2-chlorophenyl)carbamoyl)phenyl) amino)-5-fluoropyrimidin-2-yl)amino)phenyl)acetic acid (100 mg, 203.29 μmol), DIPEA (131.37 mg, 1.02 mmol) and HATU (92.76 mg, 243.95 μmoL) in DMF (2 mL) at 20° C. and then stirred for 2 hours. A trace of starting material and the desired mass were detected by LCMS. The mixture was poured with water (15 mL) and extracted with EtOAc (10 mL×4). The combined organic phase was washed with brine (10 mL), dried over Na₂SO₄, and concentrated under reduced pressure. The product was purified by flash silica gel chromatography (ISCO®; 4 g SepaFlash® Silica Flash Column, Eluent of 30-100% EtOAc/Petroleum ether gradient followed by 10% MeOH/EtOAc gradient @ 50 mL/min). The product was repurified by prep-HPLC (column: Waters Xbridge 150*25 mm*5 um; mobile phase: [water (NH₄HCO₃)-ACN]; B %: 34%-64%, 8 min) and freeze-dried to obtain Compound 20 (15.5 mg, 15.68 μmol, 7.71% yield, 91% purity) as yellow solid. MS(M+H)⁺=899.2.

¹H NMR (400 MHz, DMSO-d₆) δ=11.07 (br s, 1H), 9.88 (s, 1H), 9.65 (s, 1H), 9.27 (s, 1H), 8.18 (d, J=3.7 Hz, 1H), 8.04-8.00 (m, 2H), 7.99-7.95 (m, 2H), 7.67-7.62 (m, 2H), 7.61-7.54 (m, 3H), 7.39 (dt, J=1.3, 7.7 Hz, 1H), 7.31-7.26 (m, 2H), 7.21 (dd, J=2.1, 8.6 Hz, 1H), 7.11 (br d, J=8.4 Hz, 2H), 5.06 (dd, J=5.4, 12.8 Hz, 1H), 4.05-3.98 (m, 2H), 3.63 (s, 2H), 3.45-3.40 (m, 4H), 3.30-3.28 (m, 4H), 2.95-2.85 (m, 3H), 2.62-2.58 (m, 1H), 2.40-2.35 (m, 2H), 2.05-1.98 (m, 1H), 1.80-1.74 (m, 2H), 1.46-1.34 (m, 2H).

### Example 21: Synthesis of Compound 21

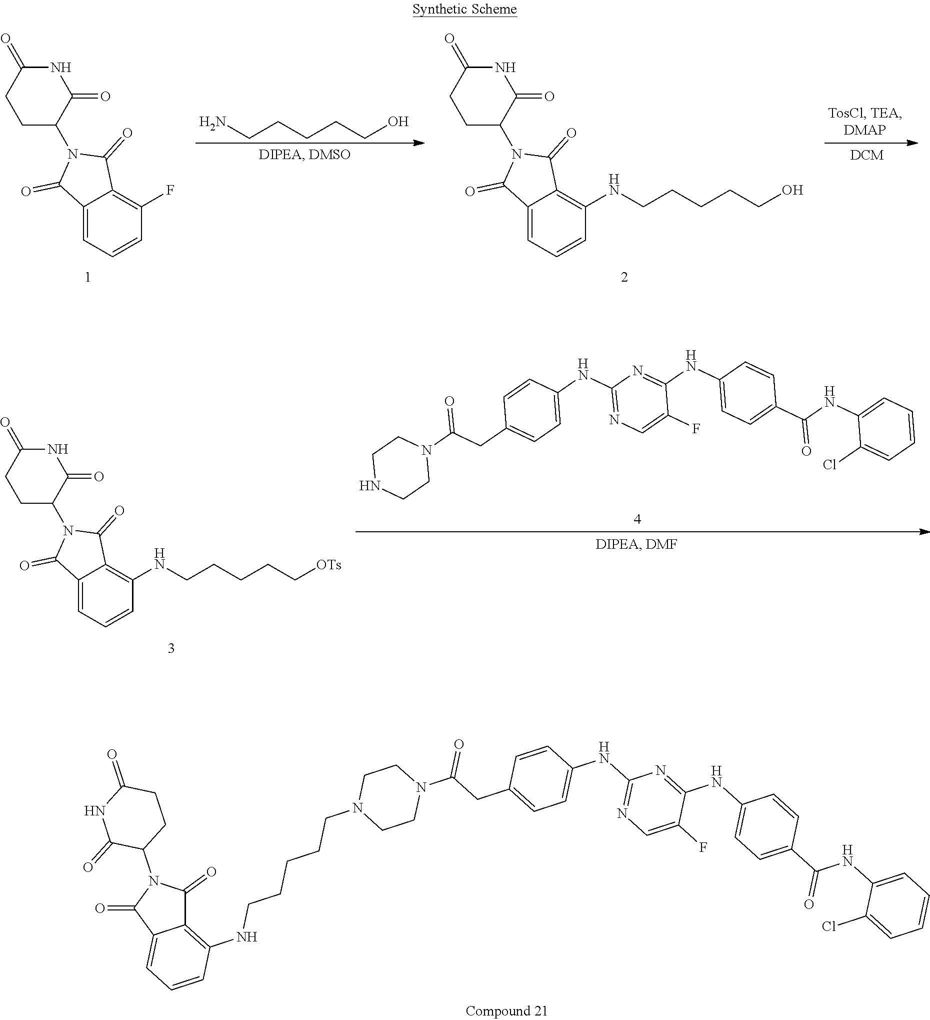 [SMILES: O=C1CCC(N2C(=O)c3cccc(F)c3C2=O)C(=O)N1 ; NCCCCCO ; O=C1CCC(N2C(=O)c3cccc(NCCCCCO)c3C2=O)C(=O)N1 ; Cc1ccc(S(=O)(=O)OCCCCCNc2cccc3c2C(=O)N(C2CCC(=O)NC2=O)C3=O)cc1 ; O=C(Nc1ccccc1Cl)c1ccc(Nc2nc(Nc3ccc(CC(=O)N4CCNCC4)cc3)ncc2F)cc1 ; O=C1CCC(N2C(=O)c3cccc(NCCCCCN4CCN(C(=O)Cc5ccc(Nc6ncc(F)c(Nc7ccc(C(=O)Nc8ccccc8Cl)cc7)n6)cc5)CC4)c3C2=O)C(=O)N1]

### Experimental Process

### Step 1: Synthesis of 2-(2,6-dioxopiperidin-3-yl)-4-((5-hydroxypentyl)amino)isoindoline-1,3-dione (2)

DIPEA (701.85 mg, 5.43 mmol, 945.89 μL) was added to a solution of 2-(2,6-dioxopiperidin-3-yl)-4-fluoroisoindoline-1,3-dione (500 mg, 1.81 mmol) and 5-aminopentan-1-ol (224.09 mg, 2.17 mmol) in DMSO (10 mL) at 100° C. and then stirred for 16 hours. The main peak of desired mass was detected by LCMS. The mixture was diluted with brine (30 mL) and then extracted with EtOAc (30 mL×5). The combined organic phase was washed with brine (10 mL×5), dried over Na₂SO₄, and filtered and concentrated under reduced pressure to obtain 2-(2,6-dioxopiperidin-3-yl)-4-((5-hydroxypentyl)amino)isoindoline-1,3-dione (650 mg, 1.81 mmol, 99.92% yield, crude) as yellow oil. MS(M+H)⁺=360.1.

### Step 2: Synthesis of 5-((2-(2,6-dioxopiperidin-3-yl)-1,3-dioxoisoindolin-4-yl)amino)pentyl 4-methylbenzenesulfonate (3)

A solution of 2-(2,6-dioxopiperidin-3-yl)-4-((5-hydroxypentyl)amino)isoindoline-1,3-dione (650 mg, 1.81 mmol), TosCl (517.23 mg, 2.71 mmol), TEA (549.06 mg, 5.43 mmol, 755.24 μL) and DMAP (22.10 mg, 180.87 μmol) in DCM (10 mL) was stirred at 20° C. for 2 hours. The main peak of desired mass was detected by LCMS. The mixture was diluted with water (20 mL) and then extracted with EtOAc (20 mL×3). The combined organic phase was washed with brine (20 mL×2), dried over Na₂SO₄, and filtered. The filtrate was concentrated under reduced pressure, and then the residue was purified by flash silica gel chromatography (Biotage; 20 g SepaFlash® Silica Flash Column, Eluent of 20-80% EtOAc/Petroleum ether gradient @ 40 mL/min) to obtain 5-((2-(2,6-dioxopiperidin-3-yl)-1,3-dioxoisoindolin-4-yl)amino)pentyl 4-methylbenzenesulfonate (500 mg, 973.59 μmol, 53.83% yield, N/A purity) as yellow solid. MS(M+H)⁺=514.2.

### Step 3: Synthesis of N-(2-chlorophenyl)-4-((2-((4-(2-(4-(5-((2-(2,6-dioxopiperidin-3 yl))-1,3-dioxoisoindolin-4-yl)amino)pentyl)piperazin-1-yl)-2-oxoethyl)phenyl)amino)-5-fluoropyrimidin-4-yl)amino)benzamide (Compound 21)

DIPEA (69.23 mg, 535.69 μmol, 93.31 μL) and NaI (2.68 mg, 17.86 μmol)) were added to a solution of 5-((2-(2,6-dioxopiperidin-3-yl)-1,3-dioxoisoindolin-4-yl)amino)pentyl 4-methylbenzenesulfonate (91.70 mg, 178.56 μmol), N-(2-chlorophenyl)-4-((5-fluoro-2-((4-(2-oxo-2-(piperazin-1-yl)ethyl)phenyl)amino)pyrimidine-4-yl)amino)benzamide (100 mg, 167.65 μmol, HCl) in DMF (1 mL). The mixture was stirred at 80° C. for 16 hours. The main peak of desired mass was detected by LCMS. The mixture was diluted with brine (30 mL) and then extracted with EtOAc (20 mL×3). The combined organic phase was washed with brine (20 mL×3), dried over Na₂SO₄, and filtered. The filtrate was concentrated under reduced pressure and then the residue was purified by prep-TLC (SiO₂, DCM:MeOH=10:1) and then repurified by reverse phase HPLC (column: Waters Xbridge 150*25 mm*5 um; mobile phase: [water (NH₄HCO₃)-ACN]; B %: 40%-70%, 8 min). The product was freeze-dried to obtain Compound 21 (19.2 mg, 19.81 μmol, 11.09% yield, 93% purity) as yellow solid. MS(M+H)⁺=901.5.

¹H NMR (400 MHz, DMSO-d₆) δ=11.08 (s, 1H), 9.88 (s, 1H), 9.65 (s, 1H), 9.27 (s, 1H), 8.18 (d, J=3.6 Hz, 1H), 8.05-7.92 (m, 4H), 7.69-7.62 (m, 1H), 7.62-7.52 (m, 4H), 7.43-7.36 (m, 1H), 7.31-7.25 (m, 1H), 7.14-7.04 (m, 3H), 7.01 (d, J=7.0 Hz, 1H), 6.54-6.48 (m, 1H), 5.11-4.98 (m, 1H), 3.67-3.57 (m, 2H), 3.47-3.39 (m, 4H), 3.27-3.24 (m, 2H), 2.94-2.80 (m, 1H), 2.63-2.52 (m, 2H), 2.26-2.16 (m, 6H), 2.06-1.97 (m, 1H), 1.61-1.49 (m, 2H), 1.46-1.37 (m, 2H), 1.35-1.25 (m, 2H).

### Example 22: Synthesis of Compound 22

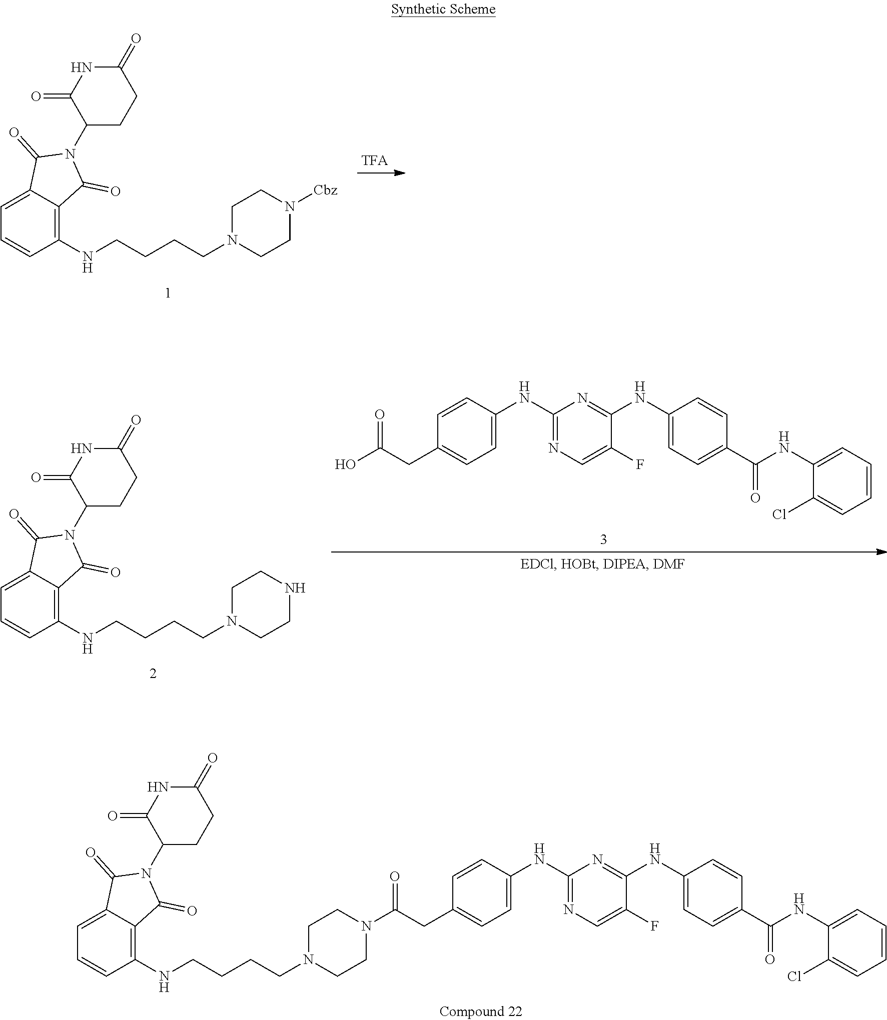 [SMILES: O=C1CCC(N2C(=O)c3cccc(NCCCCN4CCN(C(=O)OCc5ccccc5)CC4)c3C2=O)C(=O)N1 ; O=C1CCC(N2C(=O)c3cccc(NCCCCN4CCNCC4)c3C2=O)C(=O)N1 ; O=C(O)Cc1ccc(Nc2ncc(F)c(Nc3ccc(C(=O)Nc4ccccc4Cl)cc3)n2)cc1 ; O=C1CCC(N2C(=O)c3cccc(NCCCCN4CCN(C(=O)Cc5ccc(Nc6ncc(F)c(Nc7ccc(C(=O)Nc8ccccc8Cl)cc7)n6)cc5)CC4)c3C2=O)C(=O)N1]

### Experimental Process

### Step 1: Synthesis of 2-(2,6-dioxopiperidin-3 yl)-4-((4-(piperazin-1-yl)butyl)amino)isoindoline-1,3-dione (2)

A solution of benzyl 4-(4-((2-(2,6-dioxopiperidin-3-yl)-1,3-dioxoisoindolin-4-yl)amino)butyl)piperazine-1-carboxylate (100 mg, 182.61 μcool) in TFA (1 mL) was stirred at 40° C. for 1 hour. The main peak of desired mass was detected by LCMS. The mixture was concentrated under reduced pressure to obtain 2-(2,6-dioxopiperidin-3-yl)-4-((4-(piperazin-1-yl)butyl)amino)isoindoline-1,3-dione (96 mg, crude, TFA) as brown oil. MS(M+H)⁺=414.3.

### Step 2: Synthesis of N-(2-chlorophenyl)-4-((2-((4-(2-(4-(4-((2-(2,6-dioxopiperidin-3 yl))-1,3-dioxoisoindolin-4-yl)amino)butyl)piperazin-1 yl)-2-oxoethyl)phenyl)amino)-5-fluoropyrimidin-4-yl)amino)benzamide (Compound 22)

EDCI (49.06 mg, 255.93 μmol), HOBt (34.58 mg, 255.93 μmol), and DIPEA (110.25 mg, 853.09 μmol, 148.59 μL) were added to a solution of 2-(4-((4-((4-((2-chlorophenyl)carbamoyl)phenyl)amino)-5-fluoropyrimidin-2-yl) amino)phenyl) acetic acid (83.93 mg, 170.62 μmol) and 2-(2,6-dioxopiperidin-3-yl)-4-((4-(piperazin-1-yl)butyl)amino)isoindoline-1,3-dione (90 mg, 170.62 μmol, TFA) in DMF (2 mL) and then stirred at 20° C. for 16 hours. The main peak of desired mass was detected by LCMS. The mixture was diluted with water (15 mL) and then extracted with EtOAc (10 mL×3). The combined organic phase was washed with brine (10 mL×3), dried over Na₂SO₄, and filtered. The filtrate was concentrated under reduced pressure and then the residue was purified by prep-TLC (SiO₂, DCM:MeOH=10:1). The product was further purified by prep-HPLC (column: Waters Xbridge C18 150*50 mm*10 um; mobile phase: [water (NH₄HCO₃)-ACN]; B %: 40%-70%, 10 min) and then freeze-dried to obtain Compound 22 (32.6 mg, 35.64 μmol, 20.89% yield, 97% purity) as yellow solid. MS(M+H)⁺=887.3

¹H NMR (400 MHz, DMSO-d₆) δ=11.09 (s, 1H), 9.88 (s, 1H), 9.69-9.60 (m, 1H), 9.27 (s, 1H), 8.18 (d, J=3.5 Hz, 1H), 8.07-7.89 (m, 4H), 7.69-7.51 (m, 5H), 7.42-7.35 (m, 1H), 7.32-7.24 (m, 1H), 7.15-7.04 (m, 3H), 7.01 (d, J=7.0 Hz, 1H), 6.58-6.48 (m, 1H), 5.05-4.97 (m, 1H), 3.63 (s, 2H), 3.47-3.42 (m, 4H), 3.31-3.28 (m, 4H), 2.90-2.83 (m, 1H), 2.62-2.58 (m, 2H), 2.26-2.21 (m, 4H), 2.07-1.98 (m, 1H), 1.61-1.42 (m, 4H).

### Example 23: Synthesis of Compound 23

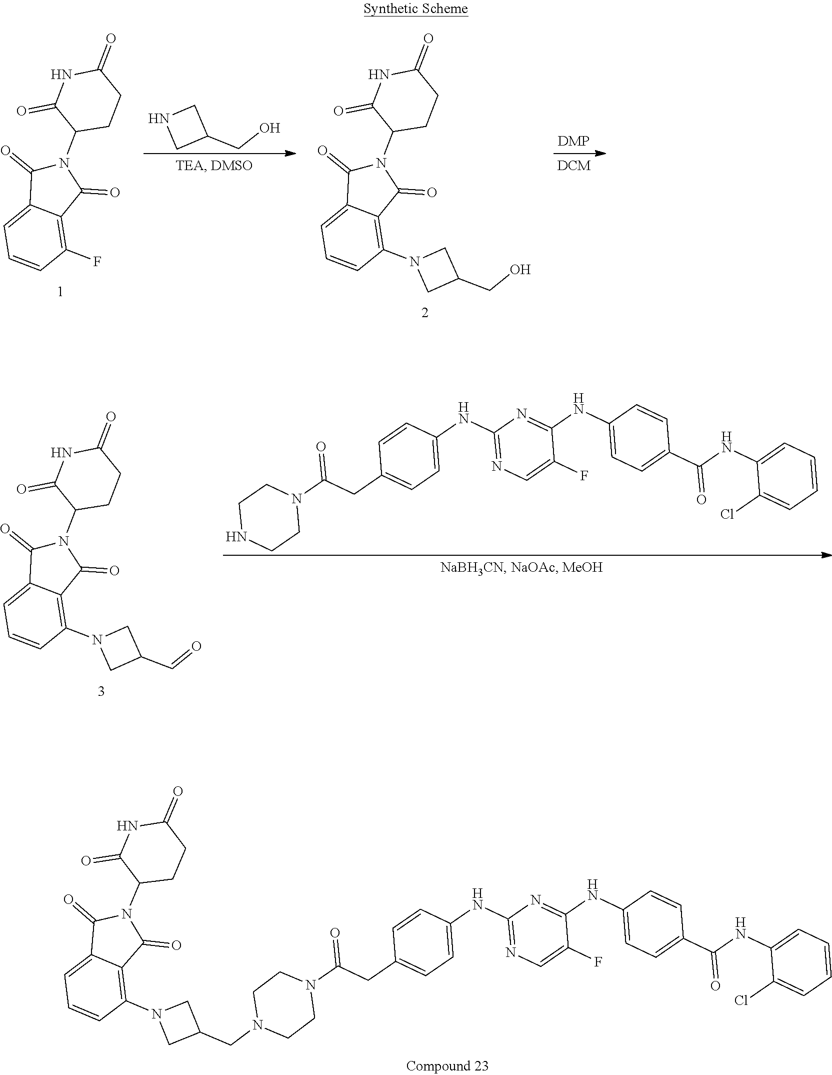 [SMILES: O=C1CCC(N2C(=O)c3cccc(F)c3C2=O)C(=O)N1 ; OCC1CNC1 ; O=C1CCC(N2C(=O)c3cccc(N4CC(CO)C4)c3C2=O)C(=O)N1 ; O=CC1CN(c2cccc3c2C(=O)N(C2CCC(=O)NC2=O)C3=O)C1 ; O=C(Nc1ccccc1Cl)c1ccc(Nc2nc(Nc3ccc(CC(=O)N4CCNCC4)cc3)ncc2F)cc1 ; O=C1CCC(N2C(=O)c3cccc(N4CC(CN5CCN(C(=O)Cc6ccc(Nc7ncc(F)c(Nc8ccc(C(=O)Nc9ccccc9Cl)cc8)n7)cc6)CC5)C4)c3C2=O)C(=O)N1]

### Experimental Process

### Step 1: Synthesis of 2-(2,6-dioxopiperidin-3 yl)-4-(3-(hydroxymethyl)azetidin-1-yl)isoindoline-1,3-dione (2)

TEA (549.51 mg, 5.43 mmol) was added to a solution of 2-(2,6-dioxo-3-piperidyl)-4-fluoro-isoindoline-1,3-dione (0.5 g, 1.81 mmol) and azetidin-3-yl methanol hydrochloride (246.07 mg, 1.99 mmol, HCl) in DMSO (5 mL) and then stirred at 120° C. for 4 hours. The main peak of desired mass was detected by LCMS. The mixture was diluted with water (50 mL) and then extracted with EtOAc (15 mL×3). The combined organic phase was washed with brine (15 mL), dried over Na₂SO₄, filtered, and concentrated under reduced pressure to obtain yellow solid (860 mg, crude), which was immediately used in the next reaction. MS(M+H)⁺=344.1.

### Step 2: Synthesis of 1-(2-(2,6-dioxopiperidin-3-yl)-1,3-dioxoisoindolin-4-yl)azetidine-3-carbaldehyde (3)

DMP (475.62 mg, 1.12 mmol) was added to a solution of 2-(2,6-dioxopiperidin-3-yl)-4-(3-(hydroxymethyl)azetidin-1-yl)isoindoline-1,3-dione (0.35 g, 1.02 mmol) in DCM (5 mL) and then stirred at 25° C. for 2 hours. It was confirmed through LCMS that the starting material was completely consumed, and the desired mass was detected. The mixture was concentrated under reduced pressure to obtain 1-(2-(2,6-dioxopiperidin-3-yl)-1,3-dioxoisoindolin-4-yl)azetidine-3-carbaldehyde (360 mg, crude) as yellow solid, which was immediately used in the next reaction. MS(M+H)⁺=342.1.

### Step 3: Synthesis of N-(2-chlorophenyl)-4-((2-((4-(2-(4-((1-(2-(2,6-dioxopiperidin-3 yl))-1,3-dioxoisoindolin-4-yl)azetidin-3-yl)methyl)piperazin-1-yl)-2-oxoethyl)phenyl)amino)-5-fluoropyrimidin-4-yl)amino)benzamide (Compound 23)

A solution of 1-(2-(2,6-dioxopiperidin-3-yl)-1,3-dioxoisoindolin-4-yl)azetidine-3-carbaldehyde (357.64 mg, 628.69 μmol), N-(2-chlorophenyl)-4-((5-fluoro-2-((4-(2-oxo-2-(piperazin-1-yl)ethyl)phenyl)amino)pyrimidin-4-yl)amino)benzamide (150 mg, 251.47 μmol, HCl) and NaOAc (41.26 mg, 502.95 μmol) in MeOH (6 mL) was stirred at 25° C. for 30 minutes. Thereafter, NaBH₃CN (47.41 mg, 754.42 μmol) was added and then stirred at 25° C. for 16 hours. The desired mass was detected by LCMS. The mixture was diluted with water (20 mL) and then extracted with EtOAc (15 mL×3). The combined organic phase was washed with brine (15 mL), dried over Na₂S 04, filtered and concentrated. The residue was purified by flash silica gel chromatography (ISCO®; 12 g SepaFlash® Silica Flash Column, Eluent of 50-100% EtOAc/Petroleum ether to 0-10% MeOH/EtOAc gradient @ 100 mL/min). The product was further purified by prep-HPLC (column: Waters Xbridge BEH C18 150*25 mm*5 um; mobile phase: [water (NH₄HCO₃)-ACN]; B %: 41%-71%, 10 min) and then freeze-dried to obtain Compound 23 (36.5 mg, 38.34 μmol, 15.25% yield, 93% purity) as yellow solid. MS(M+H)⁺=885.4

¹H NMR (400 MHz, DMSO-d₆) δ=11.06 (s, 1H), 9.89 (s, 1H), 9.65 (s, 1H), 9.28 (s, 1H), 8.17 (d, J=3.6 Hz, 1H), 8.04-8.00 (m, 2H), 7.99-7.95 (m, 2H), 7.65 (dd, J=1.5, 8.0 Hz, 1H), 7.59 (d, J=8.5 Hz, 2H), 7.56-7.51 (m, 2H), 7.38 (dt, J=1.4, 7.7 Hz, 1H), 7.30-7.24 (m, 1H), 7.12 (d, J=8.6 Hz, 2H), 7.09 (d, J=7.0 Hz, 1H), 6.73 (d, J=8.5 Hz, 1H), 5.03 (dd, J=5.5, 12.6 Hz, 1H), 4.28-4.16 (m, 2H), 3.83-3.74 (m, 2H), 3.68-3.60 (m, 2H), 3.50-3.38 (m, 6H), 2.91-2.82 (m, 2H), 2.59-2.54 (m, 2H), 2.30-2.27 (m, 4H), 2.03-1.94 (m, 1H).

### Example 24: Synthesis of Compound 24

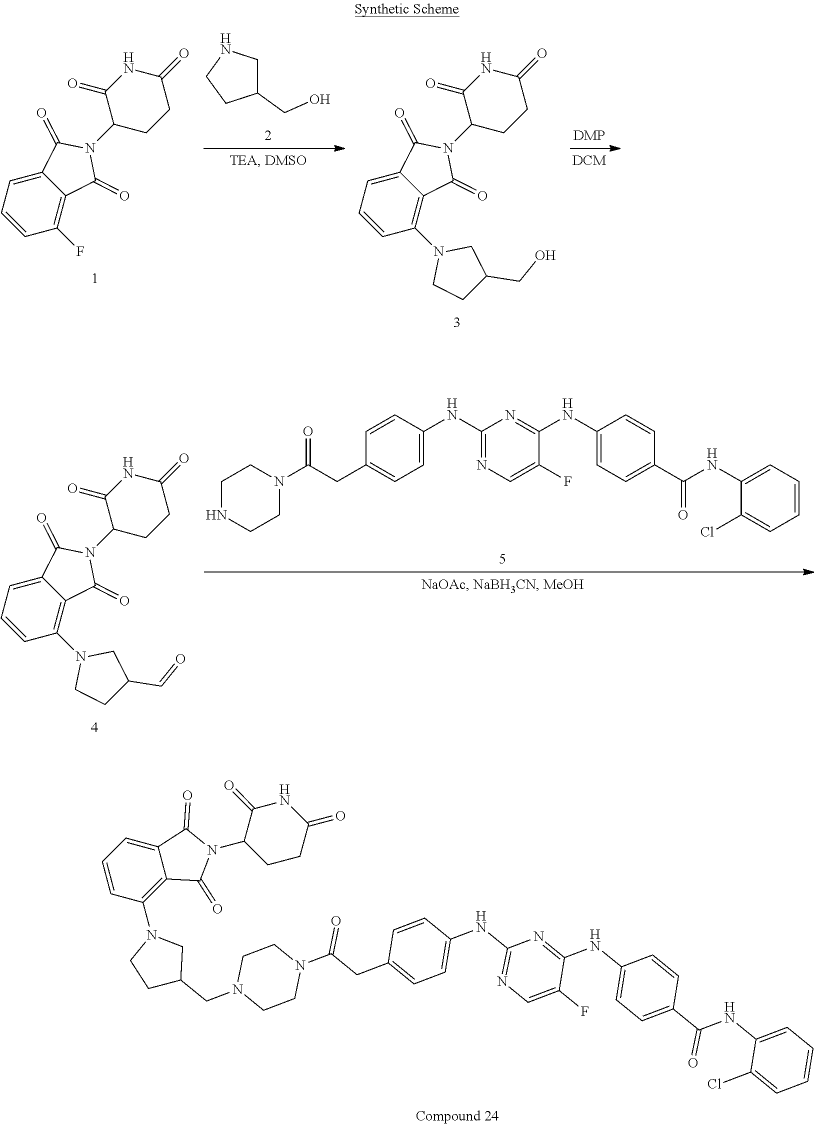

### Step 1: Synthesis of 2-(2,6-dioxopiperidin-3 yl)-4-(3-(hydroxymethyl)pyrrolidin-1-yl)isoindoline-1,3-dione (3)

TEA (219.80 mg, 2.17 mmol, 302.34 μL) was added to a solution of pyrrolidin-3-yl methanol (164.78 mg, 1.63 mmol) and 2-(2,6-dioxopiperidin-3-yl)-4-fluoroisoindoline-1,3-dione (300 mg, 1.09 mmol) in DMSO (6 mL) and then stirred at 120° C. for 12 hours. The main peak of desired mass was detected by LCMS. The mixture was washed with brine (30 mL) and then extracted with EtOAc (20 mL×3). The combined organic phase was washed with brine (10 mL×5), dried over Na₂SO₄, and filtered and concentrated under reduced pressure to obtain 2-(2,6-dioxopiperidin-3-yl)-4-(3-(hydroxymethyl)pyrrolidin-1-yl)isoindoline-1,3-dione (300 mg, crude) as yellow solid. MS(M+H)⁺=358.2.

### Step 2: Synthesis of 1-(2-(2,6-dioxopiperidin-3-yl)-1,3-dioxoisoindolin-4-yl)pyrrolidine-3-carbaldehyde (4)

DMP (142.43 mg, 335.80 μmol) was added to a solution of 2-(2,6-dioxopiperidin-3-yl)-4-(3-(hydroxymethyl)pyrrolidin-1-yl)isoindoline-1,3-dione (100 mg, 279.83 μmol) in DCM (1 mL) and then stirred at 20° C. for 1 hour. It was confirmed through TLC that the starting material was completely consumed, and one main spot was detected. The mixture was filtered through a Celite pad and the filtrate was concentrated under reduced pressure to obtain 1-(2-(2,6-dioxopiperidin-3-yl)-1,3-dioxoisoindolin-4-yl)pyrrolidine-3-carbaldehyde (99 mg, crude) as brown oil. MS(M+H)⁺=356.4.

### Step 3: Synthesis of N-(2-chlorophenyl)-4-((2-((4-(2-(4-((1-(2-(2,6-dioxopiperidin-3 yl))-1,3-dioxoisoindolin-4-yl)pyrrolidin-3-yl)methyl)piperazin-1-yl)-2-oxoethyl)phenyl)amino)-5 fluoropyrimidin-4-yl)amino)benzamide (Compound 24)

NaOAc (19.78 mg, 241.06 μmol) was added to a solution of N-(2-chlorophenyl)-4-((5-fluoro-2-((4-(2-oxo-2-(piperazin-1-yl)ethyl)phenyl)amino)pyrimidine-4-yl)amino)benzamide (90 mg, 160.71 μmol) and 1-(2-(2,6-dioxopiperidin-3-yl)-1,3-dioxoisoindolin-4-yl)pyrrolidine-3-carbaldehyde (57.11 mg, 160.71 μmol) in MeOH (2 mL) and then stirred at 20° C. for 30 minutes. Thereafter, NaBH₃CN (30.30 mg, 482.13 μmol) was added and then the mixture was stirred at 20° C. for 1 hour. The peak (12%) of desired mass was detected by LCMS. The mixture was quenched with NaHCO₃ (5 mL) and then extracted with EtOAc (10 mL×3). The combined organic phase was washed with brine (20 mL×2), dried over Na₂SO₄, and filtered. The filtrate was concentrated under reduced pressure and then the residue was purified by prep-TLC (SiO₂, DCM:MeOH=10:1) and repurified by prep-HPLC (column: Waters Xbridge 150*25 mm*Sum; mobile phase: [water (NH₄HCO₃)-ACNI; B %: 41%-71%, 8 min) and then freeze-dried to obtain Compound 24 (10.1 mg, 11.12 μmol, 6.92% yield, 94% purity) as yellow solid. MS(M+H)⁺=899.2.

¹H NMR (400 MHz, DMSO-d₆) δ=11.05 (s, 1H), 9.88 (s, 1H), 9.65 (s, 1H), 9.28 (s, 1H), 8.18 (d, J=3.5 Hz, 1H), 8.06-8.00 (m, 2H), 7.99-7.94 (m, 2H), 7.68-7.63 (m, 1H), 7.62-7.58 (m, 2H), 7.57-7.51 (m, 2H), 7.41-7.35 (m, 1H), 7.31-7.24 (m, 1H), 7.14-7.04 (m, 4H), 5.11-4.97 (m, 1H), 3.64 (s, 2H), 3.62-3.50 (m, 3H), 3.50-3.42 (m, 4H), 3.30-3.26 (m, 1H), 2.93-2.80 (m, 1H), 2.63-2.54 (m, 2H), 2.48-2.43 (m, 2H), 2.32-2.25 (m, 5H), 2.07-1.93 (m, 2H), 1.68-1.56 (m, 1H).

### Example 25: Synthesis of Compound 25

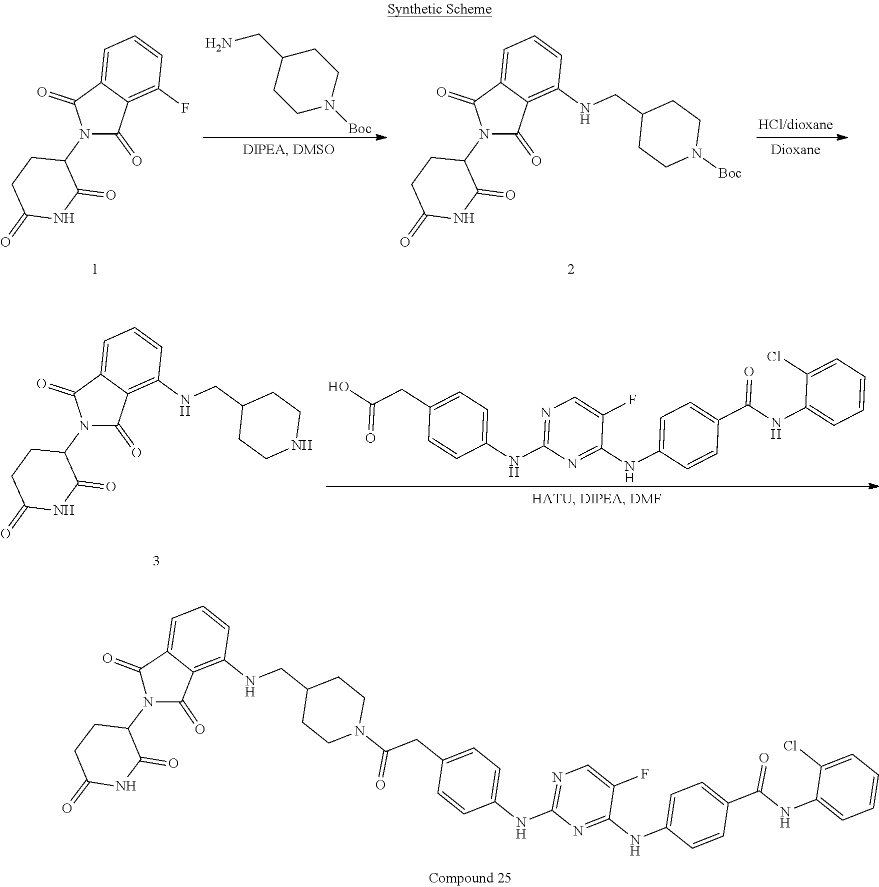 [SMILES: O=C1CCC(N2C(=O)c3cccc(F)c3C2=O)C(=O)N1 ; CC(C)(C)OC(=O)N1CCC(CN)CC1 ; CC(C)(C)OC(=O)N1CCC(CNc2cccc3c2C(=O)N(C2CCC(=O)NC2=O)C3=O)CC1 ; O=C1CCC(N2C(=O)c3cccc(NCC4CCNCC4)c3C2=O)C(=O)N1 ; O=C(O)Cc1ccc(Nc2ncc(F)c(Nc3ccc(C(=O)Nc4ccccc4Cl)cc3)n2)cc1 ; O=C1CCC(N2C(=O)c3cccc(NCC4CCN(C(=O)Cc5ccc(Nc6ncc(F)c(Nc7ccc(C(=O)Nc8ccccc8Cl)cc7)n6)cc5)CC4)c3C2=O)C(=O)N1]

### Experimental Process

### Step 1: Synthesis of tert-butyl 4-(((2-(2,6-dioxopiperidin-3 yl)-1,3-dioxoisoindolin-4-yl)amino)methyl)piperidine-1-carboxylate (2)

A solution of 2-(2,6-dioxo-3-piperidyl)-4-fluoro-isoindoline-1,3-dione (500 mg, 1.81 mmol), tert-butyl 4-(aminomethyl)piperidine-1-carboxylate (387.92 mg, 1.81 mmol) and DIPEA (467.90 mg, 3.62 mmol) in DMSO (5 mL) was stirred at 100° C. for 14 hours. The main peak of desired mass was detected by LCMS. The mixture was poured with water (15 mL) and extracted with EtOAc (8 mL×3). The combined organic phase was washed with brine (5 mL×3), dried over Na₂SO₄, and concentrated under reduced pressure to obtain tert-butyl 4-(((2-(2,6-dioxopiperidin-3-yl)-1,3-dioxoisoindolin-4-yl)amino)methyl)piperidine-1-carboxylate (820 mg, crude) as green solid. MS(M-Boc+H)⁺=371.2.

### Step 2: Synthesis of 2-(2,6-dioxopiperidin-3 yl)-4-((piperidin-4 yl methyl)amino)isoindoline-1,3-dione (3)

HCl/dioxane (4 M, 10 mL) was added to a solution of tert-butyl 4-(((2-(2,6-dioxopiperidin-3-yl)-1,3-dioxoisoindolin-4-yl)amino)methyl)piperidine-1-carboxylate (820 mg, 1.74 mmol) in dioxane (10 mL) at 20° C. and then stirred for 1 hour. It was confirmed through LCMS that the starting material was completely consumed, and a main peak with the desired mass was detected. The mixture was concentrated under reduced pressure to obtain 2-(2,6-dioxopiperidin-3-yl)-4-((piperidin-4-yl methyl)amino)isoindoline-1,3-dione (700 mg, crude, HCl) as yellow solid. MS(M+H)⁺=371.2

### Step 3: Synthesis of N-(2-chlorophenyl)-4-((2-((4-(2-(4-(((2-(2,6-dioxopiperidin-3 yl)-1,3-dioxoisoindolin-4-yl))amino)methyl)piperidin-1-yl)-2-oxoethyl)phenyl)amino)-5-fluoropyrimidin-4-yl)amino)benzamide (Compound 25)

HATU (102.80 mg, 270.36 μmol) was added to a solution of 2-(2,6-dioxopiperidin-3-yl)-4-((piperidin-4-ylmethyl)amino)isoindoline-1,3-dione (100 mg, 245.78 μmol, HCl), 2-(4-((4-((4-((2-chlorophenyl)carbamoyl)phenyl) amino)-5-fluoropyrimidin-2-yl)amino)phenyl)acetic acid (120.90 mg, 245.78 μmol) and DIPEA (158.83 mg, 1.23 mmol) in DMF (3 mL) at 20° C., and then stirred for 14 hours. The main peak of desired mass was detected by LCMS. The mixture was poured in water (10 mL) and extracted with EtOAc (8 mL×3). The combined organic phase was washed with brine (5 mL), dried over Na₂SO₄, and concentrated under reduced pressure. The product was purified by prep-HPLC (column: Phenomenex luna C18 150*25 mm*10 um; mobile phase: [water (FA)-ACN]; B %: 34%-64%, 10 min) and then freeze-dried to obtain Compound 25 (29.1 mg, 32.40 μmol, 13.18% yield, 94% purity) as yellow solid. MS(M+H)⁺=844.4.

¹H NMR (400 MHz, DMSO-d₆) δ=11.09 (s, 1H), 9.88 (s, 1H), 9.65 (s, 1H), 9.27 (s, 1H), 8.22-8.16 (m, 1H), 8.07-8.01 (m, 2H), 7.99-7.94 (m, 2H), 7.67-7.52 (m, 5H), 7.38 (t, J=7.5 Hz, 1H), 7.31-7.24 (m, 1H), 7.15-7.06 (m, 3H), 7.00 (d, J=7.1 Hz, 1H), 6.63-6.57 (m, 1H), 5.04 (dd, J=5.5, 13.0 Hz, 1H), 4.45-4.35 (m, 1H), 4.03-3.94 (m, 1H), 3.70-3.57 (m, 2H), 3.32-3.29 (m, 2H), 3.20-3.13 (m, 2H), 3.00-2.83 (m, 2H), 2.63-2.58 (m, 1H), 2.06-1.96 (m, 1H), 1.87-1.74 (m, 1H), 1.70-1.62 (m, 2H), 1.07-0.92 (m, 2H).

### Example 26: Synthesis of Compound 26

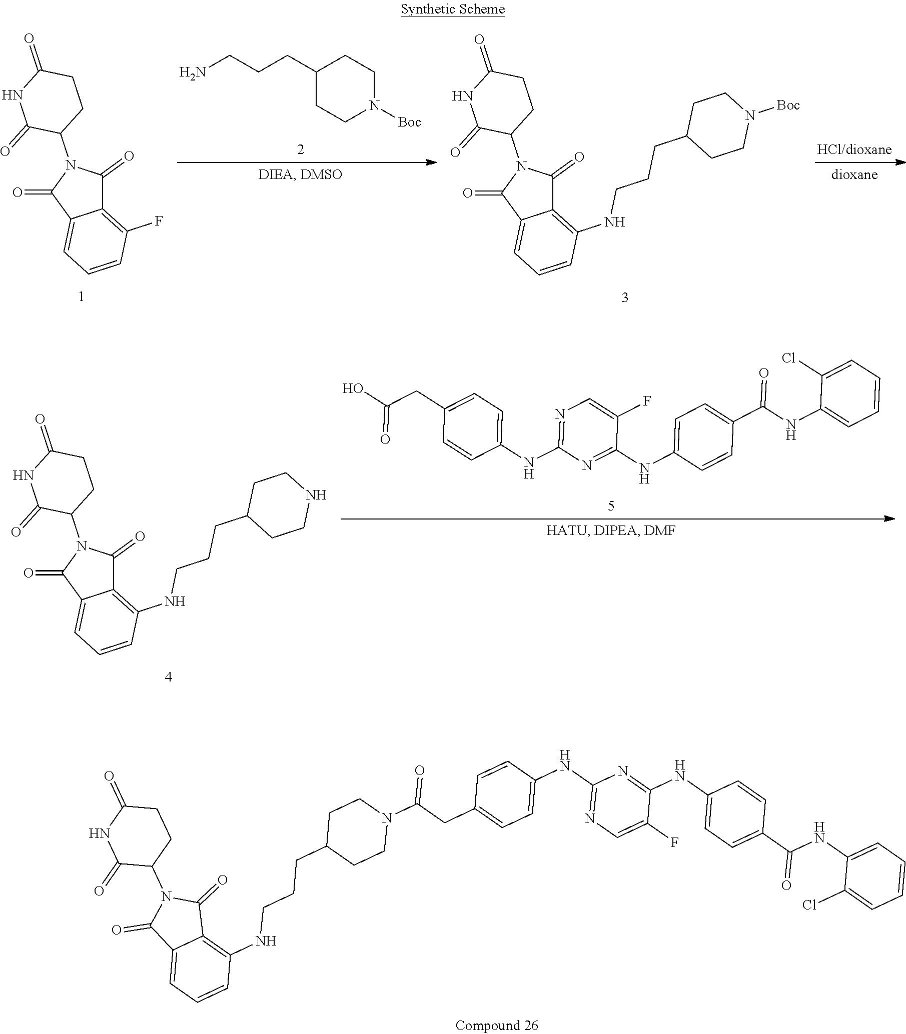 [SMILES: O=C1CCC(N2C(=O)c3cccc(F)c3C2=O)C(=O)N1 ; CC(C)(C)OC(=O)N1CCC(CCCN)CC1 ; CC(C)(C)OC(=O)N1CCC(CCCNc2cccc3c2C(=O)N(C2CCC(=O)NC2=O)C3=O)CC1 ; O=C1CCC(N2C(=O)c3cccc(NCCCC4CCNCC4)c3C2=O)C(=O)N1 ; O=C(O)Cc1ccc(Nc2ncc(F)c(Nc3ccc(C(=O)Nc4ccccc4Cl)cc3)n2)cc1 ; O=C1CCC(N2C(=O)c3cccc(NCCCC4CCN(C(=O)Cc5ccc(Nc6ncc(F)c(Nc7ccc(C(=O)Nc8ccccc8Cl)cc7)n6)cc5)CC4)c3C2=O)C(=O)N1]

### Experimental Process

### Step 1: Synthesis of tert-butyl 4-(3-((2-(2,6-dioxopiperidin-3 yl)-1,3-dioxoisoindolin-4-yl)amino)propyl)piperidine-1-carboxylate (3)

DIEA (140.37 mg, 1.09 mmol, 189.18 μL) and tert-butyl 4-(3-aminopropyl)piperidine-1-carboxylate (131.61 mg, 543.05 μcool) were added to solution of 2-(2,6-dioxopiperidin-3-yl)-4-fluoroisoindoline-1,3-dione (150 mg, 543.05 μmol) in DMSO (5 mL). The mixture was stirred at 100° C. for 12 hours. The desired mass of ˜46% was detected by LCMS. The mixture was diluted with water (20 mL) and then extracted with EtOAc (30 mL×3). The combined organic phase was washed with saturated brine (20 mL), dried over anhydrous Na₂SO₄, and filtered and concentrated under reduced pressure. The residue was purified by flash silica gel chromatography (ISCO®; 12 g SepaFlash® Silica Flash Column, Eluent of 0-40% petroleum ether:EtOAc gradient @ 60 mL/min) to obtain tert-butyl 4-(3-((2-(2,6-dioxopiperidin-3-yl)-1,3-dioxoisoindolin-4-yl)amino)propyl)piperidine-1-carboxylate (0.3 g, 385.10 μmol, 70.92% yield, 64% purity) as yellow oil. MS(M+Na)⁺=521.1.

### Step 2: Synthesis of 2-(2,6-dioxopiperidin-3 yl)-4-((3-(piperidin-4-yl)propyl)amino)isoindoline-1,3-dione (4)

HCl/dioxane (4 M, 10 mL) was added to a solution of tert-butyl 4-(3-((2-(2,6-dioxopiperidin-3-yl)-1,3-dioxoisoindolin-4-yl)amino)propyl)piperidine-1-carboxylate (0.3 g, 601.72 μmol) in dioxane (5 mL) and then stirred at 20° C. for 2 hours. It was confirmed through TLC (petroleum ether:EtOAc=1:1) that the starting material was completely consumed. The mixture was concentrated under reduced pressure to obtain 2-(2,6-dioxopiperidin-3-yl)-4-((3-(piperidin-4-yl)propyl)amino)isoindoline-1,3-dione (0.3 g, crude, HCl) as yellow solid. MS(M+H)⁺=399.2.

### Step 3: Synthesis of N-(2-chlorophenyl)-4-((2-((4-(2-(4-(3-((2-(2,6-dioxopiperidin-3 yl))-1,3-dioxoisoindolin-4-yl)amino)propyl)piperidin-1-yl)-2-oxoethyl)phenyl)amino)-5-fluoropyrimidin-4-yl)amino)benzamide (Compound 26)

HATU (173.92 mg, 457.41 μmol) and DIPEA (118.23 mg, 914.82 μmol, 159.34 μL) were added to a solution of 2-(4-((4-((4-((2-chlorophenyl)carbamoyl)phenyl)amino)-5-fluoropyrimidin-2-yl)amino)phenyl)acetic acid (150 mg, 304.94 μmol) in DMF (3 mL). After 30 minutes, 2-(2,6-dioxopiperidin-3-yl)-4-((3-(piperidin-4-yl)propyl)amino)isoindoline-1,3-dione (198.93 mg, 457.41 μmol, HCl) was added and stirred at 25° C. for 2 hours. The desired mass was detected by LCMS. The mixture was diluted with water (30 mL) and then extracted with EtOAc (20 mL×3). The combined organic phase was washed with saturated brine (20 mL), dried over Na₂SO₄, and filtered and concentrated under reduced pressure. The residue was purified by prep-HPLC (column: Phenomenex luna C18 150×25 mm×10 μm; mobile phase: [water (FA)-ACN]; B %: 43%-73%, 10 min) and prep-HPLC (column: Waters Xbridge 150×25 mm×5 μm; mobile phase: [water (NH₄HCO₃)-ACN]; B %: 49%-79%, 8 min) and then freeze-dried to obtain Compound 26 (23.5 mg, 24.84 μmol, 8.15% yield, 92.2% purity) as yellow solid. MS(M+H)⁺=872.5.

¹H NMR (400 MHz, DMSO-d₆) δ=11.09 (br s, 1H), 9.89 (s, 1H), 9.65 (br s, 1H), 9.27 (s, 1H), 8.18 (br d, J=3.4 Hz, 1H), 8.03-7.95 (m, 4H), 7.65 (d, J=7.6 Hz, 1H), 7.61-7.54 (m, 4H), 7.39 (t, J=7.1 Hz, 1H), 7.31-7.25 (m, 1H), 7.11 (d, J=8.3 Hz, 2H), 7.06 (d, J=8.8 Hz, 1H), 7.01 (d, J=7.0 Hz, 1H), 6.57-6.49 (m, 1H), 5.08-5.00 (m, 1H), 4.36-4.34 (m, 1H), 3.97-3.89 (m, 1H), 3.68-3.60 (m, 2H), 3.31-3.30 (m, 2H), 3.25-3.23 (m, 1H), 2.98-2.81 (m, 4H), 2.03-1.98 (m, 1H), 1.64-1.52 (m, 4H), 1.46-1.39 (m, 1H), 1.25-1.18 (m, 2H), 0.90-0.80 (m, 2H).

### Example 27: Synthesis of Compound 27

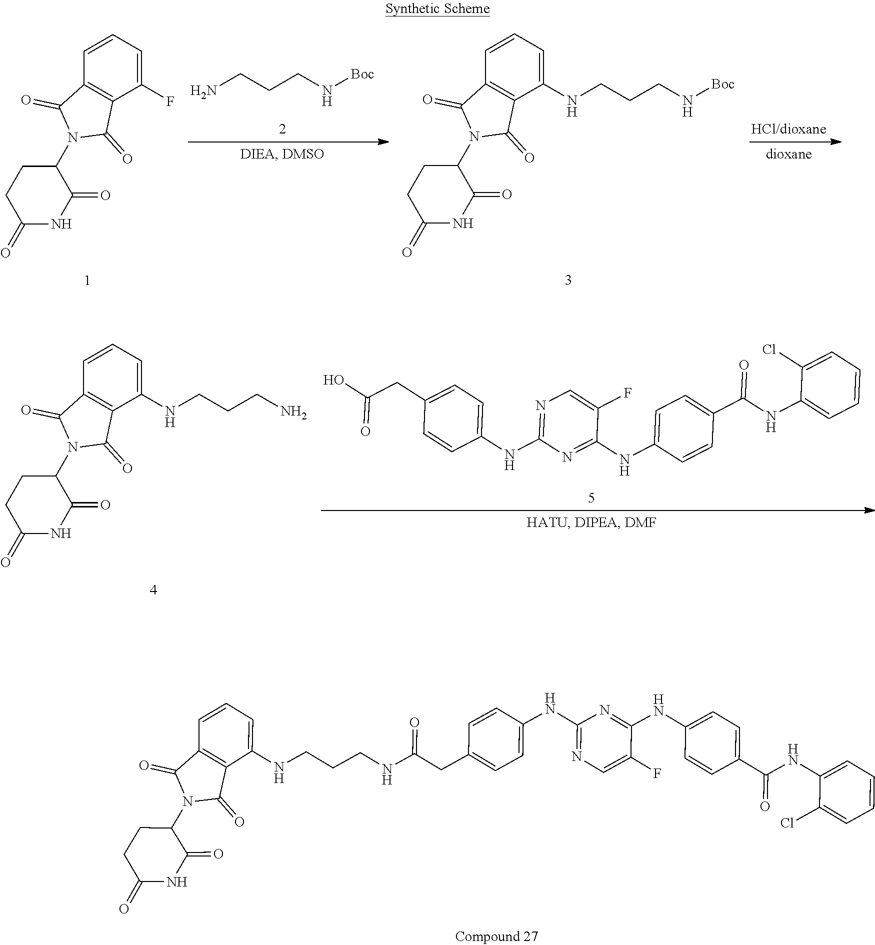

### Experimental Process

### Step 1: Synthesis of tert-butyl (3-((2-(2,6-dioxopiperidin-3 yl)-1,3-dioxoisoindolin-4-yl)amino) propyl)carbamate (3)

DIEA (280.74 mg, 2.17 mmol, 378.36 μL) and tert-butyl (3-aminopropyl)carbamate (189.24 mg, 1.09 mmol, 189.62 μL) were added to a solution of 2-(2,6-dioxopiperidin-3-yl)-4-fluoroisoindoline-1,3-dione (300 mg, 1.09 mmol) in DMSO (5 mL) and then stirred at 80° C. for 12 hours. The desired mass was detected by LCMS. The mixture was diluted with water (20 mL) and then extracted with EtOAc (30 mL×3). The combined organic phase was washed with saturated brine (20 mL), dried over anhydrous Na₂SO₄, and filtered and concentrated under reduced pressure. The residue was purified by flash silica gel chromatography (ISCO®; 20 g SepaFlash® Silica Flash Column, Eluent of 0˜40% petroleum ether:EtOAc gradient @ 60 mL/min) to obtain tert-butyl (3-((2-(2,6-dioxopiperidin-3-yl)-1,3-dioxoisoindolin-4-yl)amino) propyl)carbamate (0.2 g, 458.59 μmol, 42.22% yield, 98.7% purity) as yellow oil. MS(M+Na)⁺=453.2.

### Step 2: Synthesis of 4-((3-aminopropyl)amino)-2-(2,6-dioxopiperidin-3-yl)isoindoline-1,3-dione (4)

HCl/dioxane (4 M, 5 mL) was added to a solution of tert-butyl (3-((2-(2,6-dioxopiperidin-3-yl)-1,3-dioxoisoindolin-4-yl)amino)propyl)carbamate (0.2 g, 464.63 μmol) in dioxane (2 mL) and then stirred at 20° C. for 1 hour. It was confirmed through TLC (petroleum ether:EtOAc=1:1) that the starting material was completely consumed. The mixture was concentrated under reduced pressure to obtain 4-((3-aminopropyl)amino)-2-(2,6-dioxopiperidin-3-yl)isoindoline-1,3-dione (0.17 g, crude, HCl) as yellow solid. MS(M+H)⁺=331.0.

### Step 3: Synthesis of N-(2-chlorophenyl)-4-((2-((4-(2-((3-((2-(2,6-dioxopiperidin-3 yl)-1,3-dioxoisoindolin-4-yl))amino)propyl)amino)-2-oxoethyl)phenyl)amino)-5-fluoropyrimidin-4-yl)amino)benzamide (Compound 27)

HATU (154.60 mg, 406.59 μmol) and DIPEA (78.82 mg, 609.88 μmol, 106.23 μL) were added to a solution of 2-(4-((4-((4-((2-chlorophenyl)carbamoyl)phenyl)amino)-5-fluoropyrimidin-2-yl) amino)phenyl) acetic acid (100 mg, 203.29 μmol) in DMF (2 mL). After 30 minutes, 4-((3-aminopropyl)amino)-2-(2,6-dioxopiperidin-3-yl)isoindoline-1,3-dione (74.57 mg, 203.29 μmol, HCl) was added and then stirred at 25° C. for 2 hours. The desired mass was detected by LCMS. The mixture was diluted with water (20 mL) and then extracted with EtOAc (20 mL×3). The combined organic phase was washed with saturated brine (20 mL), dried over Na₂SO₄, and filtered and concentrated under reduced pressure. The residue was purified by prep-HPLC (column: Phenomenex luna C18 150×25 mm×10 μm; mobile phase: [water (FA)-ACN]; B %: 30%-60%, 10 min) and prep-HPLC (column: Waters Xbridge 150×25 mm×5 μm; mobile phase: [water (NH₄HCO₃)-ACN]; B %: 36%-66%, 9 min) to obtain Compound 27 (20.5 mg, 23.32 μmol, 11.47% yield, 91.5% purity) as yellow solid. MS(M+H)⁺=804.2.

¹H NMR (400 MHz, DMSO-d₆) δ=11.08 (br s, 1H), 9.90 (s, 1H), 9.64 (s, 1H), 9.26 (s, 1H), 8.17 (d, J=3.5 Hz, 1H), 8.07 (t, J=5.7 Hz, 1H), 8.04-8.00 (m, 2H), 8.00-7.94 (m, 2H), 7.66-7.63 (m, 1H), 7.59 (d, J=8.4 Hz, 2H), 7.57-7.49 (m, 2H), 7.41-7.35 (m, 1H), 7.31-7.25 (m, 1H), 7.17 (d, J=8.6 Hz, 2H), 7.01-6.95 (m, 2H), 6.64 (t, J=6.1 Hz, 1H), 5.04 (dd, J=5.1, 12.7 Hz, 1H), 3.30 (s, 2H), 3.28-3.21 (m, 2H), 3.13-3.08 (m, 2H), 2.91-2.82 (m, 1H), 2.61-2.57 (m, 2H), 2.07-1.97 (m, 1H), 1.71-1.60 (m, 2H).

### Example 28: Synthesis of Compound 28

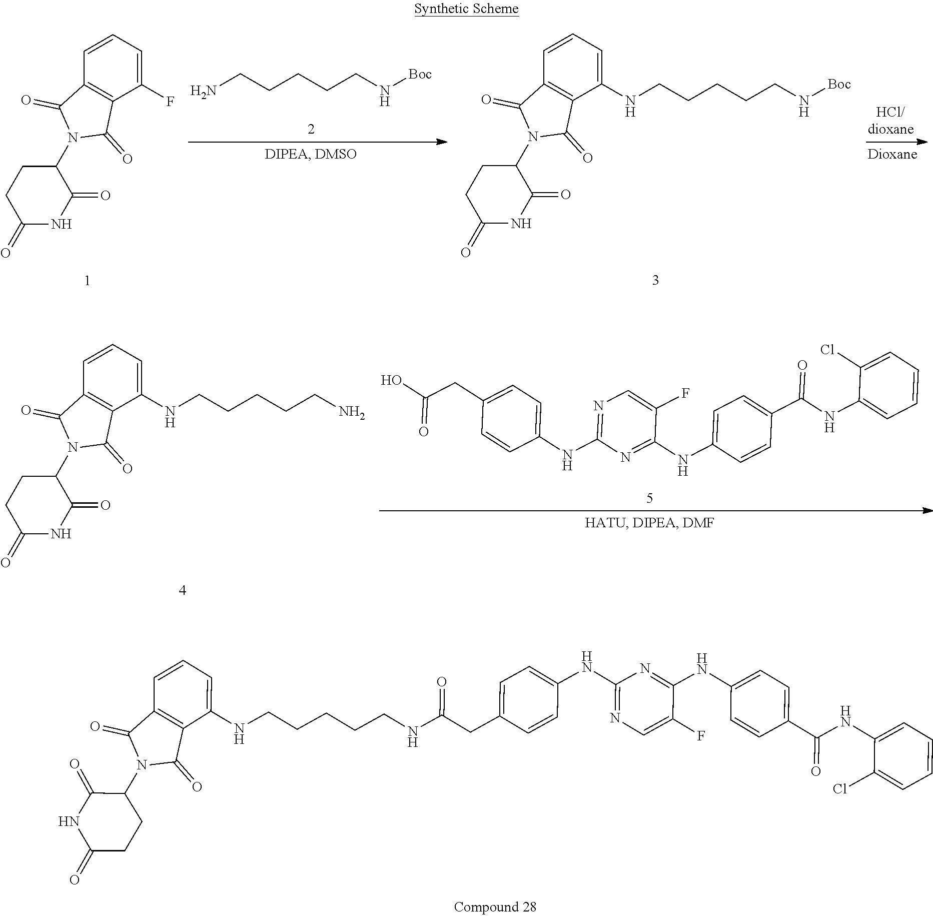

### Experimental Process

### Step 1: Synthesis of tert-butyl (5-((2-(2,6-dioxopiperidin-3 yl)-1,3-dioxoisoindolin-4-yl)amino)pentyl)carbamate (3)

DIPEA (467.89 mg, 3.62 mmol) and tert-butyl N-(5-aminopentyl)carbamate (366.18 mg, 1.81 mmol) were added to a solution of 2-(2,6-dioxo-3-piperidyl)-4-fluoro-isoindoline-1,3-dione (500 mg, 1.81 mmol) in DMSO (6 mL) and then stirred at 100° C. for 16 hours. It was confirmed through LCMS that the starting material was completely consumed, and a main peak with the desired mass was detected. The mixture was poured with water (20 mL) and extracted with EtOAc (10 mL×3). The combined organic phase was washed with brine (10 mL×3), dried over Na₂SO₄, and filtered and concentrated under reduced pressure to obtain tert-butyl (5-((2-(2,6-dioxopiperidin-3-yl)-1,3-dioxoisoindolin-4-yl)amino)pentyl)carbamate (770 mg, 1.68 mmol, 92.77% yield) as brown oil. MS(M+Na)⁺=481.3.

### Step 2: Synthesis of 4-((5-aminopentyl)amino)-2-(2,6-dioxopiperidin-3-yl)isoindoline-1,3-dione (4)

HCl/dioxane (4 M, 8 mL) was added to a solution of tert-butyl (5-((2-(2,6-dioxopiperidin-3-yl)-1,3-dioxoisoindolin-4-yl)amino)pentyl)carbamate (770 mg, 1.68 mmol) in dioxane (8 mL) and then stirred at 25° C. for 1 hour. The main peak of desired mass was detected by LCMS. The mixture was concentrated under reduced pressure to obtain 4-((5-aminopentyl)amino)-2-(2,6-dioxopiperidin-3-yl)isoindoline-1,3-dione (500 mg, 1.40 mmol, 83.07% yield) as white solid. MS(M+H)⁺=359.3.

### Step 3: Synthesis of N-(2-chlorophenyl)-4-((2-((4-(2-((5-((2-(2,6-dioxopiperidin-3 yl)-1,3-dioxoisoindolin-4-yl))amino)pentyl)amino)-2-oxoethyl)phenyl)amino)-5-fluoropyrimidin-4-yl)amino)benzamide (Compound 28)

DIPEA (360.62 mg, 2.79 mmol) and HATU (254.62 mg, 669.66 μmol) were added to a solution of 2-(4-((4-((4-((2-chlorophenyl)carbamoyl)phenyl) amino)-5-fluoropyrimidin-2-yl)amino)phenyl)acetic acid (274.51 mg, 558.05 μmol) and 4-((5-aminopentyl)amino)-2-(2,6-dioxopiperidin-3-yl)isoindoline-1,3-dione (200 mg, 558.05 μmol) in DMF (5 mL) at 25° C., and then stirred for 16 hours. The desired mass was detected by LCMS. The mixture was diluted with water (30 mL) and then extracted with EtOAc (20 mL×3). The combined organic phase was washed with brine (10 mL×3), dried over Na₂SO₄, filtered, and concentrated under reduced pressure. The residue was purified by prep-HPLC (column: Unisil 3-100 C18 μLtra 150*50 mm*3 um; mobile phase: [water (FA)-ACN]; B %: 39%-69%, 7 min) and then freeze-dried to obtain Compound 28 (83.5 mg, 92.30 μmol, 16.54% yield, 92% purity) as yellow solid. MS(M+H)⁺=832.3.

¹H NMR (400 MHz, DMSO-d₆) δ=11.10 (s, 1H), 9.92 (s, 1H), 9.64 (s, 1H), 9.26 (s, 1H), 8.16 (d, J=3.7 Hz, 1H), 8.06-7.94 (m, 5H), 7.64 (dd, J=1.5, 7.9 Hz, 1H), 7.59-7.52 (m, 4H), 7.39 (dt, J=1.4, 7.7 Hz, 1H), 7.32-7.26 (m, 1H), 7.15 (d, J=8.4 Hz, 2H), 7.07-6.98 (m, 2H), 6.50 (br t, J=5.7 Hz, 1H), 5.04 (dd, J=5.4, 12.8 Hz, 1H), 3.33 (br s, 2H), 3.21 (q, J=6.6 Hz, 2H), 3.02 (q, J=6.6 Hz, 2H), 2.93-2.82 (m, 1H), 2.62-2.52 (m, 2H), 2.06-1.97 (m, 1H), 1.56-1.48 (m, 2H), 1.44-1.36 (m, 2H), 1.31-1.23 (m, 2H).

### Example 29: Synthesis of Compound 29

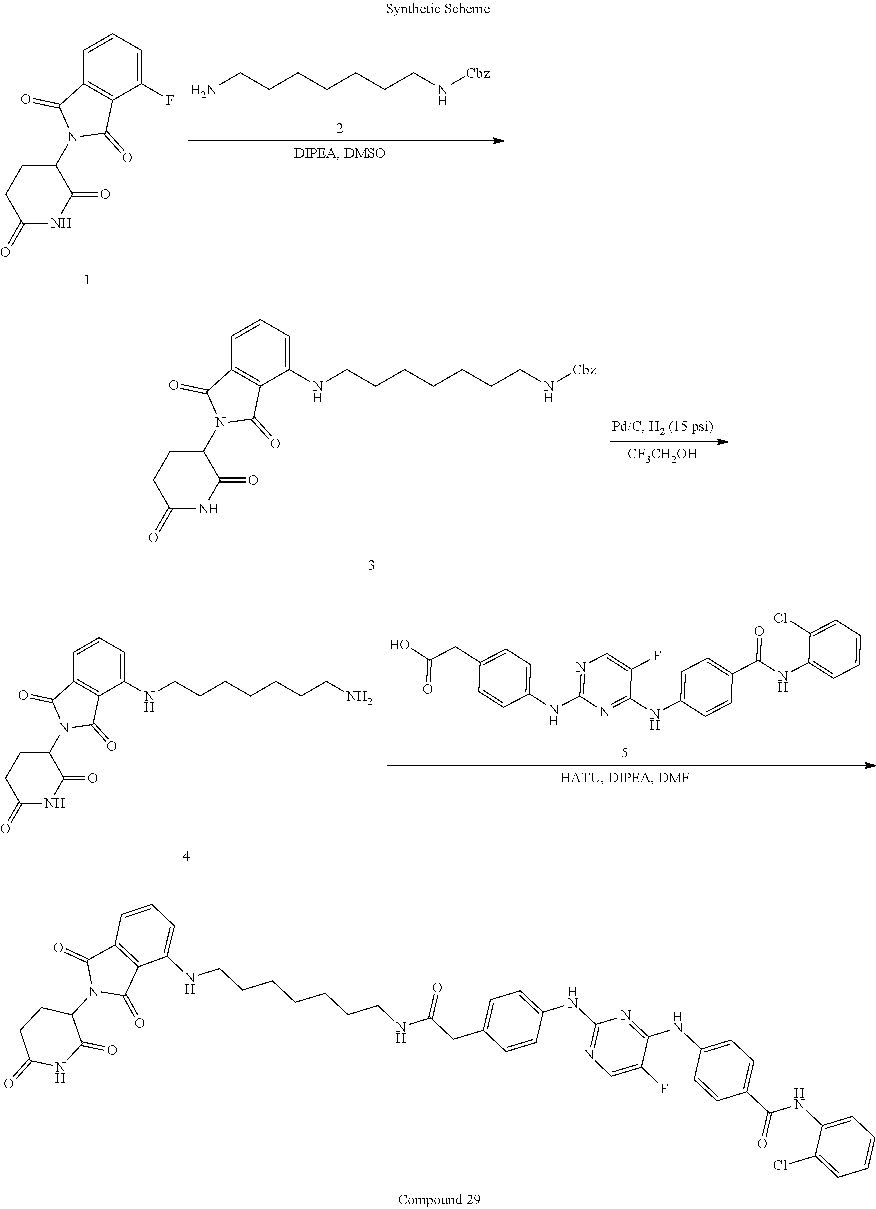

### Experimental Process

### Step 1: Synthesis of benzyl (7-((2-(2,6-dioxopiperidin-3 yl)-1,3-dioxoisoindolin-4-yl)amino)heptyl)carbamate (3)

DIEA (701.85 mg, 5.43 mmol, 945.89 μL) and benzyl(7-aminoheptyl)carbamate (589.94 mg, 1.90 mmol, FA) were added to a solution of 2-(2,6-dioxopiperidin-3-yl)-4-fluoroisoindoline-1,3-dione (0.5 g, 1.81 mmol) in DMSO (5 mL) and then stirred at 100° C. for 12 hours. The main peak of desired mass was detected by LCMS. The mixture was diluted with water (20 mL) and then extracted with EtOAc (10 mL×3). The combined organic phase was washed with saturated brine (10 mL), dried over anhydrous Na₂SO₄, and filtered and concentrated under reduced pressure. The residue was purified by flash silica gel chromatography (ISCO®; 20 g SepaFlash® Silica Flash Column, Eluent of 0-40% petroleum ether:EtOAc gradient @ 80 mL/min) to obtain benzyl (7-((2-(2,6-dioxopiperidin-3-yl)-1,3-dioxoisoindolin-4-yl)amino)heptyl)carbamate (0.8 g, 1.54 mmol, 84.90% yield, 100% purity) as yellow oil. MS(M+H)⁺=521.2.

### Step 2: Synthesis of 4-((7-aminoheptyl)amino)-2-(2,6-dioxopiperidin-3-yl)isoindoline-1,3-dione (4)

Pd/C (0.1 g, 288.14 μmol, 10% purity) was added to a solution of benzyl (7-((2-(2,6-dioxopiperidin-3-yl)-1,3-dioxoisoindolin-4-yl)amino)heptyl)carbamate (0.3 g, 576.29 μmol) in CF₃CH₂OH (10 mL) under N₂ atmosphere. The mixture was stirred for 12 hours at 25° C. under H₂ atmosphere (15 Psi). The main peak of desired mass was detected by LCMS. The mixture was concentrated under reduced pressure to obtain 4-((7-aminoheptyl)amino)-2-(2,6-dioxopiperidin-3-yl)isoindoline-1,3-dione (140 mg, 224.61 μmol, 38.98% yield, 62% purity) as yellow oil. MS(M+H)⁺=387.2.

### Step 3: Synthesis of N-(2-chlorophenyl)-4-((2-((4-(2-((7-((2-(2,6-dioxopiperidin-3 yl))-1,3-dioxoisoindolin-4-yl)amino)heptyl)amino)-2-oxoethyl)phenyl)amino)-5-fluoropyrimidin-4-yl)amino)benzamide (Compound 29)

HATU (167.48 mg, 440.47 μmol) and DIPEA (87.58 mg, 677.64 μmol, 118.03 μL) were added to a solution of 2-(4-((4-((4-((2-chlorophenyl)carbamoyl)phenyl)amino)-5-fluoropyrimidin-2-yl) amino)phenyl) acetic acid (150 mg, 304.94 μmol) in DMF (5 mL). After 30 minutes, 4-((7-aminoheptyl)amino)-2-(2,6-dioxopiperidin-3-yl)isoindoline-1,3-dione (130.94 mg, 338.82 μmol) was added and then stirred at 25° C. for 12 hours. The desired mass was detected by LCMS. The mixture was diluted with water (30 mL) and then extracted with EtOAc (10 mL×3). The combined organic phase was washed with saturated brine (10 mL), dried over anhydrous Na₂SO₄, and filtered and concentrated under reduced pressure. The residue was purified by prep-HPLC (column: Phenomenex C18 75×30 mm×3 μm; mobile phase: [water (FA)-ACN]; B %: 45%-75%, 7 min) and prep-HPLC (column: Waters Xbridge 150×25 mm×5 μm; mobile phase: [water (NH₄HCO₃)-ACNI; B %: 47%-77%, 8 min) and then freeze-dried. The product was repurified by prep-HPLC (column: Welch Ultimate XB—SiOH 250×50×10 μm; mobile phase: [Hexane-EtOH]; B %: 15%-55%, 15 min) and freeze-dried to obtain Compound 29 (9.8 mg, 10.75 μmol, 3.17% yield, 94.4% purity) as yellow solid. MS(M+H)⁺=860.2.

¹H NMR (400 MHz, DMSO-d₆) δ=11.09 (s, 1H), 9.91 (s, 1H), 9.63 (s, 1H), 9.25 (s, 1H), 8.16 (d, J=3.7 Hz, 1H), 8.03-7.94 (m, 5H), 7.67-7.62 (m, 1H), 7.60-7.54 (m, 4H), 7.41-7.36 (m, 1H), 7.31-7.25 (m, 1H), 7.15 (d, J=8.6 Hz, 2H), 7.06-6.97 (m, 2H), 6.54-6.44 (m, 1H), 5.04 (dd, J=5.3, 13.6 Hz, 1H), 3.30-3.30 (m, 2H), 3.26-3.21 (m, 2H), 3.02-2.97 (m, 2H), 2.88-2.82 (m, 1H), 2.60-2.58 (m, 2H), 2.03-1.96 (m, 1H), 1.55-1.49 (m, 2H), 1.38-1.32 (m, 2H), 1.28-1.22 (m, 6H).

### Example 30: Synthesis of Compound 30

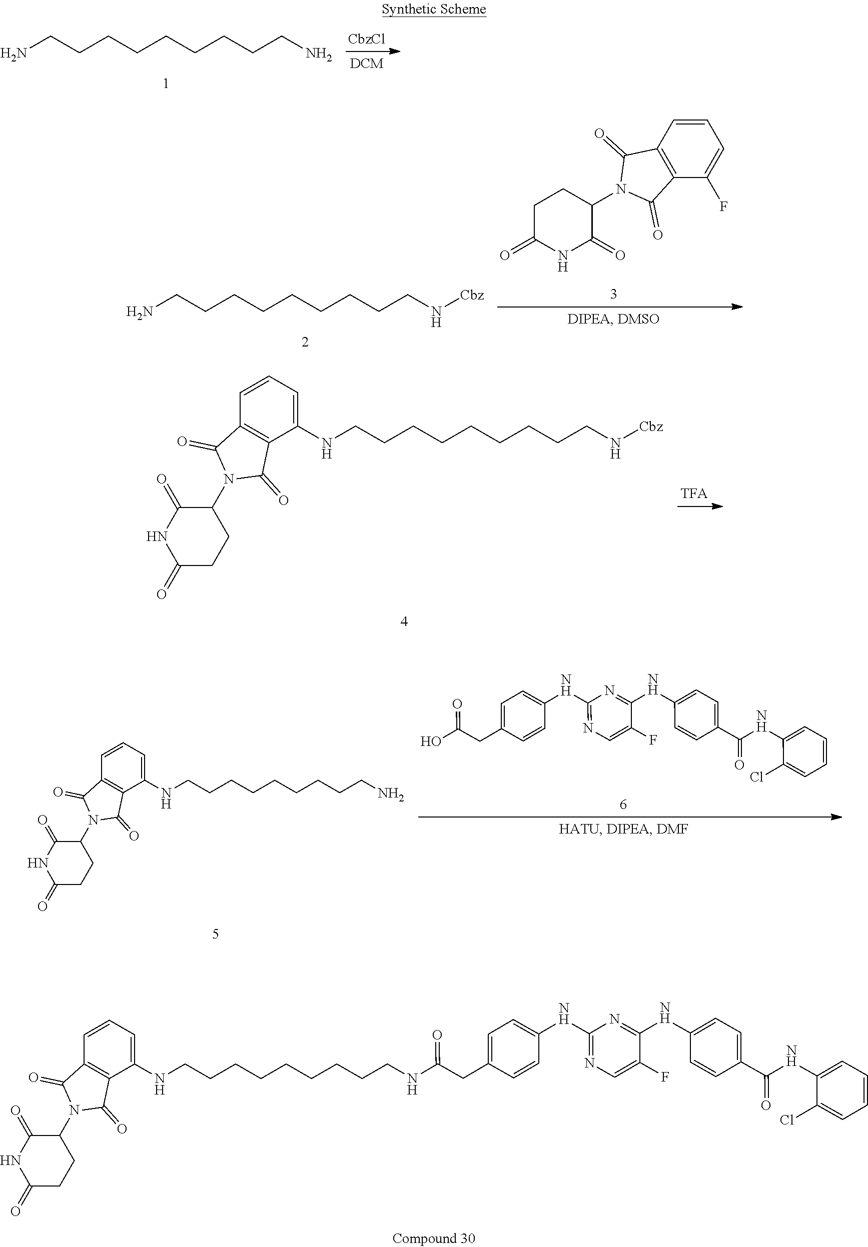

### Experimental Process

### Step 1: Synthesis of 4 benzyl(9-aminononyl)carbamate (2)

A solution of CbzCl (1.62 g, 9.48 mmol, 1.35 mL) in DCM (100 mL) was added dropwise to a solution of nonane-1,9-diamine (2.5 g, 15.79 mmol) in DCM (100 mL) for 3 hours at 0° C. The mixture was stirred at 25° C. for 12 hours. The desired mass was detected by LCMS. The mixture was filtered to remove a solid. The filtrate was concentrated under reduced pressure, purified by reverse phase flash column (column: Phenomenex Luna C18 15 μm; 100 A, solvent for sample dissolution about 3.00 grams of sample dissolved in 10 ml of MeOH; mobile phase: [water-ACN]; B %: 0%-50%; 20 min) and freeze-dried to obtain 4 benzyl(9-aminononyl)carbamate (0.9 g, 2.83 mmol, 17.93% yield, 92% purity) as white solid. MS(M+H)⁺=293.2.

### Step 2: Synthesis of benzyl (9-((2-(2,6-dioxopiperidin-3 yl)-1,3-dioxoisoindolin-4-yl)amino)nonyl)carbamate (4)

DIPEA (308.81 mg, 2.39 mmol) and benzyl(9-aminononyl)carbamate (269.56 mg, 796.47 μmol) were added to a solution of 2-(2,6-dioxopiperidin-3-yl)-4-fluoroisoindoline-1,3-dione (220 mg, 796.47 μmol) in DMSO (5 mL) and then stirred at 100° C. for 12 hours. It was confirmed through LCMS that the starting material was completely consumed, and the desired mass was detected. The mixture was poured with water (20 mL) and extracted with EtOAc (10 mL×3). The combined organic phase was washed with saturated brine (10 mL), dried over anhydrous Na₂SO₄, and filtered and concentrated under reduced pressure. The residue was purified by flash silica gel chromatography (ISCO®; 25 g SepaFlash® Silica Flash Column, Eluent of 0-40% petroleum ether:EtOAc gradient @ 80 mL/min) to obtain benzyl (9-((2-(2,6-dioxopiperidin-3-yl)-1,3-dioxoisoindolin-4-yl)amino)nonyl)carbamate (300 mg, 546.82 μmol, 68.66% yield, N/A purity) as yellow oil. MS(M+H)⁺=549.2.

### Step 3: Synthesis of 4-((9-aminononyl)amino)-2-(2,6-dioxopiperidin-3-yl)isoindoline-1,3-dione (5)

A solution of benzyl (9-((2-(2,6-dioxopiperidin-3-yl)-1,3-dioxoisoindolin-4-yl)amino)nonyl) carbamate (250 mg, 455.68 μmol) in TFA (5 mL) was stirred at 40° C. for 16 hours. The main peak of desired mass was detected by LCMS. The mixture was concentrated under reduced pressure to obtain 4-((9-aminononyl)amino)-2-(2,6-dioxopiperidin-3-yl)isoindoline-1,3-dione (240 mg, crude, TFA) as yellow oil. MS(M+H)⁺=415.2.

### Step 4: Synthesis of N-(2-chlorophenyl)-4-((2-((4-(2-((9-((2-(2,6-dioxopiperidin-3 yl))-1,3-dioxoisoindolin-4-yl)amino)nonyl)amino)-2-oxoethyl)phenyl)amino)-5-fluoropyrimidin-4-yl)amino)benzamide (Compound 30)

HATU (215.83 mg, 567.62 μmol) and DIPEA (146.72 mg, 1.14 mmol) were added to a solution of 2-(4-((4-((4-((2-chlorophenyl)carbamoyl)phenyl) amino)-5-fluoropyrimidin-2-yl)amino)phenyl)acetic acid (186.14 mg, 378.42 μmol) in DMF (1 mL) and then stirred at 20° C. for 30 minutes. Thereafter, 4-((9-aminononyl)amino)-2-(2,6-dioxopiperidin-3-yl)isoindoline-1,3-dione (200 mg, 378.42 μmol, TFA) was added and then stirred at 20° C. for 12 hours. The main peak of desired mass was detected by LCMS. The mixture was poured with water (20 mL) and extracted with EtOAc (1 mL×3). The combined organic phase was washed with brine (10 mL×2), dried over anhydrous Na₂SO₄, filtered, and concentrated under reduced pressure. The residue was purified by prep-TLC (SiO₂, DCM:MeOH=10:1) and then repurified by reverse phase HPLC (column: Phenomenex luna C18 150*25 mm*10 um; mobile phase: [water (FA)-ACN]; B %: 48%-78%, 10 min) and then freeze-dried to obtain Compound 30 (41.1 mg, 41.64 μmol, 11.00% yield, 90% purity) as yellow solid. MS(M+Na)⁺=910.2.

¹H NMR (400 MHz, DMSO-d₆) δ=11.09 (s, 1H), 9.91 (s, 1H), 9.78-9.69 (m, 1H), 9.37-9.27 (m, 1H), 8.18 (d, J=3.8 Hz, 1H), 8.04-7.91 (m, 5H), 7.67-7.61 (m, 1H), 7.59-7.53 (m, 4H), 7.42-7.35 (m, 1H), 7.32-7.25 (m, 1H), 7.16 (d, J=8.4 Hz, 2H), 7.09-6.98 (m, 2H), 6.55-6.45 (m, 1H), 5.08-5.02 (m, 1H), 3.25 (s, 2H), 3.28-3.23 (m, 2H), 3.02-2.94 (m, 2H), 2.94-2.82 (m, 1H), 2.62-2.55 (m, 2H), 2.05-1.98 (m, 1H), 1.57-1.49 (m, 2H), 1.38-1.25 (m, 6H), 1.25-1.13 (m, 6H).

### Example 31: Synthesis of Compound 31

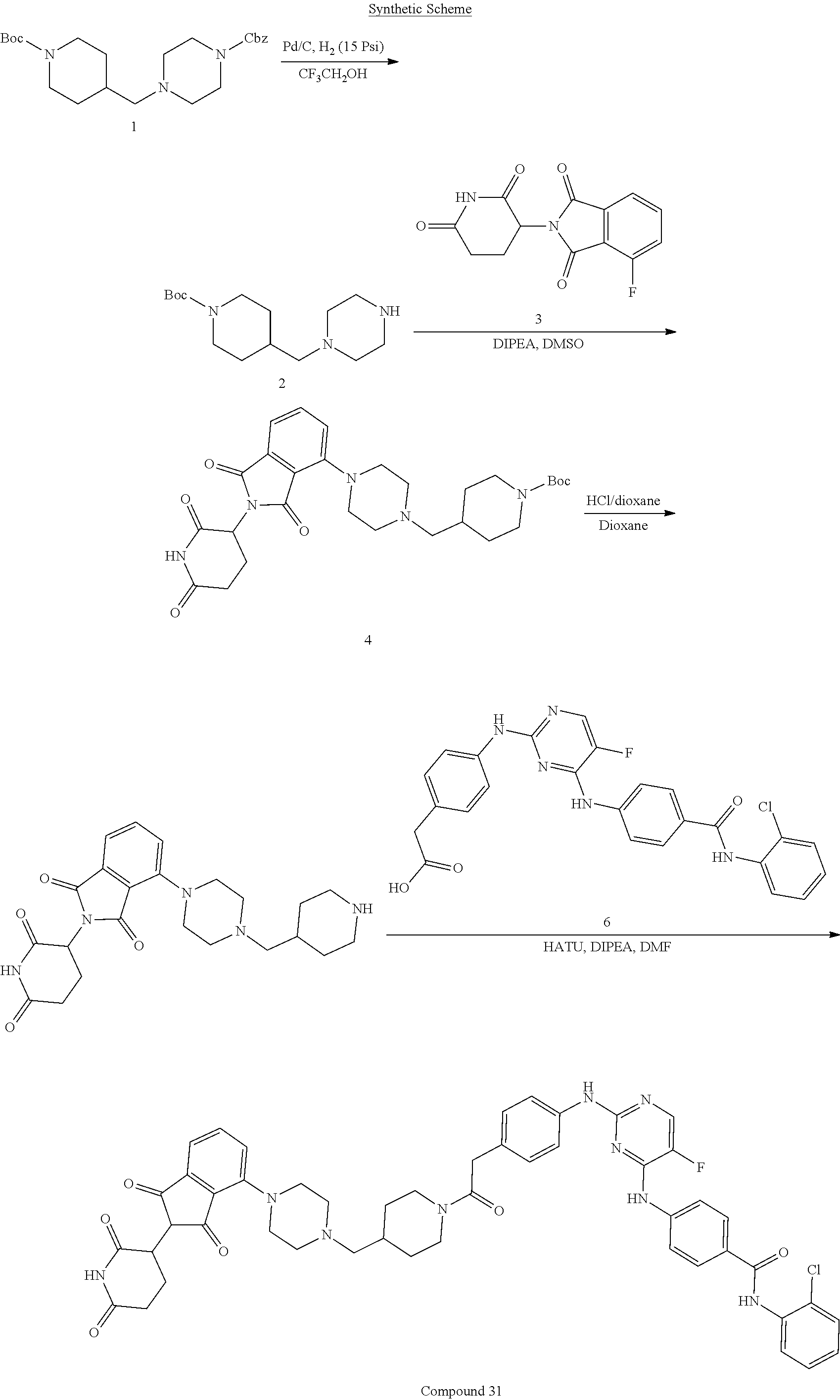

### Experimental Process

### Step 1: Synthesis of tert-butyl 4-(piperazin-1-ylmethyl)piperidine-1-carboxylate (2)

Pd/C (200 mg, 10% purity) was added to a solution of benzyl 4-((1-(tert-butoxycarbonyl)piperidin-4-yl)methyl)piperazine-1-carboxylate (1 g, 2.39 mmol) in CF₃CH₂OH (30 mL) at 20° C. under nitrogen gas. The mixture was purged and degassed three times under hydrogen gas and then stirred at 20° C. for 14 hours under H₂ atmosphere (15 Psi). It was confirmed through TLC (petroleum ether:EtOAc=1:1, R_{f}=0) and LCMS that the starting material was completely consumed. The mixture was filtered through a celite pad and the filtrate was concentrated under reduced pressure to obtain tert-butyl 4-(piperazin-1-ylmethyl)piperidine-1-carboxylate (700 mg, crude) as white solid. MS(M+H)⁺=284.4

### Step 2: Synthesis of tert-butyl 4-((4-(2-(2,6-dioxopiperidin-3 yl)-1,3-dioxoisoindolin-4-yl)piperazin-1 yl)methyl)piperidine-1-carboxylate (4)

DIPEA (655.06 mg, 5.07 mmol) was added to a solution of 2-(2,6-dioxo-3-piperidyl)-4-fluoro-isoindoline-1,3-dione (700 mg, 2.53 mmol) and tert-butyl 4-(piperazine-1-ylmethyl)piperidine-1-carboxylate (700 mg, 2.47 mmol) in DMSO (10 mL) at 20° C. and then stirred at 100° C. for 14 hours. The main peak of desired mass was detected by LCMS. The mixture was poured with water (40 mL), filtered, and washed with water (10 mL). The solid portion was collected and dried to obtain tert-butyl 4-((4-(2-(2,6-dioxopiperidin-3-yl)-1,3-dioxoisoindolin-4-yl)piperazin-1-yl)methyl)piperidine-1-carboxylate (1.5 g, crude) as yellow solid. MS(M+H)⁺=540.4

### Step 3: Synthesis of 2-(2,6-dioxopiperidin-3-yl)-4-(4-(piperidin-4-ylmethyl)piperazin-1 yl)isoindoline-1,3-dione (5)

HCl/dioxane (4 M, 2 mL) was added to a solution of tert-butyl 4-((4-(2-(2,6-dioxopiperidin-3-yl)-1,3-dioxoisoindolin-4-yl)piperazin-1-yl)methyl)piperidine-1-carboxylate (100 mg, 185.31 μmol) in dioxane (2 mL) at 20° C. and then stirred at 20° C. for 1 hour. It was confirmed through LCMS that the starting material was completely consumed, and the desired mass was detected. The mixture was concentrated under reduced pressure to obtain 2-(2,6-dioxopiperidin-3-yl)-4-(4-(piperidin-4-ylmethyl)piperazin-1-yl)isoindoline-1,3-dione (100 mg, crude, 2HCl) as yellow solid, which was immediately used in the next reaction. MS(M+H)⁺=440.3

### Step 4: Synthesis of N-(2-chlorophenyl)-4-((2-((4-(2-(4-((4-(2-(2,6-dioxopiperidin-3 yl))-1,3-dioxoisoindolin-4-yl)piperazin-1 yl)methyl)piperidin-1-yl)-2-oxoethyl)phenyl)amino)-5-fluoropyrimidin-4-yl)amino)benzamide (Compound 31)

2-(2,6-dioxopiperidin-3-yl)-4-(4-(piperidin-4-ylmethyl)piperazin-1-yl)isoindoline-1,3-dione (100 mg, 195.15 μmol, 2HCl) was added to a solution of 2-(4-((4-((4-((2-chlorophenyl)carbamoyl)phenyl)amino)-5-fluoropyrimidin-2-yl)amino)phenyl)acetic acid (95.99 mg, 195.15 μmol), DIPEA (126.11 mg, 975.75 μmol) and HATU (89.04 mg, 234.18 μmol) in DMF (2 mL) at 20° C. and then stirred for 1 hour. It was confirmed through LCMS that the starting material was completely consumed, and the desired mass was detected. The mixture was poured with water (10 mL) and extracted with EtOAc (6 mL×4). The combined organic phase was washed with brine (5 mL), dried over Na₂SO₄, concentrated under reduced pressure, and then purified by prep-TLC (DCM:MeOH=10:1, R_{f}=0.6). The product was dissolved in MeCN/H₂O and freeze-dried. The product was further purified by prep-HPLC (column: Waters Xbridge 150*25 mm*Sum; mobile phase: [water (NH₄HCO₃)-ACN]; B %: 43%-73%, 8 min) and freeze-dried to obtain Compound 31 (9.1 mg, 9.56 μmol, 4.90% yield, 96% purity) as yellow solid. MS(M+H)⁺=913.4.

¹H NMR (400 MHz, DMSO-d₆) δ=11.09 (s, 1H), 9.90 (s, 1H), 9.66 (s, 1H), 9.29 (s, 1H), 8.18 (d, J=3.5 Hz, 1H), 8.05-8.01 (m, 2H), 7.99-7.95 (m, 2H), 7.72-7.63 (m, 2H), 7.60 (d, J=8.6 Hz, 2H), 7.56 (dd, J=1.4, 8.0 Hz, 1H), 7.39 (dt, J=1.4, 7.7 Hz, 1H), 7.35 (d, J=7.1 Hz, 1H), 7.32-7.26 (m, 2H), 7.12 (d, J=8.6 Hz, 2H), 5.09 (dd, J=5.7, 12.9 Hz, 1H), 4.36 (br d, J=10.8 Hz, 1H), 3.95 (br d, J=12.0 Hz, 1H), 3.70-3.55 (m, 2H), 3.31 (s, 2H), 3.30-3.20 (m, 4H), 3.02-2.80 (m, 2H), 2.62-2.53 (m, 5H), 2.16-2.08 (m, 2H), 2.06-1.98 (m, 1H), 1.82-1.72 (m, 1H), 1.71-1.62 (m, 2H), 0.96-0.79 (m, 2H)

### Example 32: Synthesis of Compound 32

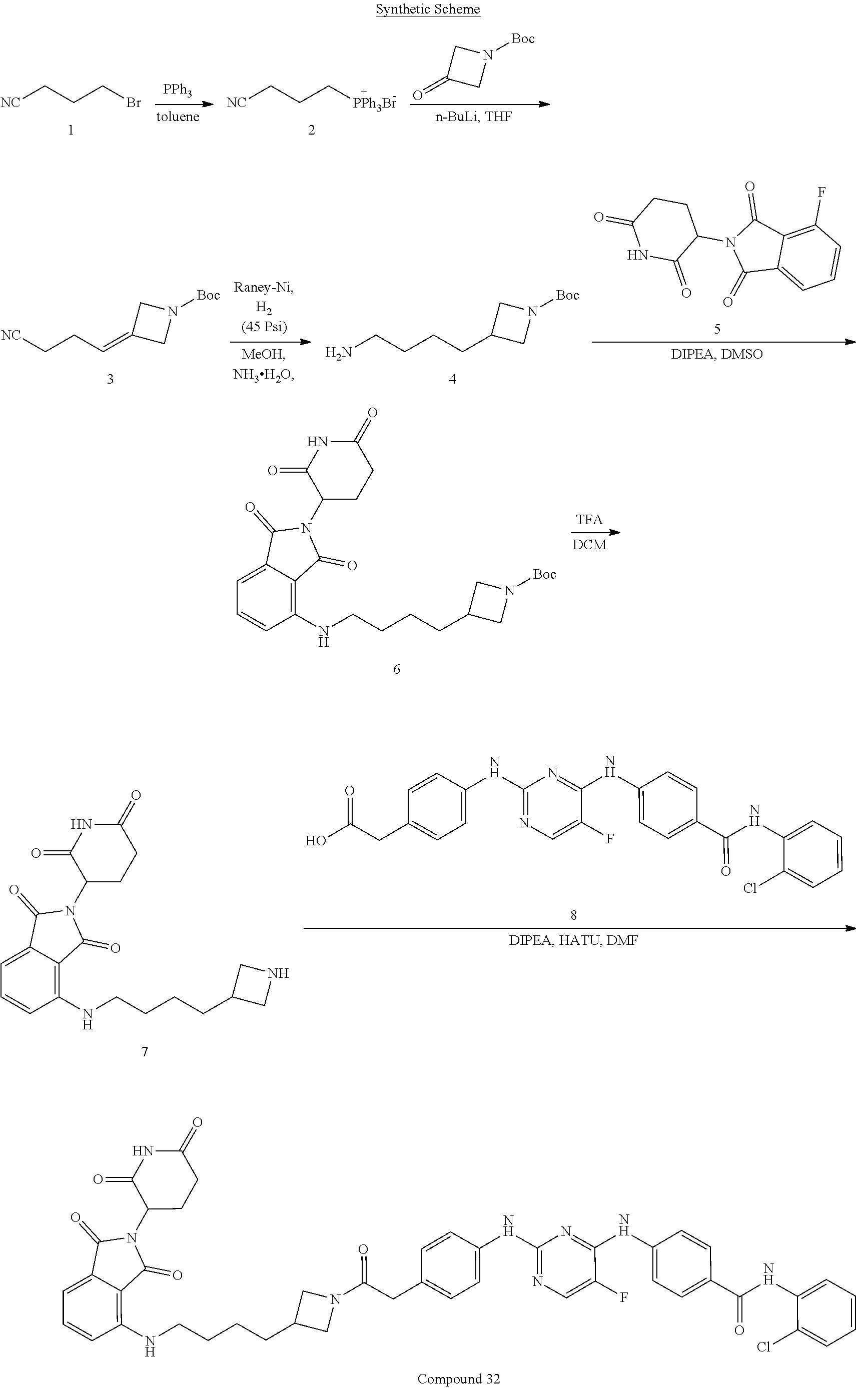

### Experimental Process

### Step 1: Synthesis of 2-(2,6-dioxopiperidin-3 yl)-4-(3-(hydroxymethyl)pyrrolidin-1-yl)isoindoline-1,3-dione (2)

A solution of 411.34-bromobutanenitrile (10.00 g, 67.57 mmol) and PPh3 (17.72 g, 67.57 mmol) in anhydrous toluene (200 mL) was stirred at 110° C. for 16 hours under N₂ atmosphere. The mixture was filtered and the solid portion was washed with petroleum ether (30 mL) to obtain 2-(2,6-dioxopiperidin-3-yl)-4-(3-(hydroxymethyl)pyrrolidin-1-yl)isoindoline-1,3-dione (6.5 g, crude) as white solid, which was immediately used in the next reaction. MS(M+H)⁺=411.3.

### Step 2: Synthesis of tert-butyl 3-(3-cyanopropylidene)azetidine-1-carboxylate (3)

n-BuLi (2.5 M, 2.14 mL) was added dropwise to a solution of (3-cyanopropyl)triphenylphosphonium bromide (2 g, 4.87 mmol) in THE (20 mL) at 20° C. under N₂ atmosphere and then stirred for 1 hour. The mixture was lowered to 0° C. and then added with tert-butyl 3-oxoazetidine-1-carboxylate (1.67 g, 9.75 mmol) and stirred for 1 hour at 20° C. under N₂ atmosphere. Thereafter, the mixture was stirred at 70° C. for 2 hours. It was confirmed through LCMS that the starting material remained, and the desired mass was not detected. The mixture was further stirred at 70° C. for 2 hours. The desired mass was not detected by LCMS. The mixture was poured with a NH₄Cl (60 mL) solution and extracted with EtOAc (20 mL×4). The combined organic phase was washed with brine (20 mL) and then dried over Na₂SO₄ and concentrated under reduced pressure. The residue was purified by flash silica gel chromatography (ISCO®; 25 g SepaFlash® Silica Flash Column, Eluent of 0-20% EtOAc/Petroleum ether gradient @ 100 mL/min) to obtain tert-butyl 3-(3-cyanopropylidene)azetidine-1-carboxylate (450 mg, 2.02 mmol, 41.53% yield) as colorless oil. MS(M+H)⁺=223.3.

### Step 3: Synthesis of tert-butyl 3-(4-aminobutyl)azetidine-1-carboxylate (4)

Raney-Ni (100 mg, 1.17 mmol) was added to a solution of tert-butyl 3-(3-cyanopropylidene)azetidine-1-carboxylate (450 mg, 2.02 mmol) in MeOH (10 mL) and NH₃·H₂O (1 mL) under N₂ atmosphere. The mixture was purged and degassed three times under hydrogen gas and then stirred at 20° C. for 14 hours under H₂ atmosphere (45 Psi). It was confirmed through TLC (petroleum ether:EtOAc=1:1, R_{f}=0) that the starting material was completely consumed. The mixture was filtered and then washed with MeOH (30 mL). The filtrate was dried with Na₂SO₄ and concentrated under reduced pressure to obtain tert-butyl 3-(4-aminobutyl)azetidine-1-carboxylate (350 mg, 1.53 mmol, 75.72% yield) as yellow solid. MS(M+H)⁺=229.3.

### Step 4: Synthesis of tert-butyl 3-(4-((2-(2,6-dioxopiperidin-3 yl)-1,3-dioxoisoindolin-4-yl)amino)butyl)azetidine-1-carboxylate (6)

DIPEA (339.61 mg, 2.63 mmol) was added to a solution of tert-butyl 3-(4-aminobutyl)azetidine-1-carboxylate (300 mg, 1.31 mmol) and 2-(2,6-dioxo-3-piperidyl)-4-fluoro-isoindoline-1,3-dione (362.92 mg, 1.31 mmol) in DMSO (5 mL) at 20° C. The mixture was stirred at 100° C. for 14 hours. The main peak of desired mass was detected by LCMS. The mixture was poured with water (20 mL) and extracted with EtOAc (8 mL×4). The combined organic phase was washed with brine (5 mL×3), dried over Na₂SO₄, and concentrated under reduced pressure to obtain tert-butyl 3-(4-((2-(2,6-dioxopiperidin-3-yl)-1,3-dioxoisoindolin-4-yl)amino)butyl)azetidine-1-carboxylate (450 mg, crude) as green solid. MS(M+Na)⁺=507.2.

### Step 5: Synthesis of 4-((4-(azetidin-3-yl)butyl)amino)-2-(2,6-dioxopiperidin-3 yl)isoindoline-1,3-dione (7)

TFA (1.54 g, 13.51 mmol, 1 mL) was added to a solution of tert-butyl 3-(4-((2-(2,6-dioxopiperidin-3-yl)-1,3-dioxoisoindolin-4-yl)amino)butyl)azetidine-1-carboxylate (250 mg, 515.95 μmol) in DCM (4 mL) at 20° C. and then stirred for 1 hour. It was confirmed through LCMS that the starting material was completely consumed, and a main peak with the desired mass was detected. The mixture was concentrated under reduced pressure to obtain 4-((4-(azetidin-3-yl)butyl)amino)-2-(2,6-dioxopiperidin-3-yl)isoindoline-1,3-dione (250 mg, crude, TFA) as black oil, which was immediately used in the next reaction. MS(M+H)⁺=385.1.

### Step 6: Synthesis of N-(2-chlorophenyl)-4-((2-((4-(2-(3-(4-((2-(2,6-dioxopiperidin-3 yl))-1,3-dioxoisoindolin-4-yl)amino)butyl)azetidin-1-yl)-2-oxoethyl)phenyl)amino)-5-fluoropyrimidin-4-yl)amino)benzamide (Compound 32)

4-((4-(azetidin-3-yl)butyl)amino)-2-(2,6-dioxopiperidin-3-yl)isoindoline-1,3-dione (250 mg, 501.55 μmol, TFA) was added to a solution of 2-(4-((4-((4-((2-chlorophenyl)carbamoyl)phenyl)amino)-5-fluoropyrimidin-2-yl) amino)phenyl) acetic acid (200 mg, 406.59 μmol), DIPEA (324.11 mg, 2.51 mmol) and HATU (190.71 mg, 501.55 μmol) in DMF (5 mL) at 20° C. and then stirred for 14 hours. It was confirmed through LCMS that the starting material was completely consumed, and a main peak with the desired mass was detected. The combined organic phase was washed with brine (10 mL) and then dried over Na₂SO₄ and concentrated under reduced pressure. The residue was purified by prep-TLC (SiO₂, DCM/MeOH=10:1, R_{f}=0.6). The product was repurified by prep-HPLC (column:Unisil 3-100 C18 μLtra 150*50 mm*3 um; mobile phase: [water (FA)-ACN]; B %: 44%-74%, 7 min) and freeze-dried to obtain Compound 32 (21.4 mg, 23.19 μmol, 4.62% yield, 93% purity) as yellow solid. MS(M+H)⁺=858.2.

¹H NMR (400 MHz, DMSO-d₆) δ=11.09 (s, 1H), 9.89 (s, 1H), 9.64 (s, 1H), 9.26 (s, 1H), 8.17 (d, J=3.5 Hz, 1H), 8.04-8.00 (m, 2H), 7.98-7.95 (m, 2H), 7.64 (br dd, J=1.4, 7.9 Hz, 1H), 7.60-7.53 (m, 4H), 7.39 (dt, J=1.4, 7.7 Hz, 1H), 7.32-7.25 (m, 1H), 7.12 (d, J=8.4 Hz, 2H), 7.07 (d, J=8.6 Hz, 1H), 7.01 (d, J=7.0 Hz, 1H), 6.52 (t, J=5.9 Hz, 1H), 5.05 (dd, J=5.3, 12.9 Hz, 1H), 4.22 (t, J=8.3 Hz, 1H), 3.85 (t, J=8.9 Hz, 1H), 3.75 (dd, J=5.7, 8.2 Hz, 1H), 3.40 (br dd, J=5.6, 9.5 Hz, 2H), 3.29-3.23 (m, 3H), 2.92-2.83 (m, 1H), 2.61-2.52 (m, 3H), 2.06-1.97 (m, 1H), 1.59-1.49 (m, 4H), 1.32-1.20 (m, 2H).

### Example 33: Synthesis of Compound 33

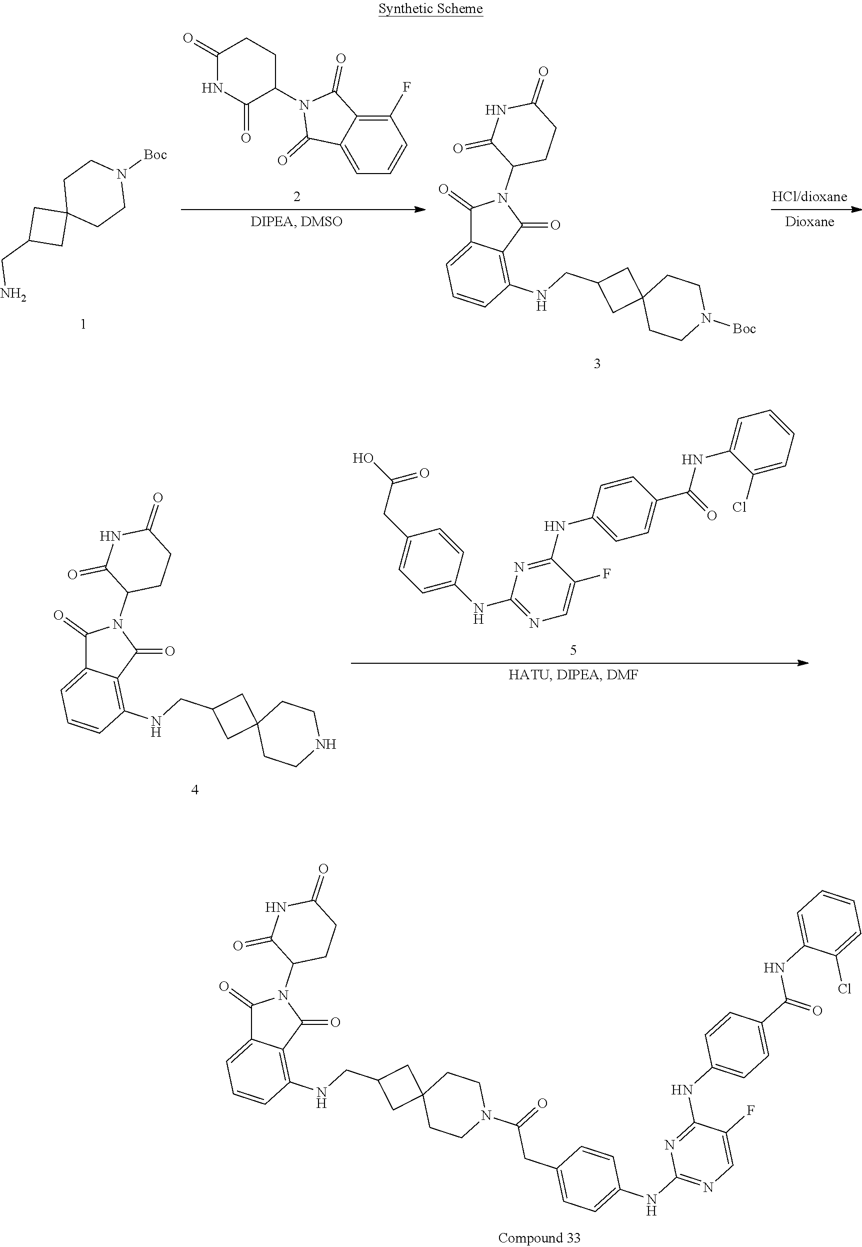 [SMILES: CC(C)(C)OC(=O)N1CCC2(CC1)CC(CN)C2 ; O=C1CCC(N2C(=O)c3cccc(F)c3C2=O)C(=O)N1 ; CC(C)(C)OC(=O)N1CCC2(CC1)CC(CNc1cccc3c1C(=O)N(C1CCC(=O)NC1=O)C3=O)C2 ; O=C1CCC(N2C(=O)c3cccc(NCC4CC5(CCNCC5)C4)c3C2=O)C(=O)N1 ; O=C(O)Cc1ccc(Nc2ncc(F)c(Nc3ccc(C(=O)Nc4ccccc4Cl)cc3)n2)cc1 ; O=C1CCC(N2C(=O)c3cccc(NCC4CC5(CCN(C(=O)Cc6ccc(Nc7ncc(F)c(Nc8ccc(C(=O)Nc9ccccc9Cl)cc8)n7)cc6)CC5)C4)c3C2=O)C(=O)N1]

### Experimental Process

### Step 1: Synthesis of tert-butyl 2-(((2-(2,6-dioxopiperidin-3 yl)-1,3-dioxoisoindolin-4-yl)amino)methyl)-7-azaspiro[3.5]nonane-7-carboxylate (3)

DIPEA (254.05 mg, 1.97 mmol) was added to a solution of tert-butyl 2-(aminomethyl)-7-azaspiro[3.5]nonane-7-carboxylate (250 mg, 982.83 μmol) and 2-(2,6-dioxopiperidin-3-yl)-4-fluoroisoindoline-1,3-dione (271.48 mg, 982.83 μmol) in DMSO (5 mL) at 20° C. The mixture was stirred at 100° C. for 14 hours. The main peak of desired mass was detected by LCMS. The mixture was poured with water (20 mL) and extracted with EtOAc (8 mL×4). The combined organic phase was washed with brine (5 mL×3), dried over Na₂SO₄, and concentrated under reduced pressure to obtain tert-butyl 2-(((2-(2,6-dioxopiperidin-3-yl)-1,3-dioxoisoindolin-4-yl)amino)methyl)-7-azaspiro[3.5]nonane-7-carboxylate (200 mg, crude) as yellow solid. MS(M+H)⁺=511.3

### Step 2: Synthesis of tert-butyl 2-(((2-(2,6-dioxopiperidin-3 yl)-1,3-dioxoisoindolin-4-yl)amino)methyl)-7-azaspiro[3.5]nonane-7-carboxylate (4)

HCl/dioxane (4 M, 4 mL) was added to a solution of tert-butyl 2-(((2-(2,6-dioxopiperidin-3-yl)-1,3-dioxoisoindolin-4-yl)amino)methyl)-7-azaspiro [3.5]nonane-7-carboxylate (200 mg, 391.71 μmol) in dioxane (2 mL) at 20° C. and then stirred for 2 hours. It was confirmed through LCMS that the starting material was completely consumed, and a main peak with the desired mass was detected. The mixture was concentrated under reduced pressure to obtain tert-butyl 2-(((2-(2,6-dioxopiperidin-3-yl)-1,3-dioxoisoindolin-4-yl)amino)methyl)-7-azaspiro[3.5]nonane-7-carboxylate (180 mg, crude, HCl) as yellow solid. MS(M+H)⁺=411.2.

### Step 3: Synthesis of N-(2-chlorophenyl)-4-((2-((4-(2-(2-((2-(2,6-dioxopiperidin-3 yl)-1,3-dioxoisoindolin-4-yl))amino)methyl)-7-azaspiro[3.5]nonan-7-yl)-2-oxoethyl)phenyl)amino)-5-fluoropyrimidin-4-yl)amino)benzamide (Compound 33)

HATU (92.76 mg, 243.95 μmol) was added to a solution of 2-(4-((4-((4-((2-chlorophenyl)carbamoyl)phenyl)amino)-5-fluoropyrimidin-2-yl) amino)phenyl) acetic acid (100 mg, 203.29 μmol), 4-(((7-azaspiro[3.5]nonan-2-yl)methyl)amino)-2-(2,6-dioxopiperidin-3-yl)isoindoline-1,3-dione (90.86 mg, 203.29 μmol, HCl) and DIPEA (131.37 mg, 1.02 mmol) in DMF (2 mL) at 20° C., and then stirred for 2 hours. It was confirmed through LCMS that the starting material was completely consumed, and a main peak with the desired mass was detected. The mixture was poured with water (20 mL) and extracted with EtOAc (10 mL×3). The combined organic phase was washed with brine (10 mL), dried over Na₂SO₄, concentrated under reduced pressure, and purified by prep-TLC (SiO₂, DCM/MeOH=10:1, R_{f}=0.6). The product was repurified by prep-HPLC (column: Unisil 3-100 C18 μLtra 150*50 mm*3 um; mobile phase: [water (FA)-ACN]; B %: 47%-77%, 7 min) and freeze-dried to obtain Compound 33 (10.6 mg, 11.03 μmol, 5.42% yield, 92% purity) as yellow solid. MS(M+H)⁺=884.2.

¹H NMR (400 MHz, DMSO-d₆) δ=11.08 (br s, 1H), 9.88 (s, 1H), 9.65 (s, 1H), 9.27 (s, 1H), 8.18 (d, J=3.5 Hz, 1H), 8.05-8.01 (m, 2H), 7.99-7.93 (m, 2H), 7.69-7.64 (m, 1H), 7.61-7.52 (m, 4H), 7.42-7.36 (m, 1H), 7.32-7.25 (m, 1H), 7.14-7.06 (m, 3H), 7.02 (d, J=6.8 Hz, 1H), 6.46 (br s, 1H), 5.04 (dd, J=5.1, 12.9 Hz, 1H), 3.64-3.59 (m, 2H), 3.41-3.37 (m, 2H), 3.30-3.29 (m, 4H), 2.91-2.83 (m, 1H), 2.62-2.55 (m, 3H), 2.06-1.98 (m, 1H), 1.91-1.81 (m, 2H), 1.54-1.40 (m, 4H), 1.39-1.29 (m, 2H).

### Example 34: Synthesis of Compound 34

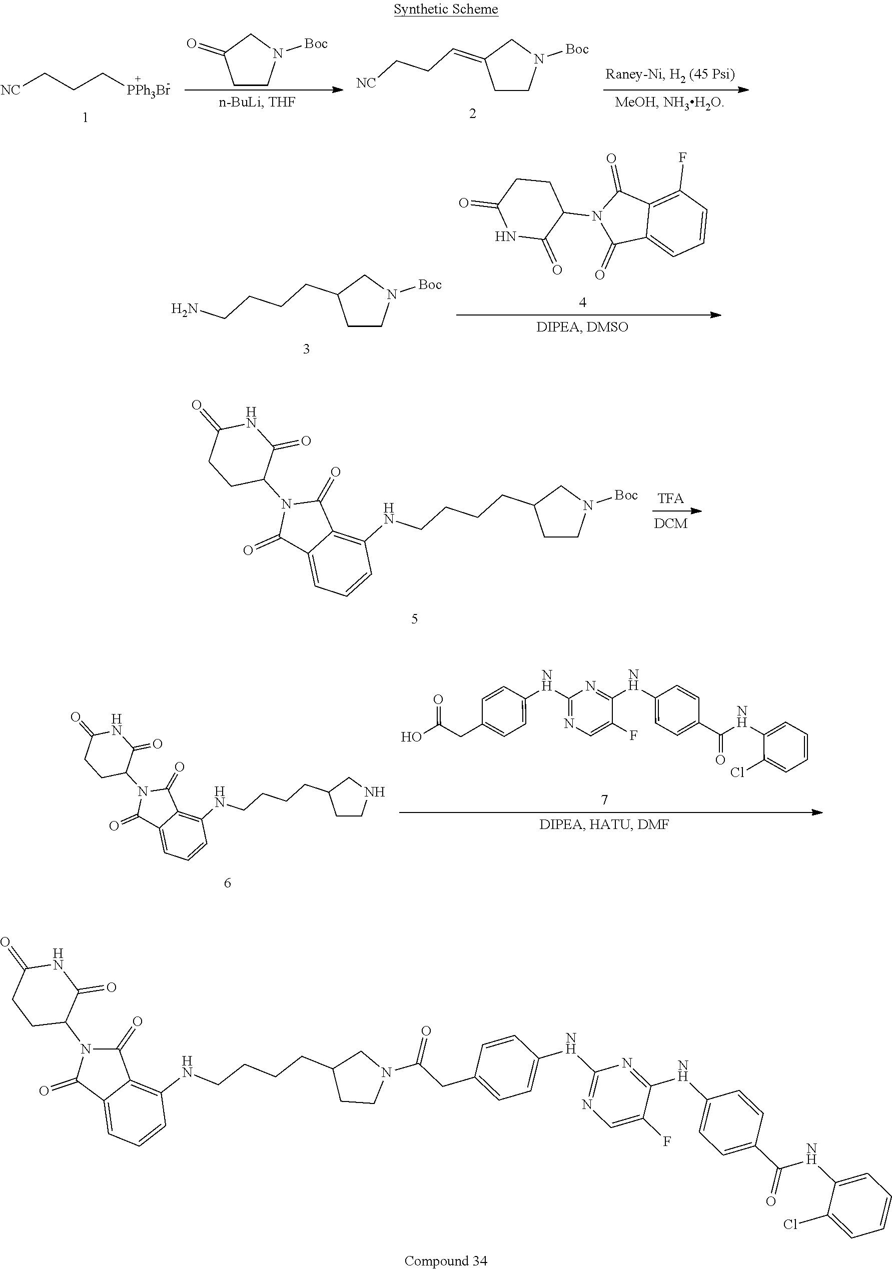

### Experimental Process

### Step 1: Synthesis of tert-butyl (E)-3-(3-cyanopropylidene)pyrrolidine-1-carboxylate (2)

A solution of (3-cyanopropyl)triphenylphosphonium bromide (3 g, 7.31 mmol) in THE (50 mL) in a 100 mL three-necked round bottom flask was prepared under N₂ atmosphere. The solution was added dropwise with n-BuLi (2.5 M, 3.22 mL) at 20° C. via an injection syringe and then stirred for 1 hour. After lowering the temperature to 0° C., tert-butyl 3-oxopyrrolidine-1-carboxylate (2.71 g, 14.62 mmol, 2 equivalents) was added. The mixture was stirred at 70° C. for 3 hours under N₂ atmosphere. Thereafter, the mixture was stirred at 20° C. for 14 hours. In TLC (petroleum ether:EtOAc=1:1, R_{f}=0.6), it was confirmed that a new spot was formed. The mixture was poured with a NH₄Cl (50 mL) solution and extracted with EtOAc (20 mL×3). The combined organic phase was washed with brine (20 mL) and then dried over Na₂SO₄ and concentrated under reduced pressure. The residue was purified by flash silica gel chromatography (ISCO®; 25 g SepaFlash® Silica Flash Column, Eluent of 0˜20% EtOAc/Petroleum ether gradient @ 100 mL/min) to obtain tert-butyl (E)-3-(3-cyanopropylidene)pyrrolidine-1-carboxylate (160 mg, 677.08 μmol, 9.26% yield) as yellow solid. MS(M+H)⁺=237.3.

### Step 2: Synthesis of tert-butyl 3-(4-aminobutyl)pyrrolidine-1-carboxylate (3)

Raney-Ni (50 mg) was added to a solution of tert-butyl (E)-3-(3-cyanopropylidene)pyrrolidine-1-carboxylate (160 mg, 677.08 μcool) in MeOH (10 mL) and NH₃·H₂O (1 mL) under N₂ atmosphere. The mixture was purged and degassed three times under H₂ atmosphere and then stirred at 20° C. for 16 hours under H₂ atmosphere (45 Psi). In TLC (petroleum ether:EtOAc=1:1, R_{f}=0), it was confirmed that a starting material was completely consumed. The mixture was filtered and then washed with MeOH (30 mL). The filtrate was dried with Na₂SO₄ and concentrated under reduced pressure to obtain tert-butyl 3-(4-aminobutyl)pyrrolidine-1-carboxylate (160 mg, crude) as yellow solid. MS(M+H)⁺=243.4.

### Step 3: Synthesis of tert-butyl 3-(4-((2-(2,6-dioxopiperidin-3 yl)-1,3-dioxoisoindolin-4-yl)amino)butyl)pyrrolidin-1-carboxylate (5)

A solution of tert-butyl 3-(4-aminobutyl)pyrrolidine-1-carboxylate (160 mg, 660.18 μmol), 2-(2,6-dioxo-3-piperidyl)-4-fluoro-isoindoline-1,3-dione (164.12 mg, 594.17 μmol) and DIPEA (170.65 mg, 1.32 mmol) in DMSO (5 mL) was stirred at 100° C. for 14 hours. The desired mass was detected by LCMS. The mixture was poured with water (20 mL) and extracted with EtOAc (10 mL×3). The combined organic phase was washed with brine (10 mL×2), dried over Na₂SO₄, and concentrated under reduced pressure to obtain tert-butyl 3-(4-((2-(2,6-dioxopiperidin-3-yl)-1,3-dioxoisoindolin-4-yl)amino)butyl)pyrrolidin-1-carboxylate (250 mg, crude) as brown solid, which was immediately used in the next reaction. MS(M+Na)⁺=521.3.

### Step 4: Synthesis of 2-(2,6-dioxopiperidin-3 yl)-4-((4-(pyrrolidin-3-yl)butyl)amino)isoindoline-1,3-dione (6)

TFA (1.54 g, 13.51 mmol, 1 mL) was added to a solution of tert-butyl 3-(4-((2-(2,6-dioxopiperidin-3-yl)-1,3-dioxoisoindolin-4-yl)amino)butyl)pyrrolidin-1-carboxylate (250 mg, 501.43 μmol) in DCM (4 mL) at 20° C. and then stirred for 1 hour. It was confirmed through LCMS that the starting material was completely consumed, and the desired mass was detected. The mixture was concentrated under reduced pressure to obtain 2-(2,6-dioxopiperidin-3-yl)-4-((4-(pyrrolidin-3-yl)butyl)amino)isoindoline-1,3-dione (250 mg, crude, TFA) as black oil. MS(M+H)⁺=399.1.

### Step 5: Synthesis of N-(2-chlorophenyl)-4-((2-((4-(2-(3-(4-((2-(2,6-dioxopiperidin-3 yl))-1,3-dioxoisoindolin-4-yl)amino)butyl)pyrrolidin-1 yl)-2-oxoethyl)phenyl)amino)-5-fluoropyrimidin-4-yl)amino)benzamide (Compound 34)

2-(2,6-dioxopiperidin-3-yl)-4-((4-(pyrrolidin-3-yl)butyl)amino)isoindoline-1,3-dione (250 mg, 487.83 μmol, TFA) was added to a solution of 2-(4-((4-((4-((2-chlorophenyl)carbamoyl)phenyl)amino)-5-fluoropyrimidin-2-yl)amino)phenyl)acetic acid (200 mg, 406.59 μmol), DIPEA (262.74 mg, 2.03 mmol) and HATU (185.52 mg, 487.90 μmol) in DMF (3 mL) at 20° C. and then stirred for 1 hour. It was confirmed through LCMS that the starting material was completely consumed, and the desired mass was detected. The mixture was poured with water (20 mL) and extracted with EtOAc (10 mL×3). The combined organic phase was washed with brine (10 mL), dried over Na₂SO₄, concentrated under reduced pressure, and then purified by prep-TLC (SiO₂, DCM/MeOH=10:1, R_{f}=0.6). The product was repurified by prep-HPLC (column: Unisil 3-100 C18 μLtra 150*50 mm*3 um; mobile phase: [water (FA)-ACN]; B %: 48%-78%, 7 min) and freeze-dried to obtain Compound 34 (6.8 mg, 7.02 μmol, 1.73% yield, 90% purity) as yellow solid. MS(M+H)⁺=872.4.

¹H NMR (400 MHz, DMSO-d₆) δ=11.09 (s, 1H), 9.89 (s, 1H), 9.65 (br s, 1H), 9.27 (s, 1H), 8.17 (t, J=3.4 Hz, 1H), 8.05-8.00 (m, 2H), 7.99-7.94 (m, 2H), 7.64 (br d, J=7.7 Hz, 1H), 7.60-7.53 (m, 4H), 7.39 (t, J=7.7 Hz, 1H), 7.32-7.26 (m, 1H), 7.14-7.04 (m, 3H), 7.01 (dd, J=1.7, 6.8 Hz, 1H), 6.58-6.48 (m, 1H), 5.07-5.01 (m, 1H), 3.69-3.58 (m, 1H), 3.57-3.48 (m, 3H), 3.20-2.94 (m, 2H), 2.93-2.72 (m, 2H), 2.63-2.55 (m, 3H), 2.20-1.89 (m, 4H), 1.58-1.50 (m, 2H), 1.40-1.27 (m, 4H).

### Example 35: Synthesis of Compound 35

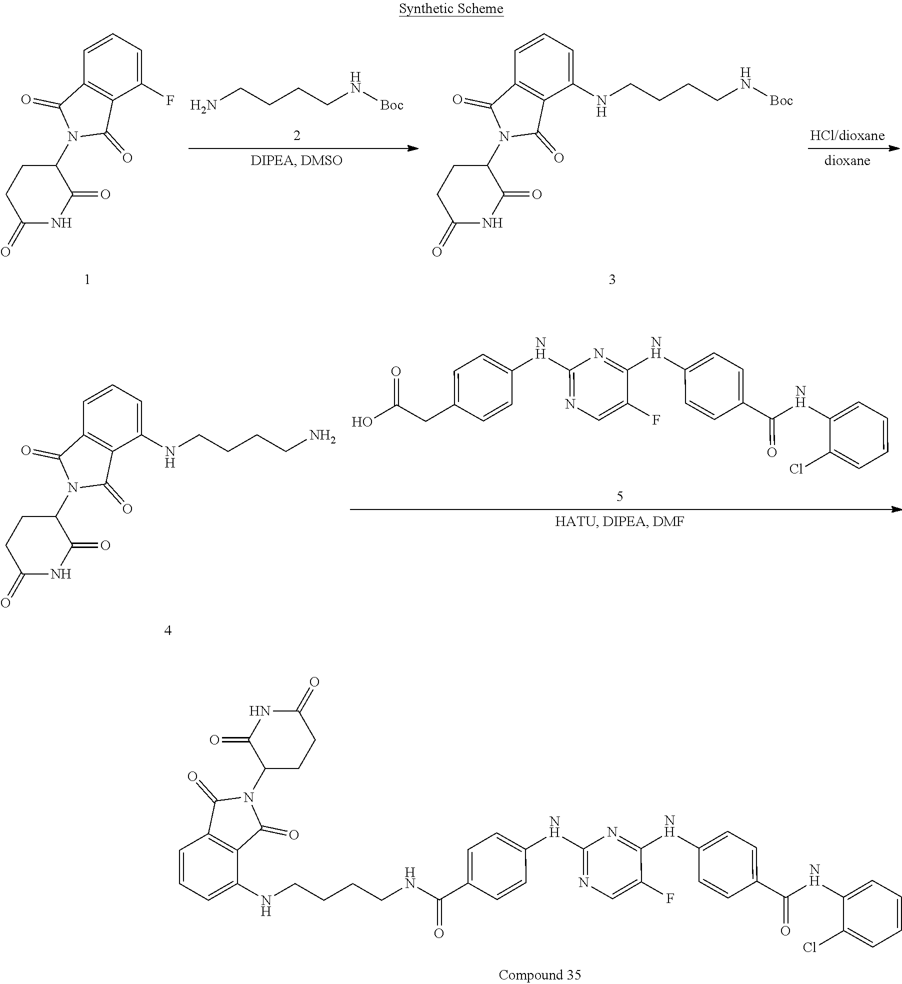

### Experimental Process

### Step 1: Synthesis of tert-butyl (4-((2-(2,6-dioxopiperidin-3 yl)-1,3-dioxoisoindolin-4-yl)amino)butyl)carbamate (3)

DIPEA (467.90 mg, 3.62 mmol, 630.59 μL) and tert-butyl(4-aminobutyl)carbamate (340.79 mg, 1.81 mmol) were added to a solution of 2-(2,6-dioxopiperidin-3-yl)-4-fluoroisoindoline-1,3-dione (0.5 g, 1.81 mmol) in DMSO (3 mL) and then stirred at 100° C. for 12 hours. The desired mass was detected by LCMS. The mixture was diluted with water (20 mL) and then extracted with EtOAc (20 mL×3). The combined organic phase was washed with saturated brine (20 mL), dried over Na₂SO₄, and filtered and concentrated under reduced pressure. The residue was purified by flash silica gel chromatography (ISCO®; 12 g SepaFlash® Silica Flash Column, Eluent of 0˜40% petroleum ether:EtOAc gradient @ 60 mL/min) to obtain tert-butyl (4-((2-(2,6-dioxopiperidin-3-yl)-1,3-dioxoisoindolin-4-yl)amino)butyl)carbamate (0.6 g, 1.12 mmol, 61.90% yield, 83% purity) as yellow oil. MS(M+Na)⁺=467.1.

### Step 2: Synthesis of 4-((4-aminobutyl)amino)-2-(2,6-dioxopiperidin-3-yl)isoindoline-1,3-dione (4)

HCl/dioxane (4 M, 10 mL) was added to a solution of tert-butyl (4-((2-(2,6-dioxopiperidin-3-yl)-1,3-dioxoisoindolin-4-yl)amino)butyl)carbamate (0.6 g, 1.35 mmol) in dioxane (5 mL) and then stirred at 25° C. for 2 hours. In TLC (petroleum ether:EtOAc=10:1), it was confirmed that a starting material was completely consumed. The mixture was filtered and the solid portion was dried to obtain 4-((4-aminobutyl)amino)-2-(2,6-dioxopiperidin-3-yl)isoindoline-1,3-dione (340 mg, 892.80 μmol, 66.14% yield, 100% purity, HCl) as yellow solid. MS(M+H)⁺=345.2.

### Step 3: Synthesis of N-(2-chlorophenyl)-4-((2-((4-((4-((2-(2,6-dioxopiperidin-3 yl)-1,3-dioxoisoindoline-4-yl)amino)butyl)carbamoyl)phenyl)amino)-5-fluoropyrimidin-4-yl)amino)benzamide (Compound 35)

DIPEA (121.70 mg, 941.67 μmol, 164.02 μL) and HATU (131.29 mg, 345.28 μmol) were added to a solution of 4-((4-((4-((2-chlorophenyl)carbamoyl)phenyl)amino)-5-fluoropyrimidin-2-yl)amino)benzoic acid (0.15 g, 313.89 μmol) in DMF (2 mL). After 30 minutes, 4-((4-Aminobutyl)amino)-2-(2,6-dioxopiperidin-3-yl)isoindoline-1,3-dione (95.63 mg, 251.11 μmol) was added and then stirred at 25° C. for 2 hours. The main peak of desired mass was detected by LCMS. The reactant was diluted with water (20 mL) and then extracted with EtOAc (10 mL×3). The combined organic phase was washed with saturated brine (10 mL), dried over Na₂SO₄, and filtered and concentrated under reduced pressure. The residue was purified by prep-HPLC (column: Phenomenex C18 75×30 mm×3 μm; mobile phase: [water (FA)-ACN]; B %: 42%-72%, 7 min) and freeze-dried to obtain Compound 35 (68.6 mg, 79.24 μmol, 25.25% yield, 92.9% purity) as yellow solid. MS(M+H)⁺=804.5.

¹H NMR (400 MHz, DMSO-d₆) δ=11.09 (s, 1H), 9.94 (br s, 1H), 9.75 (br s, 1H), 9.62 (br s, 1H), 8.32-8.21 (m, 2H), 8.06-7.98 (m, 4H), 7.77 (br s, 4H), 7.65 (d, J=7.1 Hz, 1H), 7.58-7.53 (m, 2H), 7.43-7.35 (m, 1H), 7.29 (t, J=7.5 Hz, 1H), 7.13-7.06 (m, 1H), 7.01 (d, J=7.2 Hz, 1H), 6.57 (d, J=5.4 Hz, 1H), 5.08-5.00 (m, 1H), 3.34-3.27 (m, 4H), 2.89-2.84 (m, 1H), 2.60-2.57 (m, 2H), 2.05-1.98 (m, 1H), 1.62-1.57 (m, 4H).

### Example 36: Synthesis of Compound 36

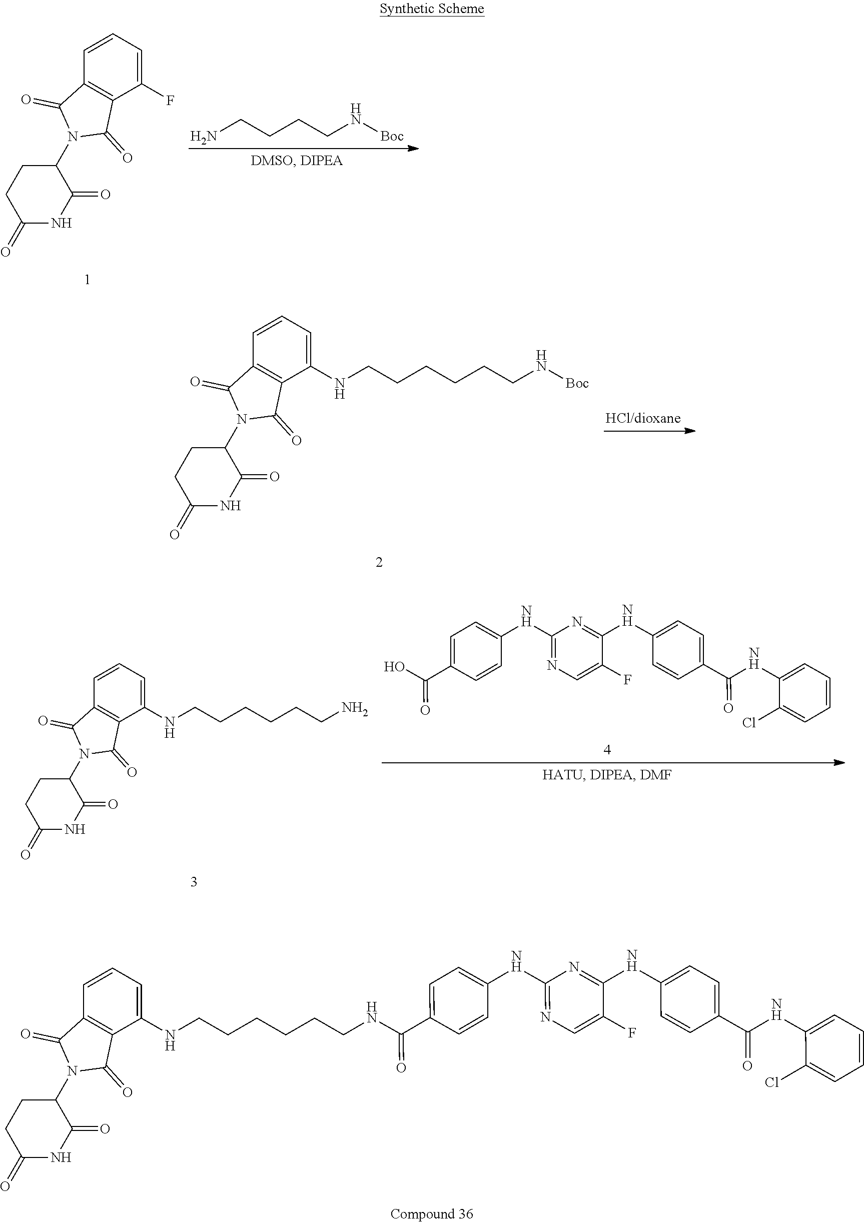 [SMILES: O=C1CCC(N2C(=O)c3cccc(F)c3C2=O)C(=O)N1 ; CC(C)(C)OC(=O)NCCCCN ; CC(C)(C)OC(=O)NCCCCCCNc1cccc2c1C(=O)N(C1CCC(=O)NC1=O)C2=O ; NCCCCCCNc1cccc2c1C(=O)N(C1CCC(=O)NC1=O)C2=O ; O=C(O)c1ccc(Nc2ncc(F)c(Nc3ccc(C(=O)Nc4ccccc4Cl)cc3)n2)cc1 ; O=C1CCC(N2C(=O)c3cccc(NCCCCCCNC(=O)c4ccc(Nc5ncc(F)c(Nc6ccc(C(=O)Nc7ccccc7Cl)cc6)n5)cc4)c3C2=O)C(=O)N1]

### Experimental Process

### Step 1: Synthesis of tert-butyl (6-((2-(2,6-dioxopiperidin-3-yl)-1,3-dioxoisoindolin-4-yl) amino) hexyl)carbamate (2)

DIPEA (350.92 mg, 2.72 mmol, 472.94 μL) and tert-butyl(6-aminohexyl)carbamate (430.73 mg, 1.99 mmol) were added to a solution of 2-(2,6-dioxopiperidin-3-yl)-4-fluoroisoindoline-1,3-dione (0.5 g, 1.81 mmol) in DMSO (5 mL) and then stirred at 100° C. for 12 hours. The main peak of desired mass was detected by LCMS. The mixture was diluted with water (30 mL) and then extracted with EtOAc (20 mL×3). The combined organic phase was washed with saturated brine (10 mL), dried over Na₂SO₄, and filtered and concentrated under reduced pressure. The residue was purified by flash silica gel chromatography (ISCO®; 20 g SepaFlash® Silica Flash Column, Eluent of 0˜40% petroleum ether:EtOAc gradient @ 80 mL/min) to obtain tert-butyl (6-((2-(2,6-dioxopiperidin-3-yl)-1,3-dioxoisoindolin-4-yl) amino)hexyl)carbamate (0.8 g, 1.37 mmol, 75.76% yield, 81% purity) as yellow oil. MS(M+Na)⁺=495.3.

### Step 2: Synthesis of 4-((6-aminohexyl)amino)-2-(2,6-dioxopiperidin-3-yl)isoindoline-1,3-dione (3)

HCl/dioxane (4 M, 10 mL) was added to a solution of tert-butyl (6-((2-(2,6-dioxopiperidin-3-yl)-1,3-dioxoisoindolin-4-yl)amino)hexyl)carbamate (0.8 g, 1.69 mmol) in dioxane (5 mL) and then stirred at 25° C. for 2 hours. In TLC (petroleum ether EtOAc=1:2), it was confirmed that a starting material was completely consumed. The mixture was concentrated under reduced pressure to obtain 4-((6-aminohexyl)amino)-2-(2,6-dioxopiperidin-3-yl)isoindoline-1,3-dione (0.63 g, crude, HCl) as yellow solid. MS(M+H)⁺=373.1.

### Step 3: Synthesis of N-(2-chlorophenyl)-4-((2-((4-((6-((2-(2,6-dioxopiperidin-3 yl)-1,3-dioxoisoindoline-4-yl)amino)hexyl)carbamoyl)phenyl)amino)-5-fluoropyrimidin-4-yl)amino)benzamide (Compound 36)

HATU (95.48 mg, 251.11 μmol) and DIPEA (81.14 mg, 627.78 μmol, 109.35 μL) were added to a solution of 4-((4-((4-((2-chlorophenyl)carbamoyl)phenyl)amino)-5-fluoropyrimidin-2-yl)amino)benzoic acid (0.1 g, 209.26 μmol) in DMF (3 mL). After 30 minutes, 4-((6-aminohexyl)amino)-2-(2,6-dioxopiperidin-3-yl)isoindoline-1,3-dione (77.01 mg, 188.33 μmol, HCl) was added and then stirred at 25° C. for 2 hours. The desired mass was detected by LCMS. The mixture was diluted with water (20 mL) and then extracted with EtOAc (20 mL×3). The combined organic phase was washed with saturated brine (20 mL), dried over Na₂SO₄, and filtered and concentrated under reduced pressure. The residue was purified by prep-HPLC (column: Phenomenex luna C18 150×25 mm×10 μm; mobile phase: [water (FA)-ACN]; B %: 48%-78%, 10 min) and then freeze-dried to obtain Compound 36 (32.5 mg, 36.12 μmol, 17.26% yield, 92.5% purity) as yellow solid. MS(M+H)⁺=832.2.

¹H NMR (400 MHz, DMSO-d₆) δ=11.10 (s, 1H), 9.94 (s, 1H), 9.76 (s, 1H), 9.62 (s, 1H), 8.28-8.20 (m, 2H), 8.06-7.99 (m, 4H), 7.79-7.75 (m, 4H), 7.65 (dd, J=1.5, 7.8 Hz, 1H), 7.60-7.54 (m, 2H), 7.41-7.37 (m, 1H), 7.31-7.26 (m, 1H), 7.08 (d, J=8.6 Hz, 1H), 7.01 (d, J=7.0 Hz, 1H), 6.58-6.51 (m, 1H), 5.05 (dd, J=5.4, 13.0 Hz, 1H), 3.30-3.20 (m, 4H), 2.93-2.84 (m, 1H), 2.62-2.57 (m, 2H), 2.05-1.99 (m, 1H), 1.60-1.49 (m, 4H), 1.39-1.32 (m, 4H).

### Example 37: Synthesis of Compound 37

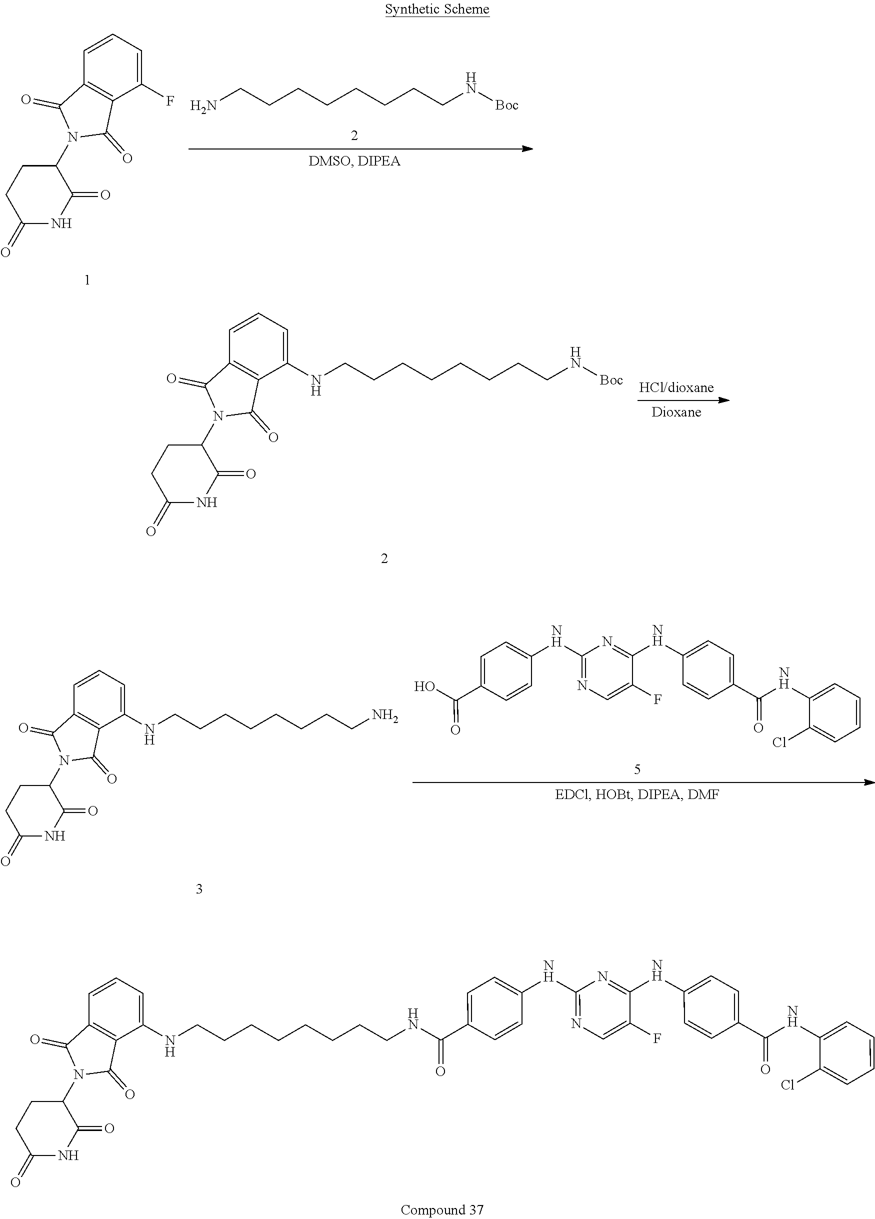

### Experimental Process

### Step 1: Synthesis of tert-butyl (8-((2-(2,6-dioxopiperidin-3 yl)-1,3-dioxoisoindolin-4-yl)amino)octyl)carbamate (2)

DIPEA (350.92 mg, 2.72 mmol, 472.94 μL) and tert-butyl (8-aminooctyl)carbamate (486.59 mg, 1.99 mmol) were added to a solution of 2-(2,6-dioxopiperidin-3-yl)-4-fluoroisoindoline-1,3-dione (0.5 g, 1.81 mmol) in DMSO (5 mL) and then stirred at 100° C. for 12 hours. The main peak of desired mass was detected by LCMS. The mixture was diluted with water (20 mL) and then extracted with EtOAc (10 mL×3). The combined organic phase was washed with saturated brine (10 mL), dried over anhydrous Na₂SO₄, and filtered and concentrated under reduced pressure. The residue was purified by flash silica gel chromatography (ISCO®; 20 g SepaFlash® Silica Flash Column, Eluent of 0˜40% petroleum ether:EtOAc gradient @ 80 mL/min) to obtain tert-butyl (8-((2-(2,6-dioxopiperidin-3-yl)-1,3-dioxoisoindolin-4-yl)amino)octyl)carbamate (900 mg, 1.52 mmol, 83.93% yield, 84.5% purity) as yellow oil. MS(M+Na)⁺=523.3.

### Step 2: Synthesis of 4-((8-aminooctyl)amino)-2-(2,6-dioxopiperidin-3-yl)isoindoline-1,3-dione (3)

HCl/dioxane (4 M, 10 mL) was added to a solution of tert-butyl (8-((2-(2,6-dioxopiperidin-3-yl)-1,3-dioxoisoindolin-4-yl)amino)octyl)carbamate (0.9 g, 1.80 mmol) in dioxane (5 mL) and then stirred at 25° C. for 2 hours. In TLC (petroleum ether:EtOAc=1:2), it was confirmed that a starting material was completely consumed. The mixture was concentrated under reduced pressure to obtain 4-((8-aminooctyl)amino)-2-(2,6-dioxopiperidin-3-yl)isoindoline-1,3-dione (1 g, crude, HCl) as yellow oil. MS(M+H)⁺=401.2.

### Step 3: Synthesis of N-(2-chlorophenyl)-4-((2-((4-((8-((2-(2,6-dioxopiperidin-3 yl)-1,3-dioxoisoindoline-4-yl)amino)octyl)carbamoyl)phenyl)amino)-5-fluoropyrimidin-4-yl)amino)benzamide (Compound 37)

EDCI (71.80 mg, 374.56 μmol), HOBt (50.61 mg, 374.56 μmol) and DIPEA (96.82 mg, 749.12 μmol, 130.48 μL) were added to a solution of 4-((8-aminooctyl)amino)-2-(2,6-dioxopiperidin-3-yl)isoindoline-1,3-dione (100 mg, 228.87 μmol, HCl) and 4-((4-((4)-((2-chlorophenyl)carbamoyl)phenyl)amino)-5-fluoropyrimidin-2-yl)amino)benzoic acid (119.33 mg, 249.71 μmol) in DMF (2 mL) and then stirred at 20° C. for 12 hours. The main peak of desired mass was detected by LCMS. The mixture was filtered through a Celite pad, and the filtrate was purified by prep-HPLC (column: Phenomenex luna C18 150*25 mm*10 um; mobile phase: [water (FA)-ACN]; B %: 57%-87%, 10 min) and freeze-dried to obtain Compound 37 (66.7 mg, 72.10 μmol, 28.87% yield, 93% purity) as yellow solid. MS(M+H)⁺=860.3.

¹H NMR (400 MHz, DMSO-d₆) δ=11.09 (s, 1H), 9.92 (s, 1H), 9.73 (s, 1H), 9.59 (s, 1H), 8.28-8.17 (m, 2H), 8.07-7.96 (m, 4H), 7.80-7.73 (m, 4H), 7.68-7.63 (m, 1H), 7.60-7.52 (m, 2H), 7.42-7.36 (m, 1H), 7.32-7.25 (m, 1H), 7.07 (d, J=8.5 Hz, 1H), 7.01 (d, J=7.0 Hz, 1H), 6.51 (t, J=5.6 Hz, 1H), 5.11-4.98 (m, 1H), 3.29-3.19 (m, 4H), 2.95-2.81 (m, 1H), 2.62-2.55 (m, 2H), 2.06-1.96 (m, 1H), 1.59-1.47 (m, 4H), 1.35-1.28 (m, 8H).

### Example 38: Synthesis of Compound 38

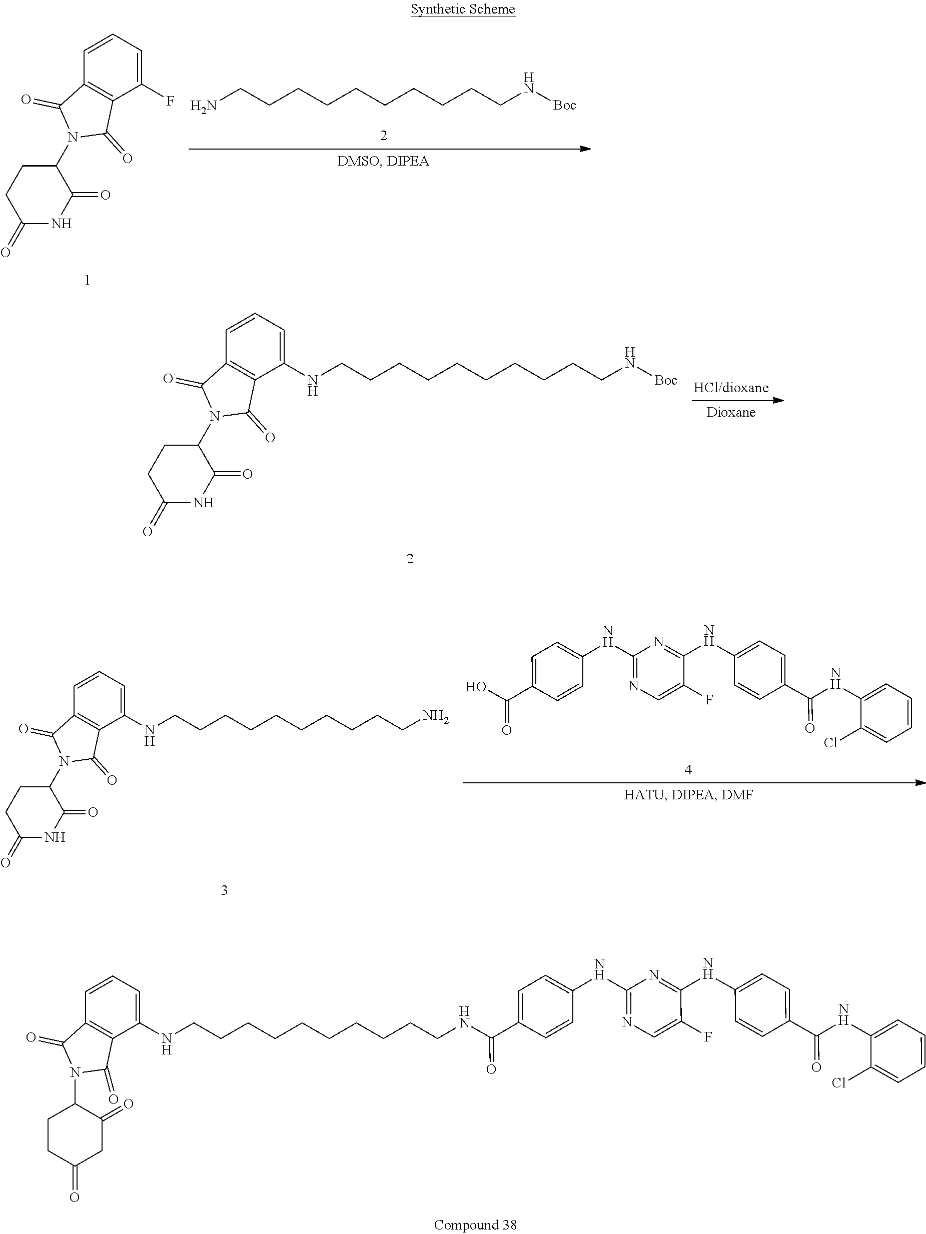

### Experimental Process

### Step 1: Synthesis of tert-butyl (10-((2-(2,6-dioxopiperidin-3 yl)-1,3-dioxoisoindolin-4-yl)amino)decyl)carbamate (2)

DIPEA (350.92 mg, 2.72 mmol, 472.94 μL) and tert-butyl(10-aminodecyl)carbamate (542.45 mg, 1.99 mmol) were added to a solution of 2-(2,6-dioxopiperidin-3-yl)-4-fluoroisoindoline-1,3-dione (0.5 g, 1.81 mmol) in DMSO (5 mL) and then stirred at 100° C. for 12 hours. 64% of the desired mass was detected by LCMS. The mixture was diluted with water (20 mL) and then extracted with EtOAc (30 mL×3). The combined organic phase was washed with saturated brine (50 mL), dried over Na₂SO₄, and filtered and concentrated under reduced pressure. The residue was purified by flash silica gel chromatography (ISCO®; 20 g SepaFlash® Silica Flash Column, Eluent of 0˜40% petroleum ether:EtOAc gradient @ 80 mL/min) to obtain tert-butyl (10-((2-(2,6-dioxopiperidin-3-yl)-1,3-dioxoisoindolin-4-yl)amino)decyl)carbamate (0.8 g, 1.24 mmol, 68.55% yield, 82% purity) as yellow oil. MS(M+Na)⁺=551.3.

### Step 2: Synthesis of 4-((10-aminodecyl)amino)-2-(2,6-dioxopiperidin-3-yl)isoindoline-1,3-dione (3)

HCl/dioxane (4 M, 10 mL) was added to a solution of tert-butyl (10-((2-(2,6-dioxopiperidin-3-yl)-1,3-dioxoisoindolin-4-yl)amino)decyl)carbamate (0.8 g, 1.51 mmol) in dioxane (5 mL) and then stirred at 25° C. for 2 hours. In TLC (petroleum ether EtOAc=1:2), it was confirmed that a starting material was completely consumed. The mixture was concentrated under reduced pressure to obtain 4-((10-aminodecyl)amino)-2-(2,6-dioxopiperidin-3-yl)isoindoline-1,3-dione (1.1 g, crude, HCl) as yellow oil. MS(M+H)⁺=429.3.

### Step 3: Synthesis of N-(2-chlorophenyl)-4-((2-((4-((10-((2-(2,6-dioxopiperidin-3 yl)-1,3-dioxoisoindoline-4-yl)amino)decyl)carbamoyl)phenyl)amino)-5-fluoropyrimidin-4-yl)amino)benzamide (Compound 38)

HATU (95.48 mg, 251.11 μmol) and DIPEA (81.14 mg, 627.78 μmol, 109.35 μL) were added to a solution of 4-((4-((4-((2-chlorophenyl)carbamoyl)phenyl)amino)-5-fluoropyrimidin-2-yl)amino)benzoic acid (0.1 g, 209.26 μmol) in DMF (3 mL). After 30 minutes, 4-((10-aminodecyl)amino)-2-(2,6-dioxopiperidin-3-yl)isoindoline-1,3-dione (87.57 mg, 188.33 μmol, HCl) was added and then stirred at 25° C. for 2 hours. 54% of the desired mass was detected by LCMS. The mixture was diluted with water (20 mL) and then extracted with EtOAc (20 mL×3). The combined organic phase was washed with saturated brine (20 mL), dried over anhydrous Na₂SO₄, and filtered and concentrated under reduced pressure. The residue was purified by prep-HPLC (column: Phenomenex Luna C18 150×25 mm×10 μm; mobile phase: [water (FA)-ACN]; B %: 60%-90%, 10 min) and freeze-dried to obtain Compound 38 (40.3 mg, 40.19 μmol, 19.21% yield, 90.7% purity) as yellow solid. MS(M+H)⁺=888.3.

¹H NMR (400 MHz, DMSO-d₆) δ=11.10 (s, 1H), 9.93 (s, 1H), 9.74 (s, 1H), 9.61 (s, 1H), 8.28-8.19 (m, 2H), 8.08-7.99 (m, 4H), 7.80-7.72 (m, 4H), 7.68-7.62 (m, 1H), 7.61-7.53 (m, 2H), 7.43-7.36 (m, 1H), 7.31-7.25 (m, 1H), 7.07 (d, J=8.7 Hz, 1H), 7.01 (d, J=7.1 Hz, 1H), 6.55-6.49 (m, 1H), 5.05 (dd, J=5.2, 12.9 Hz, 1H), 3.28-3.18 (m, 4H), 2.91-2.85 (m, 1H), 2.59-2.57 (m, 2H), 2.06-1.98 (m, 1H), 1.57-1.46 (m, 4H), 1.34-1.24 (m, 12H).

### Comparative Example 1: Alisertib-Based PROTAC

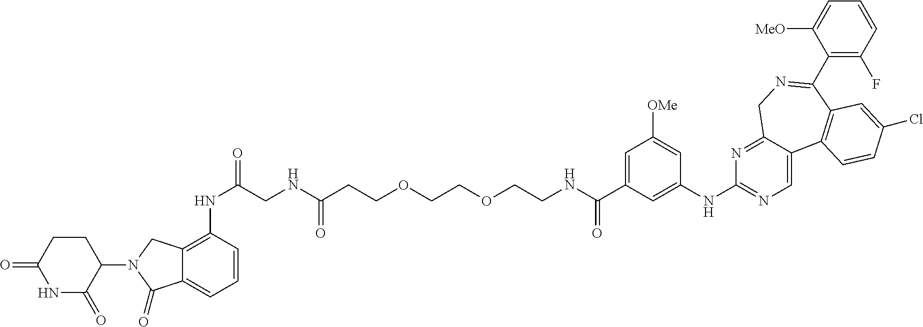 [SMILES: COc1cc(Nc2ncc3c(n2)CN=C(c2c(F)cccc2OC)c2cc(Cl)ccc2-3)cc(C(=O)NCCOCCOCCC(=O)NCC(=O)Nc2cccc3c2CN(C2CCC(=O)NC2=O)C3=O)c1]

### Comparative Example 2: Alisertib-Based PROTAC

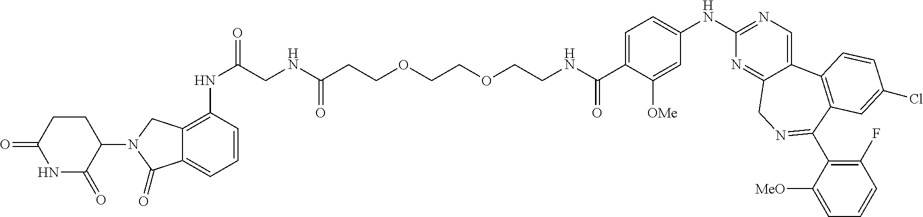 [SMILES: COc1cc(Nc2ncc3c(n2)CN=C(c2c(F)cccc2OC)c2cc(Cl)ccc2-3)ccc1C(=O)NCCOCCOCCC(=O)NCC(=O)Nc1cccc2c1CN(C1CCC(=O)NC1=O)C2=O]

### Comparative Example 3: Alisertib-Based PROTAC

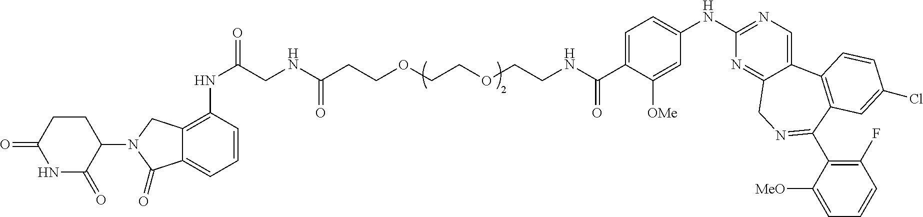

### Experimental Example

### 1. Culture of HeLa Cell Line

An MDA-MB-231 cell line was purchased from the Korea Cell Line Bank. The passage of cultured cells was maintained within P50.

For cell counting, a cell counter (Catalog #AMQAX1000) from Thermo and a 0.4% Trypan blue solution were used.

For cell culture, there were used RPMI (Gibco, Cat. No. 22400-089; Lot. No. 2323633), FBS (Gibco, Cat. No. 16000044; Lot. No. 2454168P), Penicillin/Streptomycin (PS) (Gibco, Cat. No. 15140-122; Lot. No. 2321150), 100 mm culture plate (SPL, Cat. No. 20100), 6-well culture plate (SPL, Cat. No. 30006), PBS pH7.4 (Gibco, Cat. No. 10010-023; Lot. No. 2380329), counting chamber (Hematocytometer) (Hirschmann, Cat. No. 8100204), and 0.4% trypan blue solution (DYNEBIO, Cat. No. CBT3710; Lot. No. 20211201).

2. Treatment with Compound of the Present Invention

3×10⁵ cells were seeded in each well of a 12-well plate (SPL), and the cells were cultured in a total culture medium volume of 1 mL.

A compound was completely dissolved in DMSO (Sigma, Cat. No. D2438; Lot. No. RNBK1809) and used in the experiment, and the concentration of DMSO treated in each well was unified at 0.1%, and the compound was treated to be added into the cells. The concentration treated in each well was as indicated in a Blot image.

Finally, after 24 hours of the compound treatment, the cells were washed with 0.5 ml of PBS pH7.4 (Gibco, Cat. No. 10010-023; Lot. No. 2380329), and then treated with 500 μl of TrypLE Express (Gibco, Cat. No. 12605-010; Lot. No. 2193192) to be separated. The separated cells were neutralized by adding 5 ml of RPMI (Gibco, Cat. No. 22400-089; Lot. No. 2323633). The cells and the medium were separated using a centrifuge, and then only the cells were stored and used for a Western blot experiment later.

### 3. Western Blotting

For SDS-PAGE and western blotting, there were used RIPA lysis buffer (ROCKLAND, Cat. no. MB-030-0050; Lot. no. 46352), 100× protease inhibitor cocktail (Quartett, Cat. No. PPI1015; Lot no. PC050039472), PierceTM BCA protein assay kit(ThermoScientific, Cat. No. 23225; Lot no. UJ290659), Albumin standard (ThermoScientific, Cat. No. 23209; Lot no. UB269561), 4-15% Mini-PROTEAN TGX stain-free gel (Bio-rad, Cat. No. 4561086; Lot no. 64454521), IOX Tris/Glycine/SDS buffer (Bio-rad, Cat. No. 1610772; Lot no. 64442350); Tris/Glycine buffer (Bio-rad, Cat. No. 1610771; Lot no. 64442352), IOX TBS (Bio-rad, Cat. No. 1706435; Lot no. 64446921), 10% Tween 20 solution (Cat. No. 1610781; Lot no. 64399445), Color protein standard broad range (Biolabs, Cat. P7719S; Lot no. 10118359), 4× Laemmli sample buffer (Bio-rad, Cat. No. 1610747; Lot no. 64377840), Skim milk (BD, Cat. No. 232100; Lot no. 8346795), 1 M sodium azide solution (Sigma-Aldrich, Cat. No. 52002-500G; Lot no. MKCG9565), α-Rabbit pAb to Ms IgG (abcam, Cat. No. ab97046; Lot no. GR3329375-5), α-Goat pAb to Rb IgG (CST, Cat. no. 7074S; Lot no. 30), α-AURKA (Abcam, Cat. No. ab1287; Lot no. GR3338838-6), α-AURKB (Abcam, Cat. No. ab2254; Lot no. GR3396402-2), α-GAPDH (Abcam, Cat. No. ab8245; Lot no. GR3401390-1), ECL™ Prime western blotting reagents (Cytiva, Cat. No. RPN2232; Lot no. 17246828), Ponceau S solution (Sigma-Aldrich, Cat. No. P7170; Lot no. SLBV4112), DifcoTM Skim milk (BD, Cat. No. 232100; Lot no. 232100), Acrylamide gel tank (Bio-Rad, Cat. No. 1658039), and PowerPac HC (Bio-Rad, Cat. No. 043BR80483).

For cell lysis, the cell debris was removed by adding a lysis buffer to obtain a cell lysate. Specifically, the cells were treated with 35 μL of a 1×RIPA buffer containing a protease inhibitor and incubated on ice for 30 minutes. Thereafter, the cells were centrifuged at 4° C. and 15,000 rpm for 20 minutes to obtain a cell lysate.

Next, a standard curve was obtained using the BCA assay and the protein mass in the lysate was quantified by substituting the obtained standard curve. The mixture was incubated at 37° C. for 30 minutes using 20 μL of a standard or sample solution and 200 μL of a BCA or Bradford solution, and absorbance was measured at 562 nm. Samples were prepared by adding a 4× sample buffer to be 15 μg per well.

SDS-PAGE was performed on a 4-15% Mini-PROTEAN TGX stain-free gel (15 well) at 120 V by setting a running time of 100 minutes. The samples were transferred on a nitrocellulose membrane using a transfer buffer (prepared by mixing 10×Tris/Gycine buffer:MeOH:DDW at a ratio of 1:2:7). The samples were stained using a Ponceau S solution, and then blocked for 1 hour with skim milk (BD). The samples were washed with 1×TBS containing 0.05% Tween20 and then reacted with primary antibodies, such as anti-AURKA (Abcam) antibody (1:1000), anti-AURKB (Abcam) antibody (1:1000), and anti-GAPDH (Abcam) antibody (1:5,000) in 1×TBS-T at 4° C. for 16 hours. The samples were washed with 1×TBS containing 0.05% Tween20 three times for 10 minutes and then reacted with secondary antibodies, such as anti-mouse antibody (abcam) (1:5,000) and anti-rabbit antibody (CST) in 1×TBS-T at room temperature for 1 hour. Next, the samples were washed with 1×TBS containing 0.05% Tween 20 three times for 10 minutes, and then detected with an ECL working solution (1:1).

For result analysis, final blot data were obtained using an image analyzer (GE).

### 4. Confirmation of AURKA Degradation of Compound of the Present Invention

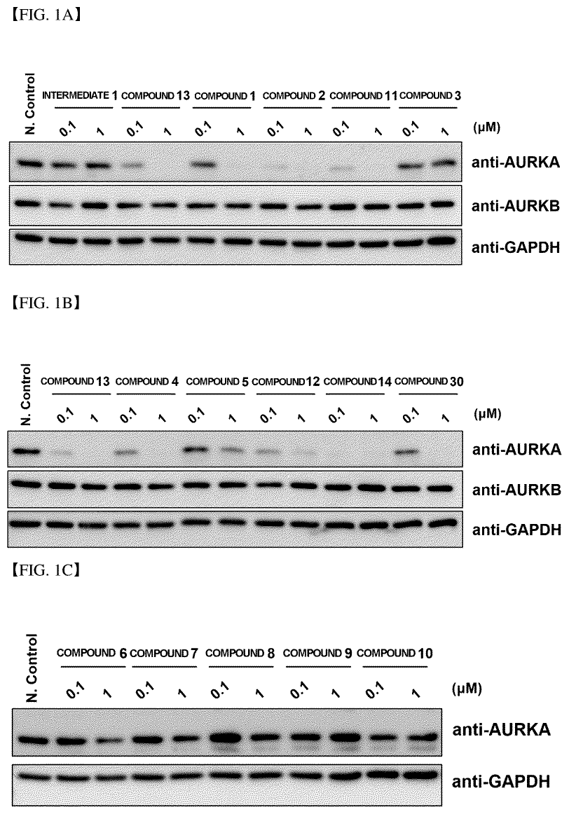

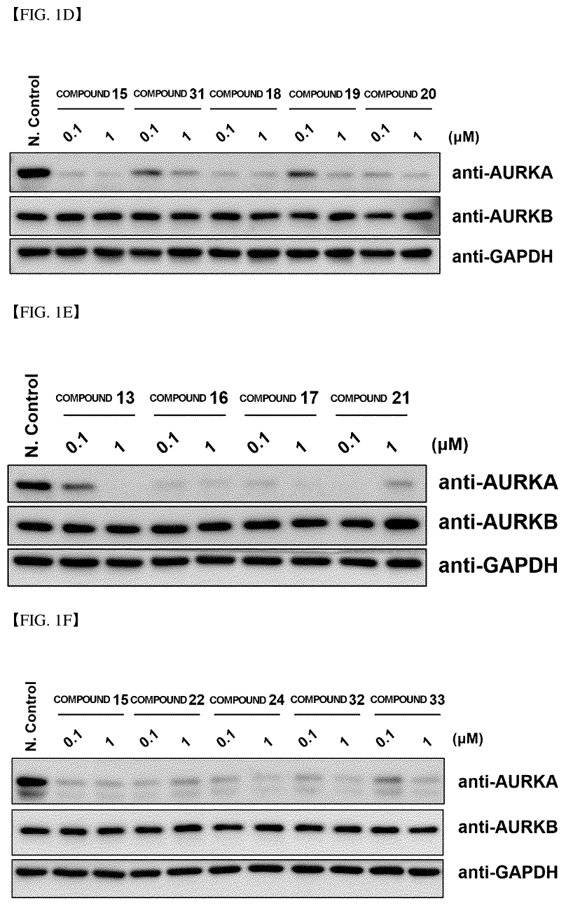

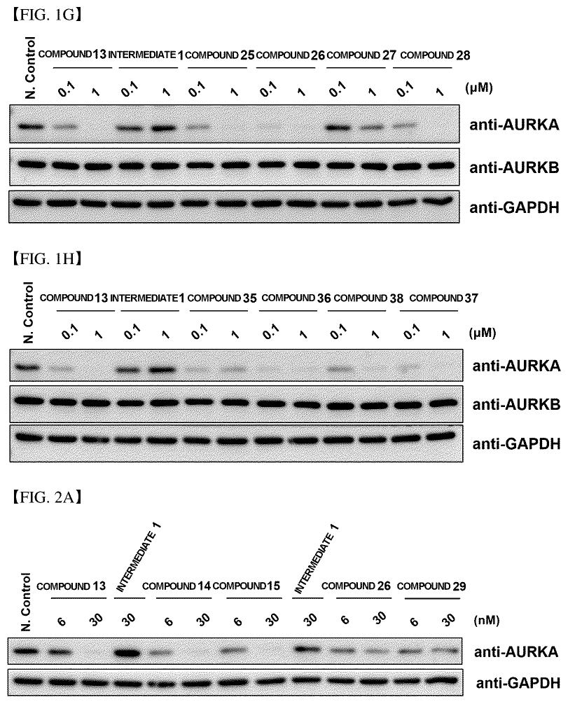

The above-described experiment results were shown in FIGS. 1 to 3.

FIGS. 1A to 1H are Western blot images showing AURKA degradation and AURKA selectivity when the concentrations of Example Compounds are 0.1 μM and 1 μM.

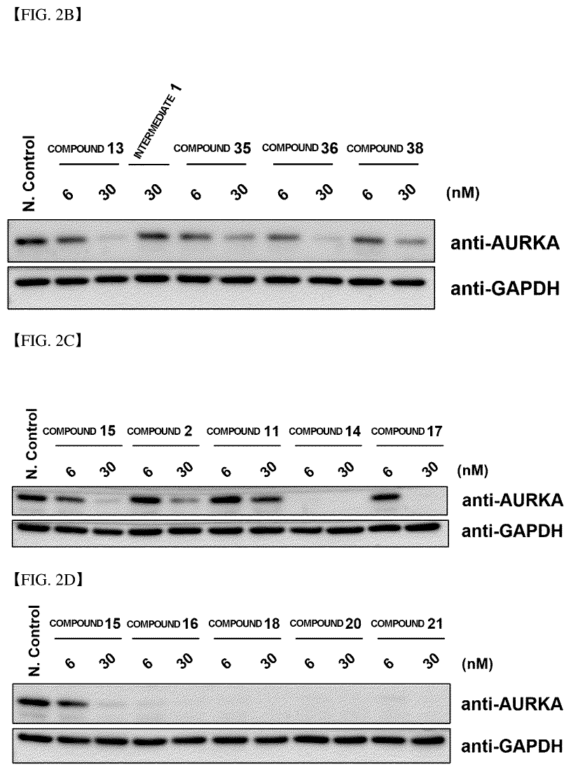

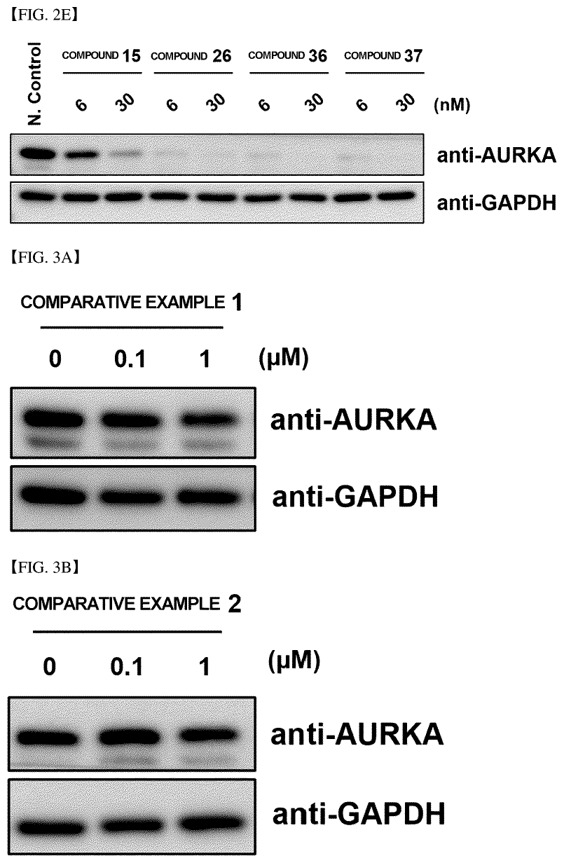

FIGS. 2A to 2E are Western blot images showing AURKA degradation when the concentrations of Example Compounds are low of 6 nM and 30 nM.

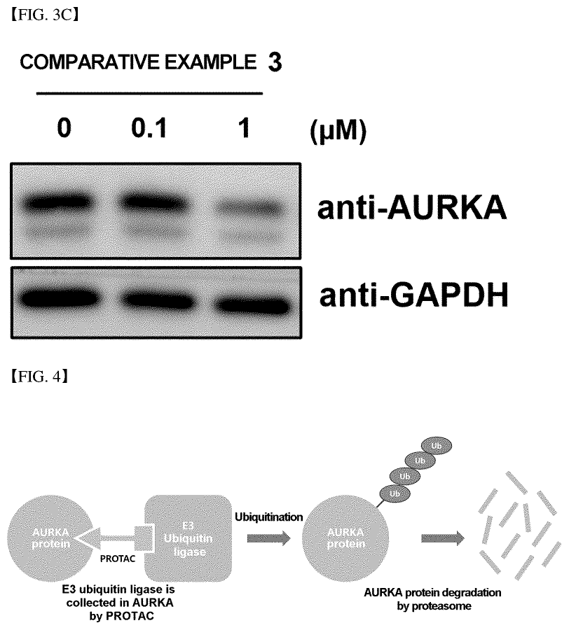

FIGS. 3A to 3C are Western blot images showing AURKA degradation when the concentrations of Alisertib-based PROTAC of Comparative Examples 1 to 3 are 0.1 μM and 1 μM.

Referring to FIGS. 1A to 1H, when the concentrations of Example Compounds are 0.1 μM, AURKA protein degradation activity of 80% or more may be confirmed, and in particular, it may be seen that Compound 14 shows excellent AURKA degradation of 95% or more. In addition, when the concentrations of Example Compounds are 1 μM, 90% or more of AURKA protein degradation activity may be confirmed.

In addition, referring to FIGS. 2A to 2E, it may be seen that 90% or more of AURKA protein degradation activity is shown even at low concentrations of 6 nM (0.006 μM) and 30 nM (0.03 μM).

On the other hand, referring to FIGS. 3A to 3C, it may be seen that the Alisertib-based PROTAC compounds of Comparative Examples 1 to 3 exhibit AURKA protein degradation activity of 30% or less.

Accordingly, it may be confirmed that Example Compounds of the present invention exhibit significantly superior AURKA protein degradation activity and AURKA degradation selectivity to conventional Alisertib-based PROTAC.

It will be appreciated by those skilled in the art that the present invention as described above may be implemented into other specific forms without departing from the technical spirit thereof or essential characteristics. Thus, it is to be appreciated that examples described above are intended to be illustrative in every sense, and not restrictive. The scope of the present invention is represented by claims to be described below rather than the detailed description, and it is to be interpreted that the meaning and scope of the claims and all the changes or modified forms derived from the equivalents thereof come within the scope of the present invention.

1. A compound represented by Chemical Formula I below, a stereoisomer thereof, or a pharmaceutically acceptable salt thereof:
ULM-Linker-PTM [Chemical Formula I]
In Chemical Formula I,
ULM is an E3 ubiquitin ligase binding moiety represented by Chemical Formula A or B below, 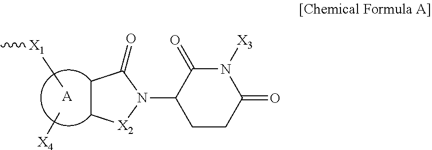
{in Chemical Formula A, [symbol]
is a ring selected from the group consisting of 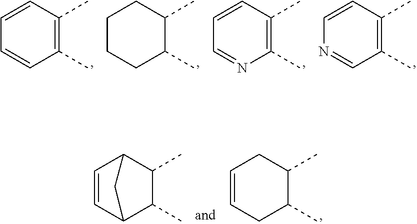
X₁ is a single bond, —CH₂—, —NH—, —O—, —CH₂CH₂—, —CC—, —CO—, —COO—, —NHCO— or —CONH—;
X₂ is —CH₂—, —CH(C₁₋₄alkyl)-, —NH—, —N(C₁₋₄alkyl)-, —O—, —CO—, —CH₂—CH₂—, —NH—CH₂—, —NH—CH(C₁₋₄alkyl)-, —N═CH—, —N═C(C₁₋₄alkyl)- or —N═N—,
X₃ is hydrogen; and
X₄ is hydrogen, halogen, C₁₋₆alkyl, CN, NH₂, NO₂, OH, COH, COOH or CF₃.} 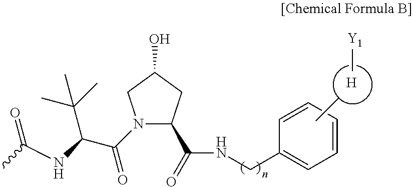
{in Chemical Formula B,
n is an integer of 1 to 3, [symbol]
is a 5- to 6-membered cycloalkyl, phenyl, 5- to 6-membered heterocycloalkyl, or 5- to 6-membered heteroaryl ring, and
Y₁ is hydrogen or C₁₋₃alkyl}
PTM is an AURKA binding moiety represented by Chemical Formula II below, 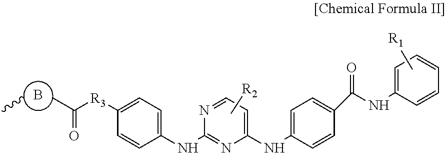
{in Chemical Formula II,
R₁ and R₂ are each independently hydrogen, —NO₂, —CN, —OH, C₁₋₆alkyl, C₂₋₆alkenyl, C₂₋₆alkynyl, C₁₋₆alkoxy, C₁₋₆thioalkoxy, C₃₋₆cycloalkyl, C₃₋₆heterocycloalkyl, C₃₋₆heteroaryl, phenyl or halogen,
R₃ is C₁₋₆alkylene, —O—, —S—, —NH—, or a direct bond, and [symbol]
is a 4- to 10-membered cycloalkyl, phenyl, 4- to 10-membered heterocycloalkyl, 5- to 6-membered heteroaryl ring or a direct bond.}
Linker is a group for chemically linking ULM and PTM, and is represented by Chemical Formula L below: 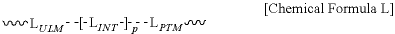
In Chemical Formula LF, [symbol] and [symbol] are bonds,
L_{ULM} binds to a ULM moiety through [symbol] linked thereto,
L_{PTM} binds to a PTM moiety through [symbol] linked thereto,
L_{ULM} and L_{PTM} are each independently single bond, —CH₂—, —NH—, —O—, —CO—, —OCO—, —CONH—, —NHCO—, —O(CH)ₙCONH— {wherein, n is an integer of 1 to 5}, or direct bond,
L_{INT} is selected from the group consisting of C₁₋₁₀ alkylene, —CH₂—, —NH—, —O(CH)ₙCONH-{where n is an integer of 1 to 5}, —(CH₂)ₗO(CH₂)ₘO(CH₂)_{q}— {wherein, l, m and q are independently integers of 1 to 5}, —CHCH—, —CC—, —CH₂CH₂O—, —OCH₂CH₂—, —CH₂CH₂S—, —SCH₂CH₂—, —COO—, —CONH—, —NHCO—, [symbol] and direct bond {wherein [symbol] is a ring selected from the group consisting of aryl, heteroaryl, 3 to 10 membered cycloalkyl, 4 to 10 membered heterocycloalkyl, 4 to 10 membered cycloalkenyl, and 4 to 10 membered heterocycloalkenyl},
L_{ULM}, L_{PTM} and Lm r may each independently be substituted with one or more of C₁₋₆alkyl, C₃₋₈cycloalkyl, C₃₋₈heterocycloalkyl, halogen, hydroxy, amine, nitro, cyano or C₁₋₈haloalkyl, and
p is an integer of 1 to 20.

2. The compound, the stereoisomer thereof, or the pharmaceutically acceptable salt thereof of claim 1, wherein ULM is an E3 ubiquitin ligase ligand represented by Chemical Formula A-1 below: 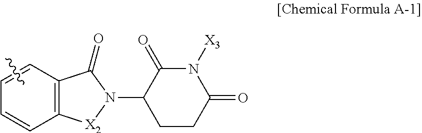
In Chemical Formula A-1,
X₂ is —CH₂—, —CH(C₁₋₄alkyl)-, —CO—, or —N═N—, and
X₃ is hydrogen.

3. The compound, the stereoisomer thereof, or the pharmaceutically acceptable salt thereof of claim 2, wherein Chemical Formula A-1 is selected from the group consisting of the following moieties:

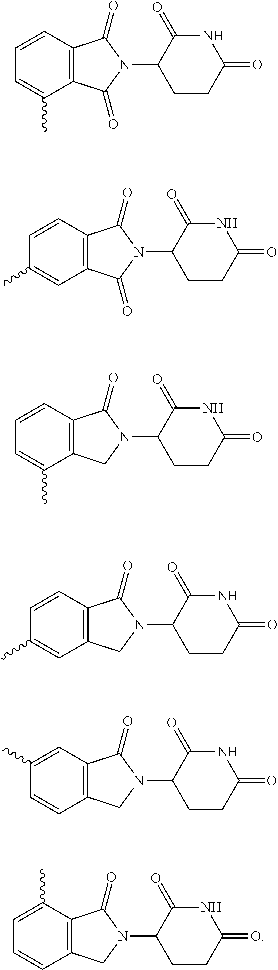

4. The compound, the stereoisomer thereof, or the pharmaceutically acceptable salt thereof of claim 1, wherein Chemical Formula B is selected from the group consisting of the following moieties:

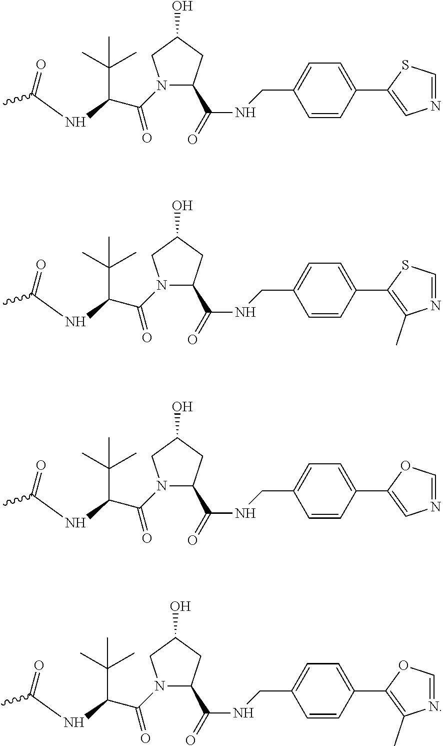

5. The compound, the stereoisomer thereof, or the pharmaceutically acceptable salt thereof of claim 1, wherein Chemical Formula II is selected from the group consisting of the following moieties: 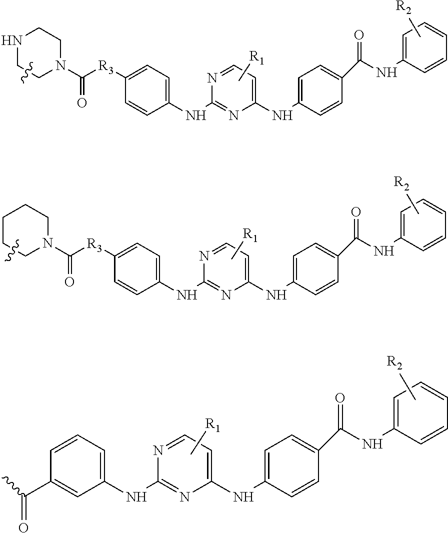
in the group consisting of the moieties,
R₁ and R₂ are each independently hydrogen, —NO₂, —CN, —OH, C₁₋₆alkyl, C₂₋₆alkenyl, C₂₋₆alkynyl, C₁₋₆alkoxy, C₁₋₆thioalkoxy, C₃₋₆cycloalkyl, C₃₋₆heterocycloalkyl, C₃₋₆heteroaryl, phenyl or halogen, and
R₃ is C₁₋₆alkylene, —O—, —S—, —NH—, or direct bond.

6. The compound, the stereoisomer thereof, or the pharmaceutically acceptable salt thereof of claim 5, wherein Chemical Formula II is selected from the group consisting of the following moieties:

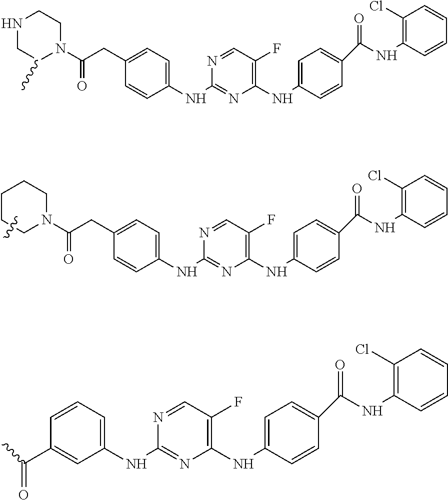

7. The compound, the stereoisomer thereof, or the pharmaceutically acceptable salt thereof of claim 1, wherein the compound, the stereoisomer thereof, or the pharmaceutically acceptable salt thereof is selected from the group consisting of compounds 1 to 38 represented by the following Table:

| Comp. | Structure |
|---|---|
| 1 | 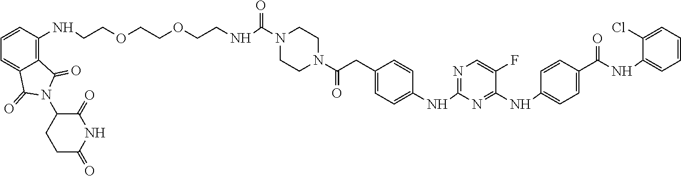 [SMILES: O=C1CCC(N2C(=O)c3cccc(NCCOCCOCCNC(=O)N4CCN(C(=O)Cc5ccc(Nc6ncc(F)c(Nc7ccc(C(=O)Nc8ccccc8Cl)cc7)n6)cc5)CC4)c3C2=O)C(=O)N1] |
| 2 | 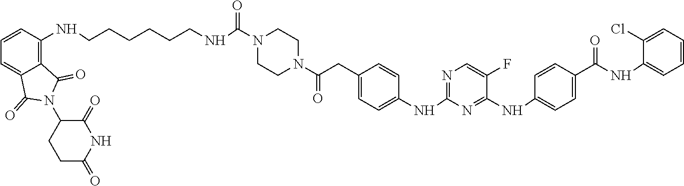 [SMILES: O=C1CCC(N2C(=O)c3cccc(NCCCCCCNC(=O)N4CCN(C(=O)Cc5ccc(Nc6ncc(F)c(Nc7ccc(C(=O)Nc8ccccc8Cl)cc7)n6)cc5)CC4)c3C2=O)C(=O)N1] |
| 3 | 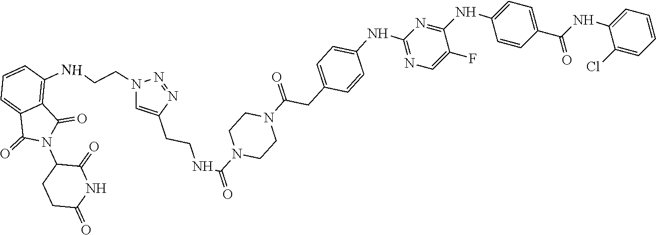 [SMILES: O=C1CCC(N2C(=O)c3cccc(NCCn4cc(CCNC(=O)N5CCN(C(=O)Cc6ccc(Nc7ncc(F)c(Nc8ccc(C(=O)Nc9ccccc9Cl)cc8)n7)cc6)CC5)nn4)c3C2=O)C(=O)N1] |
| 4 | 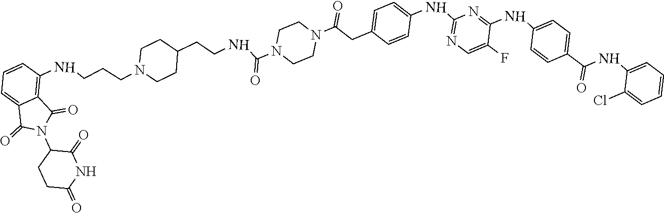 [SMILES: O=C1CCC(N2C(=O)c3cccc(NCCCN4CCC(CCNC(=O)N5CCN(C(=O)Cc6ccc(Nc7ncc(F)c(Nc8ccc(C(=O)Nc9ccccc9Cl)cc8)n7)cc6)CC5)CC4)c3C2=O)C(=O)N1] |
| 5 | 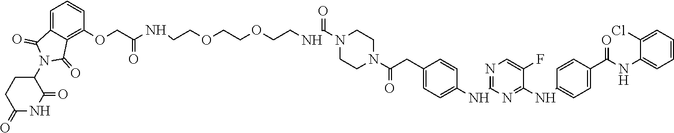 [SMILES: O=C(COc1cccc2c1C(=O)N(C1CCC(=O)NC1=O)C2=O)NCCOCCOCCNC(=O)N1CCN(C(=O)Cc2ccc(Nc3ncc(F)c(Nc4ccc(C(=O)Nc5ccccc5Cl)cc4)n3)cc2)CC1] |
| 6 | 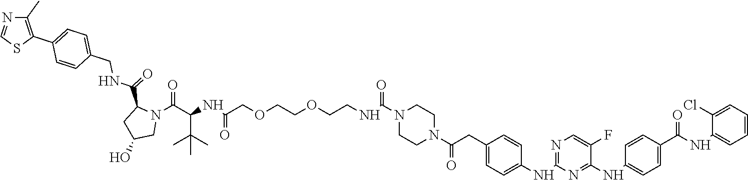 [SMILES: Cc1ncsc1-c1ccc(CNC(=O)[C@@H]2C[C@@H](O)CN2C(=O)[C@@H](NC(=O)COCCOCCNC(=O)N2CCN(C(=O)Cc3ccc(Nc4ncc(F)c(Nc5ccc(C(=O)Nc6ccccc6Cl)cc5)n4)cc3)CC2)C(C)(C)C)cc1] |
| 7 | 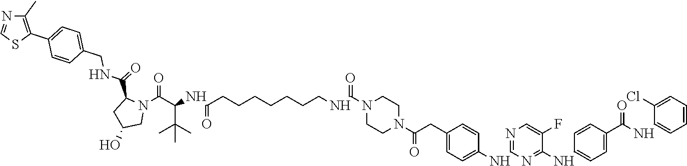 [SMILES: Cc1ncsc1-c1ccc(CNC(=O)[C@@H]2C[C@@H](O)CN2C(=O)[C@@H](NC(=O)CCCCCCCNC(=O)N2CCN(C(=O)Cc3ccc(Nc4ncc(F)c(Nc5ccc(C(=O)Nc6ccccc6Cl)cc5)n4)cc3)CC2)C(C)(C)C)cc1] |
| 8 | 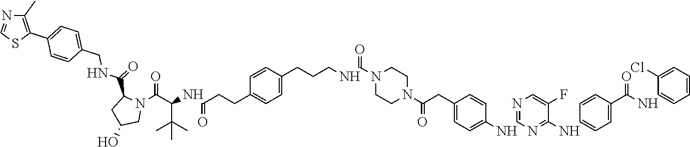 [SMILES: Cc1ncsc1-c1ccc(CNC(=O)[C@@H]2C[C@@H](O)CN2C(=O)[C@@H](NC(=O)CCc2ccc(CCCNC(=O)N3CCN(C(=O)Cc4ccc(Nc5ncc(F)c(Nc6ccc(C(=O)Nc7ccccc7Cl)cc6)n5)cc4)CC3)cc2)C(C)(C)C)cc1] |
| 9 | 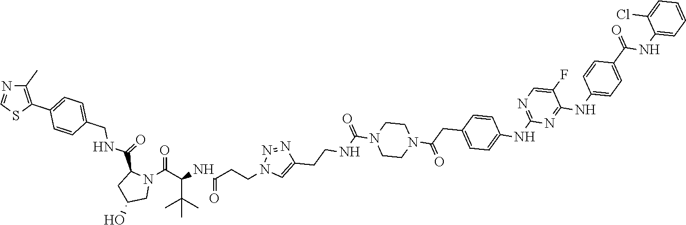 [SMILES: Cc1ncsc1-c1ccc(CNC(=O)[C@@H]2C[C@@H](O)CN2C(=O)[C@@H](NC(=O)CCn2cc(CCNC(=O)N3CCN(C(=O)Cc4ccc(Nc5ncc(F)c(Nc6ccc(C(=O)Nc7ccccc7Cl)cc6)n5)cc4)CC3)nn2)C(C)(C)C)cc1] |
| 10 | 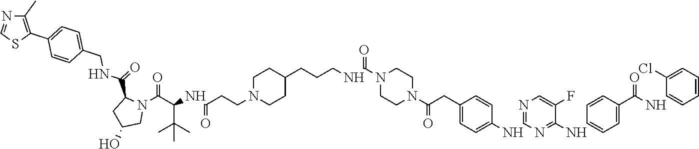 [SMILES: Cc1ncsc1-c1ccc(CNC(=O)[C@@H]2C[C@@H](O)CN2C(=O)[C@@H](NC(=O)CCN2CCC(CCCNC(=O)N3CCN(C(=O)Cc4ccc(Nc5ncc(F)c(Nc6ccc(C(=O)Nc7ccccc7Cl)cc6)n5)cc4)CC3)CC2)C(C)(C)C)cc1] |
| 11 | 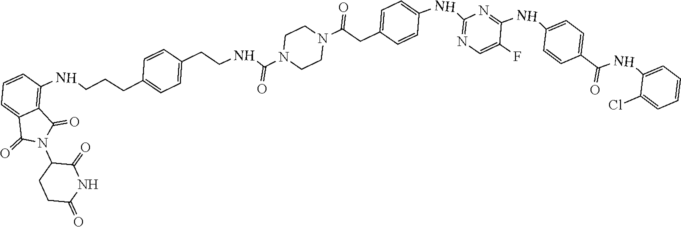 [SMILES: O=C1CCC(N2C(=O)c3cccc(NCCCc4ccc(CCNC(=O)N5CCN(C(=O)Cc6ccc(Nc7ncc(F)c(Nc8ccc(C(=O)Nc9ccccc9Cl)cc8)n7)cc6)CC5)cc4)c3C2=O)C(=O)N1] |
| 12 | 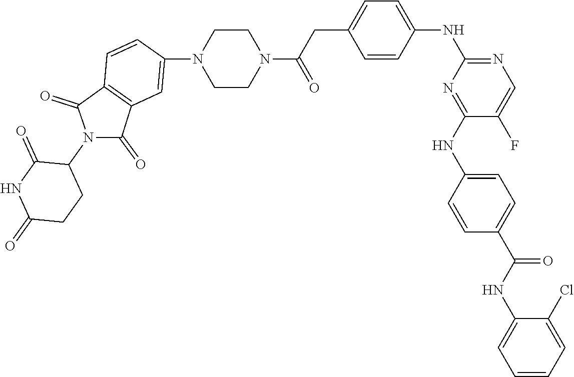 [SMILES: O=C1CCC(N2C(=O)c3ccc(N4CCN(C(=O)Cc5ccc(Nc6ncc(F)c(Nc7ccc(C(=O)Nc8ccccc8Cl)cc7)n6)cc5)CC4)cc3C2=O)C(=O)N1] |
| 13 | 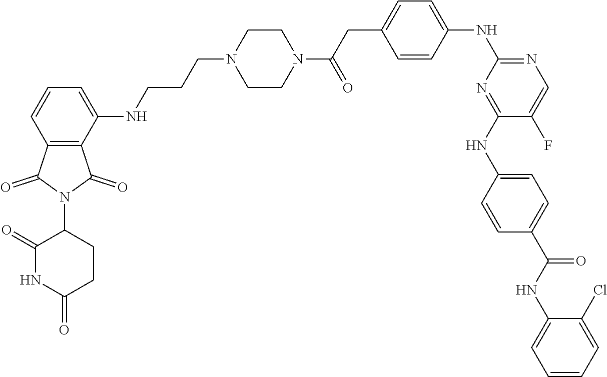 [SMILES: O=C1CCC(N2C(=O)c3cccc(NCCCN4CCN(C(=O)Cc5ccc(Nc6ncc(F)c(Nc7ccc(C(=O)Nc8ccccc8Cl)cc7)n6)cc5)CC4)c3C2=O)C(=O)N1] |
| 14 | 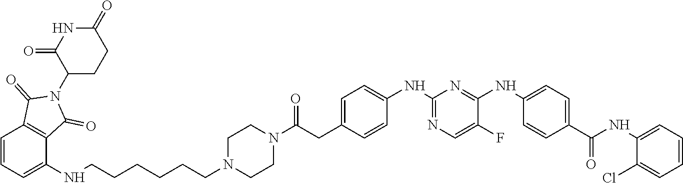 [SMILES: O=C1CCC(N2C(=O)c3cccc(NCCCCCCN4CCN(C(=O)Cc5ccc(Nc6ncc(F)c(Nc7ccc(C(=O)Nc8ccccc8Cl)cc7)n6)cc5)CC4)c3C2=O)C(=O)N1] |
| 15 | 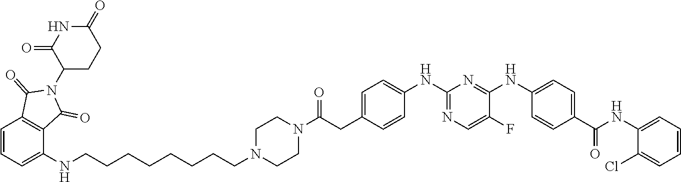 [SMILES: O=C1CCC(N2C(=O)c3cccc(NCCCCCCCCN4CCN(C(=O)Cc5ccc(Nc6ncc(F)c(Nc7ccc(C(=O)Nc8ccccc8Cl)cc7)n6)cc5)CC4)c3C2=O)C(=O)N1] |
| 16 | 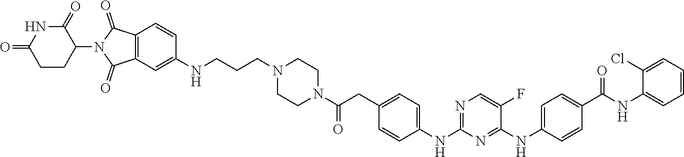 [SMILES: O=C1CCC(N2C(=O)c3ccc(NCCCN4CCN(C(=O)Cc5ccc(Nc6ncc(F)c(Nc7ccc(C(=O)Nc8ccccc8Cl)cc7)n6)cc5)CC4)cc3C2=O)C(=O)N1] |
| 17 | 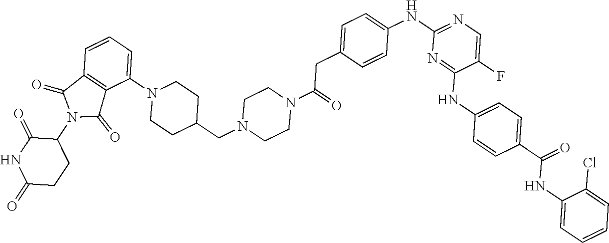 [SMILES: O=C1CCC(N2C(=O)c3cccc(N4CCC(CN5CCN(C(=O)Cc6ccc(Nc7ncc(F)c(Nc8ccc(C(=O)Nc9ccccc9Cl)cc8)n7)cc6)CC5)CC4)c3C2=O)C(=O)N1] |
| 18 | 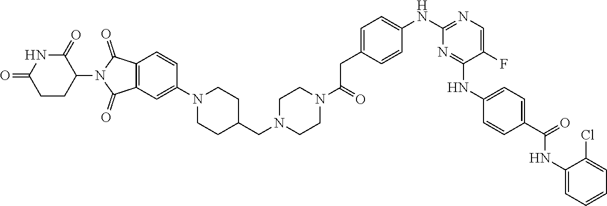 [SMILES: O=C1CCC(N2C(=O)c3ccc(N4CCC(CN5CCN(C(=O)Cc6ccc(Nc7ncc(F)c(Nc8ccc(C(=O)Nc9ccccc9Cl)cc8)n7)cc6)CC5)CC4)cc3C2=O)C(=O)N1] |
| 19 | 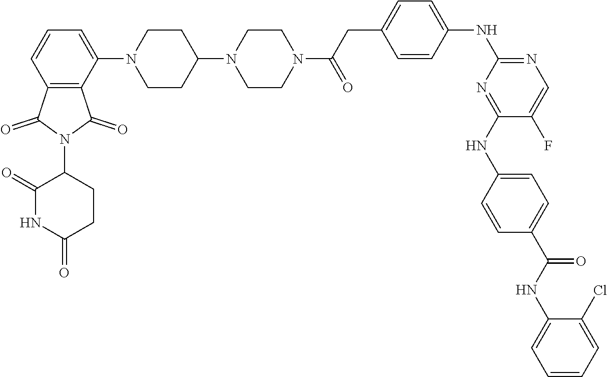 [SMILES: O=C1CCC(N2C(=O)c3cccc(N4CCC(N5CCN(C(=O)Cc6ccc(Nc7ncc(F)c(Nc8ccc(C(=O)Nc9ccccc9Cl)cc8)n7)cc6)CC5)CC4)c3C2=O)C(=O)N1] |
| 20 | 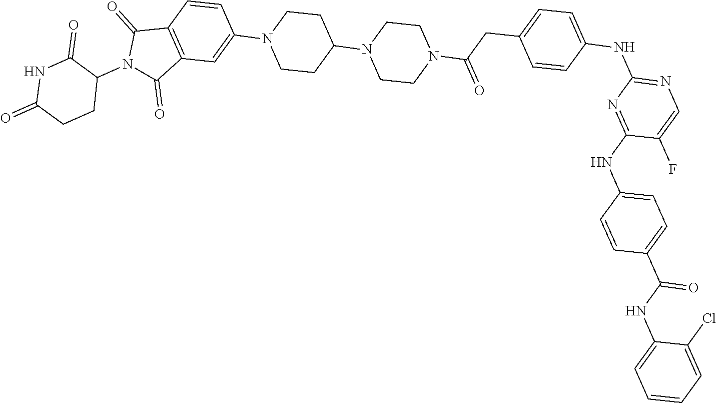 [SMILES: O=C1CCC(N2C(=O)c3ccc(N4CCC(N5CCN(C(=O)Cc6ccc(Nc7ncc(F)c(Nc8ccc(C(=O)Nc9ccccc9Cl)cc8)n7)cc6)CC5)CC4)cc3C2=O)C(=O)N1] |
| 21 | 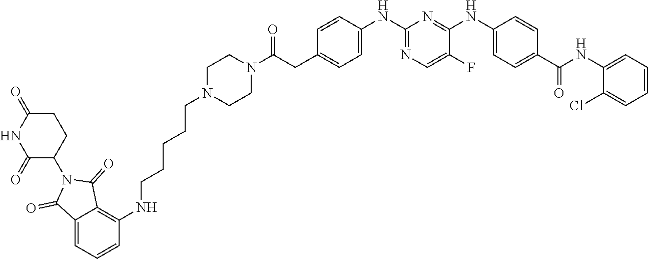 [SMILES: O=C1CCC(N2C(=O)c3cccc(NCCCCCN4CCN(C(=O)Cc5ccc(Nc6ncc(F)c(Nc7ccc(C(=O)Nc8ccccc8Cl)cc7)n6)cc5)CC4)c3C2=O)C(=O)N1] |
| 22 | 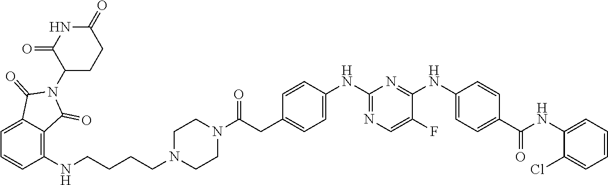 [SMILES: O=C1CCC(N2C(=O)c3cccc(NCCCCN4CCN(C(=O)Cc5ccc(Nc6ncc(F)c(Nc7ccc(C(=O)Nc8ccccc8Cl)cc7)n6)cc5)CC4)c3C2=O)C(=O)N1] |
| 23 | 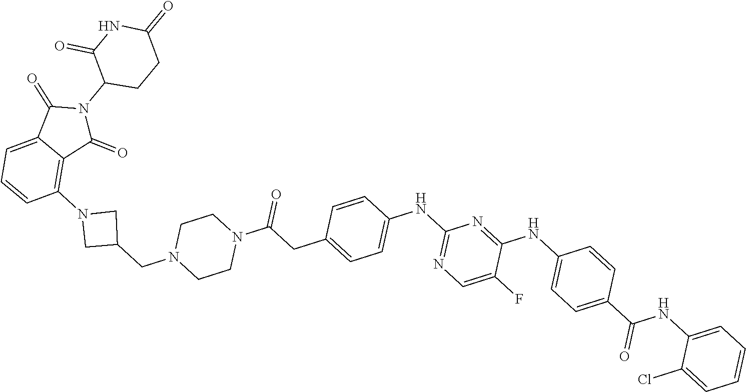 [SMILES: O=C1CCC(N2C(=O)c3cccc(N4CC(CN5CCN(C(=O)Cc6ccc(Nc7ncc(F)c(Nc8ccc(C(=O)Nc9ccccc9Cl)cc8)n7)cc6)CC5)C4)c3C2=O)C(=O)N1] |
| 24 | 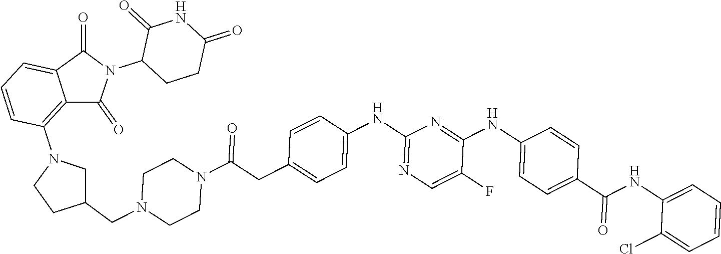 [SMILES: O=C1CCC(N2C(=O)c3cccc(N4CCC(CN5CCN(C(=O)Cc6ccc(Nc7ncc(F)c(Nc8ccc(C(=O)Nc9ccccc9Cl)cc8)n7)cc6)CC5)C4)c3C2=O)C(=O)N1] |
| 25 | 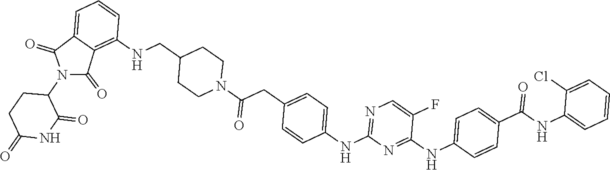 [SMILES: O=C1CCC(N2C(=O)c3cccc(NCC4CCN(C(=O)Cc5ccc(Nc6ncc(F)c(Nc7ccc(C(=O)Nc8ccccc8Cl)cc7)n6)cc5)CC4)c3C2=O)C(=O)N1] |
| 26 | 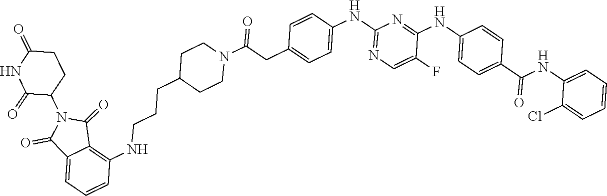 [SMILES: O=C1CCC(N2C(=O)c3cccc(NCCCC4CCN(C(=O)Cc5ccc(Nc6ncc(F)c(Nc7ccc(C(=O)Nc8ccccc8Cl)cc7)n6)cc5)CC4)c3C2=O)C(=O)N1] |
| 27 | 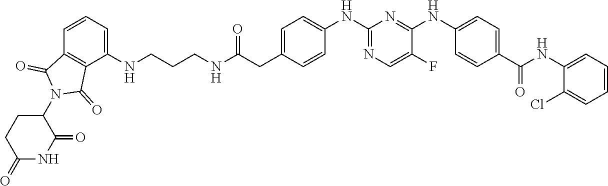 [SMILES: O=C(Cc1ccc(Nc2ncc(F)c(Nc3ccc(C(=O)Nc4ccccc4Cl)cc3)n2)cc1)NCCCNc1cccc2c1C(=O)N(C1CCC(=O)NC1=O)C2=O] |
| 28 | 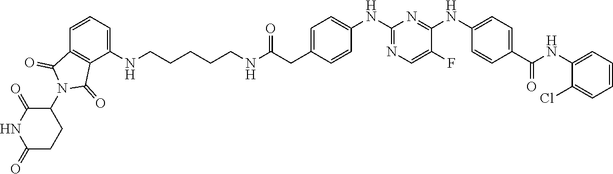 [SMILES: O=C(Cc1ccc(Nc2ncc(F)c(Nc3ccc(C(=O)Nc4ccccc4Cl)cc3)n2)cc1)NCCCCCNc1cccc2c1C(=O)N(C1CCC(=O)NC1=O)C2=O] |
| 29 | 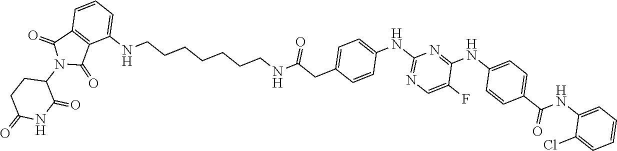 [SMILES: O=C(Cc1ccc(Nc2ncc(F)c(Nc3ccc(C(=O)Nc4ccccc4Cl)cc3)n2)cc1)NCCCCCCCNc1cccc2c1C(=O)N(C1CCC(=O)NC1=O)C2=O] |
| 30 | 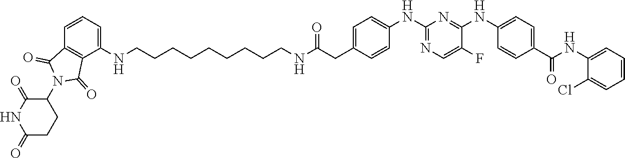 [SMILES: O=C(Cc1ccc(Nc2ncc(F)c(Nc3ccc(C(=O)Nc4ccccc4Cl)cc3)n2)cc1)NCCCCCCCCCNc1cccc2c1C(=O)N(C1CCC(=O)NC1=O)C2=O] |
| 31 | 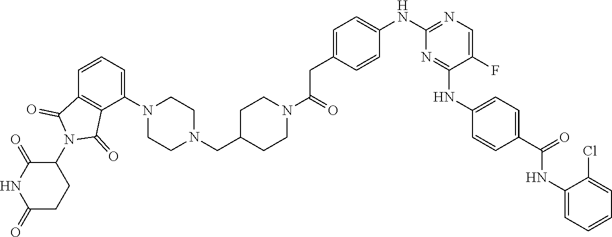 [SMILES: O=C1CCC(N2C(=O)c3cccc(N4CCN(CC5CCN(C(=O)Cc6ccc(Nc7ncc(F)c(Nc8ccc(C(=O)Nc9ccccc9Cl)cc8)n7)cc6)CC5)CC4)c3C2=O)C(=O)N1] |
| 32 | 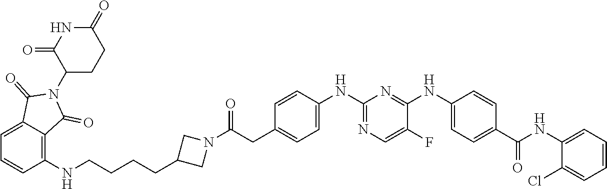 [SMILES: O=C1CCC(N2C(=O)c3cccc(NCCCCC4CN(C(=O)Cc5ccc(Nc6ncc(F)c(Nc7ccc(C(=O)Nc8ccccc8Cl)cc7)n6)cc5)C4)c3C2=O)C(=O)N1] |
| 33 | 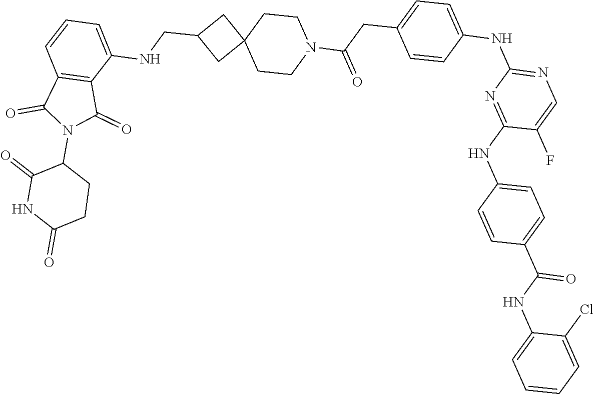 [SMILES: O=C1CCC(N2C(=O)c3cccc(NCC4CC5(CCN(C(=O)Cc6ccc(Nc7ncc(F)c(Nc8ccc(C(=O)Nc9ccccc9Cl)cc8)n7)cc6)CC5)C4)c3C2=O)C(=O)N1] |
| 34 | 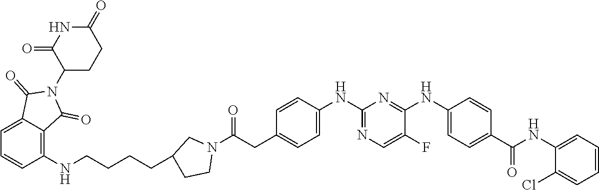 [SMILES: O=C1CCC(N2C(=O)c3cccc(NCCCCC4CCN(C(=O)Cc5ccc(Nc6ncc(F)c(Nc7ccc(C(=O)Nc8ccccc8Cl)cc7)n6)cc5)C4)c3C2=O)C(=O)N1] |
| 35 | 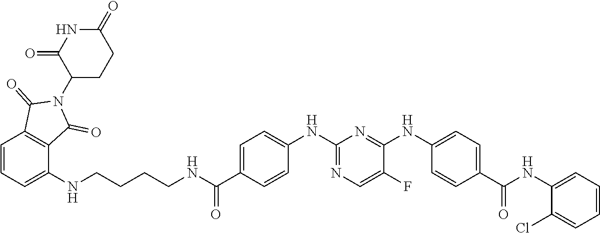 [SMILES: O=C1CCC(N2C(=O)c3cccc(NCCCCNC(=O)c4ccc(Nc5ncc(F)c(Nc6ccc(C(=O)Nc7ccccc7Cl)cc6)n5)cc4)c3C2=O)C(=O)N1] |
| 36 | 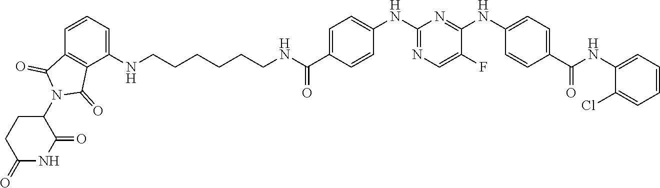 [SMILES: O=C1CCC(N2C(=O)c3cccc(NCCCCCCNC(=O)c4ccc(Nc5ncc(F)c(Nc6ccc(C(=O)Nc7ccccc7Cl)cc6)n5)cc4)c3C2=O)C(=O)N1] |
| 37 | 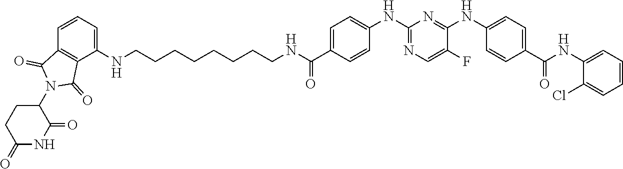 [SMILES: O=C1CCC(N2C(=O)c3cccc(NCCCCCCCCNC(=O)c4ccc(Nc5ncc(F)c(Nc6ccc(C(=O)Nc7ccccc7Cl)cc6)n5)cc4)c3C2=O)C(=O)N1] |
| 38 | 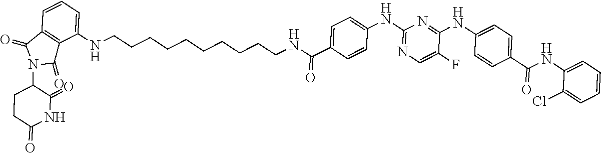 [SMILES: O=C1CCC(N2C(=O)c3cccc(NCCCCCCCCCCNC(=O)c4ccc(Nc5ncc(F)c(Nc6ccc(C(=O)Nc7ccccc7Cl)cc6)n5)cc4)c3C2=O)C(=O)N1] |

8. The compound, the stereoisomer thereof, or the pharmaceutically acceptable salt thereof of claim 1, wherein the compound represented by Chemical Formula I induces selective degradation of AURKA protein.

9. A pharmaceutical composition for preventing or treating cancer comprising the compound, the stereoisomer thereof, or the pharmaceutically acceptable salt thereof according to claim 1 as an active ingredient.

10. The pharmaceutical composition of claim 9, wherein the cancer is AURKA-related cancer selected from the group consisting of squamous cell carcinoma, small cell lung cancer, non-small cell lung cancer, adenocarcinoma of lung, squamous cell carcinoma of lung, peritoneal cancer, skin cancer, skin or intraocular melanoma, rectal cancer, anal cancer, esophageal cancer, small intestine cancer, endocrine cancer, parathyroid cancer, adrenal cancer, soft tissue sarcoma, urethral cancer, chronic or acute leukemia, lymphocytic lymphoma, myelofibrosis, hepatocellular cancer, stomach cancer, gastric cancer, pancreatic cancer, glioblastoma, cervical cancer, ovarian cancer, liver cancer, bladder cancer, liver tumor, breast cancer, colon cancer, colorectal cancer, endometrial or uterine cancer, salivary gland cancer, kidney cancer, prostate cancer, vulvar cancer, thyroid cancer, head and neck cancer, brain cancer, osteosarcoma, Barrett's esophagus, colonic adenoma and polyp, breast fibroadenoma and cyst, monoclonal gammopathy (MGUS), and monoclonal lymphocytosis.

11. A method for preventing or treating cancer comprising administering the compound, the stereoisomer thereof, or the pharmaceutically acceptable salt thereof according to claim 1 to a subject in need thereof.
